(19) United States
(12) Patent Application Publication
(10) Pub. No.: US 2015/0289512 A1
(43) Pub. Date: Oct. 15, 2015

(54) METHOD FOR PROMOTING PLANT GROWTH

(71) Applicant: SUMITOMO CHEMICAL COMPANY, LIMITED, Tokyo (JP)

(72) Inventors: Fujio Mukumoto, Takarazuka-shi (JP); Hiroaki Tamaki, Takarazuka-shi (JP); Shintaro Kusaka, Takarazuka-shi (JP); Mitsuhiko Iwakoshi, Takarazuka-shi (JP)

(73) Assignee: SUMITOMO CHEMICAL COMPANY, LIMITED, Tokyo (JP)

(21) Appl. No.: 14/441,574

(22) PCT Filed: Oct. 31, 2013
(86) PCT No.: PCT/JP2013/080173

§ 371 Date: May 8, 2015

(30) Foreign Application Priority Data
Nov. 9, 2012 (JP) 2012-247265

(51) Int. Cl. A01N 43/90; A01N 43/12; C07D 209/42; A01N 43/38; A01N 43/78; A01N 43/76; C07D 235/24; A01N 43/52; C07D 471/04; C07D 277/68; C07D 307/85; C07D 263/58

(52) U.S. Cl. CPC A01N 43/90; C07D 307/85; A01N 43/12; C07D 209/42; A01N 43/38; C07D 263/58; A01N 43/76; C07D 235/24; A01N 43/52; C07D 471/04; C07D 277/68; A01N 43/78

(57) ABSTRACT

The present invention provides a method for promoting plant growth, which comprises treating a plant with at least one compound represented by the following Formula (1)

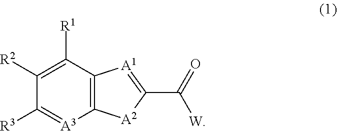

The compounds indicated by formula (1) are used to promote the growth of plants. A plant seed resulting from treating with the compound represented by formula (1) and comprising an effective quantity of the compound represented by formula (1). A composition for promoting plant growth comprising the compound represented by formula (1) and an inactive ingredient.

### TECHNICAL FIELD

The present invention relates to a method for promoting plant growth.

### BACKGROUND ART

Some chemical substances are known to exert an effect of promoting plant growth by being applied to plants. For example, when aminolevulinic acid is applied to plants, this substance exerts an effect of promoting growth of the plants.

### PRIOR ART DOCUMENT

### Non-Patent Document

<Biosynthesis, biotechnological production and applications of 5-aminolevulinic acid> K. Sasaki et al., (2002) Applied Microbial Biotechnology 58: pp 23-29

### SUMMARY OF THE INVENTION

An object of the present invention is to provide a method or the like that excellently promotes plant growth.

As a result of thorough examination, the present inventors found that the application of a certain compound to plants promotes growth of the plants and made the present invention.

That is, the present invention is as follows.

[1] A method for promoting plant growth, which comprises treating a plant with a compound represented by the following Formula (1):

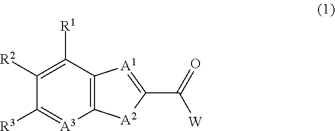

wherein

A¹ represents a nitrogen atom or —C(R⁵)═,

A² represents —N(R¹²)—, an oxygen atom, or a sulfur atom, when A¹ is a nitrogen atom,

A² represents —N(R¹²)— or an oxygen atom, when A¹ is —C(R⁵)═,

A³ represents a nitrogen atom or ═C(R⁴)—,

W represents —OR⁶, —ON═CR⁷R⁸, —SR⁹, or —NR⁷R¹⁰, when A² is —N(R¹²)— or a sulfur atom or when A² is an oxygen atom and A³ is a nitrogen atom,

W represents —OR⁶, —ON═CR⁷R⁸, —SR⁹, or —NR¹⁶R¹⁷, when A² is an oxygen atom and A³ is ═C(R⁴)—,

R¹, R², R³, and R⁴ are the same or different and each represents a hydrogen atom, a halogen atom, a cyano group, a nitro group, a C1-C6 alkyl group optionally having one or more groups selected from a group X, a C2-C6 alkenyl group optionally having one or more groups selected from the group X, a C2-C6 alkynyl group optionally having one or more groups selected from the group X, a phenyl group optionally having one or more groups selected from a group Y, a 6-membered aromatic heterocyclic group optionally having one or more groups selected from the group Y, a 5-membered aromatic heterocyclic group optionally having one or more groups selected from the group Y, a carboxy group, a C2-C6 alkylcarbonyl group optionally having one or more halogen atoms, a benzoyl group optionally having one or more groups selected from the group Y, a C2-C6 alkoxycarbonyl group, an aminocarbonyl group, —NR¹¹R¹², —S(O)₂NR⁷R¹¹, —OR¹¹, —S(O)ₘR¹¹, or —SF₅, when A² is —N(R¹²)— or a sulfur atom, or when at least one of A¹ and A³ is a nitrogen atom and A² is an oxygen atom,

R¹, R², R³, and R⁴ are the same or different and each represents a hydrogen atom, a halogen atom, a cyano group, a nitro group, a C1-C6 alkyl group optionally having one or more groups selected from the group X, a C2-C6 alkenyl group optionally having one or more groups selected from the group X, a C2-C6 alkynyl group optionally having one or more groups selected from the group X, a phenyl group optionally having one or more groups selected from the group Y, a 6-membered aromatic heterocyclic group optionally having one or more groups selected from the group Y, a 5-membered aromatic heterocyclic group optionally having one or more groups selected from the group Y, a carboxyl group, a C2-C6 alkylcarbonyl group optionally having one or more halogen atoms, a benzoyl group optionally having one or more groups selected from the group Y, a C2-C6 alkoxycarbonyl group, an aminocarbonyl group, —NR¹²R¹⁵, —S(O)₂NR⁷R¹¹, —OR¹⁵, —S(O)ₘR¹¹, or —SF₅, when A¹ is —C(R⁵)═, A² is an oxygen atom, and A³ is a ═C(R⁴)—,

R⁵ represents a hydrogen atom, a halogen atom, a cyano group, a nitro group, a C1-C6 alkyl group optionally having one or more groups selected from the group X, a carboxyl group, a C2-C6 alkoxycarbonyl group, —NR¹¹R¹², —S(O)₂NR⁷R¹¹, —OR¹³, a phenyl group optionally having one or more groups selected from the group Y, a 6-membered aromatic heterocyclic group optionally having one or more groups selected from the group Y, or a 5-membered aromatic heterocyclic group optionally having one or more groups selected from the group Y,

R⁶ represents a hydrogen atom, a C1-C6 alkyl group optionally having one or more groups selected from the group Z, a C3-C6 alkenyl group optionally having one or more groups selected from the group X, a C3-C6 alkynyl group optionally having one or more groups selected from the group X, a C4-C7 cycloalkylalkyl group optionally having one or more halogen atoms, a C3-C6 cycloalkyl group optionally having one or more halogen atoms, a phenyl group optionally having one or more groups selected from the group Y, or a C7-C9 phenylalkyl group in which a benzene ring portion may have optionally one or more groups selected from the group Y,

R⁷ and R⁸ are the same or different and each represents a C1-C6 alkyl group optionally having one or more halogen atoms, a phenyl group optionally having one or more groups selected from the group Y, or a hydrogen atom,

R⁹ represents a hydrogen atom, a C1-C6 alkyl group optionally having one or more groups selected from the group X, a C3-C6 alkenyl group optionally having one or more groups selected from the group X, a C3-C6 alkynyl group optionally having one or more groups selected from the group X, a phenyl group optionally having one or more groups selected from the group Y, or a C7-C9 phenylalkyl group in which a benzene ring portion may have optionally one or more groups selected from the group Y,

R¹⁰ represents a hydrogen atom, a cyano group, a C1-C6 alkyl group optionally having one or more groups selected from a group Z, a phenyl group optionally having one or more groups selected from the group Y, a benzyl group in which a benzene ring portion may have optionally one or more groups selected from the group Y, —OR⁷, or —NR⁷R⁸,

R¹¹ represents a C1-C6 alkyl group optionally having one or more groups selected from the group X, a C3-C6 alkenyl group optionally having one or more groups selected from the group X, a C3-C6 alkynyl group optionally having one or more groups selected from the group X, a C4-C7 cycloalkylalkyl group optionally having one or more halogen atoms, a C7-C9 phenylalkyl group in which a benzene ring portion may have optionally one or more groups selected from the group Y, a 6-membered aromatic heterocyclic C1-C3 alkyl group in which a 6-membered aromatic heterocyclic portion may have optionally one or more groups selected from the group Y, a phenyl group optionally having one or more groups selected from the group Y, a C3-C6 cycloalkyl group optionally having a halogen atom, or a hydrogen atom (provided that when m in —S(O)ₘR¹¹ is 1 or 2, R¹¹ does not represent a hydrogen atom),

R¹² represents a hydrogen atom, a C1-C4 alkyl group optionally having one or more halogen atoms, a C1-C4 alkylsulfonyl group optionally having one or more halogen atoms, a phenylsulfonyl group in which a benzene ring portion may have optionally one or more groups selected from the group Y, a C7-C9 phenylalkylsulfonyl group in which a benzene ring portion may have optionally one or more groups selected from the group Y, a C2-C6 alkoxycarbonyl group, —C(O)R¹⁴, or —C(O)NR⁷R⁸,

R¹³ represents a C1-C6 alkyl group optionally having one or more groups selected from the group X, a C3-C6 alkenyl group optionally having one or more groups selected from the group X, a C3-C6 alkynyl group optionally having one or more groups selected from the group X, a C4-C7 cycloalkylalkyl group optionally having one or more halogen atoms, a C3-C6 cycloalkyl group optionally having one or more halogen atoms, a C7-C9 phenylalkyl group in which a benzene ring portion may have optionally one or more groups selected from the group Y, a phenyl group optionally having one or more groups selected from the group Y, a C2-C6 alkylcarbonyl group optionally having one or more halogen atoms, a benzoyl group in which a benzene ring portion may have optionally one or more groups selected from the group Y, a C1-C6 alkylsulfonyl group optionally having one or more halogen atoms, a phenylsulfonyl group in which a benzene ring portion may have optionally one or more groups selected from the group Y, or a hydrogen atom,

R¹⁴ represents a hydrogen atom, a C1-C6 alkyl group optionally having one or more halogen atoms, a phenyl group optionally having one or more groups selected from the group Y, a 5-membered aromatic heterocyclic group optionally having one or more groups selected from the group Y, or a 6-membered aromatic heterocyclic group optionally having one or more groups selected from the group Y,

R¹⁵ represents a C1-C6 alkyl group optionally having one or more groups selected from the group X, a C3-C6 alkenyl group optionally having one or more groups selected from the group X, a C3-C6 alkynyl group optionally having one or more groups selected from the group X, a C4-C7 cycloalkylalkyl group optionally having one or more halogen atoms, a C7-C9 phenylalkyl group in which a benzene ring portion may have optionally one or more groups selected from the group Y, a 6-membered aromatic heterocyclic C1-C3 alkyl group in which a 6-membered aromatic heterocyclic portion may have optionally one or more groups selected from the group Y, a C3-C6 cycloalkyl group optionally having one or more halogen atoms, or a hydrogen atom,

R¹⁶ represents a C1-C6 alkyl group optionally having one or more halogen atoms, a phenyl group optionally having one or more groups selected from the group Y, or a hydrogen atom,

R¹⁷ represents a hydrogen atom, a cyano group, a C1-C6 alkyl group optionally having one or more groups selected from the group Z, a phenyl group optionally having one or more groups selected from the group Y, a benzyl group in which a benzene ring portion may have optionally one or more groups selected from the group Y, —OR⁶, or —NR⁷R⁸, when R¹⁶ is a phenyl group optionally having one or more groups selected from the group Y,

R¹⁷ represents a cyano group, a phenyl group optionally having one or more groups selected from the group Y, a benzyl group in which a benzene ring portion may have optionally one or more groups selected from the group Y, —OR⁷, or —NR⁷R⁸, when A¹ is a nitrogen atom and R¹⁶ is a C1-C6 alkyl group optionally having one or more halogen atoms or a hydrogen atom,

R¹⁷ represents a cyano group, a phenyl group optionally having one or more groups selected from the group Y, a benzyl group in which a benzene ring portion may have optionally one or more groups selected from the group Y, —OR¹⁸, or —NR⁷R⁸, when A¹ is —C(R⁵)═ and R¹⁶ is a C1-C6 alkyl group optionally having one or more halogen atoms or a hydrogen atom,

R¹⁸s are the same or different and each represents a methyl group having one or more halogen atoms, a C2-C6 alkyl group optionally having one or more halogen atoms, a phenyl group optionally having one or more groups selected from the group Y, or a hydrogen atom, and

m represents 0, 1, or 2,

the group X represents a group consisting of a halogen atom, a cyano group, and a C1-C6 alkoxy group optionally having one or more halogen atoms,

the group Y represents a group consisting of a halogen atom, a cyano group, a nitro group, a C1-C6 alkyl group optionally having one or more halogen atoms, and a C1-C6 alkoxy group optionally having one or more halogen atoms, and

the group Z represents a group consisting of a halogen atom, a hydroxyl group, a C1-C6 alkoxy group optionally having one or more halogen atoms, and a C2-C6 alkoxycarbonyl group (hereinafter, described as a “compound of the present invention”).

[2] The method according to [1], wherein the compound represented by Formula (1) is a compound in which A² represents a —N(R¹²)—, an oxygen atom, or a sulfur atom, when A¹ is a nitrogen atom,

A² represents an oxygen atom, when A¹ is —C(R⁵)═ and A³ is ═C(R⁴)—,

A² represents —N(R¹²)— or an oxygen atom, when A¹ is —C(R⁵)═ and A³ is a nitrogen atom,

R¹, R², R³, and R⁴ are the same or different and each represents a hydrogen atom, a halogen atom, a cyano group, a nitro group, a C1-C6 alkyl group optionally having one or more groups selected from the group X, a C2-C6 alkenyl group optionally having one or more groups selected from the group X, a C2-C6 alkynyl group optionally having one or more groups selected from the group X, a phenyl group optionally having one or more groups selected from the group Y, a 5-membered aromatic heterocyclic group optionally having one or more groups selected from the group Y, a carboxyl group, a C2-C6 alkylcarbonyl group optionally having one or more halogen atoms, a benzoyl group optionally having one or more groups selected from the group Y, a C2-C6 alkoxycarbonyl group, an aminocarbonyl group, —NR¹¹R¹², —S(O)₂NR⁷R¹¹, —OR¹¹, —S(O)ₘR¹¹, or —SF₅, when A² is —N(R¹²)— or a sulfur atom, or when at least one or more of A¹ and A³ are a nitrogen atom and A² is an oxygen atom, and

R¹, R², R³, and R⁴ are the same or different and each represents a hydrogen atom, a halogen atom, a cyano group, a nitro group, a C1-C6 alkyl group optionally having one or more groups selected from the group X, a C2-C6 alkenyl group optionally having one or more groups selected from the group X, a C2-C6 alkynyl group optionally having one or more groups selected from the group X, a phenyl group optionally having one or more groups selected from the group Y, a 5-membered aromatic heterocyclic group optionally having one or more groups selected from the group Y, a carboxy group, a C2-C6 alkylcarbonyl group optionally having one or more halogen atoms, a benzoyl group optionally having one or more groups selected from the group Y, a C2-C6 alkoxycarbonyl group, an aminocarbonyl group, —NR¹²R¹⁵, —S(O)₂NR⁷R¹¹, —OR¹⁵, —S(O)ₘR¹¹, or —SF₅, when A¹ is —C(R⁵)═, A² is an oxygen atom, and A³ is ═C(R⁴)—.

[3] The method according to [1] or [2], wherein the compound represented by Formula (1) is a compound in which A² represents an oxygen atom,

A³ represents ═C(R⁴)—, and

R⁶ represents a C1-C6 alkyl group optionally having one or more groups selected from the group Z, a C3-C6 alkenyl group optionally having one or more groups selected from the group X, a C3-C6 alkynyl group optionally having one or more groups selected from the group X, a C4-C7 cycloalkylalkyl group optionally having one or more halogen atoms, a C3-C6 cycloalkyl group optionally having one or more halogen atoms, a phenyl group optionally having one or more groups selected from the group Y, or a C7-C9 phenylalkyl group in which a benzene ring portion may have optionally one or more groups selected from the group Y.

[4] The method according to [1] or [2], wherein the compound represented by Formula (1) is a compound in which A¹ represents a nitrogen atom,

A² represents —N(R¹²)— or a sulfur atom,

A³ represents ═C(R⁴)—, and

R¹⁰ represents a hydrogen atom, a C1-C6 alkyl group, a phenyl group optionally having one or more groups selected from the group Y, a benzyl group in which a benzene ring portion may have optionally one or more groups selected from the group Y, or —NR⁷R⁸.

[5] The method according to [1], wherein the compound represented by Formula (1) is a compound in which A¹ represents —C(R⁵)═,

A² represents —N(R¹²)—,

A³ represents ═C(R⁴)—,

R¹, R³, and R⁴ are the same or different and each represents a hydrogen atom, a halogen atom, a cyano group, a nitro group, a C1-C6 alkyl group optionally having one or more groups selected from the group X, a C2-C6 alkenyl group optionally having one or more groups selected from the group X, a C2-C6 alkynyl group optionally having one or more groups selected from the group X, a phenyl group optionally having one or more groups selected from the group Y, a 6-membered aromatic heterocyclic group optionally having one or more groups selected from the group Y, a 5-membered aromatic heterocyclic group optionally having one or more groups selected from the group Y, a carboxyl group, a C2-C6 alkylcarbonyl group optionally having one or more halogen atoms, a benzoyl group optionally having one or more groups selected from the group Y, a C2-C6 alkoxycarbonyl group, an aminocarbonyl group, —NR¹¹R¹², —S(O)₂NR⁷R¹¹, —OR¹¹, —S(O)ₘR¹¹, or —SF₅,

R² represents a hydrogen atom, a cyano group, a nitro group, a C1-C6 alkyl group optionally having one or more groups selected from the group X, a C2-C6 alkenyl group optionally having one or more groups selected from the group X, a C2-C6 alkynyl group optionally having one or more groups selected from the group X, a phenyl group optionally having one or more groups selected from the group Y, a 6-membered aromatic heterocyclic group optionally having one or more groups selected from the group Y, a 5-membered aromatic heterocyclic group optionally having one or more groups selected from the group Y, a carboxyl group, a C2-C6 alkylcarbonyl group optionally having one or more halogen atoms, a benzoyl group optionally having one or more groups selected from the group Y, a C2-C6 alkoxycarbonyl group, an aminocarbonyl group, —NR¹¹R¹², —S(O)₂NR⁷R¹¹, —OR¹⁹, —S(O)ₘR¹¹, or —SF₅,

R⁵ represents a cyano group, a nitro group, a C1-C6 alkyl group optionally having one or more groups selected from the group X, a carboxyl group, a C2-C6 alkoxycarbonyl group, —NR¹¹R¹², —S(O)₂NR⁷R¹¹, —OR¹³, a phenyl group optionally having one or more groups selected from the group Y, a 6-membered aromatic heterocyclic group optionally having one or more groups selected from the group Y, or a 5-membered aromatic heterocyclic group optionally having one or more groups selected from the group Y, when all of R¹, R², R³, and R⁴ are hydrogen atoms,

R⁵ represents a cyano group, a nitro group, a C1-C6 alkyl group optionally having one or more groups selected from the group X, a carboxy group, a C2-C6 alkoxycarbonyl group, —NR¹¹R¹², —S(O)₂NR⁷R¹¹, —OR¹³, a phenyl group optionally having one or more groups selected from the group Y, a 6-membered aromatic heterocyclic group optionally having one or more groups selected from the group Y, a 5-membered aromatic heterocyclic group optionally having one or more groups selected from the group Y, or a hydrogen atom, when at least one of R¹, R², R³, and R⁴ is not a hydrogen atom, and

R¹⁹ represents C2-C6 alkyl group optionally having one or more groups selected from the group X, a C3-C6 alkenyl group optionally having one or more groups selected from the group X, a C3-C6 alkynyl group optionally having one or more groups selected from the group X, a C4-C7 cycloalkylalkyl group optionally having one or more halogen atoms, a C7-C9 phenylalkyl group in which a benzene ring portion may have optionally one or more groups selected from group Y, a 6-membered aromatic heterocyclic C1-C3 alkyl group in which a 6-membered aromatic heterocyclic portion may have optionally one or more groups selected from the group Y, a phenyl group optionally having one or more groups selected from the group Y, a C3-C6 cycloalkyl group optionally having one or more halogen atoms, or a hydrogen atom.

[6] The method according to [1] or [2], wherein the compound represented by Formula (1) is a compound in which A¹ represents a nitrogen atom,

A² represents —N(R¹²)—, and

A³ represents a nitrogen atom.

[7] The method for promoting plant growth according to one of [1] to [6], wherein the plant is a plant that has been or will be exposed to abiotic stress.

[8] The method according to any one of [1] to [7], wherein the application to the plant includes a spraying treatment, a soil treatment, a seed treatment, or a hydroponic treatment.

[9] The method according to any one of [1] to [7], wherein the application to the plant is a seed treatment.

[10] The method according to any one of [1] to [9], wherein the plant is rice, corn, or wheat.

[11] The method according to any one of [1] to [10], [18] and [19], wherein the plant is a transgenic plant.

[12] The method according to any one of [7] to [11], [18] and [19], wherein the abiotic stress is high-temperature stress.

[13] The method according to any one of [7] to [11], [18] and [19], wherein the abiotic stress is low-temperature stress.

[14] The method according to any one of [7] to [11], [18] and [19], wherein the abiotic stress is drought stress.

[15] Use of the compound represented by Formula (1) for promoting plant growth,

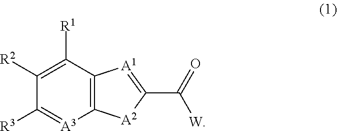

[16] A plant seed which is obtained by being treated with the compound according to [1] to [6] comprising a compound represented by the Formula (1) in an effective dose

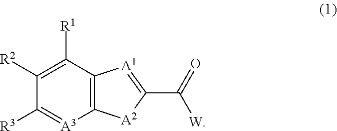

[17] A composition for promoting plant growth comprising the compound represented by the Formula (1) and an inactive ingredient

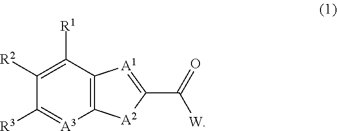

[18] The method according to any one of [1] to [9], wherein the plant is soybean.

[19] The method according to any one of [1] to [9], wherein the plant is cotton.

### MODE FOR CARRYING OUT THE INVENTION

In the present invention, the “promotion of the growth of a plant (hereinafter, described as “growth promotion” in some cases)” means the increase in the rate of seedling establishment, increase in the number of healthy leaves, increase in the height of the plant, increase in the weight of the plant, increase in the leaf area, increase in the number or weight of seeds or fruits, increase in the number of occasion of flower setting or fruit setting, and promoted growth of a root.

The growth promotion can be quantified by the following parameters.

(1) Rate of Seedling Establishment

Seeds of a plant are seeded in, for example, soil, filter paper, an agar medium, or sand and cultivated for a certain period of time. Thereafter, the proportion of the surviving seedlings is investigated.

(2) Number of Healthy Leaves or Proportion of Healthy Leaves

For each plant, the number of healthy leaves is counted, and the total number of healthy leaves is investigated. Alternatively, a ratio of the number of healthy leaves to the total number of the leaves of the plant is investigated.

(3) Plant Height

For each plant, a length from the base to the terminal branch or leave of the overground stem is measured.

(4) Plant Weight

The overground portion of each plant is cut and collected, and the weight thereof is measured to determine a fresh weight of the plant. Alternatively, the cut and collected sample is dried, and then a weight thereof is measured to determine a dry weight of the plant.

(5) Leaf Area

A plant is imaged with a digital camera, and the area of the green portion in the picture is quantified by image analysis software, for example, Win ROOF (manufactured by MITANI CORPORATION), or visually evaluated to determine a leaf area of the plant.

(6) Leaf Color

A leaf of a plant is sampled, and an amount of chlorophyll is measured using a chlorophyll meter (for example, SPAD-502, manufactured by Konica Minolta Sensing Europe B.V.) to determine the leaf color. In addition, the plant is imaged with a digital camera, and the area of the green portion in the picture is quantified by performing color extraction by using image analysis software, for example, Win ROOF (manufactured by MITANI CORPORATION), whereby the area of the green portion of the leaf of the plant is determined.

(7) Number or Weight of Seeds or Fruits

A plant is cultured until it produces seeds or fruits or until the seeds or fruits ripen, and then the number of fruits per plant or the total weight of fruits per plant is measured. Moreover, the plant is cultivated until the seeds ripen, and then constituents of the yield, for example, the number of ears, ripening rate, and thousand kennel weight, are investigated.

(8) Flower Setting Rate, Fruit Setting Rate, Fruition Rate, or Grain Filling Rate

A plant is cultivated until it fruits, and the number of set flowers and fruits are counted to determine a fruit setting rate (number of set fruit/number of set flower×100). After the seeds ripen, the number of produced fruits and number of filled grains are counted to determine a fruition rate (number of produced fruit/number of set flower×100) and a grain filling rate (number of filled grain/number of produced fruit×100).

(9) Promoted Growth of Root

A plant is cultivated in soil or cultured hydroponically, and a length of the root is measured. Alternatively, the root is cut and collected, and a fresh weight thereof or the like is measured.

When a plant is treated with the compound of the present invention by the method of the present invention, the entire plant may be treated, or a portion thereof (foliage, a sprout, a flower, a fruit, an ear, a seed, a bulb, a tuber, a root, and the like) may be treated. Moreover, the plant may be treated at various growth stages thereof (a germination period including a pre-seeding stage, a seeding stage, a post-seeding stage, pre- and post-budding stages, and the like, a period of vegetative growth including a seedling stage, a seedling transplant stage, and a cuttage stage or a seedling insertion stage, a growth stage after planting, a reproductive period including the a pre-flowering stage, a flowering stage, a post-flowering stage, a stage immediately before emergence of ear, an ear emergence stage, and the like, a harvesting period including a time before prospect of harvest, a stage before prospect of ripening, the period during which fruits start to be colored, and the like). Herein, a bulb refers to a discoid stem, a corm, a rhizome, a tuberous root, a rhizophore, and the like. In addition, a seedling includes a nursery plant raised from a seed, a cuttage, and the like.

Examples of substituents used and described in the present specification will be described below.

Examples of the “halogen atom” in the compound of the present invention include a fluorine atom, a chlorine atom, a bromine atom, an iodine atom, and the like.

Examples of the “C1-C6 alkyl group” in the compound of the present invention include a methyl group, an ethyl group, a propyl group, an isopropyl group, a butyl group, an isobutyl group, a sec-butyl group, a tert-butyl group, a pentyl group, a 2,2-dimethylpropyl group, a 3-methylbutyl group, a 2,3-dimethylbutyl group, a 3,3-dimethylbutyl group, a hexyl group, and the like.

Examples of the “C1-C6 alkyl group optionally having one or more groups selected from the group X” in the compound of the present invention include a methyl group, an ethyl group, a propyl group, an isopropyl group, a butyl group, an isobutyl group, a sec-butyl group, a tert-butyl group, a pentyl group, a 2,2-dimethylpropyl group, a 3-methylbutyl group, a 2,3-dimethylbutyl group, a 3,3-dimethylbutyl group, a hexyl group, a trichloromethyl group, a difluoromethyl group, a trifluoromethyl group, a 2,2,2-trifluoroethyl group, a pentafluoroethyl group, a heptafluoropropyl group, a heptafluoroisopropyl group, a cyanomethyl group, a 2-cyanoethyl group, a methoxymethyl group, a 2-methoxyethyl group, an ethoxymethyl group, a 2-ethoxyethyl group, a trifluoromethoxymethyl group, a 2,2,2-trifluoroethoxymethyl group, and the like.

Examples of the “C1-C6 alkyl group optionally having one or more groups selected from the group Z” in the compound of the present invention include a methyl group, an ethyl group, a propyl group, an isopropyl group, a butyl group, an isobutyl group, a sec-butyl group, a tert-butyl group, a pentyl group, a 2,2-dimethylpropyl group, a 3-methylbutyl group, a 2,3-dimethylbutyl group, 3,3-dimethylbutyl group, a hexyl group, a trichloromethyl group, a difluoromethyl group, a trifluoromethyl group, a 2,2,2-trifluoroethyl group, a pentafluoroethyl group, a heptafluoropropyl group, a heptafluoroisopropyl group, a 2-hydroxyethyl group, a methoxymethyl group, a 2-methoxyethyl group, an ethoxymethyl group, a 2-ethoxyethyl group, a trifluoromethoxymethyl group, a 2,2,2-trifluoroethoxyethyl group, a methoxycarbonylmethyl group, and the like.

Examples of the “C1-C6 alkyl group optionally having one or more halogen atoms” in the compound of the present invention include a methyl group, an ethyl group, a propyl group, an isopropyl group, a butyl group, an isobutyl group, a sec-butyl group, a tert-butyl group, a pentyl group, a 2,2-dimethylpropyl group, a 3-methylbutyl group, a 2,3-dimethylbutyl group, a 3,3-dimethylbutyl group, a hexyl group, a trichloromethyl group, a difluoromethyl group, a trifluoromethyl group, a 2,2,2-trifluoroethyl group, a pentafluoroethyl group, a heptafluoropropyl group, a heptafluoroisopropyl group, and the like.

Example of the “C1-C4 alkyl group” in the compound of the present invention include a methyl group, an ethyl group, a propyl group, an isopropyl group, a butyl group, an isobutyl group, a sec-butyl group, and a tert-butyl group.

Examples of the “C1-C4 alkyl group optionally having one or more halogen atoms” in the compound of the present invention include a methyl group, an ethyl group, a propyl group, an isopropyl group, a butyl group, an isobutyl group, a sec-butyl group, a tert-butyl group, a trichloromethyl group, a difluoromethyl group, a trifluoromethyl group, a 2,2,2-trifluoroethyl group, a pentafluoroethyl group, a heptafluoropropyl group, a heptafluoroisopropyl group, and the like.

Examples of the “C1-C3 alkyl group” in the compound of the present invention include a methyl group, an ethyl group, a propyl group, and an isopropyl group.

Examples of the “methyl group having one or more halogen atoms” in the compound of the present invention include a chloromethyl group, a trichloromethyl group, a fluoromethyl group, a difluoromethyl group, a trifluoromethyl group, and the like.

Examples of the “C2-C6 alkyl group” in the compound of the present invention include an ethyl group, a propyl group, an isopropyl group, a butyl group, an isobutyl group, a sec-butyl group, a tert-butyl group, a pentyl group, a 2,2-dimethylpropyl group, a 3-methylbutyl group, a 2,3-dimethylbutyl group, a 3,3-dimethylbutyl group, a hexyl group, and the like.

Examples of the “C2-C6 alkyl group optionally having one or more groups selected from the group X” in the compound of the present invention, include an ethyl group, a propyl group, an isopropyl group, a butyl group, an isobutyl group, a sec-butyl group, a tert-butyl group, a pentyl group, a 2,2-dimethylpropyl group, a 3-methylbutyl group, a 2,3-dimethylbutyl group, a 3,3-dimethylbutyl group, a hexyl group, a 2,2,2-trifluoroethyl group, a pentafluoroethyl group, a heptafluoropropyl group, a heptafluoroisopropyl group, a 4-chlorobutyl group, a 2-cyanoethyl group, a 2-methoxyethyl group, a 2-ethoxyethyl group, a 2-trifluoromethoxyethyl group, a 2-(2,2,2-trifluoroethoxyl)ethyl group, and the like.

Examples of the “C2-C6 alkyl group optionally having one or more halogen atoms” in the compound of the present invention include an ethyl group, a propyl group, an isopropyl group, a butyl group, an isobutyl group, a sec-butyl group, a tert-butyl group, a pentyl group, a 2,2-dimethylpropyl group, a 3-methylbutyl group, a 2,3-dimethylbutyl group, a 3,3-dimethylbutyl group, a hexyl group, a 2,2,2-trifluoroethyl group, a pentafluoroethyl group, a heptafluoropropyl group, a heptafluoroisopropyl group, and the like.

Examples of the “C4-C7 cycloalkyl alkyl group” in the compound of the present invention include a cyclopropyl methyl group, a 1-cyclopropyl ethyl group, a 2-cyclopropyl ethyl group, a cyclobutyl methyl group, a 1-cyclobutyl ethyl group, a cyclopentyl methyl group, a cyclohexyl methyl group, and the like.

Examples of the “C4-C7 cycloalkyl alkyl group optionally having one or more halogen atoms” in the compound of the present invention include a cyclopropyl methyl group, a 1-cyclopropyl ethyl group, a 2-cyclopropyl ethyl group, a cyclobutyl methyl group, a 1-cyclobutyl ethyl group, a cyclopentyl methyl group, a cyclohexyl methyl group, a 2,2-difluorocyclopropyl methyl group, a 1-(2,2-dichlorocyclopropyl)ethyl group, a 2,2-dibromocyclobutyl methyl group, a 2-chlorocyclopentyl methyl group, and the like.

Examples of the “C3-C6 cycloalkyl group” in the compound of the present invention include a cyclopropyl group, a cyclobutyl group, a cyclopentyl group, a cyclohexyl group, and the like.

Examples of the “C3-C6 cycloalkyl group optionally having one or more halogen atoms” in the compound of the present invention include a cyclopropyl group, a cyclobutyl group, a cyclopentyl group, a cyclohexyl group, a 2,2-difluorocyclopropyl group, a 2,2-dichlorocyclopropyl group, a 2-chlorocyclopentyl group, a 4-iodocyclohexyl group, and the like.

Examples of the “C2-C6 alkenyl group” in the compound of the present invention include a vinyl group, a 1-propenyl group, an allyl group, an isopropenyl group, a 2-methyl-1-propenyl group, a 1-butenyl group, a 2-butenyl group, a 3-butenyl group, a 1-pentenyl group, a 1-hexenyl group, and the like.

Examples of the “C2-C6 alkenyl group optionally having one or more groups selected from the group X” in the compound of the present invention include a vinyl group, a 1-propenyl group, an allyl group, an isopropenyl group, a 2-methyl-1-propenyl group, a 1-butenyl group, a 2-butenyl group, a 3-butenyl group, a 1-pentenyl group, a 1-hexenyl group, a 2,2-difluoroethenyl group, a 2,2-dichloroethenyl group, a 2-cyano-1-ethenyl group, a 2-methoxy-1-ethenyl group, a 2-ethoxy-1-ethenyl group, a 3,3-difluoro-2-propenyl group, a 3,3-dichloro-2-propenyl group, a 4-methoxy-2-methyl-2-butenyl group, a 3-cyano-2-butenyl group, and the like.

Examples of the “C3-C6 alkenyl group” in the compound of the present invention include an allyl group, a 1-methyl-2-propenyl group, a 2-methyl-2-propenyl group, a 2-butenyl group, a 3-butenyl group, a 2-pentenyl group, a 4-pentenyl group, a 2-hexenyl group, a 5-hexenyl group, and the like.

Examples of the “C3-C6 alkenyl group optionally having one or more groups selected from the group X” in the compound of the present invention include an allyl group, a 1-methyl-2-propenyl group, a 2-methyl-2-propenyl group, a 2-butenyl group, a 3-butenyl group, a 2-pentenyl group, a 4-pentenyl group, a 2-hexenyl group, a 5-hexenyl group, a 3,3-difluoro-2-propenyl group, a 3,3-dichloro-2-propenyl group, a 4-methoxy-2-methyl-2-butenyl group, a 3-cyano-2-butenyl group, and the like.

Examples of the “C2-C6 alkynyl group” in the compound of the present invention include an ethynyl group, a 1-propynyl group, a propargyl group, a 1-butynyl group, a 2-butynyl group, a 3-butynyl group, a 3,3-dimethyl-1-butynyl group, a 1-pentynyl group, a 2-pentynyl group, a 1-hexynyl group, and the like.

Examples of the “C2-C6 alkynyl group optionally having one or more groups selected from the group X” in the compound of the present invention include an ethynyl group, a propargyl group, a 1-propynyl group, a 1-butynyl group, a 2-butynyl group, a 3-butynyl group, a 3,3-dimethyl-1-butynyl group, a 1-pentynyl group, a 1-hexynyl group, a 4-chloro-2-butynyl group, a 4-cyano-2-butynyl group, a 5-cyano-2-pentynyl group, a 4-methoxy-2-butynyl group, a 4-(2-chloroethoxy)-2-butynyl group, and the like.

Examples of the “C3-C6 alkynyl group” in the compound of the present invention include a propargyl group, a 2-butynyl group, a 3-butynyl group, a 2-pentynyl group, a 4-pentynyl group, a 2-hexynyl group, a 5-hexynyl group, and the like.

Examples of the “C3-C6 alkynyl group optionally having one or more groups selected from the group X” in the compound of the present invention include a propargyl group, a 2-butynyl group, a 3-butynyl group, a 2-pentynyl group, a 2-hexynyl group, a 4-chloro-2-butynyl group, a 4-cyano-2-butynyl group, a 5-cyano-2-pentynyl group, a 4-methoxy-2-butynyl group, a 4-(2-chloroethoxy)-2-butynyl group, and the like.

Examples of the “phenyl group optionally having one or more groups selected from the group Y” in the compound of the present invention include a phenyl group, a 2-fluorophenyl group, a 3-fluorophenyl group, a 4-fluorophenyl group, a 2-chlorophenyl group, a 3-chlorophenyl group, a 4-chlorophenyl group, a 2-bromophenyl group, a 3-bromophenyl group, a 4-bromophenyl group, a 2-iodophenyl group, a 3-iodophenyl group, a 4-iodophenyl group, a 2-cyanophenyl group, a 3-cyanophenyl group, a 4-cyanophenyl group, a 2-nitrophenyl group, a 3-nitrophenyl group, a 4-nitrophenyl group, a 2-methylphenyl group, a 3-methylphenyl group, a 4-methylphenyl group, a 2-isopropylphenyl group, a 3-isopropylphenyl group, a 4-isopropylphenyl group, a 2-butylphenyl group, a 3-butylphenyl group, a 4-butylphenyl group, a 2-isobutylphenyl group, a 3-isobutylphenyl group, a 4-isobutylphenyl group, a 3-tert-butylphenyl group, a 4-tert-butylphenyl group, a 2-difluoromethylphenyl group, a 3-difluoromethylphenyl group, a 4-difluoromethylphenyl group, a 2-trifluoromethylphenyl group, a 3-trifluoromethylphenyl group, a 4-trifluoromethylphenyl group, a 2-(2,2,2-trifluoroethyl)phenyl group, a 3-(2,2,2-trifluoroethyl)phenyl group, a 4-(2,2,2-trifluoroethyl)phenyl group, a 2-pentafluoroethylphenyl group, a 3-pentafluoroethylphenyl group, a 4-pentafluoroethylphenyl group, a 2-heptafluoropropylphenyl group, a 3-heptafluoropropylphenyl group, a 4-heptafluoropropylphenyl group, a 2-heptafluoroisopropylphenyl group, a 3-heptafluoroisopropylphenyl group, a 4-heptafluoroisopropylphenyl group, a 2-methoxyphenyl group, a 3-methoxyphenyl group, a 4-methoxyphenyl group, a 2-tert-butoxyphenyl group, a 3-tert-butoxyphenyl group, a 4-tert-butoxyphenyl group, a 2-pentyloxyphenyl group, a 3-pentyloxyphenyl group, a 4-pentyloxyphenyl group, a 2-(2,2-dimethylpropoxyl)phenyl group, a 3-(2,2-dimethylpropoxyl)phenyl group, a 4-(2,2-dimethylpropoxyl)phenyl group, a 2-(3-methylbutoxyl)phenyl group, a 3-(3-methylbutoxyl)phenyl group, a 4-(3-methylbutoxyl)phenyl group, a 2-difluoromethoxyphenyl group, a 3-difluoromethoxyphenyl group, a 4-difluoromethoxyphenyl group, a 2-trifluoromethoxyphenyl group, a 3-trifluoromethoxyphenyl group, a 4-trifluoromethoxyphenyl group, a 4-(2,2,2-trifluoroethoxyl)phenyl group, a 2,4-dimethoxyphenyl group, a 3-chloro-4-methylphenyl group, and the like.

Examples of the “C7-C9 phenylalkyl group” in the compound of the present invention include a benzyl group, a 1-phenylethyl group, a 2-phenylethyl group, a 1-phenylpropyl group, a 2-phenylpropyl group, a 3-phenylpropyl group, a 1-methyl-1-phenylethyl group, and the like.

Examples of the “C7-C9 phenylalkyl group in which a benzene ring portion may have optionally one or more groups selected from the group Y” in the compound of the present invention include a benzyl group, a 2-fluorobenzyl group, a 3-chlorobenzyl group, a 4-bromobenzyl group, a 2-cyanobenzyl group, a 3-nitrobenzyl group, a 3-methoxybenzyl group, a 4-trifluoromethylbenzyl group, a 4-trifluoromethoxybenzyl group, a 1-(3-chlorophenyl)ethyl group, a 2-(4-bromophenyl)ethyl group, a 1-(2-cyanophenyl)propyl group, a 2-(3-nitrophenyl)propyl group, a 3-(3-methoxyphenyl)propyl group, a 1-methyl-1-(4-trifluoromethoxyphenyl)ethyl group, and the like.

Examples of the “benzyl group in which a benzene ring portion may have optionally one or more groups selected from the group Y” in the compound of the present invention include a 2-fluorobenzyl group, a 3-chlorobenzyl group, a 4-bromobenzyl group, a 2-cyanobenzyl group, a 3-nitrobenzyl group, a 3-methoxybenzyl group, a 4-chlorobenzyl group, a 4-methylbenzyl group, a 4-trifluoromethylbenzyl group, a 4-trifluoromethoxybenzyl group, and the like.

Examples of the “6-membered aromatic heterocyclic group” in the compound of the present invention include a 2-pyridyl group, a 3-pyridyl group, a 4-pyridyl group, a 3-pyridazinyl group, a 4-pyridazinyl group, a 2-pyrimidinyl group, a 4-pyrimidinyl group, a 5-pyrimidinyl group, a 2-pyrazinyl group, a 4-(1,2,3-triazinyl) group, a 5-(1,2,3-triazinyl) group, a 3-(1,2,4-triazinyl) group, a 5-(1,2,4-triazinyl) group, a 6-(1,2,4-triazinyl) group, a 2-(1,3,5-triazinyl) group, and the like.

Examples of the “6-membered aromatic heterocyclic group optionally having one or more groups selected from the group Y” in the compound of the present invention include a 2-pyridyl group, a 3-fluoro-2-pyridyl group, a 4-fluoro-2-pyridyl group, a 5-fluoro-2-pyridyl group, a 6-fluoro-2-pyridyl group, a 3-chloro-2-pyridyl group, a 4-chloro-2-pyridyl group, a 5-chloro-2-pyridyl group, a 6-chloro-2-pyridyl group, a 3-bromo-2-pyridyl group, a 4-bromo-2-pyridyl group, a 5-bromo-2-pyridyl group, a 6-bromo-2-pyridyl group, a 3-iodo-2-pyridyl group, a 4-iodo-2-pyridyl group, a 5-iodo-2-pyridyl group, a 6-iodo-2-pyridyl group, a 3-cyano-2-pyridyl group, a 4-cyano-2-pyridyl group, a 5-cyano-2-pyridyl group, a 6-cyano-2-pyridyl group, a 3-nitro-2-pyridyl group, a 4-nitro-2-pyridyl group, a 5-nitro-2-pyridyl group, a 6-nitro-2-pyridyl group, a 3-methyl-2-pyridyl group, a 4-methyl-2-pyridyl group, a 5-methyl-2-pyridyl group, a 6-methyl-2-pyridyl group, a 3-ethyl-2-pyridyl group, a 4-ethyl-2-pyridyl group, a 5-ethyl-2-pyridyl group, a 6-ethyl-2-pyridyl group, a 3-propyl-2-pyridyl group, a 4-propyl-2-pyridyl group, a 5-propyl-2-pyridyl group, a 6-propyl-2-pyridyl group, a 3-isopropyl-2-pyridyl group, a 4-isopropyl-2-pyridyl group, a 5-isopropyl-2-pyridyl group, a 6-isopropyl-2-pyridyl group, a 3-butyl-2-pyridyl group, a 4-butyl-2-pyridyl group, a 5-butyl-2-pyridyl group, a 6-butyl-2-pyridyl group, a 3-isobutyl-2-pyridyl group, a 4-isobutyl-2-pyridyl group, a 5-isobutyl-2-pyridyl group, a 6-isobutyl-2-pyridyl group, a 3-sec-butyl-2-pyridyl group, a 4-sec-butyl-2-pyridyl group, a 5-sec-butyl-2-pyridyl group, a 6-sec-butyl-2-pyridyl group, a 3-tert-butyl-2-pyridyl group, a 4-tert-butyl-2-pyridyl group, a 5-tert-butyl-2-pyridyl group, a 6-tert-butyl-2-pyridyl group, a 3-difluoromethyl-2-pyridyl group, a 4-difluoromethyl-2-pyridyl group, a 5-difluoromethyl-2-pyridyl group, a 6-difluoromethyl-2-pyridyl group, a 3-trifluoromethyl-2-pyridyl group, a 4-trifluoromethyl-2-pyridyl group, a 5-trifluoromethyl-2-pyridyl group, a 6-trifluoromethyl-2-pyridyl group, a 3-(2,2,2-trifluoroethyl)-2-pyridyl group, a 4-(2,2,2-trifluoroethyl)-2-pyridyl group, a 5-(2,2,2-trifluoroethyl)-2-pyridyl group, a 6-(2,2,2-trifluoroethyl)-2-pyridyl group, a 3-pentafluoroethyl-2-pyridyl group, a 4-pentafluoroethyl-2-pyridyl group, a 5-pentafluoroethyl-2-pyridyl group, a 6-pentafluoroethyl-2-pyridyl group, a 3-heptafluoropropyl-2-pyridyl group, a 4-heptafluoropropyl-2-pyridyl group, a 5-heptafluoropropyl-2-pyridyl group, a 6-heptafluoropropyl-2-pyridyl group, a 3-heptafluoroisopropyl-2-pyridyl group, a 4-heptafluoroisopropyl-2-pyridyl group, a 5-heptafluoroisopropyl-2-pyridyl group, a 6-heptafluoroisopropyl-2-pyridyl group, a 3-pyridyl group, a 2-methyl-3-pyridyl group, a 4-methyl-3-pyridyl group, a 5-methyl-3-pyridyl group, a 6-methyl-3-pyridyl group, a 2-ethyl-3-pyridyl group, a 4-ethyl-3-pyridyl group, a 5-ethyl-3-pyridyl group, a 6-ethyl-3-pyridyl group, a 2-propyl-3-pyridyl group, a 4-propyl-3-pyridyl group, a 5-propyl-3-pyridyl group, a 6-propyl-3-pyridyl group, a 2-isopropyl-3-pyridyl group, a 4-isopropyl-3-pyridyl group, a 5-isopropyl-3-pyridyl group, a 6-isopropyl-3-pyridyl group, a 2-butyl-3-pyridyl group, a 4-butyl-3-pyridyl group, a 5-butyl-3-pyridyl group, a 6-butyl-3-pyridyl group, a 2-isobutyl-3-pyridyl group, a 4-isobutyl-3-pyridyl group, a 5-isobutyl-3-pyridyl group, a 6-isobutyl-3-pyridyl group, a 2-sec-butyl-3-pyridyl group, a 4-sec-butyl-3-pyridyl group, a 5-sec-butyl-3-pyridyl group, a 6-sec-butyl-3-pyridyl group, a 2-tert-butyl-3-pyridyl group, a 4-tert-butyl-3-pyridyl group, a 5-tert-butyl-3-pyridyl group, a 6-tert-butyl-3-pyridyl group, a 2-difluoromethyl-3-pyridyl group, a 4-difluoromethyl-3-pyridyl group, a 5-difluoromethyl-3-pyridyl group, a 6-difluoromethyl-3-pyridyl group, a 2-trifluoromethyl-3-pyridyl group, a 4-trifluoromethyl-3-pyridyl group, a 5-trifluoromethyl-3-pyridyl group, a 6-trifluoromethyl-3-pyridyl group, a 2-(2,2,2-trifluoroethyl)-3-pyridyl group, a 4-(2,2,2-trifluoroethyl)-3-pyridyl group, a 5-(2,2,2-trifluoroethyl)-3-pyridyl group, a 6-(2,2,2-trifluoroethyl)-3-pyridyl group, a 2-pentafluoroethyl-3-pyridyl group, a 4-pentafluoroethyl-3-pyridyl group, a 5-pentafluoroethyl-3-pyridyl group, a 6-pentafluoroethyl-3-pyridyl group, a 2-heptafluoropropyl-3-pyridyl group, a 4-heptafluoropropyl-3-pyridyl group, a 5-heptafluoropropyl-3-pyridyl group, a 6-heptafluoropropyl-3-pyridyl group, a 2-heptafluoroisopropyl-3-pyridyl group, a 4-heptafluoroisopropyl-3-pyridyl group, a 5-heptafluoroisopropyl-3-pyridyl group, a 6-heptafluoroisopropyl-3-pyridyl group, a 4-pyridyl group, a 2-methyl-4-pyridyl group, a 3-methyl-4-pyridyl group, a 2-ethyl-4-pyridyl group, a 3-ethyl-4-pyridyl group, a 2-propyl-4-pyridyl group, a 3-propyl-4-pyridyl group, a 2-isopropyl-4-pyridyl group, a 3-isopropyl-4-pyridyl group, a 2-butyl-4-pyridyl group, a 3-butyl-4-pyridyl group, a 2-isobutyl-4-pyridyl group, a 3-isobutyl-4-pyridyl group, a 2-sec-butyl-4-pyridyl group, a 3-sec-butyl-4-pyridyl group, a 2-tert-butyl-4-pyridyl group, a 3-tert-butyl-4-pyridyl group, a 2-difluoromethyl-4-pyridyl group, a 3-difluoromethyl-4-pyridyl group, a 2-trifluoromethyl-4-pyridyl group, a 3-trifluoromethyl-4-pyridyl group, a 2-(2,2,2-trifluoroethyl)-4-pyridyl group, a 3-(2,2,2-trifluoroethyl)-4-pyridyl group, a 2-pentafluoroethyl-4-pyridyl group, a 3-pentafluoroethyl-4-pyridyl group, a 2-heptafluoropropyl-4-pyridyl group, a 3-heptafluoropropyl-4-pyridyl group, a 2-heptafluoroisopropyl-4-pyridyl group, a 3-heptafluoroisopropyl-4-pyridyl group, a 3-pyridazinyl group, a 4-methyl-3-pyridazinyl group, a 5-methyl-3-pyridazinyl group, a 6-methyl-3-pyridazinyl group, a 4-ethyl-3-pyridazinyl group, a 5-ethyl-3-pyridazinyl group, a 6-ethyl-3-pyridazinyl group, a 4-propyl-3-pyridazinyl group, a 5-propyl-3-pyridazinyl group, a 6-propyl-3-pyridazinyl group, a 4-isopropyl-3-pyridazinyl group, a 5-isopropyl-3-pyridazinyl group, a 6-isopropyl-3-pyridazinyl group, a 4-butyl-3-pyridazinyl group, a 5-butyl-3-pyridazinyl group, a 6-butyl-3-pyridazinyl group, a 4-isobutyl-3-pyridazinyl group, a 5-isobutyl-3-pyridazinyl group, a 6-isobutyl-3-pyridazinyl group, a 4-sec-butyl-3-pyridazinyl group, a 5-sec-butyl-3-pyridazinyl group, a 6-sec-butyl-3-pyridazinyl group, a 4-tert-butyl-3-pyridazinyl group, a 5-tert-butyl-3-pyridazinyl group, a 6-tert-butyl-3-pyridazinyl group, a 4-difluoromethyl-3-pyridazinyl group, a 5-difluoromethyl-3-pyridazinyl group, a 6-difluoromethyl-3-pyridazinyl group, a 4-trifluoromethyl-3-pyridazinyl group, a 5-trifluoromethyl-3-pyridazinyl group, a 6-trifluoromethyl-3-pyridazinyl group, a 4-(2,2,2-trifluoroethyl)-3-pyridazinyl group, a 5-(2,2,2-trifluoroethyl)-3-pyridazinyl group, a 6-(2,2,2-trifluoroethyl)-3-pyridazinyl group, a 4-pentafluoroethyl-3-pyridazinyl group, a 5-pentafluoroethyl-3-pyridazinyl group, a 6-pentafluoroethyl-3-pyridazinyl group, a 4-heptafluoropropyl-3-pyridazinyl group, a 5-heptafluoropropyl-3-pyridazinyl group, a 6-heptafluoropropyl-3-pyridazinyl group, a 4-heptafluoroisopropyl-3-pyridazinyl group, a 5-heptafluoroisopropyl-3-pyridazinyl group, a 6-heptafluoroisopropyl-3-pyridazinyl group, a 6-chloro-3-pyridazinyl group, a 6-methoxy-3-pyridazinyl group, a 6-cyano-3-pyridazinyl group, a 4-pyridazinyl group, a 3-methyl-4-pyridazinyl group, a 5-methyl-4-pyridazinyl group, a 6-methyl-4-pyridazinyl group, a 3-ethyl-4-pyridazinyl group, a 5-ethyl-4-pyridazinyl group, a 6-ethyl-4-pyridazinyl group, a 3-propyl-4-pyridazinyl group, a 5-propyl-4-pyridazinyl group, a 6-propyl-4-pyridazinyl group, a 3-isopropyl-4-pyridazinyl group, a 5-isopropyl-4-pyridazinyl group, a 6-isopropyl-4-pyridazinyl group, a 3-butyl-4-pyridazinyl group, a 5-butyl-4-pyridazinyl group, a 6-butyl-4-pyridazinyl group, a 3-isobutyl-4-pyridazinyl group, a 5-isobutyl-4-pyridazinyl group, a 6-isobutyl-4-pyridazinyl group, a 3-sec-butyl-4-pyridazinyl group, a 5-sec-butyl-4-pyridazinyl group, a 6-sec-butyl-4-pyridazinyl group, a 3-tert-butyl-4-pyridazinyl group, a 5-tert-butyl-4-pyridazinyl group, a 6-tert-butyl-4-pyridazinyl group, a 3-difluoromethyl-4-pyridazinyl group, a 5-difluoromethyl-4-pyridazinyl group, a 6-difluoromethyl-4-pyridazinyl group, a 3-trifluoromethyl-4-pyridazinyl group, a 5-trifluoromethyl-4-pyridazinyl group, a 6-trifluoromethyl-4-pyridazinyl group, a 3-(2,2,2-trifluoroethyl)-4-pyridazinyl group, a 5-(2,2,2-trifluoroethyl)-4-pyridazinyl group, a 6-(2,2,2-trifluoroethyl)-4-pyridazinyl group, a 3-pentafluoroethyl-4-pyridazinyl group, a 5-pentafluoroethyl-4-pyridazinyl group, a 6-pentafluoroethyl-4-pyridazinyl group, a 3-heptafluoropropyl-4-pyridazinyl group, a 5-heptafluoropropyl-4-pyridazinyl group, a 6-heptafluoropropyl-4-pyridazinyl group, a 3-heptafluoroisopropyl-4-pyridazinyl group, a 5-heptafluoroisopropyl-4-pyridazinyl group, a 6-heptafluoroisopropyl-4-pyridazinyl group, a 2-pyrimidinyl group, a 4-methyl-2-pyrimidinyl group, a 5-methyl-2-pyrimidinyl group, a 4-ethyl-2-pyrimidinyl group, a 5-ethyl-2-pyrimidinyl group, a 4-propyl-2-pyrimidinyl group, a 5-propyl-2-pyrimidinyl group, a 4-isopropyl-2-pyrimidinyl group, a 5-isopropyl-2-pyrimidinyl group, a 4-butyl-2-pyrimidinyl group, a 5-butyl-2-pyrimidinyl group, a 4-isobutyl-2-pyrimidinyl group, a 5-isobutyl-2-pyrimidinyl group, a 4-sec-butyl-2-pyrimidinyl group, a 5-sec-butyl-2-pyrimidinyl group, a 4-tert-butyl-2-pyrimidinyl group, a 5-tert-butyl-2-pyrimidinyl group, a 4-difluoromethyl-2-pyrimidinyl group, a 5-difluoromethyl-2-pyrimidinyl group, a 4-trifluoromethyl-2-pyrimidinyl group, a 5-trifluoromethyl-2-pyrimidinyl group, a 4-(2,2,2-trifluoroethyl)-2-pyrimidinyl group, a 5-(2,2,2-trifluoroethyl)-2-pyrimidinyl group, a 4-pentafluoroethyl-2-pyrimidinyl group, a 5-pentafluoroethyl-2-pyrimidinyl group, a 4-heptafluoropropyl-2-pyrimidinyl group, a 5-heptafluoropropyl-2-pyrimidinyl group, a 4-heptafluoroisopropyl-2-pyrimidinyl group, a 5-heptafluoroisopropyl-2-pyrimidinyl group, a 4-chloro-2-pyrimidinyl group, a 5-chloro-2-pyrimidinyl group, a 4-cyano-2-pyrimidinyl group, a 5-cyano-2-pyrimidinyl group, a 5-nitro-2-pyrimidinyl group, a 4-pyrimidinyl group, a 2-methyl-4-pyrimidinyl group, a 5-methyl-4-pyrimidinyl group, a 6-methyl-4-pyrimidinyl group, a 2-ethyl-4-pyrimidinyl group, a 5-ethyl-4-pyrimidinyl group, a 6-ethyl-4-pyrimidinyl group, a 2-propyl-4-pyrimidinyl group, a 5-propyl-4-pyrimidinyl group, a 6-propyl-4-pyrimidinyl group, a 2-isopropyl-4-pyrimidinyl group, a 5-isopropyl-4-pyrimidinyl group, a 6-isopropyl-4-pyrimidinyl group, a 2-butyl-4-pyrimidinyl group, a 5-butyl-4-pyrimidinyl group, a 6-butyl-4-pyrimidinyl group, a 2-isobutyl-4-pyrimidinyl group, a 5-isobutyl-4-pyrimidinyl group, a 6-isobutyl-4-pyrimidinyl group, a 2-sec-butyl-4-pyrimidinyl group, a 5-sec-butyl-4-pyrimidinyl group, a 6-sec-butyl-4-pyrimidinyl group, a 2-tert-butyl-4-pyrimidinyl group, a 5-tert-butyl-4-pyrimidinyl group, a 6-tert-butyl-4-pyrimidinyl group, a 2-difluoromethyl-4-pyrimidinyl group, a 5-difluoromethyl-4-pyrimidinyl group, a 6-difluoromethyl-4-pyrimidinyl group, a 2-trifluoromethyl-4-pyrimidinyl group, a 5-trifluoromethyl-4-pyrimidinyl group, a 6-trifluoromethyl-4-pyrimidinyl group, a 2-(2,2,2-trifluoroethyl)-4-pyrimidinyl group, a 5-(2,2,2-trifluoroethyl)-4-pyrimidinyl group, a 6-(2,2,2-trifluoroethyl)-4-pyrimidinyl group, a 2-pentafluoroethyl-4-pyrimidinyl group, a 5-pentafluoroethyl-4-pyrimidinyl group, a 6-pentafluoroethyl-4-pyrimidinyl group, a 2-heptafluoropropyl-4-pyrimidinyl group, a 5-heptafluoropropyl-4-pyrimidinyl group, a 6-heptafluoropropyl-4-pyrimidinyl group, a 2-heptafluoroisopropyl-4-pyrimidinyl group, a 5-heptafluoroisopropyl-4-pyrimidinyl group, a 6-heptafluoroisopropyl-4-pyrimidinyl group, a 2-chloro-4-pyrimidinyl group, a 2-cyano-4-pyrimidinyl group, a 5-chloro-4-pyrimidinyl group, a 5-cyano-4-pyrimidinyl group, a 5-nitro-4-pyrimidinyl group, a 5-pyrimidinyl group, a 2-methyl-5-pyrimidinyl group, a 4-methyl-5-pyrimidinyl group, a 2-ethyl-5-pyrimidinyl group, a 4-ethyl-5-pyrimidinyl group, a 2-propyl-5-pyrimidinyl group, a 4-propyl-5-pyrimidinyl group, a 2-isopropyl-5-pyrimidinyl group, a 4-isopropyl-5-pyrimidinyl group, a 2-butyl-5-pyrimidinyl group, a 4-butyl-5-pyrimidinyl group, a 2-isobutyl-5-pyrimidinyl group, a 4-isobutyl-5-pyrimidinyl group, a 2-sec-butyl-5-pyrimidinyl group, a 4-sec-butyl-5-pyrimidinyl group, a 2-tert-butyl-5-pyrimidinyl group, a 4-tert-butyl-5-pyrimidinyl group, a 2-difluromethyl-5-pyrimidinyl group, a 4-difluromethyl-5-pyrimidinyl group, a 2-trifluromethyl-5-pyrimidinyl group, a 4-trifluromethyl-5-pyrimidinyl group, a 2-(2,2,2-trifluoroethyl)-5-pyrimidinyl group, a 4-(2,2,2-trifluoroethyl)-5-pyrimidinyl group, a 2-pentafluoroethyl-5-pyrimidinyl group, a 4-pentafluoroethyl-5-pyrimidinyl group, a 2-heptafluoropropyl-5-pyrimidinyl group, a 4-heptafluoropropyl-5-pyrimidinyl group, a 2-heptafluoroisopropyl-5-pyrimidinyl group, a 4-heptafluoroisopropyl-5-pyrimidinyl group, a 2-pyrazinyl group, a 3-methyl-2-pyrazinyl group, a 5-methyl-2-pyrazinyl group, a 6-methyl-2-pyrazinyl group, a 3-ethyl-2-pyrazinyl group, a 5-ethyl-2-pyrazinyl group, a 6-ethyl-2-pyrazinyl group, a 3-propyl-2-pyrazinyl group, a 5-propyl-2-pyrazinyl group, a 6-propyl-2-pyrazinyl group, a 3-isopropyl-2-pyrazinyl group, a 5-isopropyl-2-pyrazinyl group, a 6-isopropyl-2-pyrazinyl group, a 3-butyl-2-pyrazinyl group, a 5-butyl-2-pyrazinyl group, a 6-butyl-2-pyrazinyl group, a 3-isobutyl-2-pyrazinyl group, a 5-isobutyl-2-pyrazinyl group, a 6-isobutyl-2-pyrazinyl group, a 3-sec-butyl-2-pyrazinyl group, a 5-sec-butyl-2-pyrazinyl group, a 6-sec-butyl-2-pyrazinyl group, a 3-tert-butyl-2-pyrazinyl group, a 5-tert-butyl-2-pyrazinyl group, a 6-tert-butyl-2-pyrazinyl group, a 3-difluoromethyl-2-pyrazinyl group, a 5-difluoromethyl-2-pyrazinyl group, a 6-difluoromethyl-2-pyrazinyl group, a 3-trifluoromethyl-2-pyrazinyl group, a 5-trifluoromethyl-2-pyrazinyl group, a 6-trifluoromethyl-2-pyrazinyl group, a 3-(2,2,2-trifluoroethyl)-2-pyrazinyl group, a 5-(2,2,2-trifluoroethyl)-2-pyrazinyl group, a 6-(2,2,2-trifluoroethyl)-2-pyrazinyl group, a 3-pentafluoroethyl-2-pyrazinyl group, a 5-pentafluoroethyl-2-pyrazinyl group, a 6-pentafluoroethyl-2-pyrazinyl group, a 3-heptafluoropropyl-2-pyrazinyl group, a 5-heptafluoropropyl-2-pyrazinyl group, a 6-heptafluoropropyl-2-pyrazinyl group, a 3-heptafluoroisopropyl-2-pyrazinyl group, a 5-heptafluoroisopropyl-2-pyrazinyl group, a 6-heptafluoroisopropyl-2-pyrazinyl group, a 3-chloro-2-pyrazinyl group, a 3-cyano-2-pyrazinyl group, a 3-nitro-2-pyrazinyl group, a 5-chloro-2-pyrazinyl group, a 5-cyano-2-pyrazinyl group, a 5-nitro-2-pyrazinyl group, a 6-chloro-2-pyrazinyl group, a 4-(1,2,3-triazinyl) group, a 5-methyl-4-(1,2,3-triazinyl) group, a 6-methyl-4-(1,2,3-triazinyl) group, a 5-ethyl-4-(1,2,3-triazinyl) group, a 6-ethyl-4-(1,2,3-triazinyl) group, a 5-propyl-4-(1,2,3-triazinyl) group, a 6-propyl-4-(1,2,3-triazinyl) group, a 5-isopropyl-4-(1,2,3-triazinyl) group, a 6-isopropyl-4-(1,2,3-triazinyl) group, a 5-butyl-4-(1,2,3-triazinyl) group, a 6-butyl-4-(1,2,3-triazinyl) group, a 5-isobutyl-4-(1,2,3-triazinyl) group, a 6-isobutyl-4-(1,2,3-triazinyl) group, a 5-sec-butyl-4-(1,2,3-triazinyl) group, a 6-sec-butyl-4-(1,2,3-triazinyl) group, a 5-tert-butyl-4-(1,2,3-triazinyl) group, a 6-tert-butyl-4-(1,2,3-triazinyl) group, a 5-difluoromethyl-4-(1,2,3-triazinyl) group, a 6-difluoromethyl-4-(1,2,3-triazinyl) group, a 5-trifluoromethyl-4-(1,2,3-triazinyl) group, a 6-trifluoromethyl-4-(1,2,3-triazinyl) group, a 5-(2,2,2-trifluoroethyl)-4-(1,2,3-triazinyl) group, a 6-(2,2,2-trifluoroethyl)-4-(1,2,3-triazinyl) group, a 5-pentafluoroethyl-4-(1,2,3-triazinyl) group, a 6-pentafluoroethyl-4-(1,2,3-triazinyl) group, a 5-heptafluoropropyl-4-(1,2,3-triazinyl) group, a 6-heptafluoropropyl-4-(1,2,3-triazinyl) group, a 5-heptafluoroisopropyl-4-(1,2,3-triazinyl) group, a 6-heptafluoroisopropyl-4-(1,2,3-triazinyl) group, a 5-(1,2,3-triazinyl) group, a 4-methyl-5-(1,2,3-triazinyl)group, a 4-ethyl-5-(1,2,3-triazinyl)group, a 4-propyl-5-(1,2,3-triazinyl)group, a 4-isopropyl-5-(1,2,3-triazinyl)group, a 4-butyl-5-(1,2,3-triazinyl)group, a 4-isobutyl-5-(1,2,3-triazinyl)group, a 4-sec-butyl-5-(1,2,3-triazinyl)group, a 4-tert-butyl-5-(1,2,3-triazinyl)group, a 4-difluoromethyl-5-(1,2,3-triazinyl)group, a 4-trifluoromethyl-5-(1,2,3-triazinyl)group, a 4-(2,2,2-trifluoroethyl)-5-(1,2,3-triazinyl)group, a 4-pentafluoroethyl-5-(1,2,3-triazinyl)group, a 4-heptafluopropyl-5-(1,2,3-triazinyl)group, a 4-heptafluoisopropyl-5-(1,2,3-triazinyl)group, a 3-(1,2,4-triazinyl)group, a 5-methyl-3-(1,2,4-triazinyl)group, a 6-methyl-3-(1,2,4-triazinyl)group, a 5-ethyl-3-(1,2,4-triazinyl)group, a 6-ethyl-3-(1,2,4-triazinyl)group, a 5-propyl-3-(1,2,4-triazinyl)group, a 6-propyl-3-(1,2,4-triazinyl)group, a 5-isopropyl-3-(1,2,4-triazinyl)group, a 6-isopropyl-3-(1,2,4-triazinyl)group, a 5-butyl-3-(1,2,4-triazinyl)group, a 6-butyl-3-(1,2,4-triazinyl)group, a 5-isobutyl-3-(1,2,4-triazinyl)group, a 6-isobutyl-3-(1,2,4-triazinyl)group, a 5-sec-butyl-3-(1,2,4-triazinyl)group, a 6-sec-butyl-3-(1,2,4-triazinyl)group, a 5-tert-butyl-3-(1,2,4-triazinyl)group, a 6-tert-butyl-3-(1,2,4-triazinyl) group, a 5-difluoromethyl-3-(1,2,4-triazinyl)group, a 6-difluoromethyl-3-(1,2,4-triazinyl)group, a 5-trifluoromethyl-3-(1,2,4-triazinyl)group, a 6-trifluoromethyl-3-(1,2,4-triazinyl)group, a 5-(2,2,2-trifluoroethyl)-3-(1,2,4-triazinyl)group, a 6-(2,2,2-trifluoroethyl)-3-(1,2,4-triazinyl) group, a 5-pentafluoroethyl-3-(1,2,4-triazinyl)group, a 6-pentafluoroethyl-3-(1,2,4-triazinyl)group, a 5-heptafluoropropyl-3-(1,2,4-triazinyl) group, a 6-heptafluoropropyl-3-(1,2,4-triazinyl) group, a 5-heptafluoroisopropyl-3-(1,2,4-triazinyl) group, a 6-heptafluoroisopropyl-3-(1,2,4-triazinyl)group, a 5-(1,2,4-triazinyl)group, a 3-methyl-5-(1,2,4-triazinyl)group, a 6-methyl-5-(1,2,4-triazinyl) group, a 3-ethyl-5-(1,2,4-triazinyl)group, a 6-ethyl-5-(1,2,4-triazinyl)group, a 3-propyl-5-(1,2,4-triazinyl)group, a 6-propyl-5-(1,2,4-triazinyl)group, a 3-isopropyl-5-(1,2,4-triazinyl)group, a 6-isopropyl-5-(1,2,4-triazinyl)group, a 3-butyl-5-(1,2,4-triazinyl)group, a 6-butyl-5-(1,2,4-triazinyl)group, a 3-isobutyl-5-(1,2,4-triazinyl) group, a 6-isobutyl-5-(1,2,4-triazinyl)group, a 3-sec-butyl-5-(1,2,4-triazinyl)group, a 6-sec-butyl-5-(1,2,4-triazinyl) group, a 3-tert-butyl-5-(1,2,4-triazinyl)group, a 6-tert-butyl-5-(1,2,4-triazinyl)group, a 3-difluoromethyl-5-(1,2,4-triazinyl)group, a 6-difluoromethyl-5-(1,2,4-triazinyl)group, a 3-trifluoromethyl-5-(1,2,4-triazinyl)group, a 6-trifluoromethyl-5-(1,2,4-triazinyl)group, a 3-(2,2,2-trifluoroethyl)-5-(1,2,4-triazinyl)group, a 6-(2,2,2-trifluoroethyl)-5-(1,2,4-triazinyl)group, a 3-pentafluoroethyl-5-(1,2,4-triazinyl)group, a 6-pentafluoroethyl-5-(1,2,4-triazinyl)group, a 3-heptafluoropropyl-5-(1,2,4-triazinyl)group, a 6-heptafluoropropyl-5-(1,2,4-triazinyl)group, a 3-heptafluoroisopropyl-5-(1,2,4-triazinyl) group, a 6-heptafluoroisopropyl-5-(1,2,4-triazinyl)group, a 6-(1,2,4-triazinyl)group, a 3-methyl-6-(1,2,4-triazinyl)group, a 5-methyl-6-(1,2,4-triazinyl)group, a 3-ethyl-6-(1,2,4-triazinyl)group, a 5-ethyl-6-(1,2,4-triazinyl)group, a 3-propyl-6-(1,2,4-triazinyl)group, a 5-propyl-6-(1,2,4-triazinyl)group, a 3-isopropyl-6-(1,2,4-triazinyl)group, a 5-isopropyl-6-(1,2,4-triazinyl)group, a 3-butyl-6-(1,2,4-triazinyl)group, a 5-butyl-6-(1,2,4-triazinyl)group, a 3-isobutyl-6-(1,2,4-triazinyl)group, a 5-isobutyl-6-(1,2,4-triazinyl)group, a 3-sec-butyl-6-(1,2,4-triazinyl)group, a 5-sec-butyl-6-(1,2,4-triazinyl)group, a 3-tert-butyl-6-(1,2,4-triazinyl)group, a 5-tert-butyl-6-(1,2,4-triazinyl) group, a 3-difluoromethyl-6-(1,2,4-triazinyl) group, a 5-difluoromethyl-6-(1,2,4-triazinyl)group, 3-trifluoromethyl-6-(1,2,4-triazinyl) group, 5-trifluoromethyl-6-(1,2,4-triazinyl)group, a 3-(2,2,2-trifluoroethyl)-6-(1,2,4-triazinyl)group, a 5-(2,2,2-trifluoroethyl)-6-(1,2,4-triazinyl)group, a 3-pentafluoroethyl-6-(1,2,4-triazinyl)group, a 5-pentafluoroethyl-6-(1,2,4-triazinyl)group, a 3-heptafluoropropyl-6-(1,2,4-triazinyl) group, a 5-heptafluoropropyl-6-(1,2,4-triazinyl)group, 3-heptafluoroisopropyl-6-(1,2,4-triazinyl)group, a 5-heptafluoroisopropyl-6-(1,2,4-triazinyl)group, a 2-(1,3,5-triazinyl)group, a 4-chloro-2-(1,3,5-triazinyl)group, a 4,6-dichloro-2-(1,3,5-triazinyl)group, a 4-methyl-2-(1,3,5-triazinyl)group, a 4-ethyl-2-(1,3,5-triazinyl)group, a 4-propyl-2-(1,3,5-triazinyl)group, a 4-isopropyl-2-(1,3,5-triazinyl)group, a 4-butyl-2-(1,3,5-triazinyl)group, a 4-isobutyl-2-(1,3,5-triazinyl)group, a 4-sec-butyl-2-(1,3,5-triazinyl)group, a 4-tert-butyl-2-(1,3,5-triazinyl)group, a 4-difluoromethyl-2-(1,3,5-triazinyl)group, a 4-trifluoromethyl-2-(1,3,5-triazinyl)group, a 4-(2,2,2-trifluoroethyl)-2-(1,3,5-triazinyl)group, 4-pentafluoroethyl-2-(1,3,5-triazinyl)group, 4-heptafluoropropyl-2-(1,3,5-triazinyl)group, 4-heptafluoroisopropyl-2-(1,3,5-triazinyl)group, and the like.

Examples of the “6-membered aromatic heterocyclic C1-C3 alkyl group” in the compound of the present invention include a 2-pyridylmethyl group, a 2-(2-pyridyl)ethyl group, a 1-(2-pyridyl)propyl group, a 3-pyridylmethyl group, a 4-pyridylmethyl group, a 3-pyridazinylmethyl group, a 2-pyrimidinylmethyl group, a 2-pyrazinylmethyl group, a 1-[4-(1,2,3-triazinyl)]ethyl group, and the like.

Examples of the “6-membered aromatic heterocyclic C1-C3 alkyl group in which a 6-membered aromatic heterocyclic portion may have optionally one or more groups selected from the group Y” in the compound of the present invention include a 2-pyridylmethyl group, a 3-fluoro-2-pyridylmethyl group, a (5-chloro-2-pyridyl)methyl group, a (5-trifluoromethyl-2-pyridyl)methyl group, a 2-(4-chloro-2-pyridyl)ethyl group, a 1-(5-bromo-2-pyridyl)propyl group, a (6-bromo-2-pyridyl)methyl group, a (3-iodo-2-pyridyl)methyl group, a (4-cyano-2-pyridyl)methyl group, a (5-nitro-2-pyridyl)methyl group, a (6-methyl-2-pyridyl)methyl group, a (3-difluoromethyl-2-pyridyl)methyl group, a (4-trifluoromethyl-2-pyridyl)methyl group, a 3-pyridylmethyl group, a (6-chloro-3-pyridyl)methyl group, a 4-pyridylmethyl group, a (2-chloro-4-pyridyl)methyl group, a (4-methyl-3-pyridazinyl)methyl group, a (6-difluoromethyl-3-pyridazinyl)methyl group, a (4-trifluoromethyl-3-pyridazinyl)methyl group, a (4-methyl-2-pyrimidinyl)methyl group, a (5-difluoromethyl-2-pyrimidinyl)methyl group, a (5-trifluoromethyl-2-pyrimidinyl)methyl group, a (5-isopropyl-2-pyrazinyl)methyl group, a (5-difluoromethyl-2-pyrazinyl)methyl group, a (6-trifluoromethyl-2-pyrazinyl)methyl group, a [3-(2,2,2-trifluoroethyl)-2-pyrazinyl]methyl group, a 1-[5-tert-butyl-4-(1,2,3-triazinyl)]ethyl group, and the like.

Examples of the “5-membered aromatic heterocyclic group” in the compound of the present invention include a 1-pyrazolyl group, a 1-imidazolyl group, a 1-pyrrolyl group, a 2-pyrrolyl group, a 3-pyrrolyl group, a 1-(1,2,4-triazolyl)group, a 1-(1,2,3,4-tetrazolyl)group, a 1-(1,2,3,5-tetrazolyl)group, a 2-furyl group, a 3-furyl group, a 2-thienyl group, a 3-thienyl group, and the like.

Examples of the “5-membered aromatic heterocyclic group optionally having one or more groups selected from the group Y” in the compound of the present invention include a 1-pyrazolyl group, a 3-chloro-1-pyrazolyl group, a 3-bromo-1-pyrazolyl group, a 3-nitro-1-pyrazolyl group, a 3-methyl-1-pyrazolyl group, a 3-trifluoromethyl-1-pyrazolyl group, a 4-methyl-1-pyrazolyl group, a 4-chloro-1-pyrazolyl group, a 4-bromo-1-pyrazolyl group, a 4-cyano-1-pyrazolyl group, a 3,5-dimethyl-1-pyrazolyl group, an 1-imidazolyl group, a 4-trifluoromethyl-1-imidazolyl group, a 1-pyrrolyl group, a 2-fluoro-1-pyrrolyl group, a 3-cyano-1-pyrrolyl group, a 2-methyl-1-pyrrolyl group, a 3-trifluoromethyl-1-pyrrolyl group, a 3-nitro-1-pyrrolyl group, a 2-pyrrolyl group, a 3-fluoro-2-pyrrolyl group, a 4-fluoro-2-pyrrolyl group, a 5-fluoro-2-pyrrolyl group, a 3-chloro-2-pyrrolyl group, a 4-chloro-2-pyrrolyl group, a 5-chloro-2-pyrrolyl group, a 3-bromo-2-pyrrolyl group, a 4-bromo-2-pyrrolyl group, a 5-bromo-2-pyrrolyl group, a 3-iodo-2-pyrrolyl group, a 4-iodo-2-pyrrolyl group, a 5-iodo-2-pyrrolyl group, a 3-cyano-2-pyrrolyl group, a 4-cyano-2-pyrrolyl group, a 5-cyano-2-pyrrolyl group, a 3-nitro-2-pyrrolyl group, a 4-nitro-2-pyrrolyl group, a 5-nitro-2-pyrrolyl group, a 3-methyl-2-pyrrolyl group, a 4-methyl-2-pyrrolyl group, a 5-methyl-2-pyrrolyl group, a 3-ethyl-2-pyrrolyl group, a 4-ethyl-2-pyrrolyl group, a 5-ethyl-2-pyrrolyl group, a 3-propyl-2-pyrrolyl group, a 4-propyl-2-pyrrolyl group, a 5-propyl-2-pyrrolyl group, a 3-isopropyl-2-pyrrolyl group, a 4-isopropyl-2-pyrrolyl group, a 5-isopropyl-2-pyrrolyl group, a 3-tert-butyl-2-pyrrolyl group, a 4-tert-butyl-2-pyrrolyl group, a 5-tert-butyl-2-pyrrolyl group, a 3-difluoromethyl-2-pyrrolyl group, a 4-difluoromethyl-2-pyrrolyl group, a 5-difluoromethyl-2-pyrrolyl group, a 3-trifluoromethyl-2-pyrrolyl group, a 4-trifluoromethyl-2-pyrrolyl group, a 5-trifluoromethyl-2-pyrrolyl group, a 3-pyrrolyl group, a 2-fluoro-3-pyrrolyl group, a 4-fluoro-3-pyrrolyl group, a 5-fluoro-3-pyrrolyl group, a 2-chloro-3-pyrrolyl group, a 4-chloro-3-pyrrolyl group, a 5-chloro-3-pyrrolyl group, a 2-bromo-3-pyrrolyl group, a 4-bromo-3-pyrrolyl group, a 5-bromo-3-pyrrolyl group, a 2-iodo-3-pyrrolyl group, a 4-iodo-3-pyrrolyl group, a 5-iodo-3-pyrrolyl group, a 2-cyano-3-pyrrolyl group, a 4-cyano-3-pyrrolyl group, a 5-cyano-3-pyrrolyl group, a 2-nitro-3-pyrrolyl group, a 4-nitro-3-pyrrolyl group, a 5-nitro-3-pyrrolyl group, a 2-methyl-3-pyrrolyl group, a 4-methyl-3-pyrrolyl group, a 5-methyl-3-pyrrolyl group, a 2-ethyl-3-pyrrolyl group, a 4-ethyl-3-pyrrolyl group, a 5-ethyl-3-pyrrolyl group, a 2-propyl-3-pyrrolyl group, a 4-propyl-3-pyrrolyl group, a 5-propyl-3-pyrrolyl group, a 2-isopropyl-3-pyrrolyl group, a 4-isopropyl-3-pyrrolyl group, a 5-isopropyl-3-pyrrolyl group, a 2-tert-butyl-3-pyrrolyl group, a 4-tert-butyl-3-pyrrolyl group, a 5-tert-butyl-3-pyrrolyl group, a 2-difluoromethyl-3-pyrrolyl group, a 4-difluoromethyl-3-pyrrolyl group, a 5-difluoromethyl-3-pyrrolyl group, a 2-trifluoromethyl-3-pyrrolyl group, a 4-trifluoromethyl-3-pyrrolyl group, a 5-trifluoromethyl-3-pyrrolyl group, a 1-(1,2,4-triazolyl)group, a 3-chloro-1-(1,2,4-triazolyl)group, a 1-(1,2,3,4-tetrazolyl)group, a 1-(1,2,3,5-tetrazolyl)group, a 2-furyl group, a 3-chloro-2-furyl group, a 5-bromo-2-furyl group, a 3-iodo-2-furyl group, a 4-cyano-2-furyl group, a 5-nitro-2-furyl group, a 3-methyl-2-furyl group, a 4-tert-butyl-2-furyl group, a 5-methyl-2-furyl group, a 5-trifluoromethyl-2-furyl group, a 3-furyl group, a 2-fluoro-3-furyl group, a 4-chloro-3-furyl group, a 2-bromo-3-furyl group, a 5-bromo-3-furyl group, a 2-iodo-3-furyl group, a 4-cyano-3-furyl group, a 4-nitro-3-furyl group, a 2-methyl-3-furyl group, a 2-tert-butyl-3-furyl group, a 4-difluoromethyl-3-furyl group, a 5-difluoromethyl-3-furyl group, a 2-trifluoromethyl-3-furyl group, a 4-trifluoromethyl-3-furyl group, a 5-trifluoromethyl-3-furyl group, a 2-thienyl group, a 3-fluoro-2-thienyl group, a 4-fluoro-2-thienyl group, a 5-fluoro-2-thienyl group, a 3-chloro-2-thienyl group, a 4-chloro-2-thienyl group, a 5-chloro-2-thienyl group, a 3-bromo-2-thienyl group, a 4-bromo-2-thienyl group, a 5-bromo-2-thienyl group, a 3-iodo-2-thienyl group, a 4-iodo-2-thienyl group, a 5-iodo-2-thienyl group, a 3-cyano-2-thienyl group, a 4-cyano-2-thienyl group, a 5-cyano-2-thienyl group, a 3-nitro-2-thienyl group, a 4-nitro-2-thienyl group, a 5-nitro-2-thienyl group, a 3-methyl-2-thienyl group, a 4-methyl-2-thienyl group, a 5-methyl-2-thienyl group, a 3-ethyl-2-thienyl group, a 4-ethyl-2-thienyl group, a 5-ethyl-2-thienyl group, a 3-propyl-2-thienyl group, a 4-propyl-2-thienyl group, a 5-propyl-2-thienyl group, a 3-isopropyl-2-thienyl group, a 4-isopropyl-2-thienyl group, a 5-isopropyl-2-thienyl group, a 3-tert-butyl-2-thienyl group, a 4-tert-butyl-2-thienyl group, a 5-tert-butyl-2-thienyl group, a 3-difluoromethyl-2-thienyl group, a 4-difluoromethyl-2-thienyl group, a 5-difluoromethyl-2-thienyl group, a 3-trifluoromethyl-2-thienyl group, a 4-trifluoromethyl-2-thienyl group, a 5-trifluoromethyl-2-thienyl group, a 3-thienyl group, a 2-fluoro-3-thienyl group, a 4-fluoro-3-thienyl group, a 5-fluoro-3-thienyl group, a 2-chloro-3-thienyl group, a 4-chloro-3-thienyl group, a 5-chloro-3-thienyl group, a 2-bromo-3-thienyl group, a 4-bromo-3-thienyl group, a 5-bromo-3-thienyl group, a 2-iodo-3-thienyl group, a 4-iodo-3-thienyl group, a 5-iodo-3-thienyl group, a 2-cyano-3-thienyl group, a 4-cyano-3-thienyl group, a 5-cyano-3-thienyl group, a 2-nitro-3-thienyl group, a 4-nitro-3-thienyl group, a 5-nitro-3-thienyl group, a 2-methyl-3-thienyl group, a 4-methyl-3-thienyl group, a 5-methyl-3-thienyl group, a 2-ethyl-3-thienyl group, a 4-ethyl-3-thienyl group, a 5-ethyl-3-thienyl group, a 2-propyl-3-thienyl group, a 4-propyl-3-thienyl group, a 5-propyl-3-thienyl group, a 2-isopropyl-3-thienyl group, a 4-isopropyl-3-thienyl group, a 5-isopropyl-3-thienyl group, a 2-tert-butyl-3-thienyl group, a 4-tert-butyl-3-thienyl group, a 5-tert-butyl-3-thienyl group, a 2-difluoromethyl-3-thienyl group, a 4-difluoromethyl-3-thienyl group, a 5-difluoromethyl-3-thienyl group, a 2-trifluoromethyl-3-thienyl group, a 4-trifluoromethyl-3-thienyl group, a 5-trifluoromethyl-3-thienyl group, and the like.

Examples of the “C2-C6 alkylcarbonyl group” in the compound of the present invention include an acetyl group, an ethylcarbonyl group, a propylcarbonyl group, an isopropylcarbonyl group, a butylcarbonyl group, an isobutylcarbonyl group, a sec-butylcarbonyl group, a tert-butylcarbonyl group, a pentylcarbonyl group, a 2,2-dimethylpropylcarbonyl group, a 3-methylbutylcarbonyl group, a pentylcarbonyl group, and the like.

Examples of the “C2-C6 alkylcarbonyl group optionally having one or more halogen atoms” in the compound of the present invention include an acetyl group, an ethylcarbonyl group, a propylcarbonyl group, an isopropylcarbonyl group, a butylcarbonyl group, an isobutylcarbonyl group, a sec-butylcarbonyl group, a tert-butylcarbonyl group, a pentylcarbonyl group, a 2,2-dimethylpropylcarbonyl group, a 3-methylbutylcarbonyl group, a trichloroacetyl group, a difluoroacetyl group, a trifluoroacetyl group, a (2,2,2-trifluoroethyl)carbonyl group, a pentafluoroethylcarbonyl group, a heptafluoropropylcarbonyl group, a heptafluoroisopropylcarbonyl group, and the like.

Examples of the “C2-C5 alkylcarbonyl group” in the compound of the present invention include an acetyl group, an ethylcarbonyl group, a propylcarbonyl group, an isopropylcarbonyl group, a butylcarbonyl group, an isobutylcarbonyl group, a sec-butylcarbonyl group, a tert-butylcarbonyl group, and the like.

Examples of the “benzoyl group optionally having one or more groups selected from the group Y” in the compound of the present invention include a benzoyl group, a 2-fluorobenzoyl group, a 3-fluorobenzoyl group, a 4-fluorobenzoyl group, a 2-chlorobenzoyl group, a 3-chlorobenzoyl group, a 4-chlorobenzoyl group, a 2-bromobenzoyl group, a 3-bromobenzoyl group, a 4-bromobenzoyl group, a 2-iodobenzoyl group, a 3-iodobenzoyl group, a 4-iodobenzoyl group, a 2-cyanobezoyl group, a 3-cyanobezoyl group, a 4-cyanobezoyl group, a 2-nitrobenzoyl group, a 3-nitrobenzoyl group, a 4-nitrobenzoyl group, a 2-methylbenzoyl group, 3-methylbenzoyl group, 4-methylbenzoyl group, 2-ethylbenzoyl group, 3-ethylbenzoyl group, 4-ethylbenzoyl group, 2-propylbenzoyl group, 3-propylbenzoyl group, a 4-propylbenzoyl group, a 2-isopropylbenzoyl group, a 3-isopropylbenzoyl group, a 4-isopropylbenzoyl group, a 2-butylbenzoyl group, a 3-butylbenzoyl group, a 4-butylbenzoyl group, a 2-isobutylbenzoyl group, a 3-isobutylbenzoyl group, a 4-isobutylbenzoyl group, a 2-sec-butylbenzoyl group, a 3-sec-butylbenzoyl group, a 4-sec-butylbenzoyl group, a 2-tert-butylbenzoyl group, a 3-tert-butylbenzoyl group, a 4-tert-butylbenzoyl group, a 2-pentylbenzoyl group, a 3-pentylbenzoyl group, a 4-pentylbenzoyl group, a 2-(2,2-dimethylpropyl)benzoyl group, a 3-(2,2-dimethylpropyl)benzoyl group, a 4-(2,2-dimethylpropyl)benzoyl group, a 2-(3-methylbutyl)benzoyl group, a 3-(3-methylbutyl)benzoyl group, a 4-(3-methylbutyl)benzoyl group, a 2-(2,3-dimethylbutyl)benzoyl group, a 3-(2,3-dimethylbutyl)benzoyl group, a 4-(2,3-dimethylbutyl)benzoyl group, a 2-(3,3-dimethylbutyl)benzoyl group, a 3-(3,3-dimethylbutyl)benzoyl group, a 4-(3,3-dimethylbutyl)benzoyl group, a 2-hexylbenzoyl group, a 3-hexylbenzoyl group, a 4-hexylbenzoyl group, a 2-trichloromethylbenzoyl group, a 3-trichloromethylbenzoyl group, a 4-trichloromethylbenzoyl group, a 2-difluoromethylbenzoyl group, a 3-difluoromethylbenzoyl group, a 4-difluoromethylbenzoyl group, a 2-trifluoromethylbenzoyl group, a 3-trifluoromethylbenzoyl group, a 4-trifluoromethylbenzoyl group, a 2-(2,2,2-trifluoroethyl)benzoyl group, a 3-(2,2,2-trifluoroethyl)benzoyl group, a 4-(2,2,2-trifluoroethyl)benzoyl group, a 2-pentafluoroethylbenzoyl group, a 3-pentafluoroethylbenzoyl group, a 4-pentafluoroethylbenzoyl group, a 2-heptafluoropropylbenzoyl group, a 3-heptafluoropropylbenzoyl group, a 4-heptafluoropropylbenzoyl group, a 2-heptafluoroisopropylbenzoyl group, a 3-heptafluoroisopropylbenzoyl group, a 4-heptafluoroisopropylbenzoyl group, a 2-methoxybenzoyl group, a 3-methoxybenzoyl group, a 4-methoxybenzoyl group, a 2-ethoxybenzoyl group, a 3-ethoxybenzoyl group, a 4-ethoxybenzoyl group, a 2-propoxybenzoyl group, a 3-propoxybenzoyl group, a 4-propoxybenzoyl group, a 2-isopropoxybenzoyl group, a 3-isopropoxybenzoyl group, a 4-isopropoxybenzoyl group, a 2-butoxybenzoyl group, a 3-butoxybenzoyl group, a 4-butoxybenzoyl group, a 2-isobutoxybenzoyl group, a 3-isobutoxybenzoyl group, a 4-isobutoxybenzoyl group, a 2-sec-butoxybenzoyl group, a 3-sec-butoxybenzoyl group, a 4-sec-butoxybenzoyl group, a 2-tert-butoxybenzoyl group, a 3-tert-butoxybenzoyl group, a 4-tert-butoxybenzoyl group, a 2-pentyloxybenzoyl group, a 3-pentyloxybenzoyl group, a 4-pentyloxybenzoyl group, a 2-(2,2-dimethylpropoxyl)benzoyl group, a 3-(2,2-dimethylpropoxyl)benzoyl group, a 4-(2,2-dimethylpropoxyl)benzoyl group, a 2-pentafluoroethoxybenzoyl group, a 3-pentafluoroethoxybenzoyl group, a 4-pentafluoroethoxybenzoyl group, a 2-heptafluoropropoxybenzoyl group, a 3-heptafluoropropoxybenzoyl group, a 4-heptafluoropropoxybenzoyl group, a 2-heptafluoroisopropoxybenzoyl group, a 3-heptafluoroisopropoxybenzoyl group, a 4-heptafluoroisopropoxybenzoyl group, a 2-(trifluoromethoxy)benzoyl group, a 3-(trifluoromethoxy)benzoyl group, a 4-(trifluoromethoxy)benzoyl group, and the like.

Examples of the “C2-C6 alkoxycarbonyl group” in the compound of the present invention include an methoxycarbonyl group, an ethoxycarbonyl group, a propoxycarbonyl group, an isopropoxycarbonyl group, a butoxycarbonyl group, an isobutoxycarbonyl group, a sec-butoxycarbonyl group, a tert-butoxycarbonyl group, a pentyloxycarbonyl group, a 2,2-dimethylpropoxycarbonyl group, a 3-methylbutoxycarbonyl group, and the like.

Examples of the “C1-C4 alkylsulfonyl group” in the compound of the present invention include a methylsulfonyl group, an ethylsulfonyl group, a propylsulfonyl group, an isopropylsulfonyl group, a butylsulfonyl group, an isobutylsulfonyl group, a sec-butylsulfonyl group, a tert-butylsulfonyl group, and the like.

Examples of the “C1-C4 alkylsulfonyl group optionally having one or more halogen atoms” in the compound of the present invention include a methylsulfonyl group, an ethylsulfonyl group, a propylsulfonyl group, an isopropylsulfonyl group, a butylsulfonyl group, an isobutylsulfonyl group, a sec-butylsulfonyl group, a tert-butylsulfonyl group, a trichloromethylsulfonyl group, a difluoromethylsulfonyl group, a trifluoromethylsulfonyl group, a 2,2,2-trifluoroethylsulfonyl group, and the like.

Examples of the “C1-C6 alkylsulfonyl group” in the compound of the present invention include a methylsulfonyl group, an ethylsulfonyl group, a propylsulfonyl group, an isopropylsulfonyl group, a butylsulfonyl group, an isobutylsulfonyl group, a sec-butylsulfonyl group, a tert-butylsulfonyl group, a pentylsulfonyl group, a 2,2-dimethylpropylsulfonyl group, a 3-methylbutylsulfonyl group, a 2,3-dimethylbutylsulfonyl group, a 3,3-dimethylbutylsulfonyl group, a hexylsulfonyl group, and the like.

Examples of the “C1-C6 alkylsulfonyl group optionally having one or more halogen atoms” in the compound of the present invention include a methylsulfonyl group, an ethylsulfonyl group, a propylsulfonyl group, an isopropylsulfonyl group, a butylsulfonyl group, an isobutylsulfonyl group, a sec-butylsulfonyl group, a tert-butylsulfonyl group, a pentylsulfonyl group, a 2,2-dimethylpropylsulfonyl group, a 3-methylbutylsulfonyl group, a 2,3-dimethylbutylsulfonyl group, a 3,3-dimethylbutylsulfonyl group, a hexylsulfonyl group, a trichloromethylsulfonyl group, a difluoromethylsulfonyl group, a trifluoromethylsulfonyl group, a 2,2,2-trifluoroethylsulfonyl group, a pentafluoroethylsulfonyl group, a heptafluoropropylsulfonyl group, a heptafluoroisopropylsulfonyl group, and the like.

Examples of the “phenylsulfonyl group optionally having one or more groups selected from the group Y” in the compound of the present invention include a 2-fluorophenylsulfonyl group, a 3-chlorophenylsulfonyl group, a 4-chlorophenylsulfonyl group, a 2-iodophenylsulfonyl group, a 3-cyanophenylsulfonyl group, a 4-nitrophenylsulfonyl group, a 2-methylphenylsulfonyl group, a 4-methylphenylsulfonyl group, a 4-tert-butylphenylsulfonyl group, a 4-difluoromethylphenylsulfonyl group, a 2-trifluoromethylphenylsulfonyl group, a 2-(2,2,2-trifluoroethyl)phenylsulfonyl group, a 4-pentafluoroethylphenylsulfonyl group, a 4-heptafluoroisopropylphenylsulfonyl group, a 2-methoxyphenylsulfonyl group, a 3-difluoromethoxyphenylsulfonyl group, a 4-difluoromethoxyphenylsulfonyl group, a 2-trifluoromethoxyphenylsulfonyl group, a 4-(2,2,2-trifluoroethoxyl)phenylsulfonyl group, and the like.

Examples of the “C7-C9 phenylalkylsulfonyl group” in the compound of the present invention include a benzylsulfonyl group, a 1-phenylethylsulfonyl group, a 2-phenylethylsulfonyl group, a 1-phenylpropylsulfonyl group, a 2-phenylpropylsulfonyl group, a 3-phenylpropylsulfonyl group, a 1-methyl-1-phenylethylsulfonyl group, and the like.

Examples of the “C7-C9 phenylalkylsulfonyl group in which a benzene ring portion may have optionally one or more groups selected from the group Y” in the compound of the present invention include a benzylsulfonyl group, a 2-fluorobenzylsulfonyl group, a 3-chlorobenzylsulfonyl group, a 4-bromobenzylsulfonyl group, a 2-cyanobenzylsulfonyl group, a 3-nitrobenzylsulfonyl group, a 3-methoxybenzylsulfonyl group, a 4-trifluoromethoxybenzylsulfonyl group, a 1-(3-chlorophenyl)ethylsulfonyl group, a 2-(4-bromophenyl)ethylsulfonyl group, a 1-(2-cyanophenyl)propylsulfonyl group, a 2-(3-nitrophenyl)propylsulfonyl group, a 3-(3-methoxyphenyl)propylsulfonyl group, a 1-methyl-1-(4-trifluoromethoxyphenyl)ethylsulfonyl group, and the like.

Examples of the “C1-C6 alkoxy group” in the compound of the present invention include a methoxy group, an ethoxy group, a propoxy group, an isopropoxy group, a butoxy group, an isobutoxy group, a sec-butoxy group, a tert-butoxy group, a pentyloxy group, a 2,2-dimethylpropoxy group, a 3-methylbutoxy group, a 2,3-dimethylbutoxy group, a 3,3-dimethylbutoxy group, a hexyloxy group, and the like.

Examples of the “C1-C6 alkoxy group optionally having one or more halogen atoms” in the compound of the present invention include a methoxy group, an ethoxy group, a propoxy group, an isopropoxy group, a butoxy group, an isobutoxy group, a sec-butoxy group, a tert-butoxy group, a pentyloxy group, a 2,2-dimethylpropoxy group, a 3-methylbutoxy group, a 2,3-dimethylbutoxy group, a 3,3-dimethylbutoxy group, a hexyloxy group, a trichloromethoxy group, a difluoromethoxy group, a trifluoromethoxy group, a 2,2,2-trifluoroethoxy group, a pentafluoroethoxy group, a heptafluoropropoxy group, a heptafluoroisopropoxy group, and the like.

Examples of the compound of the present include the following compounds.

A compound represented by Formula (1) in which A¹ represents a nitrogen atom;

a compound represented by Formula (1) in which A¹ represents a nitrogen atom, A² represents —N(R¹²)—, and A³ represents ═C(R⁴)—;

a compound represented by Formula (1) in which A¹ represents a nitrogen atom, A² represents —N(R¹²)—, and A³ represents a nitrogen atom;

a compound represented by Formula (1) in which A¹ represents a nitrogen atom and A² represents an oxygen atom;

a compound represented by Formula (1) in which A¹ represents a nitrogen atom, A² represents an oxygen atom, and A³ represents ═C(R⁴)—;

a compound represented by Formula (1) in which A¹ represents a nitrogen atom and A² represents a sulfur atom;

a compound represented by Formula (1) in which A¹ represents a nitrogen atom, A² represents a sulfur atom, and A³ represents ═C(R⁴)—;

a compound represented by Formula (1) in which A¹ represents —C(R⁵)═;

a compound represented by Formula (1) in which A¹ represents —C(R⁵)═ and A² represents —N(R¹²)—;

a compound represented by Formula (1) in which A¹ represents —C(R⁵)═, A² represents —N(R¹²)—, and A³ represents ═C(R⁴)—;

a compound represented by Formula (1) in which A¹ represents —C(R⁵)═, A² represents an oxygen atom;

a compound represented by Formula (1) in which A¹ represents —C(R⁵)═, A² represents an oxygen atom, and A³ represents ═C(R⁴)—;

a compound represented by Formula (1) in which A² represents —N(R¹²)— and R¹² represents a hydrogen atom or a C1-C4 alkyl group optionally having one or more halogen atoms;

a compound represented by Formula (1) in which A² represents —N(R¹²)— and R¹² represents a hydrogen atom;

a compound represented by Formula (1) in which A² represents —N(R¹²)— and R¹² represents a C1-C4 alkyl group optionally having one or more halogen atoms;

a compound represented by Formula (1) in which A² represents —N(R¹²)— and R¹² represents a methyl group;

a compound represented by Formula (1) in which A¹ represents a nitrogen atom, A² represents —N(R¹²)—, and R¹² represents a hydrogen atom or a C1-C4 alkyl group optionally having one or more halogen atoms;

a compound represented by Formula (1) in which A¹ represents a nitrogen atom, A² represents —N(R¹²)—, and R¹² represents a hydrogen atom;

a compound represented by Formula (1) in which A¹ represents a nitrogen atom, A² represents —N(R¹²)—, and R¹² represents a C1-C4 alkyl group optionally having one or more halogen atoms;

a compound represented by Formula (1) in which A¹ represents a nitrogen atom, A² represents —N(R¹²)—, and R¹² represents a methyl group;

a compound represented by Formula (1) in which A¹ represents a nitrogen atom, A² represents —N(R¹²)—, A³ represents ═C(R4)-, and R¹² represents a hydrogen atom or a C1-C4 alkyl group optionally having one or more halogen atoms;

a compound represented by Formula (1) in which A¹ represents a nitrogen atom, A² represents —N(R¹²)—, A³ represents ═C(R⁴)—, and R¹² represents a hydrogen atom;

a compound represented by Formula (1) in which A¹ represents a nitrogen atom, A² represents —N(R¹²)—, A³ represents ═C(R⁴)—, and R¹² represents a C1-C4 alkyl group optionally having one or more halogen atoms;

a compound represented by Formula (1) in which A¹ represents a nitrogen atom, A² represents —N(R¹²)—, A³ represents ═C(R⁴)—, and R¹² represents a methyl group;

a compound represented by Formula (1) in which A¹ represents a nitrogen atom, A² represents —N(R¹²)—, A³ represents a nitrogen atom, and R¹² represents a hydrogen atom or a C1-C4 alkyl group optionally having one or more halogen atoms;

a compound represented by Formula (1) in which A¹ represents a nitrogen atom, A² represents —N(R¹²)—, A³ represents a nitrogen atom, and R¹² represents a hydrogen atom;

a compound represented by Formula (1) in which A¹ represents a nitrogen atom, A² represents —N(R¹²)—, A³ represents a nitrogen atom, and R¹² represents a C1-C4 alkyl group optionally having one or more halogen atoms;

a compound represented by Formula (1) in which A¹ represents a nitrogen atom, A² represents —N(R¹²)—, A³ represents a nitrogen atom, and R¹² represents a methyl group;

a compound represented by Formula (1) in which A¹ represents —C(R⁵)═, A² represents —N(R¹²)—, and R¹² represents a hydrogen atom or a C1-C4 alkyl group optionally having one or more halogen atoms;

a compound represented by Formula (1) in which A¹ represents —C(R⁵)═, A² represents —N(R¹²)—, and R¹² represents a hydrogen atom;

a compound represented by Formula (1) in which A¹ represents —C(R⁵)═, A² represents —N(R¹²)—, and R¹² represents a C1-C4 alkyl group optionally having one or more halogen atoms;

a compound represented by Formula (1) in which A¹ represents —C(R⁵)═, A² represents —N(R¹²)—, and R¹² represents a methyl group;

a compound represented by Formula (1) in which A¹ represents —C(R⁵)═, A² represents —N(R¹²)—, A³ represents ═C(R⁴)—, and R¹² represents a hydrogen atom or a C1-C4 alkyl group optionally having one or more halogen atoms;

a compound represented by Formula (1) in which A¹ represents —C(R⁵)═, A² represents —N(R¹²)—, A³ represents ═C(R⁴)—, and R¹² represents a hydrogen atom;

a compound represented by Formula (1) in which A¹ represents —C(R⁵)═, A² represents —N(R¹²)—, A³ represents ═C(R⁴)—, and R¹² represents a C1-C4 alkyl group optionally having one or more halogen atoms;

a compound represented by Formula (1) in which A¹ represents —C(R⁵)═, A² represents —N(R¹²)—, A³ represents ═C(R⁴)—, and R¹² represents a methyl group;

a compound represented by Formula (1) in which A¹ represents a nitrogen atom, A² represents —N(R¹²)—, A³ represents a nitrogen atom, and R² and R³ represent a hydrogen atom;

a compound represented by Formula (1) in which A¹ represents a nitrogen atom, A² represents —N(R¹²)—, A³ represents a nitrogen atom, and R¹ and R³ represent a hydrogen atom;

a compound represented by Formula (1) in which A¹ represents a nitrogen atom, A² represents —N(R¹²)—, A³ represents a nitrogen atom, and R¹ and R² represent a hydrogen atom;

a compound represented by Formula (1) in which A¹ represents a nitrogen atom, A² represents —N(R¹²)—, A³ represents a nitrogen atom, R¹ represents a trifluoromethyl group, and R² and R³ represent a hydrogen atom;

a compound represented by Formula (1) in which A¹ represents a nitrogen atom, A² represents —N(R¹²)—, A³ represents a nitrogen atom, R² represents a trifluoromethyl group, and R¹ and R³ represent a hydrogen atom;

a compound represented by Formula (1) in which A¹ represents a nitrogen atom, A² represents —N(R¹²)—, A³ represents a nitrogen atom, R³ represents a trifluoromethyl group, and R¹ and R² represent a hydrogen atom;

a compound represented by Formula (1) in which A¹ represents a nitrogen atom, A² represents —N(R¹²)—, A³ represents a nitrogen atom, R¹ represents a trifluoromethyl group;

a compound represented by Formula (1) in which A¹ represents a nitrogen atom, A² represents —N(R¹²)—, A³ represents a nitrogen atom, R² represents a trifluoromethyl group;

a compound represented by Formula (1) in which A¹ represents a nitrogen atom, A² represents —N(R¹²)—, A³ represents a nitrogen atom, R³ represents a trifluoromethyl group;

a compound represented by Formula (1) in which A¹ represents a nitrogen atom, A² represents —N(R¹²)—, A³ represents a nitrogen atom, R¹ represents a halogen atom, and R² and R³ represent a hydrogen atom;

a compound represented by Formula (1) in which A¹ represents a nitrogen atom, A² represents —N(R¹²)—, A³ represents a nitrogen atom, R² represents a halogen atom, and R¹ and R³ represent a hydrogen atom;

a compound represented by Formula (1) in which A¹ represents a nitrogen atom, A² represents —N(R¹²)—, A³ represents a nitrogen atom, and R¹ represents a halogen atom;

a compound represented by Formula (1) in which A¹ represents a nitrogen atom, A² represents —N(R¹²)—, A³ represents a nitrogen atom, and R² represents a halogen atom;

a compound represented by Formula (1) in which A¹ represents a nitrogen atom, A² represents —N(R¹²)—, A³ represents a nitrogen atom, R¹ represents a C1-C6 alkyl group optionally having one or more groups selected from the group X, and R² and R³ represent a hydrogen atom;

a compound represented by Formula (1) in which A¹ represents a nitrogen atom, A² represents —N(R¹²)—, A³ represents a nitrogen atom, R² represents a C1-C6 alkyl group optionally having one or more groups selected from the group X, and R¹ and R³ represent a hydrogen atom;

a compound represented by Formula (1) in which A¹ represents a nitrogen atom, A² represents —N(R¹²)—, A³ represents a nitrogen atom, R³ represents a C1-C6 alkyl group optionally having one or more groups selected from the group X, and R¹ and R² represent a hydrogen atom;

a compound represented by Formula (1) in which A¹ represents a nitrogen atom, A² represents —N(R¹²)—, A³ represents a nitrogen atom, and R¹ represents a C1-C6 alkyl group optionally having one or more groups selected from the group X;

a compound represented by Formula (1) in which A¹ represents a nitrogen atom, A² represents —N(R¹²)—, A³ represents a nitrogen atom, and R² represents a C1-C6 alkyl group optionally having one or more groups selected from the group X;

a compound represented by Formula (1) in which A¹ represents a nitrogen atom, A² represents —N(R¹²)—, A³ represents a nitrogen atom, and R³ represents a C1-C6 alkyl group optionally having one or more groups selected from the group X;

a compound represented by Formula (1) in which A¹ represents a nitrogen atom, A² represents —N(R¹²)—, A³ represents a nitrogen atom, R¹ represents a phenyl group optionally having one or more groups selected from the group Y, and R² and R³ represent a hydrogen atom;

a compound represented by Formula (1) in which A¹ represents a nitrogen atom, A² represents —N(R¹²)—, A³ represents a nitrogen atom, R² represents a phenyl group optionally having one or more groups selected from the group Y, and R¹ and R³ represent a hydrogen atom;

a compound represented by Formula (1) in which A¹ represents a nitrogen atom, A² represents —N(R¹²)—, A³ represents a nitrogen atom, R³ represents a phenyl group optionally having one or more groups selected from the group Y, and R¹ and R² represent a hydrogen atom;

a compound represented by Formula (1) in which A¹ represents a nitrogen atom, A² represents —N(R¹²)—, A³ represents a nitrogen atom, and R¹ represents a phenyl group optionally having one or more groups selected from the group Y;

a compound represented by Formula (1) in which A¹ represents a nitrogen atom, A² represents —N(R¹²)—, A³ represents a nitrogen atom, and R² represents a phenyl group optionally having one or more groups selected from the group Y;

a compound represented by Formula (1) in which A¹ represents a nitrogen atom, A² represents —N(R¹²)—, A³ represents a nitrogen atom, and R³ represents a phenyl group optionally having one or more groups selected from the group Y;

a compound represented by Formula (1) in which A¹ represents a nitrogen atom, A² represents —N(R¹²)—, A³ represents a nitrogen atom, R¹ represents a 6-membered aromatic heterocyclic group optionally having one or more groups selected from the group Y or a 5-membered aromatic heterocyclic group optionally having one or more groups selected from the group Y, and R² and R³ represent a hydrogen atom;

a compound represented by Formula (1) in which A¹ represents a nitrogen atom, A² represents —N(R¹²)—, A³ represents a nitrogen atom, R² represents a 6-membered aromatic heterocyclic group optionally having one or more groups selected from the group Y or a 5-membered aromatic heterocyclic group optionally having one or more groups selected from the group Y, and R¹ and R³ represent a hydrogen atom;

a compound represented by Formula (1) in which A¹ represents a nitrogen atom, A² represents —N(R¹²)—, A³ represents a nitrogen atom, R³ represents a 6-membered aromatic heterocyclic group optionally having one or more groups selected from the group Y or a 5-membered aromatic heterocyclic group optionally having one or more groups selected from the group Y, and R¹ and R² represent a hydrogen atom;

a compound represented by Formula (1) in which A¹ represents a nitrogen atom, A² represents —N(R¹²)—, A³ represents a nitrogen atom, and R¹ represents a 6-membered aromatic heterocyclic group optionally having one or more groups selected from the group Y or a 5-membered aromatic heterocyclic group optionally having one or more groups selected from the group Y;

a compound represented by Formula (1) in which A¹ represents a nitrogen atom, A² represents —N(R¹²)—, A³ represents a nitrogen atom, and R² represents a 6-membered aromatic heterocyclic group optionally having one or more groups selected from the group Y or a 5-membered aromatic heterocyclic group optionally having one or more groups selected from the group Y;

a compound represented by Formula (1) in which A¹ represents a nitrogen atom, A² represents —N(R¹²)—, A³ represents a nitrogen atom, and R³ represents a 6-membered aromatic heterocyclic group optionally having one or more groups selected from the group Y or a 5-membered aromatic heterocyclic group optionally having one or more groups selected from the group Y;

a compound represented by Formula (1) in which A¹ represents a nitrogen atom, A² represents —N(R¹²)—, A³ represents a nitrogen atom, R¹ represents a C2-C6 alkylcarbonyl group optionally having one or more halogen atoms, and R² and R³ represent a hydrogen atom;

a compound represented by Formula (1) in which A¹ represents a nitrogen atom, A² represents —N(R¹²)—, A³ represents a nitrogen atom, R² represents a C2-C6 alkylcarbonyl group optionally having one or more halogen atoms, and R¹ and R³ represent a hydrogen atom;

a compound represented by Formula (1) in which A¹ represents a nitrogen atom, A² represents —N(R¹²)—, A³ represents a nitrogen atom, and R¹ represents a C2-C6 alkylcarbonyl group optionally having one or more halogen atoms;

a compound represented by Formula (1) in which A¹ represents a nitrogen atom, A² represents —N(R¹²)—, A³ represents a nitrogen atom, and R² represents a C2-C6 alkylcarbonyl group optionally having one or more halogen atoms;

a compound represented by Formula (1) in which A¹ represents a nitrogen atom, A² represents —N(R¹²)—, A³ represents a nitrogen atom, R¹ represents a carboxy group, and R² and R³ represent a hydrogen atom;

a compound represented by Formula (1) in which A¹ represents a nitrogen atom, A² represents —N(R¹²)—, A³ represents a nitrogen atom, R² represents a carboxy group, and R¹ and R³ represent a hydrogen atom;

a compound represented by Formula (1) in which A¹ represents a nitrogen atom, A² represents —N(R¹²)—, A³ represents a nitrogen atom, and R¹ represents a carboxy group;

a compound represented by Formula (1) in which A¹ represents a nitrogen atom, A² represents —N(R¹²)—, A³ represents a nitrogen atom, and R² represents a carboxy group;

a compound represented by Formula (1) in which A¹ represents a nitrogen atom, A² represents —N(R¹²)—, A³ represents a nitrogen atom, R¹ represents a C2-C6 alkoxycarbonyl group, and R² and R³ represent a hydrogen atom;

a compound represented by Formula (1) in which A¹ represents a nitrogen atom, A² represents —N(R¹²)—, A³ represents a nitrogen atom, R² represents a C2-C6 alkoxycarbonyl group, and R¹ and R³ represent a hydrogen atom;

a compound represented by Formula (1) in which A¹ represents a nitrogen atom, A² represents —N(R¹²)—, A³ represents a nitrogen atom, and R¹ represents a C2-C6 alkoxycarbonyl group;

a compound represented by Formula (1) in which A¹ represents a nitrogen atom, A² represents —N(R¹²)—, A³ represents a nitrogen atom, and R² represents a C2-C6 alkoxycarbonyl group;

a compound represented by Formula (1) in which A¹ represents a nitrogen atom, A² represents —N(R¹²)—, A³ represents a nitrogen atom, and R¹, R², and R³ may be the same or different and each represents a hydrogen atom, a halogen atom, a cyano group, a nitro group, a C1-C6 alkyl group optionally having one or more groups selected from the group X, a phenyl group optionally having one or more groups selected from the group Y, a carboxy group, a C2-C6 alkoxycarbonyl group, an aminocarbonyl group, —NR¹¹R¹², —OR¹¹, or —S(O)ₘR¹¹.

a compound represented by Formula (1) in which A¹ represents a nitrogen atom, A² represents —N(R¹²)—, A³ represents a nitrogen atom, R¹, R², and R³ may be the same or different and each represents a hydrogen atom, a halogen atom, a cyano group, a nitro group, a C1-C6 alkyl group optionally having one or more groups selected from the group X, a phenyl group optionally having one or more groups selected from the group Y, a carboxy group, a C2-C6 alkoxycarbonyl group, an aminocarbonyl group, —NR¹¹R¹², —OR¹¹, or —S(O)ₘR¹¹, and R¹² represents a hydrogen atom or a C1-C4 alkyl group optionally having one or more halogen atoms.

a compound represented by Formula (1) in which A¹ represents a nitrogen atom, A² represents —N(R¹²)—, A³ represents a nitrogen atom, R¹, R², and R³ may be the same or different and each represents a hydrogen atom, a halogen atom, a cyano group, a nitro group, a C1-C6 alkyl group optionally having one or more groups selected from the group X, a phenyl group optionally having one or more groups selected from the group Y, a carboxy group, a C2-C6 alkoxycarbonyl group, an aminocarbonyl group, —NR¹¹R¹², —OR¹¹, or —S(O)ₘR¹¹, R¹¹ represents a C1-C6 alkyl group optionally having one or more groups selected from the group X, and R¹² represents a hydrogen atom or a C1-C4 alkyl group optionally having one or more halogen atoms;

a compound represented by Formula (1) in which A¹ represents a nitrogen atom, A² represents —N(R¹²)—, A³ represents a nitrogen atom, R¹, R², and R³ may be the same or different and each represents a hydrogen atom, a halogen atom, a cyano group, a nitro group, a C1-C6 alkyl group optionally having one or more groups selected from the group X, a phenyl group optionally having one or more groups selected from the group Y, a carboxy group, a C2-C6 alkoxycarbonyl group, an aminocarbonyl group, —NR¹¹R¹², —OR¹¹, or —S(O)ₘR¹¹, R¹¹ represents a C1-C6 alkyl group optionally having one or more groups selected from the group X, and R¹² represents a hydrogen atom;

a compound represented by Formula (1) in which A¹ represents a nitrogen atom, A² represents —N(R¹²)—, A³ represents a nitrogen atom, R¹, R², and R³ may be the same or different and each represents a hydrogen atom, a halogen atom, a cyano group, a nitro group, a C1-C6 alkyl group optionally having one or more groups selected from the group X, a phenyl group optionally having one or more groups selected from the group Y, a carboxy group, a C2-C6 alkoxycarbonyl group, an aminocarbonyl group, —NR¹¹R¹², —OR¹¹, or —S(O)ₘR¹¹, R¹¹ represents a C1-C6 alkyl group optionally having one or more groups selected from the group X, and R¹² represents a C1-C4 alkyl group optionally having one or more halogen atoms;

a compound represented by Formula (1) in which A¹ represents a nitrogen atom, A² represents —N(R¹²)—, A³ represents a nitrogen atom, R¹, R², and R³ may be the same or different and each represents a hydrogen atom, a halogen atom, a cyano group, a nitro group, a C1-C6 alkyl group optionally having one or more groups selected from the group X, a phenyl group optionally having one or more groups selected from the group Y, or —OR¹¹, and R¹² represents a hydrogen atom or a C1-C4 alkyl group optionally having one or more halogen atoms;

a compound represented by Formula (1) in which A¹ represents a nitrogen atom, A² represents —N(R¹²)—, A³ represents a nitrogen atom, R¹, R², and R³ may be the same or different and each represents a hydrogen atom, a halogen atom, a cyano group, a nitro group, a C1-C6 alkyl group optionally having one or more groups selected from the group X, a phenyl group optionally having one or more groups selected from the group Y, or —OR¹¹, R¹¹ represents a C1-C6 alkyl group optionally having one or more groups selected from the group X, and R¹² represents a hydrogen atom or a C1-C4 alkyl group optionally having one or more halogen atoms;

a compound represented by Formula (1) in which A¹ represents a nitrogen atom, A² represents —N(R¹²)—, A³ represents a nitrogen atom, R¹, R², and R³ may be the same or different and each represents a hydrogen atom, a halogen atom, a cyano group, a nitro group, a C1-C6 alkyl group optionally having one or more groups selected from the group X, a phenyl group optionally having one or more groups selected from the group Y, or —OR¹¹, R¹¹ represents a C1-C6 alkyl group optionally having one or more groups from the group X, and R¹² represents a hydrogen atom;

a compound represented by Formula (1) in which A¹ represents a nitrogen atom, A² represents —N(R¹²)—, A³ represents a nitrogen atom, R¹, R², and R³ may be the same or different and each represents a hydrogen atom, a halogen atom, a cyano group, a nitro group, a C1-C6 alkyl group optionally having one or more groups selected from the group X, a phenyl group optionally having one or more groups selected from the group Y, or —OR¹¹, R¹¹ represents a C1-C6 alkyl group optionally having one or more groups from the group X, and R¹² represents a C1-C4 alkyl group optionally having one or more halogen atoms;

a compound represented by Formula (1) in which A¹ represents a nitrogen atom, A² represents —N(R¹²)—, A³ represents a nitrogen atom, W represents —OR⁶, and R¹, R², and R³ may be the same or different and each represents a hydrogen atom, a halogen atom, a cyano group, a nitro group, a C1-C6 alkyl group optionally having one or more groups selected from the group X, a phenyl group optionally having one or more groups selected from the group Y, a carboxy group, a C2-C6 alkoxycarbonyl group, an aminocarbonyl group, —NR¹¹R¹², —OR¹¹, or —S(O)ₘR¹¹;

a compound represented by Formula (1) in which A¹ represents a nitrogen atom, A² represents —N(R¹²)—, A³ represents a nitrogen atom, W represents —OR⁶, R¹, R², and R³ may be the same or different and each represents a hydrogen atom, a halogen atom, a cyano group, a nitro group, a C1-C6 alkyl group optionally having one or more groups selected from the group X, a phenyl group optionally having one or more groups selected from the group Y, a carboxy group, a C2-C6 alkoxycarbonyl group, an aminocarbonyl group, —NR¹¹R¹², —OR¹¹, or —S(O)ₘR¹¹, and R⁶ represents a hydrogen atom or C1-C6 alkyl group optionally having one or more groups selected from the group Z;

a compound represented by Formula (1) in which A¹ represents a nitrogen atom, A² represents —N(R¹²)—, A³ represents a nitrogen atom, W represents —OR⁶, R¹, R², and R³ may be the same or different and each represents a hydrogen atom, a halogen atom, a cyano group, a nitro group, a C1-C6 alkyl group optionally having one or more groups selected from the group X, a phenyl group optionally having one or more groups selected from the group Y, a carboxy group, a C2-C6 alkoxycarbonyl group, an aminocarbonyl group, —NR¹¹R¹², —OR¹¹, or —S(O)ₘR¹¹, and R⁶ represents a hydrogen atom or a C1-C6 alkyl group;

a compound represented by Formula (1) in which A¹ represents a nitrogen atom, A² represents —N(R¹²)—, A³ represents a nitrogen atom, W represents —OR⁶, R¹, R², and R³ may be the same or different and each represents a hydrogen atom, a halogen atom, a cyano group, a nitro group, a C1-C6 alkyl group optionally having one or more groups selected from the group X, a phenyl group optionally having one or more groups selected from the group Y, a carboxy group, a C2-C6 alkoxycarbonyl group, an aminocarbonyl group, —NR¹¹R¹², —OR¹¹, or —S(O)ₘR¹¹, and R⁶ represents a C1-C6 alkyl group;

a compound represented by Formula (1) in which A¹ represents a nitrogen atom, A² represents —N(R¹²)—, A³ represents a nitrogen atom, W represents —OR⁶, R¹, R², and R³ may be the same or different and each represents a hydrogen atom, a halogen atom, a cyano group, a nitro group, a C1-C6 alkyl group optionally having one or more groups selected from the group X, a phenyl group optionally having one or more groups selected from the group Y, a carboxy group, a C2-C6 alkoxycarbonyl group, an aminocarbonyl group, —NR¹¹R¹², —OR¹¹, or —S(O)ₘR¹¹, and R⁶ represents a hydrogen group;

a compound represented by Formula (1) in which A¹ represents a nitrogen atom, A² represents —N(R¹²)—, A³ represents a nitrogen atom, W represents —OR⁶, R¹, R², and R³ may be the same or different and each represents a hydrogen atom, a halogen atom, a cyano group, a nitro group, a C1-C6 alkyl group optionally having one or more groups selected from the group X, a phenyl group optionally having one or more groups selected from the group Y, or —OR¹¹, R⁶ represents a hydrogen group or a C1-C6 alkyl group, R¹¹ represents a C1-C6 alkyl group optionally having one or more groups selected from the group X, and R¹² represents a hydrogen atom or a C1-C4 alkyl group optionally having one or more halogen atoms;

a compound represented by Formula (1) in which A¹ represents a nitrogen atom, A² represents —N(R¹²)—, A³ represents a nitrogen atom, W represents —OR⁶, R¹, R², and R³ may be the same or different and each represents a hydrogen atom, a halogen atom, a cyano group, a nitro group, a C1-C6 alkyl group optionally having one or more groups selected from the group X, a phenyl group optionally having one or more groups selected from the group Y, or —OR¹¹, R⁶ represents a hydrogen group, R¹¹ represents a C1-C6 alkyl group optionally having one or more groups selected from the group X, and R¹² represents a hydrogen atom or a C1-C4 alkyl group optionally having one or more halogen atoms;

a compound represented by Formula (1) in which A¹ represents a nitrogen atom, A² represents —N(R¹²)—, A³ represents a nitrogen atom, W represents —OR⁶, R¹, R², and R³ may be the same or different and each represents a hydrogen atom, a halogen atom, a cyano group, a nitro group, a C1-C6 alkyl group optionally having one or more groups selected from the group X, a phenyl group optionally having one or more groups selected from the group Y, or —OR¹¹, R⁶ represents a C1-C6 alkyl group, R¹¹ represents a C1-C6 alkyl group optionally having one or more groups selected from the group X, and R¹² represents a hydrogen atom or a C1-C4 alkyl group optionally having one or more halogen atoms;

a compound represented by Formula (1) in which A¹ represents a nitrogen atom, A² represents —N(R¹²)—, A³ represents a nitrogen atom, W represents —OR⁶, R¹ represents a trifluoromethyl group, R², and R³ may be the same or different and each represents a hydrogen atom, a halogen atom, a cyano group, a nitro group, a C1-C6 alkyl group optionally having one or more groups selected from the group X, a phenyl group optionally having one or more groups selected from the group Y, or —OR¹¹, R⁶ represents a methyl group or an ethyl group, R¹¹ represents a C1-C6 alkyl group optionally having one or more groups selected from the group X, and R¹² represents a hydrogen atom or a C1-C4 alkyl group optionally having one or more halogen atoms;

a compound represented by Formula (1) in which A¹ represents a nitrogen atom, A² represents —N(R¹²)—, A³ represents a nitrogen atom, W represents —OR⁶, R¹, and R³ may be the same or different and each represents a hydrogen atom, a halogen atom, a cyano group, a nitro group, a C1-C6 alkyl group optionally having one or more groups selected from the group X, a phenyl group optionally having one or more groups selected from the group Y, or —OR¹¹, R² represents a trifluoromethyl group, R⁶ represents a methyl group or an ethyl group, R¹¹ represents a C1-C6 alkyl group optionally having one or more groups selected from the group X, and R¹² represents a hydrogen atom or a C1-C4 alkyl group optionally having one or more halogen atoms;

a compound represented by Formula (1) in which A¹ represents a nitrogen atom, A² represents —N(R¹²)—, A³ represents a nitrogen atom, W represents —OR⁶, R¹, and R² may be the same or different and each represents a hydrogen atom, a halogen atom, a cyano group, a nitro group, a C1-C6 alkyl group optionally having one or more groups selected from the group X, a phenyl group optionally having one or more groups selected from the group Y, or —OR¹¹, R³ represents a trifluoromethyl group, R⁶ represents a methyl group or an ethyl group, R¹¹ represents a C1-C6 alkyl group optionally having one or more groups selected from the group X, and R¹² represents a hydrogen atom or a C1-C4 alkyl group optionally having one or more halogen atoms;

a compound represented by Formula (1) in which A¹ represents a nitrogen atom, A² represents —N(R¹²)—, A³ represents a nitrogen atom, and W represents —ON═CR⁷R⁸;

a compound represented by Formula (1) in which A¹ represents a nitrogen atom, A² represents —N(R¹²)—, A³ represents a nitrogen atom, and W represents —SR⁹;

a compound represented by Formula (1) in which A¹ represents a nitrogen atom, A² represents —N(R¹²)—, A³ represents a nitrogen atom, W represents —SR⁹, and R⁹ represents a C1-C6 alkyl group;

a compound represented by Formula (1) in which A¹ represents a nitrogen atom, A² represents —N(R¹²)—, A³ represents a nitrogen atom, W represents —NR⁷R¹⁰, and R¹⁰ represents a hydrogen atom, a C1-C6 alkyl group, or a phenyl group optionally having one or more groups selected from a group Y;

a compound represented by Formula (1) in which A¹ represents a nitro atom, A² represents —N(R¹²)—, A³ represents a nitrogen atom, W represents —NR⁷R¹⁰, and R¹⁰ represents a hydrogen atom or a C1-C6 alkyl group;

a compound represented by Formula (1) in which A¹ represents a nitrogen atom, A² represents —N(R¹²)—, A³ represents ═C(R⁴)—, and R², R³, and R⁴ represent a hydrogen atom;

a compound represented by Formula (1) in which A¹ represents a nitrogen atom, A² represents —N(R¹²)—, A³ represents ═C(R⁴)—, and R¹, R³, and R⁴ represent a hydrogen atom;

a compound represented by Formula (1) in which A¹ represents a nitrogen atom, A² represents —N(R¹²)—, A³ represents ═C(R⁴)—, and R¹, R², and R⁴ represent a hydrogen atom;

a compound represented by Formula (1) in which A¹ represents a nitrogen atom, A² represents —N(R¹²)—, A³ represents ═C(R⁴)—, and R¹, R², and R³ represent a hydrogen atom;

a compound represented by Formula (1) in which A¹ represents a nitrogen atom, A² represents —N(R¹²)—, A³ represents ═C(R⁴)—, R¹ represents a trifluoromethyl group, and R², R³ and R⁴ represent a hydrogen atom;

a compound represented by Formula (1) in which A¹ represents a nitrogen atom, A² represents —N(R¹²)—, A³ represents ═C(R⁴)—, R² represents a trifluoromethyl group, and R¹, R³ and R⁴ represent a hydrogen atom;

a compound represented by Formula (1) in which A¹ represents a nitrogen atom, A² represents —N(R¹²)—, A³ represents ═C(R⁴)—, R³ represents a trifluoromethyl group, and R¹, R² and R⁴ represent a hydrogen atom;

a compound represented by Formula (1) in which A¹ represents a nitrogen atom, A² represents —N(R¹²)—, A³ represents ═C(R⁴)—, R⁴ represents a trifluoromethyl group, and R¹, R² and R³ represent a hydrogen atom;

a compound represented by Formula (1) in which A¹ represents a nitrogen atom, A² represents —N(R¹²)—, A³ represents ═C(R⁴)—, and R¹ represents a trifluoromethyl group;

a compound represented by Formula (1) in which A¹ represents a nitrogen atom, A² represents —N(R¹²)—, A³ represents ═C(R⁴)—, and R² represents a trifluoromethyl group;

a compound represented by Formula (1) in which A¹ represents a nitrogen atom, A² represents —N(R¹²)—, A³ represents ═C(R⁴)—, and R³ represents a trifluoromethyl group;

a compound represented by Formula (1) in which A¹ represents a nitrogen atom, A² represents —N(R¹²)—, A³ represents ═C(R⁴)—, and R⁴ represents a trifluoromethyl group;

a compound represented by Formula (1) in which A¹ represents a nitrogen atom, A² represents —N(R¹²)—, A³ represents ═C(R⁴)—, R¹ represents a halogen atom, and R², R³, and R⁴ represent a hydrogen atom;

a compound represented by Formula (1) in which A¹ represents a nitrogen atom, A² represents —N(R¹²)—, A³ represents ═C(R⁴)—, R² represents a halogen atom, and R¹, R³, and R⁴ represent a hydrogen atom;

a compound represented by Formula (1) in which A¹ represents a nitrogen atom, A² represents —N(R¹²)—, A³ represents ═C(R⁴)—, R³ represents a halogen atom, and R¹, R², and R⁴ represent a hydrogen atom;

a compound represented by Formula (1) in which A¹ represents a nitrogen atom, A² represents —N(R¹²)—, A³ represents ═C(R⁴)—, R⁴ represents a halogen atom, and R¹, R², and R³ represent a hydrogen atom;

a compound represented by Formula (1) in which A¹ represents a nitrogen atom, A² represents —N(R¹²)—, A³ represents ═C(R⁴)—, and R¹ represents a halogen atom;

a compound represented by Formula (1) in which A¹ represents a nitrogen atom, A² represents —N(R¹²)—, A³ represents ═C(R⁴)—, and R² represents a halogen atom;

a compound represented by Formula (1) in which A¹ represents a nitrogen atom, A² represents —N(R¹²)—, A³ represents ═C(R⁴)—, and R³ represents a halogen atom;

a compound represented by Formula (1) in which A¹ represents a nitrogen atom, A² represents —N(R¹²)—, A³ represents ═C(R⁴)—, and R⁴ represents a halogen atom;

a compound represented by Formula (1) in which A¹ represents a nitrogen atom, A² represents —N(R¹²)—, A³ represents ═C(R⁴)—, R¹ represents a C1-C6 alkyl group optionally having one or more groups selected from the group X, and R², R³, and R⁴ represent a hydrogen atom;

a compound represented by Formula (1) in which A¹ represents a nitrogen atom, A² represents —N(R¹²)—, A³ represents ═C(R⁴)—, R² represents a C1-C6 alkyl group optionally having one or more groups selected from the group X, and R¹, R³, and R⁴ represent a hydrogen atom;

a compound represented by Formula (1) in which A¹ represents a nitrogen atom, A² represents —N(R¹²)—, A³ represents ═C(R⁴)—, R³ represents a C1-C6 alkyl group optionally having one or more groups selected from the group X, and R¹, R², and R⁴ represent a hydrogen atom;

a compound represented by Formula (1) in which A¹ represents a nitrogen atom, A² represents —N(R¹²)—, A³ represents ═C(R⁴)—, R⁴ represents a C1-C6 alkyl group optionally having one or more groups selected from the group X, and R¹, R², and R³ represent a hydrogen atom;

a compound represented by Formula (1) in which A¹ represents a nitrogen atom, A² represents —N(R¹²)—, A³ represents ═C(R⁴)—, and R¹ represents a C1-C6 alkyl group optionally having one or more groups selected from the group X;

a compound represented by Formula (1) in which A¹ represents a nitrogen atom, A² represents —N(R¹²)—, A³ represents ═C(R⁴)—, and R² represents a C1-C6 alkyl group optionally having one or more groups selected from the group X;

a compound represented by Formula (1) in which A¹ represents a nitrogen atom, A² represents —N(R¹²)—, A³ represents ═C(R⁴)—, and R³ represents a C1-C6 alkyl group optionally having one or more groups selected from the group X;

a compound represented by Formula (1) in which A¹ represents a nitrogen atom, A² represents —N(R¹²)—, A³ represents ═C(R⁴)—, and R⁴ represents a C1-C6 alkyl group optionally having one or more groups selected from the group X;

a compound represented by Formula (1) in which A¹ represents a nitrogen atom, A² represents —N(R¹²)—, A³ represents ═C(R⁴)—, R¹ represents a phenyl group optionally having one or more groups selected from the group Y, and R², R³, and R⁴ represent a hydrogen atom;

a compound represented by Formula (1) in which A¹ represents a nitrogen atom, A² represents —N(R¹²)—, A³ represents ═C(R⁴)—, R² represents a phenyl group optionally having one or more groups selected from the group Y, and R¹, R³, and R⁴ represent a hydrogen atom;

a compound represented by Formula (1) in which A¹ represents a nitrogen atom, A² represents —N(R¹²)—, A³ represents ═C(R⁴)—, R³ represents a phenyl group optionally having one or more groups selected from the group Y, and R¹, R², and R⁴ represent a hydrogen atom;

a compound represented by Formula (1) in which A¹ represents a nitrogen atom, A² represents —N(R¹²)—, A³ represents ═C(R⁴)—, R⁴ represents a phenyl group optionally having one or more groups selected from the group Y, and R¹, R², and R³ represent a hydrogen atom;

a compound represented by Formula (1) in which A¹ represents a nitrogen atom, A² represents —N(R¹²)—, A³ represents ═C(R⁴)—, and R¹ represents a phenyl group optionally having one or more groups selected from the group Y;

a compound represented by Formula (1) in which A¹ represents a nitrogen atom, A² represents —N(R¹²)—, A³ represents ═C(R⁴)—, and R² represents a phenyl group optionally having one or more groups selected from the group Y;

a compound represented by Formula (1) in which A¹ represents a nitrogen atom, A² represents —N(R¹²)—, A³ represents ═C(R⁴)—, and R³ represents a phenyl group optionally having one or more groups selected from the group Y;

a compound represented by Formula (1) in which A¹ represents a nitrogen atom, A² represents —N(R¹²)—, A³ represents ═C(R⁴)—, and R⁴ represents a phenyl group optionally having one or more groups selected from the group Y;

a compound represented by Formula (1) in which A¹ represents a nitrogen atom, A² represents —N(R¹²)—, A³ represents ═C(R⁴)—, R¹ represents a 6-membered aromatic heterocyclic group optionally having one or more groups selected from the group Y or a 5-membered aromatic heterocyclic group optionally having one or more groups selected from the group Y, and R², R³, and R⁴ represent a hydrogen atom;

a compound represented by Formula (1) in which A¹ represents a nitrogen atom, A² represents —N(R¹²)—, A³ represents ═C(R⁴)—, R² represents a 6-membered aromatic heterocyclic group optionally having one or more groups selected from the group Y or a 5-membered aromatic heterocyclic group optionally having one or more groups selected from the group Y, and R¹, R³, and R⁴ represent a hydrogen atom;

a compound represented by Formula (1) in which A¹ represents a nitrogen atom, A² represents —N(R¹²)—, A³ represents ═C(R⁴)—, R³ represents a 6-membered aromatic heterocyclic group optionally having one or more groups selected from the group Y or a 5-membered aromatic heterocyclic group optionally having one or more groups selected from the group Y, and R¹, R², and R⁴ represent a hydrogen atom;

a compound represented by Formula (1) in which A¹ represents a nitrogen atom, A² represents —N(R¹²)—, A³ represents ═C(R⁴)—, R⁴ represents a 6-membered aromatic heterocyclic group optionally having one or more groups selected from the group Y or a 5-membered aromatic heterocyclic group optionally having one or more groups selected from the group Y, and R¹, R², and R³ represent a hydrogen atom;

a compound represented by Formula (1) in which A¹ represents a nitrogen atom, A² represents —N(R¹²)—, A³ represents ═C(R⁴)—, and R¹ represents a 6-membered aromatic heterocyclic group optionally having one or more groups selected from the group Y or a 5-membered aromatic heterocyclic group optionally having one or more groups selected from the group Y;

a compound represented by Formula (1) in which A¹ represents a nitrogen atom, A² represents —N(R¹²)—, A³ represents ═C(R⁴)—, and R² represents a 6-membered aromatic heterocyclic group optionally having one or more groups selected from the group Y or a 5-membered aromatic heterocyclic group optionally having one or more groups selected from the group Y;

a compound represented by Formula (1) in which A¹ represents a nitrogen atom, A² represents —N(R¹²)—, A³ represents ═C(R⁴)—, and R³ represents a 6-membered aromatic heterocyclic group optionally having one or more groups selected from the group Y or a 5-membered aromatic heterocyclic group optionally having one or more groups selected from the group Y;

a compound represented by Formula (1) in which A¹ represents a nitrogen atom, A² represents —N(R¹²)—, A³ represents ═C(R⁴)—, and R⁴ represents a 6-membered aromatic heterocyclic group optionally having one or more groups selected from the group Y or a 5-membered aromatic heterocyclic group optionally having one or more groups selected from the group Y;

a compound represented by Formula (1) in which A¹ represents a nitrogen atom, A² represents —N(R¹²)—, A³ represents ═C(R⁴)—, R¹ represents a C2-C6 alkylcarbonyl group optionally having one or more halogen atoms, and R², R³, and R⁴ represent a hydrogen atom;

a compound represented by Formula (1) in which A¹ represents a nitrogen atom, A² represents —N(R¹²)—, A³ represents ═C(R⁴)—, R² represents a C2-C6 alkylcarbonyl group optionally having one or more halogen atoms, and R¹, R³, and R⁴ represent a hydrogen atom;

a compound represented by Formula (1) in which A¹ represents a nitrogen atom, A² represents —N(R¹²)—, A³ represents ═C(R⁴)—, and R¹ represents a C2-C6 alkylcarbonyl group optionally having one or more halogen atoms;

a compound represented by Formula (1) in which A¹ represents a nitrogen atom, A² represents —N(R¹²)—, A³ represents ═C(R⁴)—, and R² represents a C2-C6 alkylcarbonyl group optionally having one or more halogen atoms;

a compound represented by Formula (1) in which A¹ represents a nitrogen atom, A² represents —N(R¹²)—, A³ represents ═C(R⁴)—, R¹ represents a carboxy group, and R², R³, and R⁴ represent a hydrogen atom;

a compound represented by Formula (1) in which A¹ represents a nitrogen atom, A² represents —N(R¹²)—, A³ represents ═C(R⁴)—, R² represents a carboxy group, and R¹, R³, and R⁴ represent a hydrogen atom;

a compound represented by Formula (1) in which A¹ represents a nitrogen atom, A² represents —N(R¹²)—, A³ represents ═C(R⁴)—, and R¹ represents a carboxy group;

a compound represented by Formula (1) in which A¹ represents a nitrogen atom, A² represents —N(R¹²)—, A³ represents ═C(R⁴)—, and R² represents a carboxy group;

a compound represented by Formula (1) in which A¹ represents a nitrogen atom, A² represents —N(R¹²)—, A³ represents ═C(R⁴)—, R¹ represents a C2-C6 alkoxycarbonyl group, and R², R³, and R⁴ represent a hydrogen atom;

a compound represented by Formula (1) in which A¹ represents a nitrogen atom, A² represents —N(R¹²)—, A³ represents ═C(R⁴)—, R² represents a C2-C6 alkoxycarbonyl group, and R¹, R³, and R⁴ represent a hydrogen atom;

a compound represented by Formula (1) in which A¹ represents a nitrogen atom, A² represents —N(R¹²)—, A³ represents ═C(R⁴)—, R³ represents a C2-C6 alkoxycarbonyl group, and R¹, R², and R⁴ represent a hydrogen atom;

a compound represented by Formula (1) in which A¹ represents a nitrogen atom, A² represents —N(R¹²)—, A³ represents ═C(R⁴)—, R⁴ represents a C2-C6 alkoxycarbonyl group, and R¹, R², and R³ represent a hydrogen atom;

a compound represented by Formula (1) in which A¹ represents a nitrogen atom, A² represents —N(R¹²)—, A³ represents ═C(R⁴)—, and R¹ represents a C2-C6 alkoxycarbonyl group;

a compound represented by Formula (1) in which A¹ represents a nitrogen atom, A² represents —N(R¹²)—, A³ represents ═C(R⁴)—, and R² represents a C2-C6 alkoxycarbonyl group;

a compound represented by Formula (1) in which A¹ represents a nitrogen atom, A² represents —N(R¹²)—, A³ represents ═C(R⁴)—, and R³ represents a C2-C6 alkoxycarbonyl group;

a compound represented by Formula (1) in which A¹ represents a nitrogen atom, A² represents —N(R¹²)—, A³ represents ═C(R⁴)—, and R⁴ represents a C2-C6 alkoxycarbonyl group;

a compound represented by Formula (1) in which A¹ represents a nitrogen atom, A² represents —N(R¹²)—, A³ represents ═C(R⁴)—, and R¹, R², R³, and R⁴ may be the same or different and each represents a hydrogen atom, a halogen atom, a cyano group, a nitro group, a C1-C6 alkyl group optionally having one or more groups selected from the group X, a phenyl group optionally having one or more groups selected from the group Y, a carboxy group, a C2-C6 alkoxycarbonyl group, an aminocarbonyl group, —NR¹¹R¹², —OR¹¹, or —S(O)ₘR¹¹;

a compound represented by Formula (1) in which A¹ represents a nitrogen atom, A² represents —N(R¹²)—, A³ represents ═C(R⁴)—, R¹, R², R³, and R⁴ may be the same or different and each represents a hydrogen atom, a halogen atom, a cyano group, a nitro group, a C1-C6 alkyl group optionally having one or more groups selected from the group X, a phenyl group optionally having one or more groups selected from the group Y, a carboxy group, a C2-C6 alkoxycarbonyl group, an aminocarbonyl group, —NR¹¹R¹², —OR¹¹, or —S(O)ₘR¹¹, and R¹² represents a hydrogen atom or a C1-C-4 alkyl group optionally having one or more halogen atom;

a compound represented by Formula (1) in which A¹ represents a nitrogen atom, A² represents —N(R¹²)—, A³ represents ═C(R⁴)—, R¹, R², R³, and R⁴ may be the same or different and each represents a hydrogen atom, a halogen atom, a cyano group, a nitro group, a C1-C6 alkyl group optionally having one or more groups selected from the group X, a phenyl group optionally having one or more groups selected from the group Y, a carboxy group, a C2-C6 alkoxycarbonyl group, an aminocarbonyl group, —NR¹¹R¹², —OR¹¹, or —S(O)ₘR¹¹, R¹¹ represents a C1-C6 alkyl group optionally having one or more groups selected from the group X, and R¹² represents a hydrogen atom or a C1-C-4 alkyl group optionally having one or more halogen atom;

a compound represented by Formula (1) in which A¹ represents a nitrogen atom, A² represents —N(R¹²)—, A³ represents ═C(R⁴)—, R¹, R², R³, and R⁴ may be the same or different and each represents a hydrogen atom, a halogen atom, a cyano group, a nitro group, a C1-C6 alkyl group optionally having one or more groups selected from the group X, a phenyl group optionally having one or more groups selected from the group Y, a carboxy group, a C2-C6 alkoxycarbonyl group, an aminocarbonyl group, —NR¹¹R¹², —OR¹¹, or —S(O)ₘR¹¹, R¹¹ represents a C1-C6 alkyl group optionally having one or more groups selected from the group X, and R¹² represents a hydrogen atom;

a compound represented by Formula (1) in which A¹ represents a nitrogen atom, A² represents —N(R¹²)—, A³ represents ═C(R⁴)—, R¹, R², R³, and R⁴ may be the same or different and each represents a hydrogen atom, a halogen atom, a cyano group, a nitro group, a C1-C6 alkyl group optionally having one or more groups selected from the group X, a phenyl group optionally having one or more groups selected from the group Y, a carboxy group, a C2-C6 alkoxycarbonyl group, an aminocarbonyl group, —NR¹¹R¹², —OR¹¹, or —S(O)ₘR¹¹, R¹¹ represents a C1-C6 alkyl group optionally having one or more groups selected from the group X, and R¹² represents a C1-C4 alkyl group optionally having one or more halogen atoms;

a compound represented by Formula (1) in which A¹ represents a nitrogen atom, A² represents —N(R¹²)—, A³ represents ═C(R⁴)—, R¹, R², R³, and R⁴ may be the same or different and each represents a hydrogen atom, a halogen atom, a cyano group, a nitro group, a C1-C6 alkyl group optionally having one or more groups selected from the group X, a phenyl group optionally having one or more groups selected from the group Y, or —OR¹¹, and R¹² represents a hydrogen atom or a C1-C4 alkyl group optionally having one or more halogen atoms;

a compound represented by Formula (1) in which A¹ represents a nitrogen atom, A² represents —N(R¹²)—, A³ represents ═C(R⁴)—, R¹, R², R³, and R⁴ may be the same or different and each represents a hydrogen atom, a halogen atom, a cyano group, a nitro group, a C1-C6 alkyl group optionally having one or more groups selected from the group X, a phenyl group optionally having one or more groups selected from the group Y, or —OR¹¹, R¹¹ represents a C1-C6 alkyl group optionally having one or more groups selected from the group X, and R¹² represents a hydrogen atom or a C1-C4 alkyl group optionally having one or more halogen atoms;

a compound represented by Formula (1) in which A¹ represents a nitrogen atom, A² represents —N(R¹²)—, A³ represents ═C(R⁴)—, R¹, R², R³, and R⁴ may be the same or different and each represents a hydrogen atom, a halogen atom, a cyano group, a nitro group, a C1-C6 alkyl group optionally having one or more groups selected from the group X, a phenyl group optionally having one or more groups selected from the group Y, or —OR¹¹, R¹¹ represents a C1-C6 alkyl group optionally having one or more groups selected from the group X, and R¹² represents a hydrogen atom;

a compound represented by Formula (1) in which A¹ represents a nitrogen atom, A² represents —N(R¹²)—, A³ represents ═C(R⁴)—, R¹, R², R³, and R⁴ may be the same or different and each represents a hydrogen atom, a halogen atom, a cyano group, a nitro group, a C1-C6 alkyl group optionally having one or more groups selected from the group X, a phenyl group optionally having one or more groups selected from the group Y, or —OR¹¹, R¹¹ represents a C1-C6 alkyl group optionally having one or more groups selected from the group X, and R¹² represents a C1-C4 alkyl group optionally having one or more halogen atoms;

a compound represented by Formula (1) in which A¹ represents a nitrogen atom, A² represents —N(R¹²)—, A³ represents ═C(R⁴)—, W represents —OR⁶, and R¹, R², R³, and R⁴ may be the same or different and each represents a hydrogen atom, a halogen atom, a cyano group, a nitro group, a C1-C6 alkyl group optionally having one or more groups selected from the group X, a phenyl group optionally having one or more groups selected from the group Y, a carboxy group, a C2-C6 alkoxycarbonyl group, an aminocarbonyl group, —NR¹¹R¹², —OR¹¹, or —S(O)ₘR¹¹;

a compound represented by Formula (1) in which A¹ represents a nitrogen atom, A² represents —N(R¹²)—, A³ represents ═C(R⁴)—, W represents —OR⁶, R¹, R², R³, and R⁴ may be the same or different and each represents a hydrogen atom, a halogen atom, a cyano group, a nitro group, a C1-C6 alkyl group optionally having one or more groups selected from the group X, a phenyl group optionally having one or more groups selected from the group Y, a carboxy group, a C2-C6 alkoxycarbonyl group, an aminocarbonyl group, —NR¹¹R¹², —OR¹¹, or —S(O)ₘR¹¹, and R⁶ represents a hydrogen atom or a C1-C6 alkyl group optionally having one or more groups selected from the group Z;

a compound represented by Formula (1) in which A¹ represents a nitrogen atom, A² represents —N(R¹²)—, A³ represents ═C(R⁴)—, W represents —OR⁶, R¹, R², R³, and R⁴ may be the same or different and each represents a hydrogen atom, a halogen atom, a cyano group, a nitro group, a C1-C6 alkyl group optionally having one or more groups selected from the group X, a phenyl group optionally having one or more groups selected from the group Y, a carboxy group, a C2-C6 alkoxycarbonyl group, an aminocarbonyl group, —NR¹¹R¹², —OR¹¹, or —S(O)ₘR¹¹, and R⁶ represents a hydrogen atom or a C1-C6 alkyl group;

a compound represented by Formula (1) in which A¹ represents a nitrogen atom, A² represents —N(R¹²)—, A³ represents ═C(R⁴)—, W represents —OR⁶, R¹, R², R³, and R⁴ may be the same or different and each represents a hydrogen atom, a halogen atom, a cyano group, a nitro group, a C1-C6 alkyl group optionally having one or more groups selected from the group X, a phenyl group optionally having one or more groups selected from the group Y, a carboxy group, a C2-C6 alkoxycarbonyl group, an aminocarbonyl group, —NR¹¹R¹², —OR¹¹, or —S(O)ₘR¹¹, and R⁶ represents a C1-C6 alkyl group;

a compound represented by Formula (1) in which A¹ represents a nitrogen atom, A² represents —N(R¹²)—, A³ represents ═C(R⁴)—, W represents —OR⁶, R¹, R², R³, and R⁴ may be the same or different and each represents a hydrogen atom, a halogen atom, a cyano group, a nitro group, a C1-C6 alkyl group optionally having one or more groups selected from the group X, a phenyl group optionally having one or more groups selected from the group Y, a carboxy group, a C2-C6 alkoxycarbonyl group, an aminocarbonyl group, —NR¹¹R¹², —OR¹¹, or —S(O)ₘR¹¹, and R⁶ represents a hydrogen atom;

a compound represented by Formula (1) in which A¹ represents a nitrogen atom, A² represents —N(R¹²)—, A³ represents ═C(R⁴)—, W represents —OR⁶, R¹, R², R³, and R⁴ may be the same or different and each represents a hydrogen atom, a halogen atom, a cyano group, a nitro group, a C1-C6 alkyl group optionally having one or more groups selected from the group X, a phenyl group optionally having one or more groups selected from the group Y, or —OR¹¹, R⁶ represents a hydrogen atom or a C1-C6 alkyl group, R¹¹ represents a C1-C6 alkyl group optionally having one or more groups selected from the group X, and R¹² represents a hydrogen atom or a C1-C4 alkyl group optionally having one or more halogen atoms;

a compound represented by Formula (1) in which A¹ represents a nitrogen atom, A² represents —N(R¹²)—, A³ represents ═C(R⁴)—, W represents —OR⁶, R¹, R², R³, and R⁴ may be the same or different and each represents a hydrogen atom, a halogen atom, a cyano group, a nitro group, a C1-C6 alkyl group optionally having one or more groups selected from the group X, a phenyl group optionally having one or more groups selected from the group Y, or —OR¹¹, R⁶ represents a hydrogen atom, R¹¹ represents a C1-C6 alkyl group optionally having one or more groups selected from the group X, and R¹² represents a hydrogen atom or a C1-C4 alkyl group optionally having one or more halogen atoms;

a compound represented by Formula (1) in which A¹ represents a nitrogen atom, A² represents —N(R¹²)—, A³ represents ═C(R⁴)—, W represents —OR⁶, R¹, R², R³, and R⁴ may be the same or different and each represents a hydrogen atom, a halogen atom, a cyano group, a nitro group, a C1-C6 alkyl group optionally having one or more groups selected from the group X, a phenyl group optionally having one or more groups selected from the group Y, or —OR¹¹, R⁶ represents a C1-C6 alkyl group, R¹¹ represents a C1-C6 alkyl group optionally having one or more groups selected from the group X, and R¹² represents a hydrogen atom or a C1-C4 alkyl group optionally having one or more halogen atoms;

a compound represented by Formula (1) in which A¹ represents a nitrogen atom, A² represents —N(R¹²)—, A³ represents ═C(R⁴)—, W represents —OR⁶, R¹ represents a trifluoromethyl group, R², R³, and R⁴ may be the same or different and each represents a hydrogen atom, a halogen atom, a cyano group, a nitro group, a C1-C6 alkyl group optionally having one or more groups selected from the group X, a phenyl group optionally having one or more groups selected from the group Y, or —OR¹¹, R⁶ represents a methyl group or an ethyl group, R¹¹ represents a C1-C6 alkyl group optionally having one or more groups selected from the group X, and R¹² represents a hydrogen atom or a C1-C4 alkyl group optionally having one or more halogen atoms;

a compound represented by Formula (1) in which A¹ represents a nitrogen atom, A² represents —N(R¹²)—, A³ represents ═C(R⁴)—, W represents —OR⁶, R¹, R³, and R⁴ may be the same or different and each represents a hydrogen atom, a halogen atom, a cyano group, a nitro group, a C1-C6 alkyl group optionally having one or more groups selected from the group X, a phenyl group optionally having one or more groups selected from the group Y, or —OR¹¹, R² represents a trifluoromethyl group, R⁶ represents a methyl group or an ethyl group, R¹¹ represents a C1-C6 alkyl group optionally having one or more groups selected from the group X, and R¹² represents a hydrogen atom or a C1-C4 alkyl group optionally having one or more halogen atoms;

a compound represented by Formula (1) in which A¹ represents a nitrogen atom, A² represents —N(R¹²)—, A³ represents ═C(R⁴)—, W represents —OR⁶, R¹, R², and R⁴ may be the same or different and each represents a hydrogen atom, a halogen atom, a cyano group, a nitro group, a C1-C6 alkyl group optionally having one or more groups selected from the group X, a phenyl group optionally having one or more groups selected from the group Y, or —OR¹¹, R³ represents a trifluoromethyl group, R⁶ represents a methyl group or an ethyl group, R¹¹ represents a C1-C6 alkyl group optionally having one or more groups selected from the group X, and R¹² represents a hydrogen atom or a C1-C4 alkyl group optionally having one or more halogen atoms;

a compound represented by Formula (1) in which A¹ represents a nitrogen atom, A² represents —N(R¹²)—, A³ represents ═C(R⁴)—, W represents —OR⁶, R¹, R², and R³ may be the same or different and each represents a hydrogen atom, a halogen atom, a cyano group, a nitro group, a C1-C6 alkyl group optionally having one or more groups selected from the group X, a phenyl group optionally having one or more groups selected from the group Y, or —OR¹¹, R⁴ represents a trifluoromethyl group, R⁶ represents a methyl group or an ethyl group, R¹¹ represents a C1-C6 alkyl group optionally having one or more groups selected from the group X, and R¹² represents a hydrogen atom or a C1-C4 alkyl group optionally having one or more halogen atoms;

a compound represented by Formula (1) in which A¹ represents a nitrogen atom, A² represents —N(R¹²)—, A³ represents a ═C(R⁴)—, and W represents —ON═CR⁷R⁸;

a compound represented by Formula (1) in which A¹ represents a nitrogen atom, A² represents —N(R¹²)—, A³ represents a ═C(R⁴)—, and W represents —SR⁹;

a compound represented by Formula (1) in which A¹ represents a nitrogen atom, A² represents —N(R¹²)—, A³ represents a ═C(R⁴)—, W represents —SR⁹, and R⁹ represents a C1-C6 alkyl group;

a compound represented by Formula (1) in which A¹ represents a nitrogen atom, A² represents —N(R¹²)—, A³ represents a ═C(R⁴)—, W represents —NR⁷R¹⁰, and R¹⁰ represents a hydrogen atom, a C1-C6 alkyl group or a phenyl group optionally having one or more group selected from the group Y;

a compound represented by Formula (1) in which A¹ represents a nitrogen atom, A² represents —N(R¹²)—, A³ represents a ═C(R⁴)—, W represents —NR⁷R¹⁰, and R¹⁰ represents a hydrogen atom or a C1-C6 alkyl group;

a compound represented by Formula (1) in which A¹ represents a nitrogen atom, A² represents an oxygen atom, A³ represents a nitrogen atom, and R² and R³ represent a hydrogen atom;

a compound represented by Formula (1) in which A¹ represents a nitrogen atom, A² represents an oxygen atom, A³ represents a nitrogen atom, and R¹ and R³ represent a hydrogen atom;

a compound represented by Formula (1) in which A¹ represents a nitrogen atom, A² represents an oxygen atom, A³ represents a nitrogen atom, and R¹ and R² represent a hydrogen atom;

a compound represented by Formula (1) in which A¹ represents a nitrogen atom, A² represents an oxygen atom, A³ represents a nitrogen atom, R¹ represents a trifluoromethyl group, and R² and R³ represent a hydrogen atom;

a compound represented by Formula (1) in which A¹ represents a nitrogen atom, A² represents an oxygen atom, A³ represents a nitrogen atom, R² represents a trifluoromethyl group, and R¹ and R³ represent a hydrogen atom;

a compound represented by Formula (1) in which A¹ represents a nitrogen atom, A² represents an oxygen atom, A³ represents a nitrogen atom, R³ represents a trifluoromethyl group, and R¹ and R² represent a hydrogen atom;

a compound represented by Formula (1) in which A¹ represents a nitrogen atom, A² represents an oxygen atom, A³ represents a nitrogen atom, and R¹ represents a trifluoromethyl group;

a compound represented by Formula (1) in which A¹ represents a nitrogen atom, A² represents an oxygen atom, A³ represents a nitrogen atom, and R² represents a trifluoromethyl group;

a compound represented by Formula (1) in which A¹ represents a nitrogen atom, A² represents an oxygen atom, A³ represents a nitrogen atom, and R³ represents a trifluoromethyl group;

a compound represented by Formula (1) in which A¹ represents a nitrogen atom, A² represents an oxygen atom, A³ represents a nitrogen atom, R¹ represents a halogen atom, and R² and R³ represent a hydrogen atom;

a compound represented by Formula (1) in which A¹ represents a nitrogen atom, A² represents an oxygen atom, A³ represents a nitrogen atom, R² represents a halogen atom, and R¹ and R³ represent a hydrogen atom;

a compound represented by Formula (1) in which A¹ represents a nitrogen atom, A² represents an oxygen atom, A³ represents a nitrogen atom, and R¹ represents a halogen atom;

a compound represented by Formula (1) in which A¹ represents a nitrogen atom, A² represents an oxygen atom, A³ represents a nitrogen atom, and R² represents a halogen atom;

a compound represented by Formula (1) in which A¹ represents a nitrogen atom, A² represents an oxygen atom, A³ represents a nitrogen atom, R¹ represents a C1-C6 alkyl group optionally having one or more groups selected from the group X, and R² and R³ represent a hydrogen atom;

a compound represented by Formula (1) in which A¹ represents a nitrogen atom, A² represents an oxygen atom, A³ represents a nitrogen atom, R² represents a C1-C6 alkyl group optionally having one or more groups selected from the group X, and R¹ and R³ represent a hydrogen atom;

a compound represented by Formula (1) in which A¹ represents a nitrogen atom, A² represents an oxygen atom, A³ represents a nitrogen atom, R³ represents a C1-C6 alkyl group optionally having one or more groups selected from the group X, and R¹ and R² represent a hydrogen atom;

a compound represented by Formula (1) in which A¹ represents a nitrogen atom, A² represents an oxygen atom, A³ represents a nitrogen atom, and R¹ represents a C1-C6 alkyl group optionally having one or more groups selected from the group X;

a compound represented by Formula (1) in which A¹ represents a nitrogen atom, A² represents an oxygen atom, A³ represents a nitrogen atom, and R² represents a C1-C6 alkyl group optionally having one or more groups selected from the group X;

a compound represented by Formula (1) in which A¹ represents a nitrogen atom, A² represents an oxygen atom, A³ represents a nitrogen atom, and R³ represents a C1-C6 alkyl group optionally having one or more groups selected from the group X;

a compound represented by Formula (1) in which A¹ represents a nitrogen atom, A² represents an oxygen atom, A³ represents a nitrogen atom, R¹ represents a phenyl group optionally having one or more groups selected from the group Y, and R² and R³ represent a hydrogen atom;

a compound represented by Formula (1) in which A¹ represents a nitrogen atom, A² represents an oxygen atom, A³ represents a nitrogen atom, R² represents a phenyl group optionally having one or more groups selected from the group Y, and R¹ and R³ represent a hydrogen atom;

a compound represented by Formula (1) in which A¹ represents a nitrogen atom, A² represents an oxygen atom, A³ represents a nitrogen atom, R³ represents a phenyl group optionally having one or more groups selected from the group Y, and R¹ and R² represent a hydrogen atom;

a compound represented by Formula (1) in which A¹ represents a nitrogen atom, A² represents an oxygen atom, A³ represents a nitrogen atom, R¹ represents a 6-membered aromatic heterocyclic group optionally having one or more groups selected from the group Y or a 5-membered aromatic heterocyclic group optionally having one or more groups selected from the group Y, and R² and R³ represent a hydrogen atom;

a compound represented by Formula (1) in which A¹ represents a nitrogen atom, A² represents an oxygen atom, A³ represents a nitrogen atom, R² represents a 6-membered aromatic heterocyclic group optionally having one or more groups selected from the group Y or a 5-membered aromatic heterocyclic group optionally having one or more groups selected from the group Y, and R¹ and R³ represent a hydrogen atom;

a compound represented by Formula (1) in which A¹ represents a nitrogen atom, A² represents an oxygen atom, A³ represents a nitrogen atom, R³ represents a 6-membered aromatic heterocyclic group optionally having one or more groups selected from the group Y or a 5-membered aromatic heterocyclic group optionally having one or more groups selected from the group Y, and R¹ and R² represent a hydrogen atom;

a compound represented by Formula (1) in which A¹ represents a nitrogen atom, A² represents an oxygen atom, A³ represents a nitrogen atom, and R¹, R², and R³ may be the same or different and each represents a hydrogen atom, a halogen atom, a cyano group, a nitro group, a C1-C6 alkyl group optionally having one or more groups selected from the group X, a phenyl group optionally having one or more groups selected from the group Y, a carboxy group, a C2-C6 alkoxycarbonyl group, an aminocarbonyl group, —NR¹¹R¹², —OR¹¹, or —S(O)ₘR¹¹;

a compound represented by Formula (1) in which A¹ represents a nitrogen atom, A² represents an oxygen atom, A³ represents a nitrogen atom, R¹, R², and R³ may be the same or different and each represents a hydrogen atom, a halogen atom, a cyano group, a nitro group, a C1-C6 alkyl group optionally having one or more groups selected from the group X, a phenyl group optionally having one or more groups selected from the group Y, a carboxy group, a C2-C6 alkoxycarbonyl group, an aminocarbonyl group, —NR¹¹R¹², —OR¹¹, or —S(O)ₘR¹¹, R¹¹ represents a C1-C6 alkyl group optionally having one or more groups selected from the group X, and R¹² represents a hydrogen atom or a C1-C4 alkyl group optionally having one or more halogen atoms;

a compound represented by Formula (1) in which A¹ represents a nitrogen atom, A² represents an oxygen atom, A³ represents a nitrogen atom, R¹, R², and R³ may be the same or different and each represents a hydrogen atom, a halogen atom, a cyano group, a nitro group, a C1-C6 alkyl group optionally having one or more groups selected from the group X, a phenyl group optionally having one or more groups selected from the group Y, a carboxy group, a C2-C6 alkoxycarbonyl group, an aminocarbonyl group, —NR¹¹R¹², —OR¹¹, or —S(O)ₘR¹¹, R¹¹ represents a C1-C6 alkyl group optionally having one or more groups selected from the group X, and R¹² represents a hydrogen atom;

a compound represented by Formula (1) in which A¹ represents a nitrogen atom, A² represents an oxygen atom, A³ represents a nitrogen atom, R¹, R², and R³ may be the same or different and each represents a hydrogen atom, a halogen atom, a cyano group, a nitro group, a C1-C6 alkyl group optionally having one or more groups selected from the group X, a phenyl group optionally having one or more groups selected from the group Y, a carboxy group, a C2-C6 alkoxycarbonyl group, an aminocarbonyl group, —NR¹¹R¹², OR¹¹, or —S(O)ₘR¹¹, R¹¹ represents a C1-C6 alkyl group optionally having one or more groups selected from the group X, and R¹² represents a C1-C4 alkyl group optionally having one or more halogen atoms;

a compound represented by Formula (1) in which A¹ represents a nitrogen atom, A² represents an oxygen atom, A³ represents a nitrogen atom, and R¹, R², and R³ may be the same or different and each represents a hydrogen atom, a halogen atom, a cyano group, a nitro group, a C1-C6 alkyl group optionally having one or more groups selected from the group X, a phenyl group optionally having one or more groups selected from the group Y, or OR¹¹;

a compound represented by Formula (1) in which A¹ represents a nitrogen atom, A² represents an oxygen atom, A³ represents a nitrogen atom, and R¹, R², and R³ may be the same or different and each represents a hydrogen atom, a halogen atom, a cyano group, a nitro group, a C1-C6 alkyl group optionally having one or more groups selected from the group X, a phenyl group optionally having one or more groups selected from the group Y, or OR¹¹, and R¹¹ represents a C1-C6 alkyl group optionally having one or more groups selected from the group X;

a compound represented by Formula (1) in which A¹ represents a nitrogen atom, A² represents an oxygen atom, A³ represents a nitrogen atom, W represents —OR⁶, and R¹, R², and R³ may be the same or different and each represents a hydrogen atom, a halogen atom, a cyano group, a nitro group, a C1-C6 alkyl group optionally having one or more groups selected from the group X, a phenyl group optionally having one or more groups selected from the group Y, a carboxy group, a C2-C6 alkoxycarbonyl group, an aminocarbonyl group, —NR¹¹R¹², —OR¹¹, or —S(O)ₘR¹¹;

a compound represented by Formula (1) in which A¹ represents a nitrogen atom, A² represents an oxygen atom, A³ represents a nitrogen atom, W represents —OR⁶, R¹, R², and R³ may be the same or different and each represents a hydrogen atom, a halogen atom, a cyano group, a nitro group, a C1-C6 alkyl group optionally having one or more groups selected from the group X, a phenyl group optionally having one or more groups selected from the group Y, a carboxy group, a C2-C6 alkoxycarbonyl group, an aminocarbonyl group, —NR¹¹R¹², —OR¹¹, or —S(O)ₘR¹¹, and R⁶ represents a C1-C6 alkyl group optionally having one or more groups selected from the group Z;

a compound represented by Formula (1) in which A¹ represents a nitrogen atom, A² represents an oxygen atom, A³ represents a nitrogen atom, W represents —OR⁶, R¹, R², and R³ may be the same or different and each represents a hydrogen atom, a halogen atom, a cyano group, a nitro group, a C1-C6 alkyl group optionally having one or more groups selected from the group X, a phenyl group optionally having one or more groups selected from the group Y, a carboxy group, a C2-C6 alkoxycarbonyl group, an aminocarbonyl group, —NR¹¹R¹², —OR¹¹, or —S(O)ₘR¹¹, and R⁶ represents a C1-C6 alkyl group;

a compound represented by Formula (1) in which A¹ represents a nitrogen atom, A² represents an oxygen atom, A³ represents a nitrogen atom, W represents —OR⁶, R¹, R², and R³ may be the same or different and each represents a hydrogen atom, a halogen atom, a cyano group, a nitro group, a C1-C6 alkyl group optionally having one or more groups selected from the group X, a phenyl group optionally having one or more groups selected from the group Y, or —OR¹¹, R⁶ represents a C1-C6 alkyl group, and R¹¹ represents a C1-C6 alkyl group optionally having one or more groups selected from the group X;

a compound represented by Formula (1) in which A¹ represents a nitrogen atom, A² represents an oxygen atom, A³ represents a nitrogen atom, W represents —OR⁶, R¹ represents a trifluoromethyl group, R², and R³ may be the same or different and each represents a hydrogen atom, a halogen atom, a cyano group, a nitro group, a C1-C6 alkyl group optionally having one or more groups selected from the group X, a phenyl group optionally having one or more groups selected from the group Y, or —OR¹¹, R⁶ represents a methyl group or an ethyl group, R¹¹ represents a C1-C6 alkyl group optionally having one or more groups selected from the group X, and R¹² represents a hydrogen atom or a C1-C4 alkyl group optionally having one or more halogen atoms;

a compound represented by Formula (1) in which A¹ represents a nitrogen atom, A² represents an oxygen atom, A³ represents a nitrogen atom, W represents —OR⁶, R¹, and R³ may be the same or different and each represents a hydrogen atom, a halogen atom, a cyano group, a nitro group, a C1-C6 alkyl group optionally having one or more groups selected from the group X, a phenyl group optionally having one or more groups selected from the group Y, or —OR¹¹, R² represents a trifluoromethyl group, R⁶ represents a methyl group or an ethyl group, and R¹¹ represents a C1-C6 alkyl group optionally having one or more groups selected from the group X;

a compound represented by Formula (1) in which A¹ represents a nitrogen atom, A² represents an oxygen atom, A³ represents a nitrogen atom, W represents —OR⁶, R¹, and R² may be the same or different and each represents a hydrogen atom, a halogen atom, a cyano group, a nitro group, a C1-C6 alkyl group optionally having one or more groups selected from the group X, a phenyl group optionally having one or more groups selected from the group Y, or —OR¹¹, R³ represents a trifluoromethyl group, R⁶ represents a methyl group or an ethyl group, and R¹¹ represents a C1-C6 alkyl group optionally having one or more groups selected from the group X;

a compound represented by Formula (1) in which A¹ represents a nitrogen atom, A² represents an oxygen atom, A³ represents a nitrogen atom, and W represents —ON═CR⁷R⁸;

a compound represented by Formula (1) in which A¹ represents a nitrogen atom, A² represents an oxygen atom, A³ represents a nitrogen atom, and W represents —SR⁹;

a compound represented by Formula (1) in which A¹ represents a nitrogen atom, A² represents an oxygen atom, A³ represents a nitrogen atom, W represents —SR⁹, and R⁹ represents a C1-C6 alkyl group;

a compound represented by Formula (1) in which A¹ represents a nitrogen atom, A² represents an oxygen atom, A³ represents a nitrogen atom, W represents —NR⁷R¹⁰, and R₁₀ represents a hydrogen atom, a C1-C6 alkyl group, or a phenyl group optionally having one or more groups selected from the group Y;

a compound represented by Formula (1) in which A¹ represents a nitrogen atom, A² represents an oxygen atom, A³ represents a nitrogen atom, W represents —NR⁷R¹⁰, and R¹⁰ represents a hydrogen atom, or a C1-C6 alkyl group;

a compound represented by Formula (1) in which A¹ represents a nitrogen atom, A² represents an oxygen atom, A³ represents ═C(R⁴)—, and R², R³, and R⁴ represent a hydrogen atom;

a compound represented by Formula (1) in which A¹ represents a nitrogen atom, A² represents an oxygen atom, A³ represents ═C(R⁴)—, and R¹, R³, and R⁴ represent a hydrogen atom;

a compound represented by Formula (1) in which A¹ represents a nitrogen atom, A² represents an oxygen atom, A³ represents ═C(R⁴)—, and R¹, R², and R⁴ represent a hydrogen atom;

a compound represented by Formula (1) in which A¹ represents a nitrogen atom, A² represents an oxygen atom, A³ represents ═C(R⁴)—, and R¹, R² and R³ represent a hydrogen atom;

a compound represented by Formula (1) in which A¹ represents a nitrogen atom, A² represents an oxygen atom, A³ represents ═C(R⁴)—, R¹ represents a trifluoromethyl group, and R², R³, and R⁴ represent a hydrogen atom;

a compound represented by Formula (1) in which A¹ represents a nitrogen atom, A² represents an oxygen atom, A³ represents ═C(R⁴)—, R² represents a trifluoromethyl group, and R¹, R³, and R⁴ represent a hydrogen atom;

a compound represented by Formula (1) in which A¹ represents a nitrogen atom, A² represents an oxygen atom, A³ represents ═C(R⁴)—, R³ represents a trifluoromethyl group, and R¹, R², and R⁴ represent a hydrogen atom;

a compound represented by Formula (1) in which A¹ represents a nitrogen atom, A² represents an oxygen atom, A³ represents ═C(R⁴)—, R⁴ represents a trifluoromethyl group, and R¹, R², and R³ represent a hydrogen atom;

a compound represented by Formula (1) in which A¹ represents a nitrogen atom, A² represents an oxygen atom, A³ represents ═C(R⁴)—, and R¹ represents a trifluoromethyl group;

a compound represented by Formula (1) in which A¹ represents a nitrogen atom, A² represents an oxygen atom, A³ represents ═C(R⁴)—, and R² represents a trifluoromethyl group;

a compound represented by Formula (1) in which A¹ represents a nitrogen atom, A² represents an oxygen atom, A³ represents ═C(R⁴)—, and R³ represents a trifluoromethyl group;

a compound represented by Formula (1) in which A¹ represents a nitrogen atom, A² represents an oxygen atom, A³ represents ═C(R⁴)—, and R⁴ represents a trifluoromethyl group;

a compound represented by Formula (1) in which A¹ represents a nitrogen atom, A² represents an oxygen atom, A³ represents ═C(R⁴)—, R¹ represents a halogen atom, and R², R³, and R⁴ represent a hydrogen atom;

a compound represented by Formula (1) in which A¹ represents a nitrogen atom, A² represents an oxygen atom, A³ represents ═C(R⁴)—, R² represents a halogen atom, and R¹, R³, and R⁴ represent a hydrogen atom;

a compound represented by Formula (1) in which A¹ represents a nitrogen atom, A² represents an oxygen atom, A³ represents ═C(R⁴)—, R³ represents a halogen atom, and R¹, R², and R⁴ represent a hydrogen atom;

a compound represented by Formula (1) in which A¹ represents a nitrogen atom, A² represents an oxygen atom, A³ represents ═C(R⁴)—, R⁴ represents a halogen atom, and R¹, R², and R³ represent a hydrogen atom;

a compound represented by Formula (1) in which A¹ represents a nitrogen atom, A² represents an oxygen atom, A³ represents ═C(R⁴)—, and R¹ represents a halogen atom;

a compound represented by Formula (1) in which A¹ represents a nitrogen atom, A² represents an oxygen atom, A³ represents ═C(R⁴)—, and R² represents a halogen atom;

a compound represented by Formula (1) in which A¹ represents a nitrogen atom, A² represents an oxygen atom, A³ represents ═C(R⁴)—, R¹ represents a C1-C6 alkyl group optionally having one or more groups selected from the group X, and R², R³, and R⁴ represent a hydrogen atom;

a compound represented by Formula (1) in which A¹ represents a nitrogen atom, A² represents an oxygen atom, A³ represents ═C(R⁴)—, R² represents a C1-C6 alkyl group optionally having one or more groups selected from the group X, and R¹, R³, and R⁴ represent a hydrogen atom;

a compound represented by Formula (1) in which A¹ represents a nitrogen atom, A² represents an oxygen atom, A³ represents ═C(R⁴)—, R³ represents a C1-C6 alkyl group optionally having one or more groups selected from the group X, and R¹, R², and R⁴ represent a hydrogen atom;

a compound represented by Formula (1) in which A¹ represents a nitrogen atom, A² represents an oxygen atom, A³ represents ═C(R⁴)—, R⁴ represents a C1-C6 alkyl group optionally having one or more groups selected from the group X, and R¹, R², and R³ represent a hydrogen atom;

a compound represented by Formula (1) in which A¹ represents a nitrogen atom, A² represents an oxygen atom, A³ represents ═C(R⁴)—, and R¹ represents a C1-C6 alkyl group optionally having one or more groups selected from the group X;

a compound represented by Formula (1) in which A¹ represents a nitrogen atom, A² represents an oxygen atom, A³ represents ═C(R⁴)—, and R² represents a C1-C6 alkyl group optionally having one or more groups selected from the group X;

a compound represented by Formula (1) in which A¹ represents a nitrogen atom, A² represents an oxygen atom, A³ represents ═C(R⁴)—, R¹ represents a phenyl group optionally having one or more groups selected from the group Y, and R², R³, and R⁴ represent a hydrogen atom;

a compound represented by Formula (1) in which A¹ represents a nitrogen atom, A² represents an oxygen atom, A³ represents ═C(R⁴)—, R² represents a phenyl group optionally having one or more groups selected from the group Y, and R¹, R³, and R⁴ represent a hydrogen atom;

a compound represented by Formula (1) in which A¹ represents a nitrogen atom, A² represents an oxygen atom, A³ represents ═C(R⁴)—, R³ represents a phenyl group optionally having one or more groups selected from the group Y, and R¹, R², and R⁴ represent a hydrogen atom;

a compound represented by Formula (1) in which A¹ represents a nitrogen atom, A² represents an oxygen atom, A³ represents ═C(R⁴)—, R⁴ represents a phenyl group optionally having one or more groups selected from the group Y, and R¹, R², and R³ represent a hydrogen atom;

a compound represented by Formula (1) in which A¹ represents a nitrogen atom, A² represents an oxygen atom, A³ represents ═C(R⁴)—, and R¹ represents a phenyl group optionally having one or more groups selected from the group Y;

a compound represented by Formula (1) in which A¹ represents a nitrogen atom, A² represents an oxygen atom, A³ represents ═C(R⁴)—, and R² represents a phenyl group optionally having one or more groups selected from the group Y;

a compound represented by Formula (1) in which A¹ represents a nitrogen atom, A² represents an oxygen atom, A³ represents ═C(R⁴)—, R¹ represents a 6-membered aromatic heterocyclic group optionally having one or more groups selected from the group Y or a 5-membered aromatic heterocyclic group optionally having one or more groups selected from the group Y, and R², R³, and R⁴ represent a hydrogen atom;

a compound represented by Formula (1) in which A¹ represents a nitrogen atom, A² represents an oxygen atom, A³ represents ═C(R⁴)—, R² represents a 6-membered aromatic heterocyclic group optionally having one or more groups selected from the group Y or a 5-membered aromatic heterocyclic group optionally having one or more groups selected from the group Y, and R¹, R³, and R⁴ represent a hydrogen atom;

a compound represented by Formula (1) in which A¹ represents a nitrogen atom, A² represents an oxygen atom, A³ represents ═C(R⁴)—, R³ represents a 6-membered aromatic heterocyclic group optionally having one or more groups selected from the group Y or a 5-membered aromatic heterocyclic group optionally having one or more groups selected from the group Y, and R¹, R², and R⁴ represent a hydrogen atom;

a compound represented by Formula (1) in which A¹ represents a nitrogen atom, A² represents an oxygen atom, A³ represents ═C(R⁴)—, R⁴ represents a 6-membered aromatic heterocyclic group optionally having one or more groups selected from the group Y or a 5-membered aromatic heterocyclic group optionally having one or more groups selected from the group Y, and R¹, R², and R³ represent a hydrogen atom;

a compound represented by Formula (1) in which A¹ represents a nitrogen atom, A² represents an oxygen atom, A³ represents ═C(R⁴)—, and R¹ represents a 6-membered aromatic heterocyclic group optionally having one or more groups selected from the group Y or a 5-membered aromatic heterocyclic group optionally having one or more groups selected from the group Y;

a compound represented by Formula (1) in which A¹ represents a nitrogen atom, A² represents an oxygen atom, A³ represents ═C(R⁴)—, and R² represents a 6-membered aromatic heterocyclic group optionally having one or more groups selected from the group Y or a 5-membered aromatic heterocyclic group optionally having one or more groups selected from the group Y;

a compound represented by Formula (1) in which A¹ represents a nitrogen atom, A² represents an oxygen atom, A³ represents ═C(R⁴)—, R¹ represents a C2-C6 alkylcarbonyl group optionally having one or more halogen atoms, and R², R³, and R⁴ represent a hydrogen atom;

a compound represented by Formula (1) in which A¹ represents a nitrogen atom, A² represents an oxygen atom, A³ represents ═C(R⁴)—, R² represents a C2-C6 alkylcarbonyl group optionally having one or more halogen atoms, and R¹, R³, and R⁴ represent a hydrogen atom;

a compound represented by Formula (1) in which A¹ represents a nitrogen atom, A² represents an oxygen atom, A³ represents ═C(R⁴)—, and R¹ represents a C2-C6 alkylcarbonyl group optionally having one or more halogen atoms;

a compound represented by Formula (1) in which A¹ represents a nitrogen atom, A² represents an oxygen atom, A³ represents ═C(R⁴)—, and R² represents a C2-C6 alkylcarbonyl group optionally having one or more halogen atoms;

a compound represented by Formula (1) in which A¹ represents a nitrogen atom, A² represents an oxygen atom, A³ represents ═C(R⁴)—, R¹ represents a carboxy group, and R², R³, and R⁴ represent a hydrogen atom;

a compound represented by Formula (1) in which A¹ represents a nitrogen atom, A² represents an oxygen atom, A³ represents ═C(R⁴)—, R² represents a carboxy group, and R¹, R³, and R⁴ represent a hydrogen atom;

a compound represented by Formula (1) in which A¹ represents a nitrogen atom, A² represents an oxygen atom, A³ represents ═C(R⁴)—, and R¹ represents a carboxy group;

a compound represented by Formula (1) in which A¹ represents a nitrogen atom, A² represents an oxygen atom, A³ represents ═C(R⁴)—, and R² represents a carboxy group;

a compound represented by Formula (1) in which A¹ represents a nitrogen atom, A² represents an oxygen atom, A³ represents ═C(R⁴)—, R¹ represents a C2-C6 alkoxycarbonyl group, and R², R³, and R⁴ represent a hydrogen atom;

a compound represented by Formula (1) in which A¹ represents a nitrogen atom, A² represents an oxygen atom, A³ represents ═C(R⁴)—, R² represents a C2-C6 alkoxycarbonyl group, and R¹, R³, and R⁴ represent a hydrogen atom;

a compound represented by Formula (1) in which A¹ represents a nitrogen atom, A² represents an oxygen atom, A³ represents ═C(R⁴)—, and R¹ represents a C2-C6 alkoxycarbonyl group;

a compound represented by Formula (1) in which A¹ represents a nitrogen atom, A² represents an oxygen atom, A³ represents ═C(R⁴)—, and R² represents a C2-C6 alkoxycarbonyl group;

a compound represented by Formula (1) in which A¹ represents a nitrogen atom, A² represents an oxygen atom, A represents ═C(R⁴)—, and R¹, R², R³, and R⁴ may be the same or different and each represents a hydrogen atom, a halogen atom, a cyano group, a nitro group, a C1-C6 alkyl group optionally having one or more groups selected from the group X, a phenyl group optionally having one or more groups selected from the group Y, a carboxy group, a C2-C6 alkoxycarbonyl group, an aminocarbonyl group, —NR¹¹R¹², —OR¹¹, or —S(O)ₘR¹¹;

a compound represented by Formula (1) in which A¹ represents a nitrogen atom, A² represents an oxygen atom, A³ represents ═C(R⁴)—, R¹, R², R³, and R⁴ may be the same or different and each represents a hydrogen atom, a halogen atom, a cyano group, a nitro group, a C1-C6 alkyl group optionally having one or more groups selected from the group X, a phenyl group optionally having one or more groups selected from the group Y, a carboxy group, a C2-C6 alkoxycarbonyl group, an aminocarbonyl group, —NR¹¹R¹², —OR¹¹, or —S(O)ₘR¹¹, and R¹¹ represents a C1-C6 alkyl group optionally having one or more groups selected from the group X;

a compound represented by Formula (1) in which A¹ represents a nitrogen atom, A² represents an oxygen atom, A³ represents ═C(R⁴)—, R¹, R², R³, and R⁴ may be the same or different and each represents a hydrogen atom, a halogen atom, a cyano group, a nitro group, a C1-C6 alkyl group optionally having one or more groups selected from the group X, a phenyl group optionally having one or more groups selected from the group Y, a carboxy group, a C2-C6 alkoxycarbonyl group, an aminocarbonyl group, —NR¹¹R¹², —OR¹¹, or —S(O)ₘR¹¹, R¹¹ represents a C1-C6 alkyl group optionally having one or more groups selected from the group X, and R¹² represents a hydrogen atom or a C1-C4 alkyl group optionally having one or more halogen atoms;

a compound represented by Formula (1) in which A¹ represents a nitrogen atom, A² represents an oxygen atom, A³ represents ═C(R⁴)—, R¹, R², R³, and R⁴ may be the same or different and each represents a hydrogen atom, a halogen atom, a cyano group, a nitro group, a C1-C6 alkyl group optionally having one or more groups selected from the group X, a phenyl group optionally having one or more groups selected from the group Y, or —OR¹¹, and R¹¹ represents a C1-C6 alkyl group optionally having one or more groups selected from the group X;

a compound represented by Formula (1) in which A¹ represents a nitrogen atom, A² represents an oxygen atom, A³ represents ═C(R⁴)—, W represents —OR⁶, and R¹, R², R³, and R⁴ may be the same or different and each represents a hydrogen atom, a halogen atom, a cyano group, a nitro group, a C1-C6 alkyl group optionally having one or more groups selected from the group X, a phenyl group optionally having one or more groups selected from the group Y, a carboxy group, a C2-C6 alkoxycarbonyl group, an aminocarbonyl group, —NR¹¹R¹², —OR¹¹, or —S(O)ₘR¹¹;

a compound represented by Formula (1) in which A¹ represents a nitrogen atom, A² represents an oxygen atom, A³ represents ═C(R⁴)—, W represents —OR⁶, R¹, R², R³, and R⁴ may be the same or different and each represents a hydrogen atom, a halogen atom, a cyano group, a nitro group, a C1-C6 alkyl group optionally having one or more optionally having one or more groups selected from the group Y, a carboxy group, a C2-C6 alkoxycarbonyl group, an aminocarbonyl group, —NR¹¹R¹², —OR¹¹, or —S(O)ₘR¹¹, and R⁶ represents a C1-C6 alkyl group optionally having one or more groups selected from the group Z;

a compound represented by Formula (1) in which A¹ represents a nitrogen atom, A² represents an oxygen atom, A³ represents ═C(R⁴)—, W represents —OR⁶, R¹, R², R³, and R⁴ may be the same or different and each represents a hydrogen atom, a halogen atom, a cyano group, a nitro group, a C1-C6 alkyl group optionally having one or more groups selected from the group X, a phenyl group optionally having one or more groups selected from the group Y, a carboxy group, a C2-C6 alkoxycarbonyl group, an aminocarbonyl group, —NR¹¹R¹², —OR¹¹, or —S(O)ₘR¹¹, and R⁶ represents a C1-C6 alkyl group;

a compound represented by Formula (1) in which A¹ represents a nitrogen atom, A² represents an oxygen atom, A³ represents ═C(R⁴)—, W represents —OR⁶, R¹, R², R³, and R⁴ may be the same or different and each represents a hydrogen atom, a halogen atom, a cyano group, a nitro group, a C1-C6 alkyl group optionally having one or more optionally having one or more groups selected from the group Y, or —OR¹¹, R⁶ represents a C1-C6 alkyl group, and R¹¹ represents a C1-C6 alkyl group optionally having one or more groups selected from the group X;

a compound represented by Formula (1) in which A¹ represents a nitrogen atom, A² represents an oxygen atom, A³ represents ═C(R⁴)—, W represents —OR⁶, R¹ represents a trifluoromethyl group, R², R³, and R⁴ may be the same or different and each represents a hydrogen atom, a halogen atom, a cyano group, a nitro group, a C1-C6 alkyl group optionally having one or more groups selected from the group X, a phenyl group optionally having one or more groups selected from the group Y, or —OR¹¹, R⁶ represents a methyl group or an ethyl group, and R¹¹ represents a C1-C6 alkyl group optionally having one or more groups selected from the group X;

a compound represented by Formula (1) in which A¹ represents a nitrogen atom, A² represents an oxygen atom, A³ represents ═C(R⁴)—, W represents —OR⁶, R¹, R³, and R⁴ may be the same or different and each represents a hydrogen atom, a halogen atom, a cyano group, a nitro group, a C1-C6 alkyl group optionally having one or more groups selected from the group X, a phenyl group optionally having one or more groups selected from the group Y, or —OR¹¹, R² represents a trifluoromethyl group, R⁶ represents a methyl group or an ethyl group, and R¹¹ represents a C1-C6 alkyl group optionally having one or more groups selected from the group X;

a compound represented by Formula (1) in which A¹ represents a nitrogen atom, A² represents an oxygen atom, A³ represents ═C(R⁴)—, W represents —OR⁶, R¹, R², and R⁴ may be the same or different and each represents a hydrogen atom, a halogen atom, a cyano group, a nitro group, a C1-C6 alkyl group optionally having one or more groups selected from the group X, a phenyl group optionally having one or more groups selected from the group Y, or —OR¹¹, R³ represents a trifluoromethyl group, R⁶ represents a methyl group or an ethyl group, and R¹¹ represents a C1-C6 alkyl group optionally having one or more groups selected from the group X;

a compound represented by Formula (1) in which A¹ represents a nitrogen atom, A² represents an oxygen atom, A³ represents ═C(R⁴)—, W represents —OR⁶, R¹, R², and R³ may be the same or different and each represents a hydrogen atom, a halogen atom, a cyano group, a nitro group, a C1-C6 alkyl group optionally having one or more groups selected from the group X, a phenyl group optionally having one or more groups selected from the group Y, or —OR¹¹, R⁴ represents a trifluoromethyl group, R⁶ represents a methyl group or an ethyl group, and R¹¹ represents a C1-C6 alkyl group optionally having one or more groups selected from the group X;

a compound represented by Formula (1) in which A¹ represents a nitrogen atom, A² represents an oxygen atom, A³ represents ═C(R⁴)—, and W represents —ON═CR⁷R⁸;

a compound represented by Formula (1) in which A¹ represents a nitrogen atom, A² represents an oxygen atom, A³ represents ═C(R⁴)—, and W represents —SR⁹;

a compound represented by Formula (1) in which A¹ represents a nitrogen atom, A² represents an oxygen atom, A³ represents ═C(R⁴)—, W represents —SR⁹, and R⁹ represents a C1-C6 alkyl group;

a compound represented by Formula (1) in which A¹ represents a nitrogen atom, A² represents an oxygen atom, A³ represents ═C(R⁴)—, and W represents —NR¹⁶R¹⁷;

a compound represented by Formula (1) in which A¹ represents a nitrogen atom, A² represents an oxygen atom, A³ represents ═C(R⁴)—, W represents —NR¹⁶R¹⁷, R⁷ and R⁸ may be the same or different and each represents a C1-C6 alkyl group optionally having one or more halogen atoms or a hydrogen atom, R¹⁶ represents a C1-C6 alkyl group optionally having one or more halogen atoms or a hydrogen atom, and R¹⁷ represents a cyano group, a phenyl group optionally having one or more groups selected from the group Y, —OR⁷, or —NR⁷R⁸;

a compound represented by Formula (1) in which A¹ represents a nitrogen atom, A² represents a sulfur atom, and R², R³, and R⁴ represent a hydrogen atom;

a compound represented by Formula (1) in which A¹ represents a nitrogen atom, A² represents a sulfur atom, and R¹, R³, and R⁴ represent a hydrogen atom;

a compound represented by Formula (1) in which A¹ represents a nitrogen atom, A² represents a sulfur atom, and R¹, R², and R⁴ represent a hydrogen atom;

a compound represented by Formula (1) in which A¹ represents a nitrogen atom, A² represents a sulfur atom, and R¹, R², and R³ represent a hydrogen atom;

a compound represented by Formula (1) in which A¹ represents a nitrogen atom, A² represents a sulfur atom, R¹ represents a trifluoromethyl group, and R², R³, and R⁴ represent a hydrogen atom;

a compound represented by Formula (1) in which A¹ represents a nitrogen atom, A² represents a sulfur atom, R² represents a trifluoromethyl group, and R¹, R³, and R⁴ represent a hydrogen atom;

a compound represented by Formula (1) in which A¹ represents a nitrogen atom, A² represents a sulfur atom, R³ represents a trifluoromethyl group, and R¹, R², and R⁴ represent a hydrogen atom;

a compound represented by Formula (1) in which A¹ represents a nitrogen atom, A² represents a sulfur atom, R⁴ represents a trifluoromethyl group, and R¹, R², and R³ represent a hydrogen atom;

a compound represented by Formula (1) in which A¹ represents a nitrogen atom, A² represents a sulfur atom, and R¹ represents a trifluoromethyl group;

a compound represented by Formula (1) in which A¹ represents a nitrogen atom, A² represents a sulfur atom, and R² represents a trifluoromethyl group;

a compound represented by Formula (1) in which A¹ represents a nitrogen atom, A² represents a sulfur atom, and R³ represents a trifluoromethyl group;

a compound represented by Formula (1) in which A¹ represents a nitrogen atom, A² represents a sulfur atom, and R⁴ represents a trifluoromethyl group;

a compound represented by Formula (1) in which A¹ represents a nitrogen atom, A² represents a sulfur atom, R¹ represents a halogen atom, and R², R³, and R⁴ represent a hydrogen atom;

a compound represented by Formula (1) in which A¹ represents a nitrogen atom, A² represents a sulfur atom, R² represents a halogen atom, and R¹, R³, and R⁴ represent a hydrogen atom;

a compound represented by Formula (1) in which A¹ represents a nitrogen atom, A² represents a sulfur atom, and R¹ represents a halogen atom;

a compound represented by Formula (1) in which A¹ represents a nitrogen atom, A² represents a sulfur atom, and R² represents a halogen atom;

a compound represented by Formula (1) in which A¹ represents a nitrogen atom, A² represents a sulfur atom, R¹ represents a C1-C6 alkyl group optionally having one or more groups selected from the group X, and R², R³, and R⁴ represent a hydrogen atom;

a compound represented by Formula (1) in which A¹ represents a nitrogen atom, A² represents a sulfur atom, R² represents a C1-C6 alkyl group optionally having one or more groups selected from the group X, and R¹, R³, and R⁴ represent a hydrogen atom;

a compound represented by Formula (1) in which A¹ represents a nitrogen atom, A² represents a sulfur atom, and R¹ represents a C1-C6 alkyl group optionally having one or more groups selected from the group X;

a compound represented by Formula (1) in which A¹ represents a nitrogen atom, A² represents a sulfur atom, and R² represents a C1-C6 alkyl group optionally having one or more groups selected from the group X;

a compound represented by Formula (1) in which A¹ represents a nitrogen atom, A² represents a sulfur atom, R¹ represents a phenyl group optionally having one or more groups selected from the group Y, and R², R³, and R⁴ represent a hydrogen atom;

a compound represented by Formula (1) in which A¹ represents a nitrogen atom, A² represents a sulfur atom, R² represents a phenyl group optionally having one or more groups selected from the group Y, and R¹, R³, and R⁴ represent a hydrogen atom;

a compound represented by Formula (1) in which A¹ represents a nitrogen atom, A² represents a sulfur atom, and R¹ represents a phenyl group optionally having one or more groups selected from the group Y;

a compound represented by Formula (1) in which A¹ represents a nitrogen atom, A² represents a sulfur atom, and R² represents a phenyl group optionally having one or more groups selected from the group Y;

a compound represented by Formula (1) in which A¹ represents a nitrogen atom, A² represents a sulfur atom, R¹ represents a 6-membered aromatic heterocyclic group optionally having one or more groups selected from the group Y or a 5-membered aromatic heterocyclic group optionally having one or more groups selected from the group Y, and R², R³, and R⁴ represent a hydrogen atom;

a compound represented by Formula (1) in which A¹ represents a nitrogen atom, A² represents a sulfur atom, R² represents a 6-membered aromatic heterocyclic group optionally having one or more groups selected from the group Y or a 5-membered aromatic heterocyclic group optionally having one or more groups selected from the group Y, and R¹, R³, and R⁴ represent a hydrogen atom;

a compound represented by Formula (1) in which A¹ represents a nitrogen atom, A² represents a sulfur atom, and R¹ represents a 6-membered aromatic heterocyclic group optionally having one or more groups selected from the group Y or a 5-membered aromatic heterocyclic group optionally having one or more groups selected from the group Y;

a compound represented by Formula (1) in which A¹ represents a nitrogen atom, A² represents a sulfur atom, and R² represents a 6-membered aromatic heterocyclic group optionally having one or more groups selected from the group Y or a 5-membered aromatic heterocyclic group optionally having one or more groups selected from the group Y;

a compound represented by Formula (1) in which A¹ represents a nitrogen atom, A² represents a sulfur atom, R¹ represents a C2-C6 alkylcarbonyl group optionally having one or more halogen atoms, and R², R³, and R⁴ represent a hydrogen atom;

a compound represented by Formula (1) in which A¹ represents a nitrogen atom, A² represents a sulfur atom, R² represents a C2-C6 alkylcarbonyl group optionally having one or more halogen atoms, and R¹, R³, and R⁴ represent a hydrogen atom;

a compound represented by Formula (1) in which A¹ represents a nitrogen atom, A² represents a sulfur atom, and R¹ represents a C2-C6 alkylcarbonyl group optionally having one or more halogen atoms;

a compound represented by Formula (1) in which A¹ represents a nitrogen atom, A² represents a sulfur atom, and R² represents a C2-C6 alkylcarbonyl group optionally having one or more halogen atoms;

a compound represented by Formula (1) in which A¹ represents a nitrogen atom, A² represents a sulfur atom, R¹ represents a carboxy group, and R², R³, and R⁴ represent a hydrogen atom;

a compound represented by Formula (1) in which A¹ represents a nitrogen atom, A² represents a sulfur atom, R² represents a carboxy group, and R¹, R³, and R⁴ represent a hydrogen atom;

a compound represented by Formula (1) in which A¹ represents a nitrogen atom, A² represents a sulfur atom, and R¹ represents a carboxy group;

a compound represented by Formula (1) in which A¹ represents a nitrogen atom, A² represents a sulfur atom, and R² represents a carboxy group;

a compound represented by Formula (1) in which A¹ represents a nitrogen atom, A² represents a sulfur atom, R¹ represents a C2-C6 alkoxycarbonyl group, and R², R³, and R⁴ represent a hydrogen atom;

a compound represented by Formula (1) in which A¹ represents a nitrogen atom, A² represents a sulfur atom, R² represents a C2-C6 alkoxycarbonyl group, and R¹, R³, and R⁴ represent a hydrogen atom;

a compound represented by Formula (1) in which A¹ represents a nitrogen atom, A² represents a sulfur atom, and R¹ represents a C2-C6 alkoxycarbonyl group;

a compound represented by Formula (1) in which A¹ represents a nitrogen atom, A² represents a sulfur atom, and R² represents a C2-C6 alkoxycarbonyl group;

a compound represented by Formula (1) in which A¹ represents a nitrogen atom, A² represents a sulfur atom, and R¹, R², R³, and R⁴ may be the same or different and each represents a hydrogen atom, a halogen atom, a cyano group, a nitro group, a C1-C6 alkyl group optionally having one or more groups selected from the group X, a phenyl group optionally having one or more groups selected from the group Y, a carboxy group, a C2-C6 alkoxycarbonyl group, an aminocarbonyl group, —NR¹¹R¹², —OR¹¹, or —S(O)ₘR¹¹;

a compound represented by Formula (1) in which A¹ represents a nitrogen atom, A² represents a sulfur atom, R¹, R², R³, and R⁴ may be the same or different and each represents a hydrogen atom, a halogen atom, a cyano group, a nitro group, a C1-C6 alkyl group optionally having one or more groups selected from the group X, a phenyl group optionally having one or more groups selected from the group Y, a carboxy group, a C2-C6 alkoxycarbonyl group, an aminocarbonyl group, —NR¹¹R¹², —OR¹¹, or —S(O)ₘR¹¹, and R¹¹ represents a C1-C6 alkyl group optionally having one or more groups selected from the group X;

a compound represented by Formula (1) in which A¹ represents a nitrogen atom, A² represents a sulfur atom, and R¹, R², R³, and R⁴ may be the same or different and each represents a hydrogen atom, a halogen atom, a cyano group, a nitro group, a C1-C6 alkyl group optionally having one or more groups selected from the group X, a phenyl group optionally having one or more groups selected from the group Y, or —OR¹¹;

a compound represented by Formula (1) in which A¹ represents a nitrogen atom, A² represents a sulfur atom, and R¹, R², R³, and R⁴ may be the same or different and each represents a hydrogen atom, a halogen atom, a cyano group, a nitro group, a C1-C6 alkyl group optionally having one or more groups selected from the group X, a phenyl group optionally having one or more groups selected from the group Y, or —OR¹¹, and R¹¹ represents a C1-C6 alkyl group optionally having one or more groups selected from the group X;

a compound represented by Formula (1) in which A¹ represents a nitrogen atom, A² represents a sulfur atom, W represents —OR⁶, and R¹, R², R³, and R⁴ may be the same or different and each represents a hydrogen atom, a halogen atom, a cyano group, a nitro group, a C1-C6 alkyl group optionally having one or more groups selected from the group X, a phenyl group optionally having one or more groups selected from the group Y, a carboxy group, a C2-C6 alkoxycarbonyl group, an aminocarbonyl group, —NR¹¹R¹², —OR¹¹, or —S(O)ₘR¹¹;

a compound represented by Formula (1) in which A¹ represents a nitrogen atom, A² represents a sulfur atom, W represents —OR⁶, R¹, R², R³, and R⁴ may be the same or different and each represents a hydrogen atom, a halogen atom, a cyano group, a nitro group, a C1-C6 alkyl group optionally having one or more groups selected from the group X, a phenyl group optionally having one or more groups selected from the group Y, a carboxy group, a C2-C6 alkoxycarbonyl group, an aminocarbonyl group, —NR¹¹R¹², —OR¹¹, or —S(O)ₘR¹¹, and R⁶ represents a hydrogen atom or a C1-C6 alkyl group optionally having one or more groups selected from the group Z;

a compound represented by Formula (1) in which A¹ represents a nitrogen atom, A² represents a sulfur atom, W represents —OR⁶, R¹, R², R³, and R⁴ may be the same or different and each represents a hydrogen atom, a halogen atom, a cyano group, a nitro group, a C1-C6 alkyl group optionally having one or more groups selected from the group X, a phenyl group optionally having one or more groups selected from the group Y, a carboxy group, a C2-C6 alkoxycarbonyl group, an aminocarbonyl group, —NR¹¹R¹², —OR¹¹, or —S(O)ₘR¹¹, and R⁶ represents a hydrogen atom or a C1-C6 alkyl group;

a compound represented by Formula (1) in which A¹ represents a nitrogen atom, A² represents a sulfur atom, W represents —OR⁶, R¹, R², R³, and R⁴ may be the same or different and each represents a hydrogen atom, a halogen atom, a cyano group, a nitro group, a C1-C6 alkyl group optionally having one or more groups selected from the group X, a phenyl group optionally having one or more groups selected from the group Y, a carboxy group, a C2-C6 alkoxycarbonyl group, an aminocarbonyl group, —NR¹¹R¹², —OR¹¹, or —S(O)ₘR¹¹, and R⁶ represents a C1-C6 alkyl group;

a compound represented by Formula (1) in which A¹ represents a nitrogen atom, A² represents a sulfur atom, W represents —OR⁶, R¹, R², R³, and R⁴ may be the same or different and each represents a hydrogen atom, a halogen atom, a cyano group, a nitro group, a C1-C6 alkyl group optionally having one or more groups selected from the group X, a phenyl group optionally having one or more groups selected from the group Y, a carboxy group, a C2-C6 alkoxycarbonyl group, an aminocarbonyl group, —NR¹¹R¹², —OR¹¹, or —S(O)ₘR¹¹, and R⁶ represents a hydrogen atom;

a compound represented by Formula (1) in which A¹ represents a nitrogen atom, A² represents a sulfur atom, W represents —OR⁶, R¹, R², R³, and R⁴ may be the same or different and each represents a hydrogen atom, a halogen atom, a cyano group, a nitro group, a C1-C6 alkyl group optionally having one or more groups selected from the group X, a phenyl group optionally having one or more groups selected from the group Y, or —OR¹¹, R⁶ represents a hydrogen atom or a C1-C6 alkyl group, and R¹¹ represents a C1-C6 alkyl group optionally having one or more groups selected from the group X;

a compound represented by Formula (1) in which A¹ represents a nitrogen atom, A² represents a sulfur atom, W represents —OR⁶, R¹, R², R³, and R⁴ may be the same or different and each represents a hydrogen atom, a halogen atom, a cyano group, a nitro group, a C1-C6 alkyl group optionally having one or more groups selected from the group X, a phenyl group optionally having one or more groups selected from the group Y, or —OR¹¹, R⁶ represents a hydrogen atom, and R¹¹ represents a C1-C6 alkyl group optionally having one or more groups selected from the group X;

a compound represented by Formula (1) in which A¹ represents a nitrogen atom, A² represents a sulfur atom, W represents —OR⁶, R¹, R², R³, and R⁴ may be the same or different and each represents a hydrogen atom, a halogen atom, a cyano group, a nitro group, a C1-C6 alkyl group optionally having one or more groups selected from the group X, a phenyl group optionally having one or more groups selected from the group Y, or —OR¹¹, R⁶ represents a C1-C6 alkyl group, and R¹¹ represents a C1-C6 alkyl group optionally having one or more groups selected from the group X;

a compound represented by Formula (1) in which A¹ represents a nitrogen atom, A² represents a sulfur atom, W represents —OR⁶, R¹ represents a trifluoromethyl group, R², R³, and R⁴ may be the same or different and each represents a hydrogen atom, a halogen atom, a cyano group, a nitro group, a C1-C6 alkyl group optionally having one or more groups selected from the group X, a phenyl group optionally having one or more groups selected from the group Y, or —OR¹¹, R⁶ represents a hydrogen atom, a methyl group, or an ethyl group, and R¹¹ represents a C1-C6 alkyl group optionally having one or more groups selected from the group X;

a compound represented by Formula (1) in which A¹ represents a nitrogen atom, A² represents a sulfur atom, W represents —OR⁶, R¹, R³, and R⁴ may be the same or different and each represents a hydrogen atom, a halogen atom, a cyano group, a nitro group, a C1-C6 alkyl group optionally having one or more groups selected from the group X, a phenyl group optionally having one or more groups selected from the group Y, or —OR¹¹, R² represents a trifluoromethyl group, R⁶ represents a hydrogen atom, a methyl group, or an ethyl group, and R¹¹ represents a C1-C6 alkyl group optionally having one or more groups selected from the group X;

a compound represented by Formula (1) in which A¹ represents a nitrogen atom, A² represents a sulfur atom, W represents —OR⁶, R¹, R², and R⁴ may be the same or different and each represents a hydrogen atom, a halogen atom, a cyano group, a nitro group, a C1-C6 alkyl group optionally having one or more groups selected from the group X, a phenyl group optionally having one or more groups selected from the group Y, or —OR¹¹, R³ represents a trifluoromethyl group, R⁶ represents a hydrogen atom, a methyl group, or an ethyl group, and R¹¹ represents a C1-C6 alkyl group optionally having one or more groups selected from the group X;

a compound represented by Formula (1) in which A¹ represents a nitrogen atom, A² represents a sulfur atom, W represents —OR⁶, R¹, R², and R³ may be the same or different and each represents a hydrogen atom, a halogen atom, a cyano group, a nitro group, a C1-C6 alkyl group optionally having one or more groups selected from the group X, a phenyl group optionally having one or more groups selected from the group Y, or —OR¹¹, R⁴ represents a trifluoromethyl group, R⁶ represents a hydrogen atom, a methyl group, or an ethyl group, and R¹¹ represents a C1-C6 alkyl group optionally having one or more groups selected from the group X;

a compound represented by Formula (1) in which A¹ represents a nitrogen atom, A² represents a sulfur atom, W represents —OR⁶, R¹ represents a trifluoromethyl group, R², R³, and R⁴ may be the same or different and each represents a hydrogen atom, a halogen atom, a cyano group, a nitro group, a C1-C6 alkyl group optionally having one or more groups selected from the group X, a phenyl group optionally having one or more groups selected from the group Y, or —OR¹¹, R⁶ represents a methyl group or an ethyl group, and R¹¹ represents a C1-C6 alkyl group optionally having one or more groups selected from the group X;

a compound represented by Formula (1) in which A¹ represents a nitrogen atom, A² represents a sulfur atom, W represents —OR⁶, R¹, R³, and R⁴ may be the same or different and each represents a hydrogen atom, a halogen atom, a cyano group, a nitro group, a C1-C6 alkyl group optionally having one or more groups selected from the group X, a phenyl group optionally having one or more groups selected from the group Y, or —OR¹¹, R² represents a trifluoromethyl group, R⁶ represents a methyl group or an ethyl group, and R¹¹ represents a C1-C6 alkyl group optionally having one or more groups selected from the group X;

a compound represented by Formula (1) in which A¹ represents a nitrogen atom, A² represents a sulfur atom, W represents —OR⁶, R¹, R², and R⁴ may be the same or different and each represents a hydrogen atom, a halogen atom, a cyano group, a nitro group, a C1-C6 alkyl group optionally having one or more groups selected from the group X, a phenyl group optionally having one or more groups selected from the group Y, or —OR¹¹, R³ represents a trifluoromethyl group, R⁶ represents a methyl group or an ethyl group, and R¹¹ represents a C1-C6 alkyl group optionally having one or more groups selected from the group X;

a compound represented by Formula (1) in which A¹ represents a nitrogen atom, A² represents a sulfur atom, W represents —OR⁶, R¹, R², and R³ may be the same or different and each represents a hydrogen atom, a halogen atom, a cyano group, a nitro group, a C1-C6 alkyl group optionally having one or more groups selected from the group X, a phenyl group optionally having one or more groups selected from the group Y, or —OR¹¹, R⁴ represents a trifluoromethyl group, R⁶ represents a methyl group or an ethyl group, and R¹¹ represents a C1-C6 alkyl group optionally having one or more groups selected from the group X;

a compound represented by Formula (1) in which A¹ represents a nitrogen atom, A² represents a sulfur atom, and W represents —ON═CR⁷R⁸;

a compound represented by Formula (1) in which A¹ represents a nitrogen atom, A² represents a sulfur atom, and W represents —SR⁹;

a compound represented by Formula (1) in which A¹ represents a nitrogen atom, A² represents a sulfur atom, W represents —SR⁹, and R⁹ represents a C1-C6 alkyl group;

a compound represented by Formula (1) in which A¹ represents a nitrogen atom, A² represents a sulfur atom, W represents —NR⁷R¹⁰, and R¹⁰ represents a hydrogen atom, a C1-C6 alkyl group, or a phenyl group optionally having one or more groups selected from the group Y;

a compound represented by Formula (1) in which A¹ represents a nitrogen atom, A² represents a sulfur atom, W represents —NR⁷R¹⁰, and R¹⁰ represents a hydrogen atom or a C1-C6 alkyl group;

a compound represented by Formula (1) in which A¹ represents a nitrogen atom, A² represents a sulfur atom, A³ represents a nitrogen atom, and R² and R³ represent a hydrogen atom;

a compound represented by Formula (1) in which A¹ represents a nitrogen atom, A² represents a sulfur atom, A³ represents a nitrogen atom, and R¹ and R³ represent a hydrogen atom;

a compound represented by Formula (1) in which A¹ represents a nitrogen atom, A² represents a sulfur atom, A³ represents a nitrogen atom, and R¹ and R² represent a hydrogen atom;

a compound represented by Formula (1) in which A¹ represents a nitrogen atom, A² represents a sulfur atom, A³ represents a nitrogen atom, R¹ represents a trifluoromethyl group, and R² and R³ represent a hydrogen atom;

a compound represented by Formula (1) in which A¹ represents a nitrogen atom, A² represents a sulfur atom, A³ represents a nitrogen atom, R² represents a trifluoromethyl group, and R¹ and R³ represent a hydrogen atom;

a compound represented by Formula (1) in which A¹ represents a nitrogen atom, A² represents a sulfur atom, A³ represents a nitrogen atom, R³ represents a trifluoromethyl group, and R¹ and R² represent a hydrogen atom;

a compound represented by Formula (1) in which A¹ represents a nitrogen atom, A² represents a sulfur atom, A³ represents a nitrogen atom, and R¹ represents a trifluoromethyl group;

a compound represented by Formula (1) in which A¹ represents a nitrogen atom, A² represents a sulfur atom, A³ represents a nitrogen atom, and R² represents a trifluoromethyl group;

a compound represented by Formula (1) in which A¹ represents a nitrogen atom, A² represents a sulfur atom, A³ represents a nitrogen atom, and R³ represents a trifluoromethyl group;

a compound represented by Formula (1) in which A¹ represents a nitrogen atom, A² represents a sulfur atom, A³ represents a nitrogen atom, R¹ represents a halogen atom, and R² and R³ represent a hydrogen atom;

a compound represented by Formula (1) in which A¹ represents a nitrogen atom, A² represents a sulfur atom, A³ represents a nitrogen atom, R² represents a halogen atom, and R¹ and R³ represent a hydrogen atom;

a compound represented by Formula (1) in which A¹ represents a nitrogen atom, A² represents a sulfur atom, A³ represents a nitrogen atom, R³ represents a halogen atom, and R¹ and R² represent a hydrogen atom;

a compound represented by Formula (1) in which A¹ represents a nitrogen atom, A² represents a sulfur atom, A³ represents a nitrogen atom, and R¹ represents a halogen atom;

a compound represented by Formula (1) in which A¹ represents a nitrogen atom, A² represents a sulfur atom, A³ represents a nitrogen atom, and R² represents a halogen atom;

a compound represented by Formula (1) in which A¹ represents a nitrogen atom, A² represents a sulfur atom, A³ represents a nitrogen atom, and R³ represents a halogen atom;

a compound represented by Formula (1) in which A¹ represents a nitrogen atom, A² represents a sulfur atom, A³ represents a nitrogen atom, R¹ represents a C1-C6 alkyl group optionally having one or more groups selected from the group X, and R² and R³ represent a hydrogen atom;

a compound represented by Formula (1) in which A¹ represents a nitrogen atom, A² represents a sulfur atom, A³ represents a nitrogen atom, R² represents a C1-C6 alkyl group optionally having one or more groups selected from the group X, and R¹ and R³ represent a hydrogen atom;

a compound represented by Formula (1) in which A¹ represents a nitrogen atom, A² represents a sulfur atom, A³ represents a nitrogen atom, R³ represents a C1-C6 alkyl group optionally having one or more groups selected from the group X, and R¹ and R² represent a hydrogen atom;

a compound represented by Formula (1) in which A¹ represents a nitrogen atom, A² represents a sulfur atom, A³ represents a nitrogen atom, and R¹ represents a C1-C6 alkyl group optionally having one or more groups selected from the group X;

a compound represented by Formula (1) in which A¹ represents a nitrogen atom, A² represents a sulfur atom, A³ represents a nitrogen atom, and R² represents a C1-C6 alkyl group optionally having one or more groups selected from the group X;

a compound represented by Formula (1) in which A¹ represents a nitrogen atom, A² represents a sulfur atom, A³ represents a nitrogen atom, and R³ represents a C1-C6 alkyl group optionally having one or more groups selected from the group X;

a compound represented by Formula (1) in which A¹ represents a nitrogen atom, A² represents a sulfur atom, A³ represents a nitrogen atom, R¹ represents a phenyl group optionally having one or more groups selected from the group Y, and R² and R³ represent a hydrogen atom;

a compound represented by Formula (1) in which A¹ represents a nitrogen atom, A² represents a sulfur atom, A³ represents a nitrogen atom, R² represents a phenyl group optionally having one or more groups selected from the group Y, and R¹ and R³ represent a hydrogen atom;

a compound represented by Formula (1) in which A¹ represents a nitrogen atom, A² represents a sulfur atom, A³ represents a nitrogen atom, R³ represents a phenyl group optionally having one or more groups selected from the group Y, and R¹ and R² represent a hydrogen atom;

a compound represented by Formula (1) in which A¹ represents a nitrogen atom, A² represents a sulfur atom, A³ represents a nitrogen atom, and R¹ represents a phenyl group optionally having one or more groups selected from the group Y;

a compound represented by Formula (1) in which A¹ represents a nitrogen atom, A² represents a sulfur atom, A³ represents a nitrogen atom, and R² represents a phenyl group optionally having one or more groups selected from the group Y;

a compound represented by Formula (1) in which A¹ represents a nitrogen atom, A² represents a sulfur atom, A³ represents a nitrogen atom, and R³ represents a phenyl group optionally having one or more groups selected from the group Y;

a compound represented by Formula (1) in which A¹ represents a nitrogen atom, A² represents a sulfur atom, A³ represents a nitrogen atom, R¹ represents a 6-membered aromatic heterocyclic group optionally having one or more groups selected from the group Y or a 5-membered aromatic heterocyclic group optionally having one or more groups selected from the group Y, and R² and R³ represent a hydrogen atom;

a compound represented by Formula (1) in which A¹ represents a nitrogen atom, A² represents a sulfur atom, A³ represents a nitrogen atom, R² represents a 6-membered aromatic heterocyclic group optionally having one or more groups selected from the group Y or a 5-membered aromatic heterocyclic group optionally having one or more groups selected from the group Y, and R¹ and R³ represent a hydrogen atom;

a compound represented by Formula (1) in which A¹ represents a nitrogen atom, A² represents a sulfur atom, A³ represents a nitrogen atom, R³ represents a 6-membered aromatic heterocyclic group optionally having one or more groups selected from the group Y or a 5-membered aromatic heterocyclic group optionally having one or more groups selected from the group Y, and R¹ and R² represent a hydrogen atom;

a compound represented by Formula (1) in which A¹ represents a nitrogen atom, A² represents a sulfur atom, A³ represents a nitrogen atom, and R¹ represents a 6-membered aromatic heterocyclic group optionally having one or more groups selected from the group Y or a 5-membered aromatic heterocyclic group optionally having one or more groups selected from the group Y;

a compound represented by Formula (1) in which A¹ represents a nitrogen atom, A² represents a sulfur atom, A³ represents a nitrogen atom, and R² represents a 6-membered aromatic heterocyclic group optionally having one or more groups selected from the group Y or a 5-membered aromatic heterocyclic group optionally having one or more groups selected from the group Y;

a compound represented by Formula (1) in which A¹ represents a nitrogen atom, A² represents a sulfur atom, A³ represents a nitrogen atom, R¹ represents a C2-C6 alkylcarbonyl group optionally having one or more halogen atoms, and R² and R³ represent a hydrogen atom;

a compound represented by Formula (1) in which A¹ represents a nitrogen atom, A² represents a sulfur atom, A³ represents a nitrogen atom, R² represents a C2-C6 alkylcarbonyl group optionally having one or more halogen atoms, and R¹ and R³ represent a hydrogen atom;

a compound represented by Formula (1) in which A¹ represents a nitrogen atom, A² represents a sulfur atom, A³ represents a nitrogen atom, and R¹ represents a C2-C6 alkylcarbonyl group optionally having one or more halogen atoms;

a compound represented by Formula (1) in which A¹ represents a nitrogen atom, A² represents a sulfur atom, A³ represents a nitrogen atom, and R² represents a C2-C6 alkylcarbonyl group optionally having one or more halogen atoms;

a compound represented by Formula (1) in which A¹ represents a nitrogen atom, A² represents a sulfur atom, A³ represents a nitrogen atom, R¹ represents a carboxy group, and R² and R³ represent a hydrogen atom;

a compound represented by Formula (1) in which A¹ represents a nitrogen atom, A² represents a sulfur atom, A³ represents a nitrogen atom, R² represents a carboxy group, and R¹ and R³ represent a hydrogen atom;

a compound represented by Formula (1) in which A¹ represents a nitrogen atom, A² represents a sulfur atom, A³ represents a nitrogen atom, and R¹ represents a carboxy group;

a compound represented by Formula (1) in which A¹ represents a nitrogen atom, A² represents a sulfur atom, A³ represents a nitrogen atom, and R² represents a carboxy group;

a compound represented by Formula (1) in which A¹ represents a nitrogen atom, A² represents a sulfur atom, A³ represents a nitrogen atom, R¹ represents a C2-C6 alkoxycarbonyl group, and R² and R³ represent a hydrogen group;

a compound represented by Formula (1) in which A¹ represents a nitrogen atom, A² represents a sulfur atom, A³ represents a nitrogen atom, R² represents a C2-C6 alkoxycarbonyl group, and R¹ and R³ represent a hydrogen group;

a compound represented by Formula (1) in which A¹ represents a nitrogen atom, A² represents a sulfur atom, A³ represents a nitrogen atom, and R¹ represents a C2-C6 alkoxycarbonyl group;

a compound represented by Formula (1) in which A¹ represents a nitrogen atom, A² represents a sulfur atom, A³ represents a nitrogen atom, and R² represents a C2-C6 alkoxycarbonyl group;

a compound represented by Formula (1) in which A¹ represents a nitrogen atom, A² represents a sulfur atom, A³ represents a nitrogen atom, and R¹, R², and R³ may be the same or different and each represents a hydrogen atom, a halogen atom, a cyano group, a nitro group, a C1-C6 alkyl group optionally having one or more groups selected from the group X, a phenyl group optionally having one or more groups selected from the group Y, a carboxy group, a C2-C6 alkoxycarbonyl group, an aminocarbonyl group, —NR¹¹R¹², —OR¹¹, or —S(O)ₘR¹¹;

a compound represented by Formula (1) in which A¹ represents a nitrogen atom, A² represents a sulfur atom, A³ represents a nitrogen atom, R¹, R², and R³ may be the same or different and each represents a hydrogen atom, a halogen atom, a cyano group, a nitro group, a C1-C6 alkyl group optionally having one or more groups selected from the group X, a phenyl group optionally having one or more groups selected from the group Y, a carboxy group, a C2-C6 alkoxycarbonyl group, an aminocarbonyl group, —NR¹¹R¹², —OR¹¹, or —S(O)ₘR¹¹, and R¹¹ represents a C1-C6 alkyl group optionally having one or more groups selected from the group X;

a compound represented by Formula (1) in which A¹ represents a nitrogen atom, A² represents a sulfur atom, A³ represents a nitrogen atom, and R¹, R², and R³ may be the same or different and each represents a hydrogen atom, a halogen atom, a cyano group, a nitro group, a C1-C6 alkyl group optionally having one or more groups selected from the group X, a phenyl group optionally having one or more groups selected from the group Y, or —OR¹¹;

a compound represented by Formula (1) in which A¹ represents a nitrogen atom, A² represents a sulfur atom, A³ represents a nitrogen atom, R¹, R², and R³ may be the same or different and each represents a hydrogen atom, a halogen atom, a cyano group, a nitro group, a C1-C6 alkyl group optionally having one or more groups selected from the group X, a phenyl group optionally having one or more groups selected from the group Y, or —OR¹¹, and R¹¹ represents a C1-C6 alkyl group optionally having one or more groups selected from the group X;

a compound represented by Formula (1) in which A¹ represents a nitrogen atom, A² represents a sulfur atom, A³ represents a nitrogen atom, W represents —OR⁶, and R¹, R², and R³ may be the same or different and each represents a hydrogen atom, a halogen atom, a cyano group, a nitro group, a C1-C6 alkyl group optionally having one or more groups selected from the group X, a phenyl group optionally having one or more groups selected from the group Y, a carboxy group, a C2-C6 alkoxycarbonyl group, an aminocarbonyl group, —NR¹¹R¹², —OR¹¹, or —S(O)ₘR¹¹;

a compound represented by Formula (1) in which A¹ represents a nitrogen atom, A² represents a sulfur atom, A³ represents a nitrogen atom, W represents —OR⁶, R¹, R², and R³ may be the same or different and each represents a hydrogen atom, a halogen atom, a cyano group, a nitro group, a C1-C6 alkyl group optionally having one or more groups selected from the group X, a phenyl group optionally having one or more groups selected from the group Y, a carboxy group, a C2-C6 alkoxycarbonyl group, an aminocarbonyl group, —NR¹¹R¹², —OR¹¹, or —S(O)ₘR¹¹, and R⁶ represents a hydrogen atom or a C1-C6 alkyl group optionally having one or more groups selected from the group Z;

a compound represented by Formula (1) in which A¹ represents a nitrogen atom, A² represents a sulfur atom, A³ represents a nitrogen atom, W represents —OR⁶, R¹, R², and R³ may be the same or different and each represents a hydrogen atom, a halogen atom, a cyano group, a nitro group, a C1-C6 alkyl group optionally having one or more optionally having one or more groups selected from the group Y, a carboxy group, a C2-C6 alkoxycarbonyl group, an aminocarbonyl group, —NR¹¹R¹², —OR¹¹, or —S(O)ₘR¹¹, and R⁶ represents a hydrogen atom or a C1-C6 alkyl group;

a compound represented by Formula (1) in which A¹ represents a nitrogen atom, A² represents a sulfur atom, A³ represents a nitrogen atom, W represents —OR⁶, R¹, R², and R³ may be the same or different and each represents a hydrogen atom, a halogen atom, a cyano group, a nitro group, a C1-C6 alkyl group optionally having one or more groups selected from the group X, a phenyl group optionally having one or more groups selected from the group Y, a carboxy group, a C2-C6 alkoxycarbonyl group, an aminocarbonyl group, —NR¹¹R¹², —OR¹¹, or —S(O)ₘR¹¹, and R⁶ represents a C1-C6 alkyl group;

a compound represented by Formula (1) in which A¹ represents a nitrogen atom, A² represents a sulfur atom, A³ represents a nitrogen atom, W represents —OR⁶, R¹, R², and R³ may be the same or different and each represents a hydrogen atom, a halogen atom, a cyano group, a nitro group, a C1-C6 alkyl group optionally having one or more groups selected from the group X, a phenyl group optionally having one or more groups selected from the group Y, or —OR¹¹, R⁶ represents a hydrogen atom or a C1-C6 alkyl group, and R¹¹ represents a C1-C6 alkyl group optionally having one or more groups selected from the group X;

a compound represented by Formula (1) in which A¹ represents a nitrogen atom, A² represents a sulfur atom, A³ represents a nitrogen atom, W represents —OR⁶, R¹, R², and R³ may be the same or different and each represents a hydrogen atom, a halogen atom, a cyano group, a nitro group, a C1-C6 alkyl group optionally having one or more groups selected from the group X, a phenyl group optionally having one or more groups selected from the group Y, or —OR¹¹, R⁶ represents a C1-C6 alkyl group, and R¹¹ represents a C1-C6 alkyl group optionally having one or more groups selected from the group X;

a compound represented by Formula (1) in which A¹ represents a nitrogen atom, A² represents a sulfur atom, A³ represents a nitrogen atom, W represents —OR⁶, R¹ represents a trifluoromethyl group, R² and R³ may be the same or different and each represents a hydrogen atom, a halogen atom, a cyano group, a nitro group, a C1-C6 alkyl group optionally having one or more groups selected from the group X, a phenyl group optionally having one or more groups selected from the group Y, or —OR¹¹, R⁶ represents a hydrogen atom, a methyl group, or an ethyl group, and R¹¹ represents a C1-C6 alkyl group optionally having one or more groups selected from the group X;

a compound represented by Formula (1) in which A¹ represents a nitrogen atom, A² represents a sulfur atom, A³ represents a nitrogen atom, W represents —OR⁶, R¹ and R³ may be the same or different and each represents a hydrogen atom, a halogen atom, a cyano group, a nitro group, a C1-C6 alkyl group optionally having one or more groups selected from the group X, a phenyl group optionally having one or more groups selected from the group Y, or —OR¹¹, R² represents a trifluoromethyl group, R⁶ represents a hydrogen atom, a methyl group, or an ethyl group, and R¹¹ represents a C1-C6 alkyl group optionally having one or more groups selected from the group X;

a compound represented by Formula (1) in which A¹ represents a nitrogen atom, A² represents a sulfur atom, A³ represents a nitrogen atom, W represents —OR⁶, R¹ and R² may be the same or different and each represents a hydrogen atom, a halogen atom, a cyano group, a nitro group, a C1-C6 alkyl group optionally having one or more optionally having one or more groups selected from the group Y, or —OR¹¹, R³ represents a trifluoromethyl group, R⁶ represents a hydrogen atom, a methyl group, or an ethyl group, and R¹¹ represents a C1-C6 alkyl group optionally having one or more groups selected from the group X;

a compound represented by Formula (1) in which A¹ represents a nitrogen atom, A² represents a sulfur atom, A³ represents a nitrogen atom, W represents —OR⁶, R¹ represents a trifluoromethyl group, R² and R³ may be the same or different and each represents a hydrogen atom, a halogen atom, a cyano group, a nitro group, a C1-C6 alkyl group optionally having one or more groups selected from the group X, a phenyl group optionally having one or more groups selected from the group Y, or —OR¹¹, R⁶ represents a methyl group or an ethyl group, and R¹¹ represents a C1-C6 alkyl group optionally having one or more groups selected from the group X;

a compound represented by Formula (1) in which A¹ represents a nitrogen atom, A² represents a sulfur atom, A³ represents a nitrogen atom, W represents —OR⁶, R¹ and R³ may be the same or different and each represents a hydrogen atom, a halogen atom, a cyano group, a nitro group, a C1-C6 alkyl group optionally having one or more groups selected from the group X, a phenyl group optionally having one or more groups selected from the group Y, or —OR¹¹, R² represents a trifluoromethyl group, R⁶ represents a methyl group or an ethyl group, and R¹¹ represents a C1-C6 alkyl group optionally having one or more groups selected from the group X;

a compound represented by Formula (1) in which A¹ represents a nitrogen atom, A² represents a sulfur atom, A³ represents a nitrogen atom, W represents —OR⁶, R¹ and R² may be the same or different and each represents a hydrogen atom, a halogen atom, a cyano group, a nitro group, a C1-C6 alkyl group optionally having one or more groups selected from the group X, a phenyl group optionally having one or more groups selected from the group Y, or —OR¹¹, R³ represents a trifluoromethyl group, R⁶ represents a methyl group or an ethyl group, and R¹¹ represents a C1-C6 alkyl group optionally having one or more groups selected from the group X;

a compound represented by Formula (1) in which A¹ represents a nitrogen atom, A² represents a sulfur atom, A³ represents a nitrogen atom, and W represents —ON═CR⁷R⁸;

a compound represented by Formula (1) in which A¹ represents a nitrogen atom, A² represents a sulfur atom, A³ represents a nitrogen atom, and W represents —SR⁹;

a compound represented by Formula (1) in which A¹ represents a nitrogen atom, A² represents a sulfur atom, A³ represents a nitrogen atom, W represents —SR⁹, and R⁹ represents a C1-C6 alkyl group;

a compound represented by Formula (1) in which A¹ represents a nitrogen atom, A² represents a sulfur atom, A³ represents a nitrogen atom, W represents —NR⁷R¹⁰, and R¹⁰ represents a hydrogen atom, a C1-C6 alkyl group, or a phenyl group optionally having one or more groups selected from the group Y;

a compound represented by Formula (1) in which A¹ represents a nitrogen atom, A² represents a sulfur atom, A³ represents a nitrogen atom, W represents —NR⁷R¹⁰, and R¹⁰ represents a hydrogen atom or a C1-C6 alkyl group;

a compound represented by Formula (1) in which A¹ represents a nitrogen atom, A² represents a sulfur atom, A³ represents ═C(R⁴)—, and R², R³, and R⁴ represent a hydrogen atom;

a compound represented by Formula (1) in which A¹ represents a nitrogen atom, A² represents a sulfur atom, A³ represents ═C(R⁴)—, and R¹, R³, and R⁴ represent a hydrogen atom;

a compound represented by Formula (1) in which A¹ represents a nitrogen atom, A² represents a sulfur atom, A³ represents ═C(R⁴)—, and R¹, R², and R⁴ represent a hydrogen atom;

a compound represented by Formula (1) in which A¹ represents a nitrogen atom, A² represents a sulfur atom, A³ represents ═C(R⁴)—, and R¹, R², and R⁴ represent a hydrogen atom;

a compound represented by Formula (1) in which A¹ represents a nitrogen atom, A² represents a sulfur atom, A³ represents ═C(R⁴)—, R¹ represents a trifluoromethyl group, and R², R³, and R⁴ represent a hydrogen atom;

a compound represented by Formula (1) in which A¹ represents a nitrogen atom, A² represents a sulfur atom, A³ represents ═C(R⁴)—, R² represents a trifluoromethyl group, and R¹, R³, and R⁴ represent a hydrogen atom;

a compound represented by Formula (1) in which A¹ represents a nitrogen atom, A² represents a sulfur atom, A³ represents ═C(R⁴)—, R³ represents a trifluoromethyl group, and R¹, R², and R⁴ represent a hydrogen atom;

a compound represented by Formula (1) in which A¹ represents a nitrogen atom, A² represents a sulfur atom, A³ represents ═C(R⁴)—, R⁴ represents a trifluoromethyl group, and R¹, R², and R³ represent a hydrogen atom;

a compound represented by Formula (1) in which A¹ represents a nitrogen atom, A² represents a sulfur atom, A³ represents ═C(R⁴)—, and R¹ represents a trifluoromethyl group;

a compound represented by Formula (1) in which A¹ represents a nitrogen atom, A² represents a sulfur atom, A³ represents ═C(R⁴)—, and R² represents a trifluoromethyl group;

a compound represented by Formula (1) in which A¹ represents a nitrogen atom, A² represents a sulfur atom, A³ represents ═C(R⁴)—, and R³ represents a trifluoromethyl group;

a compound represented by Formula (1) in which A¹ represents a nitrogen atom, A² represents a sulfur atom, A³ represents ═C(R⁴)—, and R⁴ represents a trifluoromethyl group;

a compound represented by Formula (1) in which A¹ represents a nitrogen atom, A² represents a sulfur atom, A³ represents ═C(R⁴)—, R¹ represents a halogen atom, and R², R³, and R⁴ represent a hydrogen atom;

a compound represented by Formula (1) in which A¹ represents a nitrogen atom, A² represents a sulfur atom, A³ represents ═C(R⁴)—, R² represents a halogen atom, and R¹, R³, and R⁴ represent a hydrogen atom;

a compound represented by Formula (1) in which A¹ represents a nitrogen atom, A² represents a sulfur atom, A³ represents ═C(R⁴)—, R³ represents a halogen atom, and R¹, R², and R⁴ represent a hydrogen atom;

a compound represented by Formula (1) in which A¹ represents a nitrogen atom, A² represents a sulfur atom, A³ represents ═C(R⁴)—, R⁴ represents a halogen atom, and R¹, R², and R³ represent a hydrogen atom;

a compound represented by Formula (1) in which A¹ represents a nitrogen atom, A² represents a sulfur atom, A³ represents ═C(R⁴)—, and R¹ represents a halogen atom;

a compound represented by Formula (1) in which A¹ represents a nitrogen atom, A² represents a sulfur atom, A³ represents ═C(R⁴)—, and R² represents a halogen atom;

a compound represented by Formula (1) in which A¹ represents a nitrogen atom, A² represents a sulfur atom, A³ represents ═C(R⁴)—, and R³ represents a halogen atom;

a compound represented by Formula (1) in which A¹ represents a nitrogen atom, A² represents a sulfur atom, A³ represents ═C(R⁴)—, and R⁴ represents a halogen atom;

a compound represented by Formula (1) in which A¹ represents a nitrogen atom, A² represents a sulfur atom, A³ represents ═C(R⁴)—, R¹ represents a C1-C6 alkyl group optionally having one or more groups selected from the group X, and R², R³, and R⁴ represent a hydrogen atom;

a compound represented by Formula (1) in which A¹ represents a nitrogen atom, A² represents a sulfur atom, A³ represents ═C(R⁴)—, R² represents a C1-C6 alkyl group optionally having one or more groups selected from the group X, and R¹, R³, and R⁴ represent a hydrogen atom;

a compound represented by Formula (1) in which A¹ represents a nitrogen atom, A² represents a sulfur atom, A³ represents ═C(R⁴)—, R³ represents a C1-C6 alkyl group optionally having one or more groups selected from the group X, and R¹, R², and R⁴ represent a hydrogen atom;

a compound represented by Formula (1) in which A¹ represents a nitrogen atom, A² represents a sulfur atom, A³ represents ═C(R⁴)—, R⁴ represents a C1-C6 alkyl group optionally having one or more groups selected from the group X, and R¹, R², and R³ represent a hydrogen atom;

a compound represented by Formula (1) in which A¹ represents a nitrogen atom, A² represents a sulfur atom, A³ represents ═C(R⁴)—, and R¹ represents a C1-C6 alkyl group optionally having one or more groups selected from the group X;

a compound represented by Formula (1) in which A¹ represents a nitrogen atom, A² represents a sulfur atom, A³ represents ═C(R⁴)—, and R² represents a C1-C6 alkyl group optionally having one or more groups selected from the group X;

a compound represented by Formula (1) in which A¹ represents a nitrogen atom, A² represents a sulfur atom, A³ represents ═C(R⁴)—, and R³ represents a C1-C6 alkyl group optionally having one or more groups selected from the group X;

a compound represented by Formula (1) in which A¹ represents a nitrogen atom, A² represents a sulfur atom, A³ represents ═C(R⁴)—, and R⁴ represents a C1-C6 alkyl group optionally having one or more groups selected from the group X;

a compound represented by Formula (1) in which A¹ represents a nitrogen atom, A² represents a sulfur atom, A³ represents ═C(R⁴)—, R¹ represents a phenyl group optionally having one or more groups selected from the group Y, and R², R³, and R⁴ represent a hydrogen atom;

a compound represented by Formula (1) in which A¹ represents a nitrogen atom, A² represents a sulfur atom, A³ represents ═C(R⁴)—, R² represents a phenyl group optionally having one or more groups selected from the group Y, and R¹, R³, and R⁴ represent a hydrogen atom;

a compound represented by Formula (1) in which A¹ represents a nitrogen atom, A² represents a sulfur atom, A³ represents ═C(R⁴)—, R³ represents a phenyl group optionally having one or more groups selected from the group Y, and R¹, R², and R⁴ represent a hydrogen atom;

a compound represented by Formula (1) in which A¹ represents a nitrogen atom, A² represents a sulfur atom, A³ represents ═C(R⁴)—, R⁴ represents a phenyl group optionally having one or more groups selected from the group Y, and R¹, R², and R³ represent a hydrogen atom;

a compound represented by Formula (1) in which A¹ represents a nitrogen atom, A² represents a sulfur atom, A³ represents ═C(R⁴)—, and R¹ represents a phenyl group optionally having one or more groups selected from the group Y;

a compound represented by Formula (1) in which A¹ represents a nitrogen atom, A² represents a sulfur atom, A³ represents ═C(R⁴)—, and R² represents a phenyl group optionally having one or more groups selected from the group Y;

a compound represented by Formula (1) in which A¹ represents a nitrogen atom, A² represents a sulfur atom, A³ represents ═C(R⁴)—, and R³ represents a phenyl group optionally having one or more groups selected from the group Y;

a compound represented by Formula (1) in which A¹ represents a nitrogen atom, A² represents a sulfur atom, A³ represents ═C(R⁴)—, and R⁴ represents a phenyl group optionally having one or more groups selected from the group Y;

a compound represented by Formula (1) in which A¹ represents a nitrogen atom, A² represents a sulfur atom, A³ represents ═C(R⁴)—, R¹ represents a 6-membered aromatic heterocyclic group optionally having one or more groups selected from the group Y or a 5-membered aromatic heterocyclic group optionally having one or more groups selected from the group Y, and R², R³, and R⁴ represent a hydrogen atom;

a compound represented by Formula (1) in which A¹ represents a nitrogen atom, A² represents a sulfur atom, A³ represents ═C(R⁴)—, R² represents a 6-membered aromatic heterocyclic group optionally having one or more groups selected from the group Y or a 5-membered aromatic heterocyclic group optionally having one or more groups selected from the group Y, and R¹, R³, and R⁴ represent a hydrogen atom;

a compound represented by Formula (1) in which A¹ represents a nitrogen atom, A² represents a sulfur atom, A³ represents ═C(R⁴)—, and R¹ represents a 6-membered aromatic heterocyclic group optionally having one or more groups selected from the group Y or a 5-membered aromatic heterocyclic group optionally having one or more groups selected from the group Y;

a compound represented by Formula (1) in which A¹ represents a nitrogen atom, A² represents a sulfur atom, A³ represents ═C(R⁴)—, and R² represents a 6-membered aromatic heterocyclic group optionally having one or more groups selected from the group Y or a 5-membered aromatic heterocyclic group optionally having one or more groups selected from the group Y;

a compound represented by Formula (1) in which A¹ represents a nitrogen atom, A² represents a sulfur atom, A³ represents ═C(R⁴)—, R¹ represents a C2-C6 alkylcarbonyl group optionally having one or more halogen atoms, and R², R³, and R⁴ represent a hydrogen atom;

a compound represented by Formula (1) in which A¹ represents a nitrogen atom, A² represents a sulfur atom, A³ represents ═C(R⁴)—, R² represents a C2-C6 alkylcarbonyl group optionally having one or more halogen atoms, and R¹, R³, and R⁴ represent a hydrogen atom;

a compound represented by Formula (1) in which A¹ represents a nitrogen atom, A² represents a sulfur atom, A³ represents ═C(R⁴)—, and R¹ represents a C2-C6 alkylcarbonyl group optionally having one or more halogen atoms;

a compound represented by Formula (1) in which A¹ represents a nitrogen atom, A² represents a sulfur atom, A³ represents ═C(R⁴)—, and R² represents a C2-C6 alkylcarbonyl group optionally having one or more halogen atoms;

a compound represented by Formula (1) in which A¹ represents a nitrogen atom, A² represents a sulfur atom, A³ represents ═C(R⁴)—, R¹ represents a carboxy group, and R², R³, and R⁴ represent a hydrogen atom;

a compound represented by Formula (1) in which A¹ represents a nitrogen atom, A² represents a sulfur atom, A³ represents ═C(R⁴)—, R² represents a carboxy group, and R¹, R³, and R⁴ represent a hydrogen atom;

a compound represented by Formula (1) in which A¹ represents a nitrogen atom, A² represents a sulfur atom, A³ represents ═C(R⁴)—, and R¹ represents a carboxy group;

a compound represented by Formula (1) in which A¹ represents a nitrogen atom, A² represents a sulfur atom, A³ represents ═C(R⁴)—, and R² represents a carboxy group;

a compound represented by Formula (1) in which A¹ represents a nitrogen atom, A² represents a sulfur atom, A³ represents ═C(R⁴)—, R¹ represents a C2-C6 alkoxycarbonyl group, and R², R³, and R⁴ represent a hydrogen atom;

a compound represented by Formula (1) in which A¹ represents a nitrogen atom, A² represents a sulfur atom, A³ represents ═C(R⁴)—, R² represents a C2-C6 alkoxycarbonyl group, and R¹, R³, and R⁴ represent a hydrogen atom;

a compound represented by Formula (1) in which A¹ represents a nitrogen atom, A² represents a sulfur atom, A³ represents ═C(R⁴)—, and R¹ represents a C2-C6 alkoxycarbonyl group;

a compound represented by Formula (1) in which A¹ represents a nitrogen atom, A² represents a sulfur atom, A³ represents ═C(R⁴)—, and R² represents a C2-C6 alkoxycarbonyl group;

a compound represented by Formula (1) in which A¹ represents a nitrogen atom, A² represents a sulfur atom, A³ represents ═C(R⁴)—, and R¹, R², R³, and R⁴ may be the same or different and each represents a hydrogen atom, a halogen atom, a cyano group, a nitro group, a C1-C6 alkyl group optionally having one or more groups selected from the group X, a phenyl group optionally having one or more groups selected from the group Y, a carboxy group, a C2-C6 alkoxycarbonyl group, an aminocarbonyl group, —NR¹¹R¹², —OR¹¹, or —S(O)ₘR¹¹;

a compound represented by Formula (1) in which A¹ represents a nitrogen atom, A² represents a sulfur atom, A³ represents ═C(R⁴)—, R¹, R², R³, and R⁴ may be the same or different and each represents a hydrogen atom, a halogen atom, a cyano group, a nitro group, a C1-C6 alkyl group optionally having one or more groups selected from the group X, a phenyl group optionally having one or more groups selected from the group Y, a carboxy group, a C2-C6 alkoxycarbonyl group, an aminocarbonyl group, —NR¹¹R¹², —OR¹¹, or —S(O)ₘR¹¹, and R¹¹ represents a C1-C6 alkyl group optionally having one or more groups selected from the group X;

a compound represented by Formula (1) in which A¹ represents a nitrogen atom, A² represents a sulfur atom, A³ represents ═C(R⁴)—, and R¹, R², R³, and R⁴ may be the same or different and each represents a hydrogen atom, a halogen atom, a cyano group, a nitro group, a C1-C6 alkyl group optionally having one or more groups selected from the group X, a phenyl group optionally having one or more groups selected from the group Y, or —OR¹¹;

a compound represented by Formula (1) in which A¹ represents a nitrogen atom, A² represents a sulfur atom, A³ represents ═C(R⁴)—, R¹, R², R³, and R⁴ may be the same or different and each represents a hydrogen atom, a halogen atom, a cyano group, a nitro group, a C1-C6 alkyl group optionally having one or more groups selected from the group X, a phenyl group optionally having one or more groups selected from the group Y, or —OR¹¹, and R¹¹ represents a C1-C6 alkyl group optionally having one or more groups selected from the group X;

a compound represented by Formula (1) in which A¹ represents a nitrogen atom, A² represents a sulfur atom, A³ represents ═C(R⁴)—, W represents —OR⁶, and R¹, R², R³, and R⁴ may be the same or different and each represents a hydrogen atom, a halogen atom, a cyano group, a nitro group, a C1-C6 alkyl group optionally having one or more groups selected from the group X, a phenyl group optionally having one or more groups selected from the group Y, a carboxy group, a C2-C6 alkoxycarbonyl group, an aminocarbonyl group, —NR¹¹R¹², —OR¹¹, or —S(O)ₘR¹¹;

a compound represented by Formula (1) in which A¹ represents a nitrogen atom A² represents a sulfur atom, A³ represents ═C(R⁴)—, W represents —OR⁶, R¹, R², R³, and R⁴ may be the same or different and each represents a hydrogen atom, a halogen atom, a cyano group, a nitro group, a C1-C6 alkyl group optionally having one or more groups selected from the group X, a phenyl group optionally having one or more groups selected from the group Y, a carboxy group, a C2-C6 alkoxycarbonyl group, an aminocarbonyl group, —NR¹¹R¹², —OR¹¹, or —S(O)ₘR¹¹, and R⁶ represents a hydrogen atom or a C1-C6 alkyl group optionally having one or more groups selected from the group Z;

a compound represented by Formula (1) in which A¹ represents a nitrogen atom, A² represents a sulfur atom, A³ represents ═C(R⁴)—, W represents —OR⁶, R¹, R², R³, and R⁴ may be the same or different and each represents a hydrogen atom, a halogen atom, a cyano group, a nitro group, a C1-C6 alkyl group optionally having one or more groups selected from the group X, a phenyl group optionally having one or more groups selected from the group Y, a carboxy group, a C2-C6 alkoxycarbonyl group, an aminocarbonyl group, —NR¹¹R¹², —OR¹¹, or —S(O)ₘR¹¹, and R⁶ represents a hydrogen atom or a C1-C6 alkyl group;

a compound represented by Formula (1) in which A¹ represents a nitrogen atom, A² represents a sulfur atom, A³ represents ═C(R⁴)—, W represents —OR⁶, R¹, R², R³, and R⁴ may be the same or different and each represents a hydrogen atom, a halogen atom, a cyano group, a nitro group, a C1-C6 alkyl group optionally having one or more groups selected from the group X, a phenyl group optionally having one or more groups selected from the group Y, a carboxy group, a C2-C6 alkoxycarbonyl group, an aminocarbonyl group, —NR¹¹R¹², —OR¹¹, or —S(O)ₘR¹¹, and R⁶ represents a C1-C6 alkyl group;

a compound represented by Formula (1) in which A¹ represents a nitrogen atom, A² represents a sulfur atom, A³ represents ═C(R⁴)—, W represents —OR⁶, R¹, R², R³, and R⁴ may be the same or different and each represents a hydrogen atom, a halogen atom, a cyano group, a nitro group, a C1-C6 alkyl group optionally having one or more groups selected from the group X, a phenyl group optionally having one or more groups selected from the group Y, a carboxy group, a C2-C6 alkoxycarbonyl group, an aminocarbonyl group, —NR¹¹R¹², —OR¹¹, or —S(O)ₘR¹¹, and R⁶ represents a hydrogen atom;

a compound represented by Formula (1) in which A¹ represents a nitrogen atom, A² represents a sulfur atom, A³ represents ═C(R⁴)—, W represents —OR⁶, R¹, R², R³, and R⁴ may be the same or different and each represents a hydrogen atom, a halogen atom, a cyano group, a nitro group, a C1-C6 alkyl group optionally having one or more groups selected from the group X, a phenyl group optionally having one or more groups selected from the group Y, or —OR¹¹, R⁶ represents a hydrogen atom or a C1-C6 alkyl group, and R¹¹ represents a C1-C6 alkyl group optionally having one or more groups selected from the group X;

a compound represented by Formula (1) in which A¹ represents a nitrogen atom, A² represents a sulfur atom, A³ represents ═C(R⁴)—, W represents —OR⁶, R¹, R², R³, and R⁴ may be the same or different and each represents a hydrogen atom, a halogen atom, a cyano group, a nitro group, a C1-C6 alkyl group optionally having one or more groups selected from the group X, a phenyl group optionally having one or more groups selected from the group Y, or —OR¹¹, R⁶ represents a hydrogen atom, and R¹¹ represents a C1-C6 alkyl group optionally having one or more groups selected from the group X;

a compound represented by Formula (1) in which A¹ represents a nitrogen atom, A² represents a sulfur atom, A³ represents ═C(R⁴)—, W represents —OR⁶, R¹, R², R³, and R⁴ may be the same or different and each represents a hydrogen atom, a halogen atom, a cyano group, a nitro group, a C1-C6 alkyl group optionally having one or more groups selected from the group X, a phenyl group optionally having one or more groups selected from the group Y, or —OR¹¹, R⁶ represents a C1-C6 alkyl group, and R¹¹ represents a C1-C6 alkyl group optionally having one or more groups selected from the group X;

a compound represented by Formula (1) in which A¹ represents a nitrogen atom, A² represents a sulfur atom, A³ represents ═C(R⁴)—, W represents —OR⁶, R¹ represents a trifluoromethyl group, R², R³, and R⁴ may be the same or different and each represents a hydrogen atom, a halogen atom, a cyano group, a nitro group, a C1-C6 alkyl group optionally having one or more groups selected from the group X, a phenyl group optionally having one or more groups selected from the group Y, or —OR¹¹, R⁶ represents a hydrogen atom, a methyl group, or an ethyl group, and R¹¹ represents a C1-C6 alkyl group optionally having one or more groups selected from the group X;

a compound represented by Formula (1) in which A¹ represents a nitrogen atom, A² represents a sulfur atom, A³ represents ═C(R⁴)—, W represents —OR⁶, R¹, R³, and R⁴ may be the same or different and each represents a hydrogen atom, a halogen atom, a cyano group, a nitro group, a C1-C6 alkyl group optionally having one or more groups selected from the group X, a phenyl group optionally having one or more groups selected from the group Y, or —OR¹¹, R² represents a trifluoromethyl group, R⁶ represents a hydrogen atom, a methyl group, or an ethyl group, and R¹¹ represents a C1-C6 alkyl group optionally having one or more groups selected from the group X;

a compound represented by Formula (1) in which A¹ represents a nitrogen atom, A² represents a sulfur atom, A³ represents ═C(R⁴)—, W represents —OR⁶, R¹, R², and R⁴ may be the same or different and each represents a hydrogen atom, a halogen atom, a cyano group, a nitro group, a C1-C6 alkyl group optionally having one or more groups selected from the group X, a phenyl group optionally having one or more groups selected from the group Y, or —OR¹¹, R³ represents a trifluoromethyl group, R⁶ represents a hydrogen atom, a methyl group, or an ethyl group, and R¹¹ represents a C1-C6 alkyl group optionally having one or more groups selected from the group X;

a compound represented by Formula (1) in which A¹ represents a nitrogen atom, A² represents a sulfur atom, A³ represents ═C(R⁴)—, W represents —OR⁶, R¹, R², and R³ may be the same or different and each represents a hydrogen atom, a halogen atom, a cyano group, a nitro group, a C1-C6 alkyl group optionally having one or more groups selected from the group X, a phenyl group optionally having one or more groups selected from the group Y, or —OR¹¹, R⁴ represents a trifluoromethyl group, R⁶ represents a hydrogen atom, a methyl group, or an ethyl group, and R¹¹ represents a C1-C6 alkyl group optionally having one or more groups selected from the group X;

a compound represented by Formula (1) in which A¹ represents a nitrogen atom, A² represents a sulfur atom, A³ represents ═C(R⁴)—, W represents —OR⁶, R¹ represents a trifluoromethyl group, R², R³, and R⁴ may be the same or different and each represents a hydrogen atom, a halogen atom, a cyano group, a nitro group, a C1-C6 alkyl group optionally having one or more groups selected from the group X, a phenyl group optionally having one or more groups selected from the group Y, or —OR¹¹, R⁶ represents a methyl group or an ethyl group, and R¹¹ represents a C1-C6 alkyl group optionally having one or more groups selected from the group X;

a compound represented by Formula (1) in which A¹ represents a nitrogen atom, A² represents a sulfur atom, A³ represents ═C(R⁴)—, W represents —OR⁶, R¹, R³, and R⁴ may be the same or different and each represents a hydrogen atom, a halogen atom, a cyano group, a nitro group, a C1-C6 alkyl group optionally having one or more groups selected from the group X, a phenyl group optionally having one or more groups selected from the group Y, or —OR¹¹, R² represents a trifluoromethyl group, R⁶ represents a methyl group or an ethyl group, and R¹¹ represents a C1-C6 alkyl group optionally having one or more groups selected from the group X;

a compound represented by Formula (1) in which A¹ represents a nitrogen atom, A² represents a sulfur atom, A³ represents ═C(R⁴)—, W represents —OR⁶, R¹, R², and R⁴ may be the same or different and each represents a hydrogen atom, a halogen atom, a cyano group, a nitro group, a C1-C6 alkyl group optionally having one or more groups selected from the group X, a phenyl group optionally having one or more groups selected from the group Y, or —OR¹¹, R³ represents a trifluoromethyl group, R⁶ represents a methyl group or an ethyl group, and R¹¹ represents a C1-C6 alkyl group optionally having one or more groups selected from the group X;

a compound represented by Formula (1) in which A¹ represents a nitrogen atom, A² represents a sulfur atom, A³ represents ═C(R⁴)—, W represents —OR⁶, R¹, R², and R³ may be the same or different and each represents a hydrogen atom, a halogen atom, a cyano group, a nitro group, a C1-C6 alkyl group optionally having one or more groups selected from the group X, a phenyl group optionally having one or more groups selected from the group Y, or —OR¹¹, R⁴ represents a trifluoromethyl group, R⁶ represents a methyl group or an ethyl group, and R¹¹ represents a C1-C6 alkyl group optionally having one or more groups selected from the group X;

a compound represented by Formula (1) in which A¹ represents a nitrogen atom, A² represents a sulfur atom, A³ represents ═C(R⁴)—, and W represents —ON═CR⁷R⁸;

a compound represented by Formula (1) in which A¹ represents a nitrogen atom, A² represents a sulfur atom, A³ represents ═C(R⁴)—, and W represents —SR⁹;

a compound represented by Formula (1) in which A¹ represents a nitrogen atom, A² represents a sulfur atom, A³ represents ═C(R⁴)—, W represents —SR⁹, and R⁹ represents a C1-C6 alkyl group;

a compound represented by Formula (1) in which A¹ represents a nitrogen atom, A² represents a sulfur atom, A³ represents ═C(R⁴)—, W represents —NR⁷R¹⁰, and R¹⁰ represents a hydrogen atom, a C1-C6 alkyl group, or a phenyl group optionally having one or more groups selected from the group Y;

a compound represented by Formula (1) in which A¹ represents a nitrogen atom, A² represents a sulfur atom, A³ represents ═C(R⁴)—, W represents —NR⁷R¹⁰, and R¹⁰ represents a hydrogen atom, or a C1-C6 alkyl group;

a compound represented by Formula (1) in which A¹ represents —C(R⁵)═, A² represents —N(R¹²)—, A³ represents ═C(R⁴)—, R¹ represents a trifluoromethyl group, and R², R³, R⁴, and R⁵ represent a hydrogen atom;

a compound represented by Formula (1) in which A¹ represents —C(R⁵)═, A² represents —N(R¹²)—, A³ represents ═C(R⁴)—, R² represents a trifluoromethyl group, and R¹, R³, R⁴, and R⁵ represent a hydrogen atom;

a compound represented by Formula (1) in which A¹ represents —C(R⁵)═, A² represents —N(R¹²)—, A³ represents ═C(R⁴)—, R³ represents a trifluoromethyl group, and R¹, R², R⁴, and R⁵ represent a hydrogen atom;

a compound represented by Formula (1) in which A¹ represents —C(R⁵)═, A² represents —N(R¹²)—, A³ represents ═C(R⁴)—, R⁴ represents a trifluoromethyl group, and R¹, R², R³, and R⁵ represent a hydrogen atom;

a compound represented by Formula (1) in which A¹ represents —C(R⁵)═, A² represents —N(R¹²)—, A³ represents ═C(R⁴)—, R⁵ represents a trifluoromethyl group, and R¹, R², R³, and R⁴ represent a hydrogen atom;

a compound represented by Formula (1) in which A¹ represents —C(R⁵)═, A² represents —N(R¹²)—, A³ represents ═C(R⁴)—, R¹ represents a trifluoromethyl group, R², R³, R⁴, and R⁵ represent a hydrogen atom, and R¹² represents a hydrogen atom;

a compound represented by Formula (1) in which A¹ represents —C(R⁵)═, A² represents —N(R¹²)—, A³ represents ═C(R⁴)—, R² represents a trifluoromethyl group, R¹, R³, R⁴, and R⁵ represent a hydrogen atom, and R¹² represents a hydrogen atom;

a compound represented by Formula (1) in which A¹ represents —C(R⁵)═, A² represents —N(R¹²)—, A³ represents ═C(R⁴)—, R³ represents a trifluoromethyl group, R¹, R², R⁴, and R⁵ represent a hydrogen atom, and R¹² represents a hydrogen atom;

a compound represented by Formula (1) in which A¹ represents —C(R⁵)═, A² represents —N(R¹²)—, A³ represents ═C(R⁴)—, R⁴ represents a trifluoromethyl group, R¹, R², R³, and R⁵ represent a hydrogen atom, and R¹² represents a hydrogen atom;

a compound represented by Formula (1) in which A¹ represents —C(R⁵)═, A² represents —N(R¹²)—, A³ represents ═C(R⁴)—, R⁵ represents a trifluoromethyl group, R¹, R², R³, and R⁴ represent a hydrogen atom, and R¹² represents a hydrogen atom;

a compound represented by Formula (1) in which A¹ represents —C(R⁵)═, A² represents —N(R¹²)—, A³ represents ═C(R⁴)—, R¹ represents a trifluoromethyl group, R², R³, R⁴, and R⁵ represent a hydrogen atom, and R¹² represents a methyl group;

a compound represented by Formula (1) in which A¹ represents —C(R⁵)═, A² represents —N(R¹²)—, A³ represents ═C(R⁴)—, R² represents a trifluoromethyl group, R¹, R³, R⁴, and R⁵ represent a hydrogen atom, and R¹² represents a methyl group;

a compound represented by Formula (1) in which A¹ represents —C(R⁵)═, A² represents —N(R¹²)—, A³ represents ═C(R⁴)—, R³ represents a trifluoromethyl group, R¹, R², R⁴, and R⁵ represent a hydrogen atom, and R¹² represents a methyl group;

a compound represented by Formula (1) in which A¹ represents —C(R⁵)═, A² represents —N(R¹²)—, A³ represents ═C(R⁴)—, R⁴ represents a trifluoromethyl group, R¹, R², R³, and R⁵ represent a hydrogen atom, and R¹² represents a methyl group;

a compound represented by Formula (1) in which A¹ represents —C(R⁵)═, A² represents —N(R¹²)—, A³ represents ═C(R⁴)—, R⁵ represents a trifluoromethyl group, R¹, R², R³, and R⁴ represent a hydrogen atom, and R¹² represents a methyl group;

a compound represented by Formula (1) in which A¹ represents —C(R⁵)═, A² represents —N(R¹²)—, A³ represents ═C(R⁴)—, and R¹ represents a trifluoromethyl group;

a compound represented by Formula (1) in which A¹ represents —C(R⁵)═, A² represents —N(R¹²)—, A³ represents ═C(R⁴)—, and R² represents a trifluoromethyl group;

a compound represented by Formula (1) in which A¹ represents —C(R⁵)═, A² represents —N(R¹²)—, A³ represents ═C(R⁴)—, and R³ represents a trifluoromethyl group;

a compound represented by Formula (1) in which A¹ represents —C(R⁵)═, A² represents —N(R¹²)—, A³ represents ═C(R⁴)—, and R⁴ represents a trifluoromethyl group;

a compound represented by Formula (1) in which A¹ represents —C(R⁵)═, A² represents —N(R¹²)—, A³ represents ═C(R⁴)—, and R⁵ represents a trifluoromethyl group;

a compound represented by Formula (1) in which A¹ represents —C(R⁵)═, A² represents —N(R¹²)—, A³ represents ═C(R⁴)—, R¹ represents a trifluoromethyl group, and R¹² represents a hydrogen atom or a C1-C4 alkyl group optionally having one or more halogen atoms;

a compound represented by Formula (1) in which A¹ represents —C(R⁵)═, A² represents —N(R¹²)—, A³ represents ═C(R⁴)—, R² represents a trifluoromethyl group, and R¹² represents a hydrogen atom or a C1-C4 alkyl group optionally having one or more halogen atoms;

a compound represented by Formula (1) in which A¹ represents —C(R⁵)═, A² represents —N(R¹²)—, A³ represents ═C(R⁴)—, R³ represents a trifluoromethyl group, and R¹² represents a hydrogen atom or a C1-C4 alkyl group optionally having one or more halogen atoms;

a compound represented by Formula (1) in which A¹ represents —C(R⁵)═, A² represents —N(R¹²)—, A³ represents ═C(R⁴)—, R⁴ represents a trifluoromethyl group, and R¹² represents a hydrogen atom or a C1-C4 alkyl group optionally having one or more halogen atoms;

a compound represented by Formula (1) in which A¹ represents —C(R⁵)═, A² represents —N(R¹²)—, A³ represents ═C(R⁴)—, R⁵ represents a trifluoromethyl group, and R¹² represents a hydrogen atom or a C1-C4 alkyl group optionally having one or more halogen atoms;

a compound represented by Formula (1) in which A¹ represents —C(R⁵)═, A² represents —N(R¹²)—, A³ represents ═C(R⁴)—, R¹ represents a halogen atom, and R², R³, R⁴, and R⁵ represents a hydrogen atom;

a compound represented by Formula (1) in which A¹ represents —C(R⁵)═, A² represents —N(R¹²)—, A³ represents ═C(R⁴)—, R³ represents a halogen atom, and R¹, R², R⁴, and R⁵ represents a hydrogen atom;

a compound represented by Formula (1) in which A¹ represents —C(R⁵)═, A² represents —N(R¹²)—, A³ represents ═C(R⁴)—, R⁴ represents a halogen atom, and R¹, R², R³, and R⁵ represents a hydrogen atom;

a compound represented by Formula (1) in which A¹ represents —C(R⁵)═, A² represents —N(R¹²)—, A³ represents ═C(R⁴)—, and R¹ represents a halogen atom;

a compound represented by Formula (1) in which A¹ represents —C(R⁵)═, A² represents —N(R¹²)—, A³ represents ═C(R⁴)—, and R³ represents a halogen atom;

a compound represented by Formula (1) in which A¹ represents —C(R⁵)═, A² represents —N(R¹²)—, A³ represents ═C(R⁴)—, and R⁴ represents a halogen atom;

a compound represented by Formula (1) in which A¹ represents —C(R⁵)═, A² represents —N(R¹²)—, A³ represents ═C(R⁴)—, and R⁵ represents a halogen atom;

a compound represented by Formula (1) in which A¹ represents —C(R⁵)═, A² represents —N(R¹²)—, A³ represents ═C(R⁴)—, R¹ represents a C1-C6 alkyl group optionally having one or more groups selected from the group X, and R², R³, R⁴, and R⁵ represent a hydrogen atom;

a compound represented by Formula (1) in which A¹ represents —C(R⁵)═, A² represents —N(R¹²)—, A³ represents ═C(R⁴)—, R² represents a C1-C6 alkyl group optionally having one or more groups selected from the group X, and R¹, R³, R⁴, and R⁵ represent a hydrogen atom;

a compound represented by Formula (1) in which A¹ represents —C(R⁵)═, A² represents —N(R¹²)—, A³ represents ═C(R⁴)—, R³ represents a C1-C6 alkyl group optionally having one or more groups selected from the group X, and R¹, R², R⁴, and R⁵ represent a hydrogen atom;

a compound represented by Formula (1) in which A¹ represents —C(R⁵)═, A² represents —N(R¹²)—, A³ represents C(R⁴)—, R⁴ represents a C1-C6 alkyl group optionally having one or more groups selected from the group X, and R¹, R², R³, and R⁵ represent a hydrogen atom;

a compound represented by Formula (1) in which A¹ represents —C(R⁵)═, A² represents —N(R¹²)—, A³ represents ═C(R⁴)—, R⁵ represents a C1-C6 alkyl group optionally having one or more groups selected from the group X, and R¹, R², R³, and R⁴ represent a hydrogen atom;

a compound represented by Formula (1) in which A¹ represents —C(R⁵)═, A² represents —N(R¹²)—, A³ represents ═C(R⁴)—, and R¹ represents a C1-C6 alkyl group optionally having one or more groups selected from the group X;

a compound represented by Formula (1) in which A¹ represents —C(R⁵)═, A² represents —N(R¹²)—, A³ represents ═C(R⁴)—, and R² represents a C1-C6 alkyl group optionally having one or more groups selected from the group X;

a compound represented by Formula (1) in which A¹ represents —C(R⁵)═, A² represents —N(R¹²)—, A³ represents ═C(R⁴)—, and R³ represents a C1-C6 alkyl group optionally having one or more groups selected from the group X;

a compound represented by Formula (1) in which A¹ represents —C(R⁵)═, A² represents —N(R¹²)—, A³ represents ═C(R⁴)—, and R⁴ represents a C1-C6 alkyl group optionally having one or more groups selected from the group X;

a compound represented by Formula (1) in which A¹ represents —C(R⁵)═, A² represents —N(R¹²)—, A³ represents ═C(R⁴)—, and R⁵ represents a C1-C6 alkyl group optionally having one or more groups selected from the group X;

a compound represented by Formula (1) in which A¹ represents —C(R⁵)═, A² represents —N(R¹²)—, A³ represents ═C(R⁴)—, R¹ represents a phenyl group optionally having one or more groups selected from the group Y, and R², R³, R⁴, and R⁵ represent a hydrogen atom;

a compound represented by Formula (1) in which A¹ represents —C(R⁵)═, A² represents —N(R¹²)—, A³ represents ═C(R⁴)—, R² represents a phenyl group optionally having one or more groups selected from the group Y, and R¹, R³, R⁴, and R⁵ represent a hydrogen atom;

a compound represented by Formula (1) in which A¹ represents —C(R⁵)═, A² represents —N(R¹²)—, A³ represents ═C(R⁴)—, R³ represents a phenyl group optionally having one or more groups selected from the group Y, and R¹, R², R⁴, and R⁵ represent a hydrogen atom;

a compound represented by Formula (1) in which A¹ represents —C(R⁵)═, A² represents —N(R¹²)—, A³ represents ═C(R⁴)—, R⁴ represents a phenyl group optionally having one or more groups selected from the group Y, and R¹, R², R³, and R⁵ represent a hydrogen atom;

a compound represented by Formula (1) in which A¹ represents —C(R⁵)═, A² represents —N(R¹²)—, A³ represents ═C(R⁴)—, R⁵ represents a phenyl group optionally having one or more groups selected from the group Y, and R¹, R², R³, and R⁴ represent a hydrogen atom;

a compound represented by Formula (1) in which A¹ represents —C(R⁵)═, A² represents —N(R¹²)—, A³ represents ═C(R⁴)—, and R¹ represents a phenyl group optionally having one or more groups selected from the group Y;

a compound represented by Formula (1) in which A¹ represents —C(R⁵)═, A² represents —N(R¹²)—, A³ represents C(R⁴)—, and R² represents a phenyl group optionally having one or more groups selected from the group Y;

a compound represented by Formula (1) in which A¹ represents —C(R⁵)═, A² represents —N(R¹²)—, A³ represents ═C(R⁴)—, and R³ represents a phenyl group optionally having one or more groups selected from the group Y;

a compound represented by Formula (1) in which A¹ represents —C(R⁵)═, A² represents —N(R¹²)—, A³ represents ═C(R⁴)—, and R⁴ represents a phenyl group optionally having one or more groups selected from the group Y;

a compound represented by Formula (1) in which A¹ represents —C(R⁵)═, A² represents —N(R¹²)—, A³ represents ═C(R⁴)—, and R⁵ represents a phenyl group optionally having one or more groups selected from the group Y;

a compound represented by Formula (1) in which A¹ represents —C(R⁵)═, A² represents —N(R¹²)—, A³ represents ═C(R⁴)—, R¹ represents a 6-membered aromatic heterocyclic group optionally having one or more groups selected from the group Y or a 5-membered aromatic heterocyclic group optionally having one or more groups selected from the group Y, and R², R³, R⁴, and R⁵ represent a hydrogen atom;

a compound represented by Formula (1) in which A¹ represents —C(R⁵)═, A² represents —N(R¹²)—, A³ represents ═C(R⁴)—, R² represents a 6-membered aromatic heterocyclic group optionally having one or more groups selected from the group Y or a 5-membered aromatic heterocyclic group optionally having one or more groups selected from the group Y, and R¹, R³, R⁴, and R⁵ represent a hydrogen atom;

a compound represented by Formula (1) in which A¹ represents —C(R⁵)═, A² represents —N(R¹²)—, A³ represents ═C(R⁴)—, R³ represents a 6-membered aromatic heterocyclic group optionally having one or more groups selected from the group Y or a 5-membered aromatic heterocyclic group optionally having one or more groups selected from the group Y, and R¹, R², R⁴, and R⁵ represent a hydrogen atom;

a compound represented by Formula (1) in which A¹ represents —C(R⁵)═, A² represents —N(R¹²)—, A³ represents ═C(R⁴)—, R⁴ represents a 6-membered aromatic heterocyclic group optionally having one or more groups selected from the group Y or a 5-membered aromatic heterocyclic group optionally having one or more groups selected from the group Y, and R¹, R², R³, and R⁵ represent a hydrogen atom;

a compound represented by Formula (1) in which A¹ represents —C(R⁵)═, A² represents —N(R¹²)—, A³ represents ═C(R⁴)—, R⁵ represents a 6-membered aromatic heterocyclic group optionally having one or more groups selected from the group Y or a 5-membered aromatic heterocyclic group optionally having one or more groups selected from the group Y, and R¹, R², R³, and R⁴ represent a hydrogen atom;

a compound represented by Formula (1) in which A¹ represents —C(R⁵)═, A² represents —N(R¹²)—, A³ represents ═C(R⁴)—, and R¹ represents a 6-membered aromatic heterocyclic group optionally having one or more groups selected from the group Y or a 5-membered aromatic heterocyclic group optionally having one or more groups selected from the group Y;

a compound represented by Formula (1) in which A¹ represents —C(R⁵)═, A² represents —N(R¹²)—, A³ represents ═C(R⁴)—, and R² represents a 6-membered aromatic heterocyclic group optionally having one or more groups selected from the group Y or a 5-membered aromatic heterocyclic group optionally having one or more groups selected from the group Y;

a compound represented by Formula (1) in which A¹ represents —C(R⁵)═, A² represents —N(R¹²)—, A³ represents ═C(R⁴)—, and R³ represents a 6-membered aromatic heterocyclic group optionally having one or more groups selected from the group Y or a 5-membered aromatic heterocyclic group optionally having one or more groups selected from the group Y;

a compound represented by Formula (1) in which A¹ represents —C(R⁵)═, A² represents —N(R¹²)—, A³ represents ═C(R⁴)—, and R⁴ represents a 6-membered aromatic heterocyclic group optionally having one or more groups selected from the group Y or a 5-membered aromatic heterocyclic group optionally having one or more groups selected from the group Y;

a compound represented by Formula (1) in which A¹ represents —C(R⁵)═, A² represents —N(R¹²)—, A³ represents ═C(R⁴)—, and R⁵ represents a 6-membered aromatic heterocyclic group optionally having one or more groups selected from the group Y or a 5-membered aromatic heterocyclic group optionally having one or more groups selected from the group Y;

a compound represented by Formula (1) in which A¹ represents —C(R⁵)═, A² represents —N(R¹²)—, A³ represents ═C(R⁴)—, R¹ represents a C2-C6 alkylcarbonyl group optionally having one or more halogen atoms, and R², R³, R⁴, and R⁵ represent a hydrogen atom;

a compound represented by Formula (1) in which A¹ represents —C(R⁵)═, A² represents —N(R¹²)—, A³ represents ═C(R⁴)—, R² represents a C2-C6 alkylcarbonyl group optionally having one or more halogen atoms, and R¹, R³, R⁴, and R⁵ represent a hydrogen atom;

a compound represented by Formula (1) in which A¹ represents —C(R⁵)═, A² represents —N(R¹²)—, A³ represents ═C(R⁴)—, and R¹ represents a C2-C6 alkylcarbonyl group optionally having one or more halogen atoms;

a compound represented by Formula (1) in which A¹ represents —C(R⁵)═, A² represents —N(R¹²)—, A³ represents ═C(R⁴)—, and R² represents a C2-C6 alkylcarbonyl group optionally having one or more halogen atoms;

a compound represented by Formula (1) in which A¹ represents —C(R⁵)═, A² represents —N(R¹²)—, A³ represents ═C(R⁴)—, R¹ represents a carboxy group, and R², R³, R⁴, and R⁵ represent a hydrogen atom;

a compound represented by Formula (1) in which A¹ represents —C(R⁵)═, A² represents —N(R¹²)—, A³ represents ═C(R⁴)—, R² represents a carboxy group, and R¹, R³, R⁴, and R⁵ represent a hydrogen atom;

a compound represented by Formula (1) in which A¹ represents —C(R⁵)═, A² represents —N(R¹²)—, A³ represents ═C(R⁴)—, R³ represents a carboxy group, and R¹, R², R⁴, and R⁵ represent a hydrogen atom;

a compound represented by Formula (1) in which A¹ represents —C(R⁵)═, A² represents —N(R¹²)—, A³ represents ═C(R⁴)—, R⁴ represents a carboxy group, and R¹, R², R³, and R⁵ represent a hydrogen atom;

a compound represented by Formula (1) in which A¹ represents —C(R⁵)═, A² represents —N(R¹²)—, A³ represents ═C(R⁴)—, and R¹ represents a carboxy group;

a compound represented by Formula (1) in which A¹ represents —C(R⁵)═, A² represents —N(R¹²)—, A³ represents ═C(R⁴)—, and R² represents a carboxy group;

a compound represented by Formula (1) in which A¹ represents —C(R⁵)═, A² represents —N(R¹²)—, A³ represents ═C(R⁴)—, and R³ represents a carboxy group;

a compound represented by Formula (1) in which A¹ represents —C(R⁵)═, A² represents —N(R¹²)—, A³ represents ═C(R⁴)—, and R⁴ represents a carboxy group;

a compound represented by Formula (1) in which A¹ represents —C(R⁵)═, A² represents —N(R¹²)—, A³ represents ═C(R⁴)—, R¹ represents a C2-C6 alkoxycarbonyl group, and R², R³, R⁴, and R⁵ represent a hydrogen atom;

a compound represented by Formula (1) in which A¹ represents —C(R⁵)═, A² represents —N(R¹²)—, A³ represents ═C(R⁴)—, R² represents a C2-C6 alkoxycarbonyl group, and R¹, R³, R⁴, and R⁵ represent a hydrogen atom;

a compound represented by Formula (1) in which A¹ represents —C(R⁵)═, A² represents —N(R¹²)—, A³ represents ═C(R⁴)—, R³ represents a C2-C6 alkoxycarbonyl group, and R¹, R², R⁴, and R⁵ represent a hydrogen atom;

a compound represented by Formula (1) in which A¹ represents —C(R⁵)═, A² represents —N(R¹²)—, A³ represents ═C(R⁴)—, R⁴ represents a C2-C6 alkoxycarbonyl group, and R¹, R², R³, and R⁵ represent a hydrogen atom;

a compound represented by Formula (1) in which A¹ represents —C(R⁵)═, A² represents —N(R¹²)—, A³ represents ═C(R⁴)—, and R¹ represents a C2-C6 alkoxycarbonyl group;

a compound represented by Formula (1) in which A¹ represents —C(R⁵)═, A² represents —N(R¹²)—, A³ represents ═C(R⁴)—, and R² represents a C2-C6 alkoxycarbonyl group;

a compound represented by Formula (1) in which A¹ represents —C(R⁵)═, A² represents —N(R¹²)—, A³ represents ═C(R⁴)—, and R³ represents a C2-C6 alkoxycarbonyl group;

a compound represented by Formula (1) in which A¹ represents —C(R⁵)═, A² represents —N(R¹²)—, A³ represents ═C(R⁴)—, and R⁴ represents a C2-C6 alkoxycarbonyl group;

a compound represented by Formula (1) in which A¹ represents —C(R⁵)═, A² represents —N(R¹²)—, A³ represents ═C(R⁴)—, R¹ represents a trifluoromethyl group, and R², R³, and R⁴ represent a hydrogen atom;

a compound represented by Formula (1) in which A¹ represents —C(R⁵)═, A² represents —N(R¹²)—, A³ represents ═C(R⁴)—, R² represents a trifluoromethyl group, and R¹, R³, and R⁴ represent a hydrogen atom;

a compound represented by Formula (1) in which A¹ represents —C(R⁵)═, A² represents —N(R¹²)—, A³ represents ═C(R⁴)—, R³ represents a trifluoromethyl group, and R¹, R², and R⁴ represent a hydrogen atom;

a compound represented by Formula (1) in which A¹ represents —C(R⁵)═, A² represents —N(R¹²)—, A³ represents ═C(R⁴)—, R⁴ represents a trifluoromethyl group, and R¹, R², and R³ represent a hydrogen atom;

a compound represented by Formula (1) in which A¹ represents —C(R⁵)═, A² represents —N(R¹²)—, A³ represents ═C(R⁴)—, R⁵ represents a trifluoromethyl group, and R², R³, and R⁴ represent a hydrogen atom;

a compound represented by Formula (1) in which A¹ represents —C(R⁵)═, A² represents —N(R¹²)—, A³ represents ═C(R⁴)—, R⁵ represents a trifluoromethyl group, and R¹, R³, and R⁴ represent a hydrogen atom;

a compound represented by Formula (1) in which A¹ represents —C(R⁵)═, A² represents —N(R¹²)—, A³ represents ═C(R⁴)—, R⁵ represents a trifluoromethyl group, and R¹, R², and R⁴ represent a hydrogen atom;

a compound represented by Formula (1) in which A¹ represents —C(R⁵)═, A² represents —N(R¹²)—, A³ represents ═C(R⁴)—, R⁵ represents a trifluoromethyl group, and R¹, R², and R³ represent a hydrogen atom;

a compound represented by Formula (1) in which A¹ represents —C(R⁵)═, A² represents —N(R¹²)—, A³ represents ═C(R⁴)—, R¹ represents a halogen atom, and R², R³, and R⁴ represent a hydrogen atom;

a compound represented by Formula (1) in which A¹ represents —C(R⁵)═, A² represents —N(R¹²)—, A³ represents ═C(R⁴)—, R³ represents a halogen atom, and R¹, R³, and R⁴ represent a hydrogen atom;

a compound represented by Formula (1) in which A¹ represents —C(R⁵)═, A² represents —N(R¹²)—, A³ represents ═C(R⁴)—, R⁴ represents a halogen atom, and R¹, R², and R³ represent a hydrogen atom;

a compound represented by Formula (1) in which A¹ represents —C(R⁵)═, A² represents —N(R¹²)—, A³ represents ═C(R⁴)—, and R¹ represents a halogen atom;

a compound represented by Formula (1) in which A¹ represents —C(R⁵)═, A² represents —N(R¹²)—, A³ represents ═C(R⁴)—, and R³ represents a halogen atom;

a compound represented by Formula (1) in which A¹ represents —C(R⁵)═, A² represents —N(R¹²)—, A³ represents ═C(R⁴)—, and R⁴ represents a halogen atom;

a compound represented by Formula (1) in which A¹ represents —C(R⁵)═, A² represents —N(R¹²)—, A³ represents ═C(R⁴)—, R¹ represents a C1-C6 alkyl group optionally having one or more groups selected from the group X, and R², R³, and R⁴ represent a hydrogen atom;

a compound represented by Formula (1) in which A¹ represents —C(R⁵)═, A² represents —N(R¹²)—, A³ represents ═C(R⁴)—, R² represents a C1-C6 alkyl group optionally having one or more groups selected from the group X, and R¹, R³, and R⁴ represent a hydrogen atom;

a compound represented by Formula (1) in which A¹ represents —C(R⁵)═, A² represents —N(R¹²)—, A³ represents ═C(R⁴)—, R³ represents a C1-C6 alkyl group optionally having one or more groups selected from the group X, and R¹, R², and R⁴ represent a hydrogen atom;

a compound represented by Formula (1) in which A¹ represents —C(R⁵)═, A² represents —N(R¹²)—, A³ represents ═C(R⁴)—, R⁴ represents a C1-C6 alkyl group optionally having one or more groups selected from the group X, and R¹, R², and R³ represent a hydrogen atom;

a compound represented by Formula (1) in which A¹ represents —C(R⁵)═, A² represents —N(R¹²)—, A³ represents ═C(R⁴)—, and R¹ represents a C1-C6 alkyl group optionally having one or more groups selected from the group X;

a compound represented by Formula (1) in which A¹ represents —C(R⁵)═, A² represents —N(R¹²)—, A³ represents ═C(R⁴)—, and R² represents a C1-C6 alkyl group optionally having one or more groups selected from the group X;

a compound represented by Formula (1) in which A¹ represents —C(R⁵)═, A² represents —N(R¹²)—, A³ represents ═C(R⁴)—, and R³ represents a C1-C6 alkyl group optionally having one or more groups selected from the group X;

a compound represented by Formula (1) in which A¹ represents —C(R⁵)═, A² represents —N(R¹²)—, A³ represents ═C(R⁴)—, and R⁴ represents a C1-C6 alkyl group optionally having one or more groups selected from the group X;

a compound represented by Formula (1) in which A¹ represents —C(R⁵)═, A² represents —N(R¹²)—, A³ represents ═C(R⁴)—, R¹ represents a phenyl group optionally having one or more groups selected from the group Y, and R², R³, and R⁴ represent a hydrogen atom;

a compound represented by Formula (1) in which A¹ represents —C(R⁵)═, A² represents —N(R¹²)—, A³ represents ═C(R⁴)—, R² represents a phenyl group optionally having one or more groups selected from the group Y, and R¹, R³, and R⁴ represent a hydrogen atom;

a compound represented by Formula (1) in which A¹ represents —C(R⁵)═, A² represents —N(R¹²)—, A³ represents ═C(R⁴)—, R³ represents a phenyl group optionally having one or more groups selected from the group Y, and R¹, R², and R⁴ represent a hydrogen atom;

a compound represented by Formula (1) in which A¹ represents —C(R⁵)═, A² represents —N(R¹²)—, A³ represents ═C(R⁴)—, R⁴ represents a phenyl group optionally having one or more groups selected from the group Y, and R¹, R², and R³ represent a hydrogen atom;

a compound represented by Formula (1) in which A¹ represents —C(R⁵)═, A² represents —N(R¹²)—, A³ represents ═C(R⁴)—, and R¹ represents a phenyl group optionally having one or more groups selected from the group Y;

a compound represented by Formula (1) in which A¹ represents —C(R⁵)═, A² represents —N(R¹²)—, A³ represents ═C(R⁴)—, and R² represents a phenyl group optionally having one or more groups selected from the group Y;

a compound represented by Formula (1) in which A¹ represents —C(R⁵)═, A² represents —N(R¹²)—, A³ represents ═C(R⁴)—, and R³ represents a phenyl group optionally having one or more groups selected from the group Y;

a compound represented by Formula (1) in which A¹ represents —C(R⁵)═, A² represents —N(R¹²)—, A³ represents ═C(R⁴)—, and R⁴ represents a phenyl group optionally having one or more groups selected from the group Y;

a compound represented by Formula (1) in which A¹ represents —C(R⁵)═, A² represents —N(R¹²)—, A³ represents ═C(R⁴)—, R¹ represents a 6-membered aromatic heterocyclic group optionally having one or more groups selected from the group Y or a 5-membered aromatic heterocyclic group optionally having one or more groups selected from the group Y, and R², R³, and R⁴ represent a hydrogen atom;

a compound represented by Formula (1) in which A¹ represents —C(R⁵)═, A² represents —N(R¹²)—, A³ represents ═C(R⁴)—, R² represents a 6-membered aromatic heterocyclic group optionally having one or more groups selected from the group Y or a 5-membered aromatic heterocyclic group optionally having one or more groups selected from the group Y, and R¹, R³, and R⁴ represent a hydrogen atom;

a compound represented by Formula (1) in which A¹ represents —C(R⁵)═, A² represents —N(R¹²)—, A³ represents ═C(R⁴)—, and R¹ represents a 6-membered aromatic heterocyclic group optionally having one or more groups selected from the group Y or a 5-membered aromatic heterocyclic group optionally having one or more groups selected from the group Y;

a compound represented by Formula (1) in which A¹ represents —C(R⁵)═, A² represents —N(R¹²)—, A³ represents ═C(R⁴)—, and R² represents a 6-membered aromatic heterocyclic group optionally having one or more groups selected from the group Y or a 5-membered aromatic heterocyclic group optionally having one or more groups selected from the group Y;

a compound represented by Formula (1) in which A¹ represents —C(R⁵)═, A² represents —N(R¹²)—, A³ represents ═C(R⁴)—, R¹ represents a C2-C6 alkylcarbonyl group optionally having one or more halogen atoms, and R², R³, and R⁴ represent a hydrogen atom;

a compound represented by Formula (1) in which A¹ represents —C(R⁵)═, A² represents —N(R¹²)—, A³ represents ═C(R⁴)—, R² represents a C2-C6 alkylcarbonyl group optionally having one or more halogen atoms, and R¹, R³, and R⁴ represent a hydrogen atom;

a compound represented by Formula (1) in which A¹ represents —C(R⁵)═, A² represents —N(R¹²)—, A³ represents ═C(R⁴)—, and R¹ represents a C2-C6 alkylcarbonyl group optionally having one or more halogen atoms;

a compound represented by Formula (1) in which A¹ represents —C(R⁵)═, A² represents —N(R¹²)—, A³ represents ═C(R⁴)—, and R² represents a C2-C6 alkylcarbonyl group optionally having one or more halogen atoms;

a compound represented by Formula (1) in which A¹ represents —C(R⁵)═, A² represents —N(R¹²)—, A³ represents ═C(R⁴)—, R¹ represents a carboxy group, and R², R³, and R⁴ represent a hydrogen atom;

a compound represented by Formula (1) in which A¹ represents —C(R⁵)═, A² represents —N(R¹²)—, A³ represents ═C(R⁴)—, R² represents a carboxy group, and R¹, R³, and R⁴ represent a hydrogen atom;

a compound represented by Formula (1) in which A¹ represents —C(R⁵)═, A² represents —N(R¹²)—, A³ represents ═C(R⁴)—, R³ represents a carboxy group, and R¹, R², and R⁴ represent a hydrogen atom;

a compound represented by Formula (1) in which A¹ represents —C(R⁵)═, A² represents —N(R¹²)—, A³ represents ═C(R⁴)—, R⁴ represents a carboxy group, and R¹, R², and R³ represent a hydrogen atom;

a compound represented by Formula (1) in which A¹ represents —C(R⁵)═, A² represents —N(R¹²)—, A³ represents ═C(R⁴)—, and R¹ represents a carboxy group;

a compound represented by Formula (1) in which A¹ represents —C(R⁵)═, A² represents —N(R¹²)—, A³ represents ═C(R⁴)—, and R² represents a carboxy group;

a compound represented by Formula (1) in which A¹ represents —C(R⁵)═, A² represents —N(R¹²)—, A³ represents ═C(R⁴)—, and R³ represents a carboxy group;

a compound represented by Formula (1) in which A¹ represents —C(R⁵)═, A² represents —N(R¹²)—, A³ represents ═C(R⁴)—, and R⁴ represents a carboxy group;

a compound represented by Formula (1) in which A¹ represents —C(R⁵)═, A² represents —N(R¹²)—, A³ represents ═C(R⁴)—, R¹ represents a C2-C6 alkoxycarbonyl group, and R², R³, and R⁴ represent a hydrogen atom;

a compound represented by Formula (1) in which A¹ represents —C(R⁵)═, A² represents —N(R¹²)—, A³ represents ═C(R⁴)—, R² represents a C2-C6 alkoxycarbonyl group, and R¹, R³, and R⁴ represent a hydrogen atom;

a compound represented by Formula (1) in which A¹ represents —C(R⁵)═, A² represents —N(R¹²)—, A³ represents ═C(R⁴)—, R³ represents a C2-C6 alkoxycarbonyl group, and R¹, R², and R⁴ represent a hydrogen atom;

a compound represented by Formula (1) in which A¹ represents —C(R⁵)═, A² represents —N(R¹²)—, A³ represents ═C(R⁴)—, R⁴ represents a C2-C6 alkoxycarbonyl group, and R¹, R², and R³ represent a hydrogen atom;

a compound represented by Formula (1) in which A¹ represents —C(R⁵)═, A² represents —N(R¹²)—, A³ represents ═C(R⁴)—, and R¹ represents a C2-C6 alkoxycarbonyl group;

a compound represented by Formula (1) in which A¹ represents —C(R⁵)═, A² represents —N(R¹²)—, A³ represents ═C(R⁴)—, and R² represents a C2-C6 alkoxycarbonyl group;

a compound represented by Formula (1) in which A¹ represents —C(R⁵)═, A² represents —N(R¹²)—, A³ represents ═C(R⁴)—, and R³ represents a C2-C6 alkoxycarbonyl group;

a compound represented by Formula (1) in which A¹ represents —C(R⁵)═, A² represents —N(R¹²)—, A³ represents ═C(R⁴)—, and R⁴ represents a C2-C6 alkoxycarbonyl group;

a compound represented by Formula (1) in which A¹ represents —C(R⁵)═, A² represents —N(R¹²)—, A³ represents ═C(R⁴)—, R¹, R³, and R⁴ may be the same or different and each represents a hydrogen atom, a halogen atom, a cyano group, a nitro group, a C1-C6 alkyl group optionally having one or more groups selected from the group X, a phenyl group optionally having one or more groups selected from the group Y, a carboxy group, a C2-C6 alkoxycarbonyl group, an aminocarbonyl group, —NR¹¹R¹², —OR¹¹, or —S(O)ₘR¹¹, and R² represents a hydrogen atom, a cyano group, a nitro group, a C1-C6 alkyl group optionally having one or more groups selected from the group X, a phenyl group optionally having one or more groups selected from the group Y, a carboxy group, a C2-C6 alkoxycarbonyl group, an aminocarbonyl group, —NR¹¹R¹², —OR¹⁹, or —S(O)ₘR¹¹;

a compound represented by Formula (1) in which A¹ represents —C(R⁵)═, A² represents —N(R¹²)—, A³ represents ═C(R⁴)—, R¹, R³, and R⁴ may be the same or different and each represents a hydrogen atom, a halogen atom, a cyano group, a nitro group, a C1-C6 alkyl group optionally having one or more groups selected from the group X, a phenyl group optionally having one or more groups selected from the group Y, a carboxy group, a C2-C6 alkoxycarbonyl group, an aminocarbonyl group, —NR¹¹R¹², —OR¹¹, or —S(O)ₘR¹¹, R² represents a hydrogen atom, a cyano group, a nitro group, a C1-C6 alkyl group optionally having one or more groups selected from the group X, a phenyl group optionally having one or more groups selected from the group Y, a carboxy group, a C2-C6 alkoxycarbonyl group, an aminocarbonyl group, —NR¹¹R¹², —OR⁹, or —S(O)ₘR¹¹, and R¹² represents a hydrogen atom or a C1-C4 alkyl group optionally having one or more halogen atoms;

a compound represented by Formula (1) in which A¹ represents —C(R⁵)═, A² represents —N(R¹²)—, A³ represents ═C(R⁴)—, R¹, R³, and R⁴ may be the same or different and each represents a hydrogen atom, a halogen atom, a cyano group, a nitro group, a C1-C6 alkyl group optionally having one or more groups selected from the group X, a phenyl group optionally having one or more groups selected from the group Y, a carboxy group, a C2-C6 alkoxycarbonyl group, an aminocarbonyl group, —NR¹¹R¹², —OR¹¹, or —S(O)ₘR¹¹, R² represents a hydrogen atom, a cyano group, a nitro group, a C1-C6 alkyl group optionally having one or more groups selected from the group X, a phenyl group optionally having one or more groups selected from the group Y, a carboxy group, a C2-C6 alkoxycarbonyl group, an aminocarbonyl group, —NR¹¹R¹², —OR¹⁹, or —S(O)ₘR¹¹, R¹¹ represents a C1-C6 alkyl group optionally having one or more groups selected from the group X, R¹² represents a hydrogen atom or a C1-C4 alkyl group optionally having one or more halogen atoms, and R¹⁹ represents a C2-C6 alkyl group optionally having one or more groups selected from the group X;

a compound represented by Formula (1) in which A¹ represents —C(R⁵)═, A² represents —N(R¹²)—, A³ represents ═C(R⁴)—, R¹, R³, and R⁴ may be the same or different and each represents a hydrogen atom, a halogen atom, a cyano group, a nitro group, a C1-C6 alkyl group optionally having one or more groups selected from the group X, a phenyl group optionally having one or more groups selected from the group Y, a carboxy group, a C2-C6 alkoxycarbonyl group, an aminocarbonyl group, —NR¹¹R¹², —OR¹¹, or —S(O)ₘR¹¹, R² represents a hydrogen atom, a cyano group, a nitro group, a C1-C6 alkyl group optionally having one or more groups selected from the group X, a phenyl group optionally having one or more groups selected from the group Y, a carboxy group, a C2-C6 alkoxycarbonyl group, an aminocarbonyl group, —NR¹¹R¹², —OR¹⁹, or —S(O)ₘR¹¹, R⁵ represents a hydrogen atom, R¹¹ represents a C1-C6 alkyl group optionally having one or more groups selected from the group X, R¹² represents a hydrogen atom or a C1-C4 alkyl group optionally having one or more halogen atoms, and R¹⁹ represents a C2-C6 alkyl group optionally having one or more groups selected from the group X;

a compound represented by Formula (1) in which A¹ represents —C(R⁵)═, A² represents —N(R¹²)—, A³ represents ═C(R⁴)—, R¹, R³, and R⁴ may be the same or different and each represents a hydrogen atom, a halogen atom, a cyano group, a nitro group, a C1-C6 alkyl group optionally having one or more groups selected from the group X, a phenyl group optionally having one or more groups selected from the group Y, a carboxy group, a C2-C6 alkoxycarbonyl group, an aminocarbonyl group, —NR¹¹R¹², —OR¹¹, or —S(O)ₘR¹¹, R² represents a hydrogen atom, a cyano group, a nitro group, a C1-C6 alkyl group optionally having one or more groups selected from the group X, a phenyl group optionally having one or more groups selected from the group Y, a carboxy group, a C2-C6 alkoxycarbonyl group, an aminocarbonyl group, —NR¹¹R¹², —OR¹⁹, or —S(O)ₘR¹¹, R¹¹ represents a C1-C6 alkyl group optionally having one or more groups selected from the group X, R¹² represents a hydrogen atom, and R¹⁹ represents a C2-C6 alkyl group optionally having one or more groups selected from the group X;

a compound represented by Formula (1) in which A¹ represents —C(R⁵)═, A² represents —N(R¹²)—, A³ represents ═C(R⁴)—, R¹, R³, and R⁴ may be the same or different and each represents a hydrogen atom, a halogen atom, a cyano group, a nitro group, a C1-C6 alkyl group optionally having one or more groups selected from the group X, a phenyl group optionally having one or more groups selected from the group Y, a carboxy group, a C2-C6 alkoxycarbonyl group, an aminocarbonyl group, —NR¹¹R¹², —OR¹¹, or —S(O)ₘR¹¹, R² represents a hydrogen atom, a cyano group, a nitro group, a C1-C6 alkyl group optionally having one or more groups selected from the group X, a phenyl group optionally having one or more groups selected from the group Y, a carboxy group, a C2-C6 alkoxycarbonyl group, an aminocarbonyl group, —NR¹¹R¹², —OR¹⁹, or —S(O)ₘR¹¹, R¹¹ represents a C1-C6 alkyl group optionally having one or more groups selected from the group X, R¹² represents a C1-C4 alkyl group optionally having one or more halogen atoms, and R¹⁹ represents a C2-C6 alkyl group optionally having one or more groups selected from the group X;

a compound represented by Formula (1) in which A¹ represents —C(R⁵)═, A² represents —N(R¹²)—, A³ represents ═C(R⁴)—, R¹, R³, and R⁴ may be the same or different and each represents a hydrogen atom, a halogen atom, a cyano group, a nitro group, a C1-C6 alkyl group optionally having one or more groups selected from the group X, a phenyl group optionally having one or more groups selected from the group Y, or —OR¹¹, R² represents a hydrogen atom, a cyano group, a nitro group, a C1-C6 alkyl group optionally having one or more groups selected from the group X, a phenyl group optionally having one or more groups selected from the group Y, or —OR¹⁹, and R¹² represents a hydrogen atom or a C1-C4 alkyl group optionally having one or more halogen atoms;

a compound represented by Formula (1) in which A¹ represents —C(R⁵)═, A² represents —N(R¹²)—, A³ represents ═C(R⁴)—, R¹, R³, and R⁴ may be the same or different and each represents a hydrogen atom, a halogen atom, a cyano group, a nitro group, a C1-C6 alkyl group optionally having one or more groups selected from the group X, a phenyl group optionally having one or more groups selected from the group Y, or —OR¹¹, R² represents a hydrogen atom, a cyano group, a nitro group, a C1-C6 alkyl group optionally having one or more groups selected from the group X, or a phenyl group optionally having one or more groups selected from the group Y, R¹¹ represents a C1-C6 alkyl group optionally having one or more groups selected from the group X, and R¹² represents a hydrogen atom or a C1-C4 alkyl group optionally having one or more halogen atoms;

a compound represented by Formula (1) in which A¹ represents —C(R⁵)═, A² represents —N(R¹²)—, A³ represents ═C(R⁴)—, R¹, R³, and R⁴ may be the same or different and each represents a hydrogen atom, a halogen atom, a cyano group, a nitro group, a C1-C6 alkyl group optionally having one or more groups selected from the group X, a phenyl group optionally having one or more groups selected from the group Y, or —OR¹¹, R² represents a hydrogen atom, a cyano group, a nitro group, a C1-C6 alkyl group optionally having one or more groups selected from the group X, or a phenyl group optionally having one or more groups selected from the group Y, R⁵ represents a hydrogen atom, R¹¹ represents a C1-C6 alkyl group optionally having one or more groups selected from the group X, and R¹² represents a hydrogen atom or a C1-C4 alkyl group optionally having one or more halogen atoms;

a compound represented by Formula (1) in which A¹ represents —C(R⁵)═, A² represents —N(R¹²)—, A³ represents ═C(R⁴)—, R¹, R³, and R⁴ may be the same or different and each represents a hydrogen atom, a halogen atom, a cyano group, a nitro group, a C1-C6 alkyl group optionally having one or more groups selected from the group X, a phenyl group optionally having one or more groups selected from the group Y, or —OR¹¹, R² represents a hydrogen atom, a cyano group, a nitro group, a C1-C6 alkyl group optionally having one or more groups selected from the group X, or a phenyl group optionally having one or more groups selected from the group Y, R¹¹ represents a C1-C6 alkyl group optionally having one or more groups selected from the group X, and R¹² represents a hydrogen atom;

a compound represented by Formula (1) in which A¹ represents —C(R⁵)═, A² represents —N(R¹²)—, A³ represents ═C(R⁴)—, R¹, R³, and R⁴ may be the same or different and each represents a hydrogen atom, a halogen atom, a cyano group, a nitro group, a C1-C6 alkyl group optionally having one or more groups selected from the group X, a phenyl group optionally having one or more groups selected from the group Y, or —OR¹¹, R² represents a hydrogen atom, a cyano group, a nitro group, a C1-C6 alkyl group optionally having one or more groups selected from the group X, or a phenyl group optionally having one or more groups selected from the group Y, R¹¹ represents a C1-C6 alkyl group optionally having one or more groups selected from the group X, and R¹² represents a C1-C4 alkyl group optionally having one or more halogen atoms;

a compound represented by Formula (1) in which A¹ represents —C(R⁵)═, A² represents —N(R¹²)—, A³ represents ═C(R⁴)—, W represents —OR⁶, R¹, R³, and R⁴ may be the same or different and each represents a hydrogen atom, a halogen atom, a cyano group, a nitro group, a C1-C6 alkyl group optionally having one or more groups selected from the group X, a phenyl group optionally having one or more groups selected from the group Y, a carboxy group, a C2-C6 alkoxycarbonyl group, an aminocarbonyl group, —NR¹¹R¹², OR¹¹, or —S(O)ₘR¹¹, and R² represents a hydrogen atom, a cyano group, a nitro group, a C1-C6 alkyl group optionally having one or more groups selected from the group X, a phenyl group optionally having one or more groups selected from the group Y, a carboxy group, a C2-C6 alkoxycarbonyl group, an aminocarbonyl group, —NR¹¹R¹², —OR¹⁹, or —S(O)ₘR¹¹;

a compound represented by Formula (1) in which A¹ represents —C(R⁵)═, A² represents —N(R¹²)—, A³ represents ═C(R⁴)—, W represents —OR⁶, R¹, R³, and R⁴ may be the same or different and each represents a hydrogen atom, a halogen atom, a cyano group, a nitro group, a C1-C6 alkyl group optionally having one or more groups selected from the group X, a phenyl group optionally having one or more groups selected from the group Y, a carboxy group, a C2-C6 alkoxycarbonyl group, an aminocarbonyl group, —NR¹¹R¹², —OR¹¹, or —S(O)ₘR¹¹, R² represents a hydrogen atom, a cyano group, a nitro group, a C1-C6 alkyl group optionally having one or more groups selected from the group X, a phenyl group optionally having one or more groups selected from the group Y, a carboxy group, a C2-C6 alkoxycarbonyl group, an aminocarbonyl group, —NR¹¹R¹², —OR¹⁹, or —S(O)ₘR¹¹, and R⁵ represent a hydrogen atom;

a compound represented by Formula (1) in which A¹ represents —C(R⁵)═, A² represents —N(R¹²)—, A³ represents ═C(R⁴)—, W represents —OR⁶, R¹, R³, and R⁴ may be the same or different and each represents a hydrogen atom, a halogen atom, a cyano group, a nitro group, a C1-C6 alkyl group optionally having one or more groups selected from the group X, a phenyl group optionally having one or more groups selected from the group Y, a carboxy group, a C2-C6 alkoxycarbonyl group, an aminocarbonyl group, —NR¹¹R¹², —OR¹¹, or —S(O)ₘR¹¹, R² represents a hydrogen atom, a cyano group, a nitro group, a C1-C6 alkyl group optionally having one or more groups selected from the group X, a phenyl group optionally having one or more groups selected from the group Y, a carboxy group, a C2-C6 alkoxycarbonyl group, an aminocarbonyl group, —NR¹¹R¹², —OR¹⁹, or —S(O)ₘR¹¹, and R⁶ represent a hydrogen atom or a C1-C6 alkyl group optionally having one or more groups selected from the group Z;

a compound represented by Formula (1) in which A¹ represents —C(R⁵)═, A² represents —N(R¹²)—, A³ represents ═C(R⁴)—, W represents —OR⁶, R¹, R³, and R⁴ may be the same or different and each represents a hydrogen atom, a halogen atom, a cyano group, a nitro group, a C1-C6 alkyl group optionally having one or more groups selected from the group X, a phenyl group optionally having one or more groups selected from the group Y, a carboxy group, a C2-C6 alkoxycarbonyl group, an aminocarbonyl group, —NR¹¹R¹², —OR¹¹, or —S(O)ₘR¹¹, R² represents a hydrogen atom, a cyano group, a nitro group, a C1-C6 alkyl group optionally having one or more groups selected from the group X, a phenyl group optionally having one or more groups selected from the group Y, a carboxy group, a C2-C6 alkoxycarbonyl group, an aminocarbonyl group, —NR¹¹R¹², —OR¹⁹, or —S(O)ₘR¹¹, and R⁶ represent a hydrogen atom or a C1-C6 alkyl group;

a compound represented by Formula (1) in which A¹ represents —C(R⁵)═, A² represents —N(R¹²)—, A³ represents ═C(R⁴)—, W represents —OR⁶, R¹, R³, and R⁴ may be the same or different and each represents a hydrogen atom, a halogen atom, a cyano group, a nitro group, a C1-C6 alkyl group optionally having one or more groups selected from the group X, a phenyl group optionally having one or more groups selected from the group Y, a carboxy group, a C2-C6 alkoxycarbonyl group, an aminocarbonyl group, —NR¹¹R¹², —OR¹¹, or —S(O)ₘR¹¹, R² represents a hydrogen atom, a cyano group, a nitro group, a C1-C6 alkyl group optionally having one or more groups selected from the group X, a phenyl group optionally having one or more groups selected from the group Y, a carboxy group, a C2-C6 alkoxycarbonyl group, an aminocarbonyl group, —NR¹¹R¹², —OR¹⁹, or —S(O)ₘR¹¹, and R⁶ represent a C1-C6 alkyl group;

a compound represented by Formula (1) in which A¹ represents —C(R⁵)═, A² represents —N(R¹²)—, A³ represents ═C(R⁴)—, W represents —OR⁶, R¹, R³, and R⁴ may be the same or different and each represents a hydrogen atom, a halogen atom, a cyano group, a nitro group, a C1-C6 alkyl group optionally having one or more groups selected from the group X, a phenyl group optionally having one or more groups selected from the group Y, a carboxy group, a C2-C6 alkoxycarbonyl group, an aminocarbonyl group, —NR¹¹R¹², —OR¹¹, or —S(O)ₘR¹¹, R² represents a hydrogen atom, a cyano group, a nitro group, a C1-C6 alkyl group optionally having one or more groups selected from the group X, a phenyl group optionally having one or more groups selected from the group Y, a carboxy group, a C2-C6 alkoxycarbonyl group, an aminocarbonyl group, —NR¹¹R¹², —OR¹⁹, or —S(O)ₘR¹¹, and R⁶ represent a hydrogen atom;

a compound represented by Formula (1) in which A¹ represents —C(R⁵)═, A² represents —N(R¹²)—, A³ represents ═C(R⁴)—, W represents —OR⁶, R¹, R³, and R⁴ may be the same or different and each represents a hydrogen atom, a halogen atom, a cyano group, a nitro group, a C1-C6 alkyl group optionally having one or more groups selected from the group X, a phenyl group optionally having one or more groups selected from the group Y, or —OR¹¹, R² represents a hydrogen atom, a cyano group, a nitro group, a C1-C6 alkyl group optionally having one or more groups selected from the group X, or a phenyl group optionally having one or more groups selected from the group Y, R⁶ represents a hydrogen atom or a C1-C6 alkyl group, R¹¹ represents a C1-C6 alkyl group optionally having one or more groups selected from the group X, and R¹² represents a hydrogen atom or a C1-C4 alkyl group optionally having one or more halogen atoms;

a compound represented by Formula (1) in which A¹ represents —C(R⁵)═, A² represents —N(R¹²)—, A³ represents ═C(R⁴)—, W represents —OR⁶, R¹, R³, and R⁴ may be the same or different and each represents a hydrogen atom, a halogen atom, a cyano group, a nitro group, a C1-C6 alkyl group optionally having one or more groups selected from the group X, a phenyl group optionally having one or more groups selected from the group Y, or —OR¹¹, R² represents a hydrogen atom, a cyano group, a nitro group, a C1-C6 alkyl group optionally having one or more groups selected from the group X, or a phenyl group optionally having one or more groups selected from the group Y, R⁵ represents a hydrogen atom, R⁶ represents a hydrogen atom or a C1-C6 alkyl group, R¹¹ represents a C1-C6 alkyl group optionally having one or more groups selected from the group X, and R¹² represents a hydrogen atom or a C1-C4 alkyl group optionally having one or more halogen atoms;

a compound represented by Formula (1) in which A¹ represents —C(R⁵)═, A² represents —N(R¹²)—, W represents —OR⁶, R¹, R³, and R⁴ may be the same or different and each represents a hydrogen atom, a halogen atom, a cyano group, a nitro group, a C1-C6 alkyl group optionally having one or more groups selected from the group X, a phenyl group optionally having one or more groups selected from the group Y, or —OR¹¹, R² represents a hydrogen atom, a cyano group, a nitro group, a C1-C6 alkyl group optionally having one or more groups selected from the group X, or a phenyl group optionally having one or more groups selected from the group Y, R⁶ represents a hydrogen atom or a C1-C6 alkyl group, R¹¹ represents a C1-C6 alkyl group optionally having one or more groups selected from the group X, and R¹² represents a hydrogen atom or a C1-C4 alkyl group optionally having one or more halogen atoms;

a compound represented by Formula (1) in which A¹ represents —C(R⁵)═, A² represents —N(R¹²)—, A³ represents ═C(R⁴)—, W represents —OR⁶, R¹, R³, and R⁴ may be the same or different and each represents a hydrogen atom, a halogen atom, a cyano group, a nitro group, a C1-C6 alkyl group optionally having one or more groups selected from the group X, a phenyl group optionally having one or more groups selected from the group Y, or —OR¹¹, R² represents a hydrogen atom, a cyano group, a nitro group, a C1-C6 alkyl group optionally having one or more groups selected from the group X, or a phenyl group optionally having one or more groups selected from the group Y, R⁶ represents a hydrogen atom, R¹¹ represents a C1-C6 alkyl group optionally having one or more groups selected from the group X, and R¹² represents a hydrogen atom or a C1-C4 alkyl group optionally having one or more halogen atoms;

a compound represented by Formula (1) in which A¹ represents —C(R⁵)═, A² represents —N(R¹²)—, A³ represents ═C(R⁴)—, W represents —OR⁶, R¹, R³, and R⁴ may be the same or different and each represents a hydrogen atom, a halogen atom, a cyano group, a nitro group, a C1-C6 alkyl group optionally having one or more groups selected from the group X, a phenyl group optionally having one or more groups selected from the group Y, or —OR¹¹, R² represents a hydrogen atom, a cyano group, a nitro group, a C1-C6 alkyl group optionally having one or more groups selected from the group X, or a phenyl group optionally having one or more groups selected from the group Y, R⁶ represents a C1-C6 alkyl group, R¹¹ represents a C1-C6 alkyl group optionally having one or more groups selected from the group X, and R¹² represents a hydrogen atom or a C1-C4 alkyl group optionally having one or more halogen atoms;

a compound represented by Formula (1) in which A¹ represents —C(R⁵)═, A² represents —N(R¹²)—, A³ represents ═C(R⁴)—, W represents —OR⁶, R¹ represents a trifluoromethyl group, R³ and R⁴ may be the same or different and each represents a hydrogen atom, a halogen atom, a cyano group, a nitro group, a C1-C6 alkyl group optionally having one or more groups selected from the group X, a phenyl group optionally having one or more groups selected from the group Y, or —OR¹¹, R² represents a hydrogen atom, a cyano group, a nitro group, a C1-C6 alkyl group optionally having one or more groups selected from the group X, or a phenyl group optionally having one or more groups selected from the group Y, R⁶ represents a methyl group or an ethyl group, R¹¹ represents a C1-C6 alkyl group optionally having one or more groups selected from the group X, and R¹² represents a hydrogen atom or a C1-C4 alkyl group optionally having one or more halogen atoms;

a compound represented by Formula (1) in which A¹ represents —C(R⁵)═, A² represents —N(R¹²)—, A³ represents ═C(R⁴)—, W represents —OR⁶, R¹, R³, and R⁴ may be the same or different and each represents a hydrogen atom, a halogen atom, a cyano group, a nitro group, a C1-C6 alkyl group optionally having one or more groups selected from the group X, a phenyl group optionally having one or more groups selected from the group Y, or —OR¹¹, R² represents a trifluoromethyl group, R⁶ represents a methyl group or an ethyl group, R¹¹ represents a C1-C6 alkyl group optionally having one or more groups selected from the group X, and R¹² represents a hydrogen atom or a C1-C4 alkyl group optionally having one or more halogen atoms;

a compound represented by Formula (1) in which A¹ represents —C(R⁵)═, A² represents —N(R¹²)—, A³ represents ═C(R⁴)—, W represents —OR⁶, R¹ and R⁴ may be the same or different and each represents a hydrogen atom, a halogen atom, a cyano group, a nitro group, a C1-C6 alkyl group optionally having one or more groups selected from the group X, a phenyl group optionally having one or more groups selected from the group Y, or —OR¹¹, R² represents a hydrogen atom, a cyano group, a nitro group, a C1-C6 alkyl group optionally having one or more groups selected from the group X, or a phenyl group optionally having one or more groups selected from the group Y, R³ represents a trifluoromethyl group, R⁶ represents a methyl group or an ethyl group, R¹¹ represents a C1-C6 alkyl group optionally having one or more groups selected from the group X, and R¹² represents a hydrogen atom or a C1-C4 alkyl group optionally having one or more halogen atoms;

a compound represented by Formula (1) in which A¹ represents —C(R⁵)═, A² represents —N(R¹²)—, A³ represents ═C(R⁴)—, W represents —OR⁶, R¹ and R³ may be the same or different and each represents a hydrogen atom, a halogen atom, a cyano group, a nitro group, a C1-C6 alkyl group optionally having one or more groups selected from the group X, a phenyl group optionally having one or more groups selected from the group Y, or —OR¹¹, R² represents a hydrogen atom, a cyano group, a nitro group, a C1-C6 alkyl group optionally having one or more groups selected from the group X, or a phenyl group optionally having one or more groups selected from the group Y, R⁴ represents a trifluoromethyl group, R⁶ represents a methyl group or an ethyl group, R¹¹ represents a C1-C6 alkyl group optionally having one or more groups selected from the group X, and R¹² represents a hydrogen atom or a C1-C4 alkyl group optionally having one or more halogen atoms;

a compound represented by Formula (1) in which A¹ represents —C(R⁵)═, A² represents —N(R¹²)—, A³ represents ═C(R⁴)—, W represents —OR⁶, R¹ represents a trifluoromethyl group, R³ and R⁴ may be the same or different and each represents a hydrogen atom, a halogen atom, a cyano group, a nitro group, a C1-C6 alkyl group optionally having one or more groups selected from the group X, a phenyl group optionally having one or more groups selected from the group Y, or —OR¹¹, R² represents a hydrogen atom, a cyano group, a nitro group, a C1-C6 alkyl group optionally having one or more groups selected from the group X, or a phenyl group optionally having one or more groups selected from the group Y, R⁶ represents a hydrogen atom, R¹ represents a C1-C6 alkyl group optionally having one or more groups selected from the group X, and R¹² represents a hydrogen atom or a C1-C4 alkyl group optionally having one or more halogen atoms;

a compound represented by Formula (1) in which A¹ represents —C(R⁵)═, A² represents —N(R¹²)—, A³ represents ═C(R⁴)—, W represents —OR⁶, R¹, R³ and R⁴ may be the same or different and each represents a hydrogen atom, a halogen atom, a cyano group, a nitro group, a C1-C6 alkyl group optionally having one or more groups selected from the group X, a phenyl group optionally having one or more groups selected from the group Y, or —OR¹¹, R² represents a trifluoromethyl group, R⁶ represents a hydrogen atom, R¹¹ represents a C1-C6 alkyl group optionally having one or more groups selected from the group X, and R¹² represents a hydrogen atom or a C1-C4 alkyl group optionally having one or more halogen atoms;

a compound represented by Formula (1) in which A¹ represents —C(R⁵)═, A² represents —N(R¹²)—, A³ represents ═C(R⁴)—, W represents —OR⁶, R¹ and R⁴ may be the same or different and each represents a hydrogen atom, a halogen atom, a cyano group, a nitro group, a C1-C6 alkyl group optionally having one or more groups selected from the group X, a phenyl group optionally having one or more groups selected from the group Y, or —OR¹¹, R² represents a hydrogen atom, a cyano group, a nitro group, a C1-C6 alkyl group optionally having one or more groups selected from the group X, or a phenyl group optionally having one or more groups selected from the group Y, R3 represents a trifluoromethyl group, R⁶ represents a hydrogen atom, R¹¹ represents a C1-C6 alkyl group optionally having one or more groups selected from the group X, and R¹² represents a hydrogen atom or a C1-C4 alkyl group optionally having one or more halogen atoms;

a compound represented by Formula (1) in which A¹ represents —C(R⁵)═, A² represents —N(R¹²)—, A³ represents ═C(R⁴)—, W represents —OR⁶, R¹ and R³ may be the same or different and each represents a hydrogen atom, a halogen atom, a cyano group, a nitro group, a C1-C6 alkyl group optionally having one or more groups selected from the group X, a phenyl group optionally having one or more groups selected from the group Y, or —OR¹¹, R² represents a hydrogen atom, a cyano group, a nitro group, a C1-C6 alkyl group optionally having one or more groups selected from the group X, or a phenyl group optionally having one or more groups selected from the group Y, R⁴ represents a trifluoromethyl group, R⁶ represents a hydrogen atom, R¹¹ represents a C1-C6 alkyl group optionally having one or more groups selected from the group X, and R¹² represents a hydrogen atom or a C1-C4 alkyl group optionally having one or more halogen atoms;

a compound represented by Formula (1) in which A¹ represents —C(R⁵)═, A² represents —N(R¹²)—, A³ represents ═C(R⁴)—, W represents —OR⁶, R¹ represents a trifluoromethyl group, R³ and R⁴ may be the same or different and each represents a hydrogen atom, a halogen atom, a cyano group, a nitro group, a C1-C6 alkyl group optionally having one or more groups selected from the group X, a phenyl group optionally having one or more groups selected from the group Y, or —OR¹¹, R² represents a hydrogen atom, a cyano group, a nitro group, a C1-C6 alkyl group optionally having one or more groups selected from the group X, or a phenyl group optionally having one or more groups selected from the group Y, R⁵ represents a hydrogen atom, R⁶ represents a methyl group or an ethyl group, R¹¹ represents a C1-C6 alkyl group optionally having one or more groups selected from the group X, and R¹² represents a hydrogen atom or a C1-C4 alkyl group optionally having one or more halogen atoms;

a compound represented by Formula (1) in which A¹ represents —C(R⁵)═, A² represents —N(R¹²)—, A³ represents ═C(R⁴)—, W represents —OR⁶, R¹, R³ and R⁴ may be the same or different and each represents a hydrogen atom, a halogen atom, a cyano group, a nitro group, a C1-C6 alkyl group optionally having one or more groups selected from the group X, a phenyl group optionally having one or more groups selected from the group Y, or —OR¹¹, R² represents a trifluoromethyl group, R⁵ represents a hydrogen atom, R⁶ represents a methyl group or an ethyl group, R¹¹ represents a C1-C6 alkyl group optionally having one or more groups selected from the group X, and R¹² represents a hydrogen atom or a C1-C4 alkyl group optionally having one or more halogen atoms;

a compound represented by Formula (1) in which A¹ represents —C(R⁵)═, A² represents —N(R¹²)—, A³ represents ═C(R⁴)—, W represents —OR⁶, R¹ and R⁴ may be the same or different and each represents a hydrogen atom, a halogen atom, a cyano group, a nitro group, a C1-C6 alkyl group optionally having one or more groups selected from the group X, a phenyl group optionally having one or more groups selected from the group Y, or —OR¹¹, R² represents a hydrogen atom, a cyano group, a nitro group, a C1-C6 alkyl group optionally having one or more groups selected from the group X, or a phenyl group optionally having one or more groups selected from the group Y, R³ represents a trifluoromethyl group, R⁵ represents a hydrogen atom, R⁶ represents a methyl group or an ethyl group, R¹¹ represents a C1-C6 alkyl group optionally having one or more groups selected from the group X, and R¹² represents a hydrogen atom or a C1-C4 alkyl group optionally having one or more halogen atoms;

a compound represented by Formula (1) in which A¹ represents —C(R⁵)═, A² represents —N(R¹²)—, A³ represents ═C(R⁴)—, W represents —OR⁶, R¹ and R³ may be the same or different and each represents a hydrogen atom, a halogen atom, a cyano group, a nitro group, a C1-C6 alkyl group optionally having one or more groups selected from the group X, a phenyl group optionally having one or more groups selected from the group Y, or —OR¹¹, R² represents a hydrogen atom, a cyano group, a nitro group, a C1-C6 alkyl group optionally having one or more groups selected from the group X, or a phenyl group optionally having one or more groups selected from the group Y, R⁴ represents a trifluoromethyl group, R⁵ represents a hydrogen atom, R⁶ represents a methyl group or an ethyl group, R¹¹ represents a C1-C6 alkyl group optionally having one or more groups selected from the group X, and R¹² represents a hydrogen atom or a C1-C4 alkyl group optionally having one or more halogen atoms;

a compound represented by Formula (1) in which A¹ represents —C(R⁵)═, A² represents —N(R¹²)—, A³ represents ═C(R⁴)—, W represents —OR⁶, R¹ represents a trifluoromethyl group, R³ and R⁴ may be the same or different and each represents a hydrogen atom, a halogen atom, a cyano group, a nitro group, a C1-C6 alkyl group optionally having one or more groups selected from the group X, a phenyl group optionally having one or more groups selected from the group Y, or —OR¹¹, R² represents a hydrogen atom, a cyano group, a nitro group, a C1-C6 alkyl group optionally having one or more groups selected from the group X, or a phenyl group optionally having one or more groups selected from the group Y, R⁵ represents a hydrogen atom, R⁶ represents a hydrogen atom, R¹¹ represents a C1-C6 alkyl group optionally having one or more groups selected from the group X, and R¹² represents a hydrogen atom or a C1-C4 alkyl group optionally having one or more halogen atoms;

a compound represented by Formula (1) in which A¹ represents —C(R⁵)═, A² represents —N(R¹²)—, A³ represents ═C(R⁴)—, W represents —OR⁶, R¹, R³ and R⁴ may be the same or different and each represents a hydrogen atom, a halogen atom, a cyano group, a nitro group, a C1-C6 alkyl group optionally having one or more groups selected from the group X, a phenyl group optionally having one or more groups selected from the group Y, or —OR¹¹, R² represents a trifluoromethyl group, R⁵ represents a hydrogen atom, R⁶ represents a hydrogen atom, R¹¹ represents a C1-C6 alkyl group optionally having one or more groups selected from the group X, and R¹² represents a hydrogen atom or a C1-C4 alkyl group optionally having one or more halogen atoms;

a compound represented by Formula (1) in which A¹ represents —C(R⁵)═, A² represents —N(R¹²)—, A³ represents ═C(R⁴)—, W represents —OR⁶, R¹ and R⁴ may be the same or different and each represents a hydrogen atom, a halogen atom, a cyano group, a nitro group, a C1-C6 alkyl group optionally having one or more groups selected from the group X, a phenyl group optionally having one or more groups selected from the group Y, or —OR¹¹, R² represents a hydrogen atom, a cyano group, a nitro group, a C1-C6 alkyl group optionally having one or more groups selected from the group X, or a phenyl group optionally having one or more groups selected from the group Y, R³ represents a trifluoromethyl group, R⁵ represents a hydrogen atom, R⁶ represents a hydrogen atom, R¹¹ represents a C1-C6 alkyl group optionally having one or more groups selected from the group X, and R¹² represents a hydrogen atom or a C1-C4 alkyl group optionally having one or more halogen atoms;

a compound represented by Formula (1) in which A¹ represents —C(R⁵)═, A² represents —N(R¹²)—, A³ represents ═C(R⁴)—, W represents —OR⁶, R¹ and R³ may be the same or different and each represents a hydrogen atom, a halogen atom, a cyano group, a nitro group, a C1-C6 alkyl group optionally having one or more groups selected from the group X, a phenyl group optionally having one or more groups selected from the group Y, or —OR¹¹, R² represents a hydrogen atom, a cyano group, a nitro group, a C1-C6 alkyl group optionally having one or more groups selected from the group X, or a phenyl group optionally having one or more groups selected from the group Y, R⁴ represents a trifluoromethyl group, R⁵ represents a hydrogen atom, R⁶ represents a hydrogen atom, R¹¹ represents a C1-C6 alkyl group optionally having one or more groups selected from the group X, and R¹² represents a hydrogen atom or a C1-C4 alkyl group optionally having one or more halogen atoms;

a compound represented by Formula (1) in which A¹ represents —C(R⁵)═, A² represents —N(R¹²)—, A³ represents ═C(R⁴)—, and W represents —ON═CR⁷R⁸;

a compound represented by Formula (1) in which A¹ represents —C(R⁵)═, A² represents —N(R¹²)—, A³ represents ═C(R⁴)—, and W represents —SR⁹;

a compound represented by Formula (1) in which A¹ represents —C(R⁵)═, A² represents —N(R¹²)—, A³ represents ═C(R⁴)—, W represents —SR⁹, and R⁹ represents a C1-C6 alkyl group;

a compound represented by Formula (1) in which A¹ represents —C(R⁵)═, A² represents —N(R¹²)—, A³ represents ═C(R⁴)—, W represents —NR⁷R¹⁰, and R¹⁰ represents a hydrogen atom, a C1-C6 alkyl group, or a phenyl group optionally having one or more groups selected from the group Y;

a compound represented by Formula (1) in which A¹ represents —C(R⁵)═, A² represents —N(R¹²)—, A³ represents ═C(R⁴)—, W represents —NR⁷R¹⁰, and R¹⁰ represents a hydrogen atom or a C1-C6 alkyl group;

a compound represented by Formula (1) in which A¹ represents —C(R⁵)═, A² represents an oxygen atom, A³ represents ═C(R⁴)—, R¹ represents a trifluoromethyl group, and R², R³, R⁴, and R⁵ represent a hydrogen atom;

a compound represented by Formula (1) in which A¹ represents —C(R⁵)═, A² represents an oxygen atom, A³ represents ═C(R⁴)—, R² represents a trifluoromethyl group, and R¹, R³, R⁴, and R⁵ represent a hydrogen atom;

a compound represented by Formula (1) in which A¹ represents —C(R⁵)═, A² represents an oxygen atom, A³ represents ═C(R⁴)—, R³ represents a trifluoromethyl group, and R¹, R², R⁴, and R⁵ represent a hydrogen atom;

a compound represented by Formula (1) in which A¹ represents —C(R⁵)═, A² represents an oxygen atom, A³ represents ═C(R⁴)—, R⁴ represents a trifluoromethyl group, and R¹, R², R³, and R⁵ represent a hydrogen atom;

a compound represented by Formula (1) in which A¹ represents —C(R⁵)═, A² represents an oxygen atom, A³ represents ═C(R⁴)—, R⁵ represents a trifluoromethyl group, and R¹, R², R³, and R⁴ represent a hydrogen atom;

a compound represented by Formula (1) in which A¹ represents —C(R⁵)═, A² represents an oxygen atom, A³ represents ═C(R⁴)—, and R¹ represents a trifluoromethyl group;

a compound represented by Formula (1) in which A¹ represents —C(R⁵)═, A² represents an oxygen atom, A³ represents ═C(R⁴)—, and R² represents a trifluoromethyl group;

a compound represented by Formula (1) in which A¹ represents —C(R⁵)═, A² represents an oxygen atom, A³ represents ═C(R⁴)—, and R³ represents a trifluoromethyl group;

a compound represented by Formula (1) in which A¹ represents —C(R⁵)═, A² represents an oxygen atom, A³ represents ═C(R⁴)—, and R⁴ represents a trifluoromethyl group;

a compound represented by Formula (1) in which A¹ represents —C(R⁵)═, A² represents an oxygen atom, A³ represents ═C(R⁴)—, and R⁵ represents a trifluoromethyl group;

a compound represented by Formula (1) in which A¹ represents —C(R⁵)═, A² represents an oxygen atom, A³ represents ═C(R⁴)—, R¹ represents a halogen atom, and R², R³, R⁴, and R⁵ represent a hydrogen atom;

a compound represented by Formula (1) in which A¹ represents —C(R⁵)═, A² represents an oxygen atom, A³ represents ═C(R⁴)—, R² represents a halogen atom, and R¹, R³, R⁴, and R⁵ represent a hydrogen atom;

a compound represented by Formula (1) in which A¹ represents —C(R⁵)═, A² represents an oxygen atom, A³ represents ═C(R⁴)—, R³ represents a halogen atom, and R¹, R², R⁴, and R⁵ represent a hydrogen atom;

a compound represented by Formula (1) in which A¹ represents —C(R⁵)═, A² represents an oxygen atom, A³ represents ═C(R⁴)—, R⁴ represents a halogen atom, and R¹, R², R³, and R⁵ represent a hydrogen atom;

a compound represented by Formula (1) in which A¹ represents —C(R⁵)═, A² represents an oxygen atom, A³ represents ═C(R⁴)—, R⁵ represents a halogen atom, and R¹, R², R³, and R⁴ represent a hydrogen atom;

a compound represented by Formula (1) in which A¹ represents —C(R⁵)═, A² represents an oxygen atom, A³ represents ═C(R⁴)—, and R¹ represents a halogen atom;

a compound represented by Formula (1) in which A¹ represents —C(R⁵)═, A² represents an oxygen atom, A³ represents ═C(R⁴)—, and R² represents a halogen atom;

a compound represented by Formula (1) in which A¹ represents —C(R⁵)═, A² represents an oxygen atom, A³ represents ═C(R⁴)—, and R³ represents a halogen atom;

a compound represented by Formula (1) in which A¹ represents —C(R⁵)═, A² represents an oxygen atom, A³ represents ═C(R⁴)—, and R⁴ represents a halogen atom;

a compound represented by Formula (1) in which A¹ represents —C(R⁵)═, A² represents an oxygen atom, A³ represents ═C(R⁴)—, and R⁵ represents a halogen atom;

a compound represented by Formula (1) in which A¹ represents —C(R⁵)═, A² represents an oxygen atom, A³ represents ═C(R⁴)—, R¹ represents a C1-C6 alkyl group optionally having one or more groups selected from the group X, and R², R³, R⁴, and R⁵ represent a hydrogen atom;

a compound represented by Formula (1) in which A¹ represents —C(R⁵)═, A² represents an oxygen atom, A³ represents C(R⁴)—, R² represents a C1-C6 alkyl group optionally having one or more groups selected from the group X, and R¹, R³, R⁴, and R⁵ represent a hydrogen atom;

a compound represented by Formula (1) in which A¹ represents —C(R⁵)═, A² represents an oxygen atom, A³ represents ═C(R⁴)—, R³ represents a C1-C6 alkyl group optionally having one or more groups selected from the group X, and R¹, R², R⁴, and R⁵ represent a hydrogen atom;

a compound represented by Formula (1) in which A¹ represents —C(R⁵)═, A² represents an oxygen atom, A³ represents ═C(R⁴)—, R⁴ represents a C1-C6 alkyl group optionally having one or more groups selected from the group X, and R¹, R², R³, and R⁵ represent a hydrogen atom;

a compound represented by Formula (1) in which A¹ represents —C(R⁵)═, A² represents an oxygen atom, A³ represents ═C(R⁴)—, R⁵ represents a C1-C6 alkyl group optionally having one or more groups selected from the group X, and R¹, R², R³, and R⁴ represent a hydrogen atom;

a compound represented by Formula (1) in which A¹ represents —C(R⁵)═, A² represents an oxygen atom, A³ represents ═C(R⁴)—, and R¹ represents a C1-C6 alkyl group optionally having one or more groups selected from the group X;

a compound represented by Formula (1) in which A¹ represents —C(R⁵)═, A² represents an oxygen atom, A³ represents ═C(R⁴)—, and R² represents a C1-C6 alkyl group optionally having one or more groups selected from the group X;

a compound represented by Formula (1) in which A¹ represents —C(R⁵)═, A² represents an oxygen atom, A³ represents ═C(R⁴)—, and R³ represents a C1-C6 alkyl group optionally having one or more groups selected from the group X;

a compound represented by Formula (1) in which A¹ represents —C(R⁵)═, A² represents an oxygen atom, A³ represents ═C(R⁴)—, and R⁴ represents a C1-C6 alkyl group optionally having one or more groups selected from the group X;

a compound represented by Formula (1) in which A¹ represents —C(R⁵)═, A² represents an oxygen atom, A³ represents ═C(R⁴)—, and R⁵ represents a C1-C6 alkyl group optionally having one or more groups selected from the group X;

a compound represented by Formula (1) in which A¹ represents —C(R⁵)═, A² represents an oxygen atom, A³ represents ═C(R⁴)—, R¹ represents a phenyl group optionally having one or more groups selected from the group Y, and R², R³, R⁴, and R⁵ represent a hydrogen atom;

a compound represented by Formula (1) in which A¹ represents —C(R⁵)═, A² represents an oxygen atom, A³ represents ═C(R⁴)—, R² represents a phenyl group optionally having one or more groups selected from the group Y, and R¹, R³, R⁴, and R⁵ represent a hydrogen atom;

a compound represented by Formula (1) in which A¹ represents —C(R⁵)═, A² represents an oxygen atom, A³ represents ═C(R⁴)—, R³ represents a phenyl group optionally having one or more groups selected from the group Y, and R¹, R², R⁴, and R⁵ represent a hydrogen atom;

a compound represented by Formula (1) in which A¹ represents —C(R⁵)═, A² represents an oxygen atom, A³ represents ═C(R⁴)—, R⁴ represents a phenyl group optionally having one or more groups selected from the group Y, and R¹, R², R³, and R⁵ represent a hydrogen atom;

a compound represented by Formula (1) in which A¹ represents —C(R⁵)═, A² represents an oxygen atom, A³ represents ═C(R⁴)—, R⁵ represents a phenyl group optionally having one or more groups selected from the group Y, and R¹, R², R³, and R⁴ represent a hydrogen atom;

a compound represented by Formula (1) in which A¹ represents —C(R⁵)═, A² represents an oxygen atom, A³ represents ═C(R⁴)—, and R¹ represents a phenyl group optionally having one or more groups selected from the group Y;

a compound represented by Formula (1) in which A¹ represents —C(R⁵)═, A² represents an oxygen atom, A³ represents ═C(R⁴)—, R² represents a phenyl group optionally having one or more groups selected from the group Y;

a compound represented by Formula (1) in which A¹ represents —C(R⁵)═, A² represents an oxygen atom, A³ represents ═C(R⁴)—, R³ represents a phenyl group optionally having one or more groups selected from the group Y;

a compound represented by Formula (1) in which A¹ represents —C(R⁵)═, A² represents an oxygen atom, A³ represents ═C(R⁴)—, R⁴ represents a phenyl group optionally having one or more groups selected from the group Y;

a compound represented by Formula (1) in which A¹ represents —C(R⁵)═, A² represents an oxygen atom, A³ represents ═C(R⁴)—, R⁵ represents a phenyl group optionally having one or more groups selected from the group Y;

a compound represented by Formula (1) in which A¹ represents —C(R⁵)═, A² represents an oxygen atom, A³ represents ═C(R⁴)—, R¹ represents a 6-membered aromatic heterocyclic group optionally having one or more groups selected from the group Y or a 5-membered aromatic heterocyclic group optionally having one or more groups selected from the group Y, and R², R³, R⁴, and R⁵ represent a hydrogen atom;

a compound represented by Formula (1) in which A¹ represents —C(R⁵)═, A² represents an oxygen atom, A³ represents ═C(R⁴)—, R² represents a 6-membered aromatic heterocyclic group optionally having one or more groups selected from the group Y or a 5-membered aromatic heterocyclic group optionally having one or more groups selected from the group Y, and R¹, R³, R⁴, and R⁵ represent a hydrogen atom;

a compound represented by Formula (1) in which A¹ represents —C(R⁵)═, A² represents an oxygen atom, A³ represents ═C(R⁴)—, R³ represents a 6-membered aromatic heterocyclic group optionally having one or more groups selected from the group Y or a 5-membered aromatic heterocyclic group optionally having one or more groups selected from the group Y, and R¹, R², R⁴, and R⁵ represent a hydrogen atom;

a compound represented by Formula (1) in which A¹ represents —C(R⁵)═, A² represents an oxygen atom, A³ represents ═C(R⁴)—, R⁴ represents a 6-membered aromatic heterocyclic group optionally having one or more groups selected from the group Y or a 5-membered aromatic heterocyclic group optionally having one or more groups selected from the group Y, and R¹, R², R³, and R⁵ represent a hydrogen atom;

a compound represented by Formula (1) in which A¹ represents —C(R⁵)═, A² represents an oxygen atom, A³ represents ═C(R⁴)—, R⁵ represents a 6-membered aromatic heterocyclic group optionally having one or more groups selected from the group Y or a 5-membered aromatic heterocyclic group optionally having one or more groups selected from the group Y, and R¹, R², R³, and R⁴ represent a hydrogen atom;

a compound represented by Formula (1) in which A¹ represents —C(R⁵)═, A² represents an oxygen atom, A³ represents ═C(R⁴)—, and R¹ represents a 6-membered aromatic heterocyclic group optionally having one or more groups selected from the group Y or a 5-membered aromatic heterocyclic group optionally having one or more groups selected from the group Y;

a compound represented by Formula (1) in which A¹ represents —C(R⁵)═, A² represents an oxygen atom, A³ represents ═C(R⁴)—, and R² represents a 6-membered aromatic heterocyclic group optionally having one or more groups selected from the group Y or a 5-membered aromatic heterocyclic group optionally having one or more groups selected from the group Y;

a compound represented by Formula (1) in which A¹ represents —C(R⁵)═, A² represents an oxygen atom, A³ represents ═C(R⁴)—, and R³ represents a 6-membered aromatic heterocyclic group optionally having one or more groups selected from the group Y or a 5-membered aromatic heterocyclic group optionally having one or more groups selected from the group Y;

a compound represented by Formula (1) in which A¹ represents —C(R⁵)═, A² represents an oxygen atom, A³ represents ═C(R⁴)—, and R⁴ represents a 6-membered aromatic heterocyclic group optionally having one or more groups selected from the group Y or a 5-membered aromatic heterocyclic group optionally having one or more groups selected from the group Y;

a compound represented by Formula (1) in which A¹ represents —C(R⁵)═, A² represents an oxygen atom, A³ represents ═C(R⁴)—, and R⁵ represents a 6-membered aromatic heterocyclic group optionally having one or more groups selected from the group Y or a 5-membered aromatic heterocyclic group optionally having one or more groups selected from the group Y;

a compound represented by Formula (1) in which A¹ represents —C(R⁵)═, A² represents an oxygen atom, A³ represents ═C(R⁴)—, R¹ represents a C2-C6 alkylcarbonyl group optionally having one or more halogen atoms, and R², R³, R⁴, and R⁵ represent a hydrogen atom;

a compound represented by Formula (1) in which A¹ represents —C(R⁵)═, A² represents an oxygen atom, A³ represents ═C(R⁴)—, R² represents a C2-C6 alkylcarbonyl group optionally having one or more halogen atoms, and R¹, R³, R⁴, and R⁵ represent a hydrogen atom;

a compound represented by Formula (1) in which A¹ represents —C(R⁵)═, A² represents an oxygen atom, A³ represents ═C(R⁴)—, and R¹ represents a C2-C6 alkylcarbonyl group optionally having one or more halogen atoms;

a compound represented by Formula (1) in which A¹ represents —C(R⁵)═, A² represents an oxygen atom, A³ represents ═C(R⁴)—, and R² represents a C2-C6 alkylcarbonyl group optionally having one or more halogen atoms;

a compound represented by Formula (1) in which A¹ represents —C(R⁵)═, A² represents an oxygen atom, A³ represents ═C(R⁴)—, R¹ represents a carboxy group, and R², R³, R⁴, and R⁵ represent a hydrogen atom;

a compound represented by Formula (1) in which A¹ represents —C(R⁵)═, A² represents an oxygen atom, A³ represents ═C(R⁴)—, R² represents a carboxy group, and R¹, R³, R⁴, and R⁵ represent a hydrogen atom;

a compound represented by Formula (1) in which A¹ represents —C(R⁵)═, A² represents an oxygen atom, A³ represents ═C(R⁴)—, R³ represents a carboxy group, and R¹, R², R⁴, and R⁵ represent a hydrogen atom;

a compound represented by Formula (1) in which A¹ represents —C(R⁵)═, A² represents an oxygen atom, A³ represents ═C(R⁴)—, R⁴ represents a carboxy group, and R¹, R², R³, and R⁵ represent a hydrogen atom;

a compound represented by Formula (1) in which A¹ represents —C(R⁵)═, A² represents an oxygen atom, A³ represents ═C(R⁴)—, R⁵ represents a carboxy group, and R¹, R², R³, and R⁴ represent a hydrogen atom;

a compound represented by Formula (1) in which A¹ represents —C(R⁵)═, A² represents an oxygen atom, A³ represents ═C(R⁴)—, and R¹ represents a carboxy group;

a compound represented by Formula (1) in which A¹ represents —C(R⁵)═, A² represents an oxygen atom, A³ represents ═C(R⁴)—, and R² represents a carboxy group;

a compound represented by Formula (1) in which A¹ represents —C(R⁵)═, A² represents an oxygen atom, A³ represents ═C(R⁴)—, and R³ represents a carboxy group;

a compound represented by Formula (1) in which A¹ represents —C(R⁵)═, A² represents an oxygen atom, A³ represents ═C(R⁴)—, and R⁴ represents a carboxy group;

a compound represented by Formula (1) in which A¹ represents —C(R⁵)═, A² represents an oxygen atom, A³ represents ═C(R⁴)—, R¹ represents a C2-C6 alkoxycarbonyl group, and R², R³, R⁴, and R⁵ represent a hydrogen atom;

a compound represented by Formula (1) in which A¹ represents —C(R⁵)═, A² represents an oxygen atom, A³ represents ═C(R⁴)—, R² represents a C2-C6 alkoxycarbonyl group, and R¹, R³, R⁴, and R⁵ represent a hydrogen atom;

a compound represented by Formula (1) in which A¹ represents —C(R⁵)═, A² represents an oxygen atom, A³ represents ═C(R⁴)—, R³ represents a C2-C6 alkoxycarbonyl group, and R¹, R², R⁴, and R⁵ represent a hydrogen atom;

a compound represented by Formula (1) in which A¹ represents —C(R⁵)═, A² represents an oxygen atom, A³ represents ═C(R⁴)—, R⁴ represents a C2-C6 alkoxycarbonyl group, and R¹, R², R³, and R⁵ represent a hydrogen atom;

a compound represented by Formula (1) in which A¹ represents —C(R⁵)═, A² represents an oxygen atom, A³ represents ═C(R⁴)—, R⁵ represents a C2-C6 alkoxycarbonyl group, and R¹, R², R³, and R⁴ represent a hydrogen atom;

a compound represented by Formula (1) in which A¹ represents —C(R⁵)═, A² represents an oxygen atom, A³ represents ═C(R⁴)—, and R¹ represents a C2-C6 alkoxycarbonyl group;

a compound represented by Formula (1) in which A¹ represents —C(R⁵)═, A² represents an oxygen atom, A³ represents ═C(R⁴)—, and R² represents a C2-C6 alkoxycarbonyl group;

a compound represented by Formula (1) in which A¹ represents —C(R⁵)═, A² represents an oxygen atom, A³ represents ═C(R⁴)—, and R³ represents a C2-C6 alkoxycarbonyl group;

a compound represented by Formula (1) in which A¹ represents —C(R⁵)═, A² represents an oxygen atom, A³ represents ═C(R⁴)—, and R⁴ represents a C2-C6 alkoxycarbonyl group;

a compound represented by Formula (1) in which A¹ represents —C(R⁵)═, A² represents an oxygen atom, A³ represents ═C(R⁴)—, R¹ represents a trifluoromethyl group, and R², R³, and R⁴ represent a hydrogen atom;

a compound represented by Formula (1) in which A¹ represents —C(R⁵)═, A² represents an oxygen atom, A³ represents ═C(R⁴)—, R² represents a trifluoromethyl group, and R¹, R³, and R⁴ represent a hydrogen atom;

a compound represented by Formula (1) in which A¹ represents —C(R⁵)═, A² represents an oxygen atom, A³ represents ═C(R⁴)—, R³ represents a trifluoromethyl group, and R¹, R², and R⁴ represent a hydrogen atom;

a compound represented by Formula (1) in which A¹ represents —C(R⁵)═, A² represents an oxygen atom, A³ represents ═C(R⁴)—, R⁴ represents a trifluoromethyl group, and R¹, R², and R³ represent a hydrogen atom;

a compound represented by Formula (1) in which A¹ represents —C(R⁵)═, A² represents an oxygen atom, A³ represents ═C(R⁴)—, R⁵ represents a trifluoromethyl group, and R², R³, and R⁴ represent a hydrogen atom;

a compound represented by Formula (1) in which A¹ represents —C(R⁵)═, A² represents an oxygen atom, A³ represents ═C(R⁴)—, R⁵ represents a trifluoromethyl group, and R¹, R³, and R⁴ represent a hydrogen atom;

a compound represented by Formula (1) in which A¹ represents —C(R⁵)═, A² represents an oxygen atom, A³ represents ═C(R⁴)—, R⁵ represents a trifluoromethyl group, and R¹, R², and R⁴ represent a hydrogen atom;

a compound represented by Formula (1) in which A¹ represents —C(R⁵)═, A² represents an oxygen atom, A³ represents ═C(R⁴)—, R⁵ represents a trifluoromethyl group, and R¹, R², and R³ represent a hydrogen atom;

a compound represented by Formula (1) in which A¹ represents —C(R⁵)═, A² represents an oxygen atom, A³ represents ═C(R⁴)—, R¹ represents a halogen atom, and R², R³, and R⁴ represent a hydrogen atom;

a compound represented by Formula (1) in which A¹ represents —C(R⁵)═, A² represents an oxygen atom, A³ represents ═C(R⁴)—, R² represents a halogen atom, and R¹, R³, and R⁴ represent a hydrogen atom;

a compound represented by Formula (1) in which A¹ represents —C(R⁵)═, A² represents an oxygen atom, A³ represents ═C(R⁴)—, R³ represents a halogen atom, and R¹, R², and R⁴ represent a hydrogen atom;

a compound represented by Formula (1) in which A¹ represents —C(R⁵)═, A² represents an oxygen atom, A³ represents ═C(R⁴)—, R⁴ represents a halogen atom, and R¹, R², and R³ represent a hydrogen atom;

a compound represented by Formula (1) in which A¹ represents —C(R⁵)═, A² represents an oxygen atom, A³ represents ═C(R⁴)—, and R¹ represents a halogen atom;

a compound represented by Formula (1) in which A¹ represents —C(R⁵)═, A² represents an oxygen atom, A³ represents ═C(R⁴)—, and R² represents a halogen atom;

a compound represented by Formula (1) in which A¹ represents —C(R⁵)═, A² represents an oxygen atom, A³ represents ═C(R⁴)—, and R³ represents a halogen atom;

a compound represented by Formula (1) in which A¹ represents —C(R⁵)═, A² represents an oxygen atom, A³ represents ═C(R⁴)—, and R⁴ represents a halogen atom;

a compound represented by Formula (1) in which A¹ represents —C(R⁵)═, A² represents an oxygen atom, A³ represents ═C(R⁴)—, R¹ represents a C1-C6 alkyl group optionally having one or more groups selected from the group X, and R², R³, and R⁴ represent a hydrogen atom;

a compound represented by Formula (1) in which A¹ represents —C(R⁵)═, A² represents an oxygen atom, A³ represents ═C(R⁴)—, R² represents a C1-C6 alkyl group optionally having one or more groups selected from the group X, and R¹, R³, and R⁴ represent a hydrogen atom;

a compound represented by Formula (1) in which A¹ represents —C(R⁵)═, A² represents an oxygen atom, A³ represents ═C(R⁴)—, R³ represents a C1-C6 alkyl group optionally having one or more groups selected from the group X, and R¹, R², and R⁴ represent a hydrogen atom;

a compound represented by Formula (1) in which A¹ represents —C(R⁵)═, A² represents an oxygen atom, A³ represents ═C(R⁴)—, R⁴ represents a C1-C6 alkyl group optionally having one or more groups selected from the group X, and R¹, R², and R³ represent a hydrogen atom;

a compound represented by Formula (1) in which A¹ represents —C(R⁵)═, A² represents an oxygen atom, A³ represents ═C(R⁴)—, and R¹ represents a C1-C6 alkyl group optionally having one or more groups selected from the group X;

a compound represented by Formula (1) in which A¹ represents —C(R⁵)═, A² represents an oxygen atom, A³ represents ═C(R⁴)—, and R² represents a C1-C6 alkyl group group X;

a compound represented by Formula (1) in which A¹ represents —C(R⁵)═, A² represents an oxygen atom, A³ represents ═C(R⁴)—, and R³ represents a C1-C6 alkyl group optionally having one or more groups selected from the group X;

a compound represented by Formula (1) in which A¹ represents —C(R⁵)═, A² represents an oxygen atom, A³ represents ═C(R⁴)—, and R⁴ represents a C1-C6 alkyl group optionally having one or more groups selected from the group X;

a compound represented by Formula (1) in which A¹ represents —C(R⁵)═, A² represents an oxygen atom, A³ represents ═C(R⁴)—, and R⁵ represents a C1-C6 alkyl group optionally having one or more groups selected from the group X;

a compound represented by Formula (1) in which A¹ represents —C(R⁵)═, A² represents an oxygen atom, A³ represents ═C(R⁴)—, R¹ represents a phenyl group optionally having one or more groups selected from the group Y, and R², R³, and R⁴ represent a hydrogen atom;

a compound represented by Formula (1) in which A¹ represents —C(R⁵)═, A² represents an oxygen atom, A³ represents ═C(R⁴)—, R² represents a phenyl group optionally having one or more groups selected from the group Y, and R¹, R³, and R⁴ represent a hydrogen atom;

a compound represented by Formula (1) in which A¹ represents —C(R⁵)═, A² represents an oxygen atom, A³ represents ═C(R⁴)—, R³ represents a phenyl group optionally having one or more groups selected from the group Y, and R¹, R², and R⁴ represent a hydrogen atom;

a compound represented by Formula (1) in which A¹ represents —C(R⁵)═, A² represents an oxygen atom, A³ represents ═C(R⁴)—, R⁴ represents a phenyl group optionally having one or more groups selected from the group Y, and R¹, R², and R³ represent a hydrogen atom;

a compound represented by Formula (1) in which A¹ represents —C(R⁵)═, A² represents an oxygen atom, A³ represents ═C(R⁴)—, and R¹ represents a phenyl group optionally having one or more groups selected from the group Y;

a compound represented by Formula (1) in which A¹ represents —C(R⁵)═, A² represents an oxygen atom, A³ represents ═C(R⁴)—, and R² represents a phenyl group optionally having one or more groups selected from the group Y;

a compound represented by Formula (1) in which A¹ represents —C(R⁵)═, A² represents an oxygen atom, A³ represents ═C(R⁴)—, and R³ represents a phenyl group optionally having one or more groups selected from the group Y;

a compound represented by Formula (1) in which A¹ represents —C(R⁵)═, A² represents an oxygen atom, A³ represents ═C(R⁴)—, and R⁴ represents a phenyl group optionally having one or more groups selected from the group Y;

a compound represented by Formula (1) in which A¹ represents —C(R⁵)═, A² represents an oxygen atom, A³ represents ═C(R⁴)—, and R⁵ represents a phenyl group optionally having one or more groups selected from the group Y;

a compound represented by Formula (1) in which A¹ represents —C(R⁵)═, A² represents an oxygen atom, A³ represents ═C(R⁴)—, R¹ represents a 6-membered aromatic heterocyclic group optionally having one or more groups selected from the group Y or a 5-membered aromatic heterocyclic group optionally having one or more groups selected from the group Y, and R², R³, and R⁴ represent a hydrogen atom;

a compound represented by Formula (1) in which A¹ represents —C(R⁵)═, A² represents an oxygen atom, A³ represents ═C(R⁴)—, R² represents a 6-membered aromatic heterocyclic group optionally having one or more groups selected from the group Y or a 5-membered aromatic heterocyclic group optionally having one or more groups selected from the group Y, and R¹, R³, and R⁴ represent a hydrogen atom;

a compound represented by Formula (1) in which A¹ represents —C(R⁵)═, A² represents an oxygen atom, A³ represents ═C(R⁴)—, R³ represents a 6-membered aromatic heterocyclic group optionally having one or more groups selected from the group Y or a 5-membered aromatic heterocyclic group optionally having one or more groups selected from the group Y, and R¹, R², and R⁴ represent a hydrogen atom;

a compound represented by Formula (1) in which A¹ represents —C(R⁵)═, A² represents an oxygen atom, A³ represents ═C(R⁴)—, R⁴ represents a 6-membered aromatic heterocyclic group optionally having one or more groups selected from the group Y or a 5-membered aromatic heterocyclic group optionally having one or more groups selected from the group Y, and R¹, R², and R³ represent a hydrogen atom;

a compound represented by Formula (1) in which A¹ represents —C(R⁵)═, A² represents an oxygen atom, A³ represents ═C(R⁴)—, and R¹ represents a 6-membered aromatic heterocyclic group optionally having one or more groups selected from the group Y or a 5-membered aromatic heterocyclic group optionally having one or more groups selected from the group Y;

a compound represented by Formula (1) in which A¹ represents —C(R⁵)═, A² represents an oxygen atom, A³ represents ═C(R⁴)—, and R² represents a 6-membered aromatic heterocyclic group optionally having one or more groups selected from the group Y or a 5-membered aromatic heterocyclic group optionally having one or more groups selected from the group Y;

a compound represented by Formula (1) in which A¹ represents —C(R⁵)═, A² represents an oxygen atom, A³ represents ═C(R⁴)—, and R³ represents a 6-membered aromatic heterocyclic group optionally having one or more groups selected from the group Y or a 5-membered aromatic heterocyclic group optionally having one or more groups selected from the group Y;

a compound represented by Formula (1) in which A¹ represents —C(R⁵)═, A² represents an oxygen atom, A³ represents ═C(R⁴)—, and R⁴ represents a 6-membered aromatic heterocyclic group optionally having one or more groups selected from the group Y or a 5-membered aromatic heterocyclic group optionally having one or more groups selected from the group Y;

a compound represented by Formula (1) in which A¹ represents —C(R⁵)═, A² represents an oxygen atom, A³ represents ═C(R⁴)—, and R⁵ represents a 6-membered aromatic heterocyclic group optionally having one or more groups selected from the group Y or a 5-membered aromatic heterocyclic group optionally having one or more groups selected from the group Y;

a compound represented by Formula (1) in which A¹ represents —C(R⁵)═, A² represents an oxygen atom, A³ represents ═C(R⁴)—, R¹ represents a C2-C6 alkylcarbonyl group optionally having one or more halogen atoms, and R², R³, and R⁴ represent a hydrogen atom;

a compound represented by Formula (1) in which A¹ represents —C(R⁵)═, A² represents an oxygen atom, A³ represents ═C(R⁴)—, R² represents a C2-C6 alkylcarbonyl group optionally having one or more halogen atoms, and R¹, R³, and R⁴ represent a hydrogen atom;

a compound represented by Formula (1) in which A¹ represents —C(R⁵)═, A² represents an oxygen atom, A³ represents ═C(R⁴)—, and R¹ represents a C2-C6 alkylcarbonyl group optionally having one or more halogen atoms;

a compound represented by Formula (1) in which A¹ represents —C(R⁵)═, A² represents an oxygen atom, A³ represents ═C(R⁴)—, and R² represents a C2-C6 alkylcarbonyl group optionally having one or more halogen atoms;

a compound represented by Formula (1) in which A¹ represents —C(R⁵)═, A² represents an oxygen atom, A³ represents ═C(R⁴)—, R¹ represents a carboxy group, and R², R³, and R⁴ represent a hydrogen atom;

a compound represented by Formula (1) in which A¹ represents —C(R⁵)═, A² represents an oxygen atom, A³ represents ═C(R⁴)—, R² represents a carboxy group, and R¹, R³, and R⁴ represent a hydrogen atom;

a compound represented by Formula (1) in which A¹ represents —C(R⁵)═, A² represents an oxygen atom, A³ represents ═C(R⁴)—, R³ represents a carboxy group, and R¹, R², and R⁴ represent a hydrogen atom;

a compound represented by Formula (1) in which A¹ represents —C(R⁵)═, A² represents an oxygen atom, A³ represents ═C(R⁴)—, R⁴ represents a carboxy group, and R¹, R², and R³ represent a hydrogen atom;

a compound represented by Formula (1) in which A¹ represents —C(R⁵)═, A² represents an oxygen atom, A³ represents ═C(R⁴)—, and R¹ represents a carboxy group;

a compound represented by Formula (1) in which A¹ represents —C(R⁵)═, A² represents an oxygen atom, A³ represents ═C(R⁴)—, and R² represents a carboxy group;

a compound represented by Formula (1) in which A¹ represents —C(R⁵)═, A² represents an oxygen atom, A³ represents ═C(R⁴)—, and R³ represents a carboxy group;

a compound represented by Formula (1) in which A¹ represents —C(R⁵)═, A² represents an oxygen atom, A³ represents ═C(R⁴)—, and R⁴ represents a carboxy group;

a compound represented by Formula (1) in which A¹ represents —C(R⁵)═, A² represents an oxygen atom, A³ represents ═C(R⁴)—, and R⁵ represents a carboxy group;

a compound represented by Formula (1) in which A¹ represents —C(R⁵)═, A² represents an oxygen atom, A³ represents ═C(R⁴)—, R¹ represents a C2-C6 alkoxycarbonyl group, and R², R³, and R⁴ represent a hydrogen atom;

a compound represented by Formula (1) in which A¹ represents —C(R⁵)═, A² represents an oxygen atom, A³ represents ═C(R⁴)—, R² represents a C2-C6 alkoxycarbonyl group, and R¹, R³, and R⁴ represent a hydrogen atom;

a compound represented by Formula (1) in which A¹ represents —C(R⁵)═, A² represents an oxygen atom, A³ represents ═C(R⁴)—, R³ represents a C2-C6 alkoxycarbonyl group, and R¹, R², and R⁴ represent a hydrogen atom;

a compound represented by Formula (1) in which A¹ represents —C(R⁵)═, A² represents an oxygen atom, A³ represents ═C(R⁴)—, R⁴ represents a C2-C6 alkoxycarbonyl group, and R¹, R², and R³ represent a hydrogen atom;

a compound represented by Formula (1) in which A¹ represents —C(R⁵)═, A² represents an oxygen atom, A³ represents ═C(R⁴)—, and R¹ represents a C2-C6 alkoxycarbonyl group;

a compound represented by Formula (1) in which A¹ represents —C(R⁵)═, A² represents an oxygen atom, A³ represents ═C(R⁴)—, and R² represents a C2-C6 alkoxycarbonyl group;

a compound represented by Formula (1) in which A¹ represents —C(R⁵)═, A² represents an oxygen atom, A³ represents ═C(R⁴)—, and R³ represents a C2-C6 alkoxycarbonyl group;

a compound represented by Formula (1) in which A¹ represents —C(R⁵)═, A² represents an oxygen atom, A³ represents ═C(R⁴)—, and R⁴ represents a C2-C6 alkoxycarbonyl group;

a compound represented by Formula (1) in which A¹ represents —C(R⁵)═, A² represents an oxygen atom, A³ represents ═C(R⁴)—, and R⁵ represents a C2-C6 alkoxycarbonyl group;

a compound represented by Formula (1) in which A¹ represents —C(R⁵)═, A² represents an oxygen atom, A³ represents a nitrogen atom, R² represents a C1-C6 alkyl group optionally having one or more groups selected from the group X, and R¹, R³, R⁴, and R⁵ represent a hydrogen atom;

a compound represented by Formula (1) in which A¹ represents —C(R⁵)═, A² represents an oxygen atom, A³ represents a nitrogen atom, R² represents a trifluoromethyl group, and R¹, R³, R⁴, and R⁵ represent a hydrogen atom;

a compound represented by Formula (1) in which A¹ represents —C(R⁵)═, A² represents an oxygen atom, A³ represents ═C(R⁴)—, and R¹, R², R³, and R⁴ may be the same or different and each represents a hydrogen atom, a halogen atom, a cyano group, a nitro group, a C1-C6 alkyl group optionally having one or more groups selected from the group X, a phenyl group optionally having one or more groups selected from the group Y, a carboxy group, a C2-C6 alkoxycarbonyl group, an aminocarbonyl group, —NR¹¹R¹⁵, —OR¹¹, or —S(O)ₘR¹¹;

a compound represented by Formula (1) in which A¹ represents —C(R⁵)═, A² represents an oxygen atom, A³ represents ═C(R⁴)—, R¹ represents a halogen atom, a cyano group, a nitro group, a C1-C6 alkyl group optionally having one or more groups selected from the group X, a phenyl group optionally having one or more groups selected from the group Y, a carboxy group, a C2-C6 alkoxycarbonyl group, an aminocarbonyl group, —NR¹²R¹⁵, —OR¹⁵, or —S(O)ₘR¹¹, R², R³, and R⁴ may be the same or different and each represents a hydrogen atom, a halogen atom, a cyano group, a nitro group, a C1-C6 alkyl group optionally having one or more groups selected from the group X, a phenyl group optionally having one or more groups selected from the group Y, a carboxy group, a C2-C6 alkoxycarbonyl group, an aminocarbonyl group, —NR¹²R¹⁵, —OR¹⁵, or —S(O)ₘR¹¹, and R⁵ represents a hydrogen atom, a halogen atom, a C1-C6 alkyl group optionally having one or more groups selected from the group X, —NR¹¹R¹², —OR¹³, or a phenyl group optionally having one or more groups selected from the group Y;

a compound represented by Formula (1) in which A¹ represents —C(R⁵)═, A² represents an oxygen atom, A³ represents ═C(R⁴)—, R¹, R³, and R⁴ may be the same or different and each represents a hydrogen atom, a halogen atom, a cyano group, a nitro group, a C1-C6 alkyl group optionally having one or more groups selected from the group X, a phenyl group optionally having one or more groups selected from the group Y, a carboxy group, a C2-C6 alkoxycarbonyl group, an aminocarbonyl group, —NR¹²R¹⁵, —OR¹⁵, or —S(O)ₘR¹¹, R² represents a halogen atom, a cyano group, a nitro group, a C1-C6 alkyl group optionally having one or more groups selected from the group X, a phenyl group optionally having one or more groups selected from the group Y, a carboxy group, a C2-C6 alkoxycarbonyl group, an aminocarbonyl group, —NR¹²R¹⁵, —OR¹⁵, or —S(O)ₘR¹¹, and R⁵ represents a hydrogen atom, a halogen atom, a C1-C6 alkyl group optionally having one or more groups selected from the group X, —NR¹¹R¹², —OR¹³, or a phenyl group optionally having one or more groups selected from the group Y;

a compound represented by Formula (1) in which A¹ represents —C(R⁵)═, A² represents an oxygen atom, A³ represents ═C(R⁴)—, R¹, R², and R⁴ may be the same or different and each represents a hydrogen atom, a halogen atom, a cyano group, a nitro group, a C1-C6 alkyl group optionally having one or more groups selected from the group X, a phenyl group optionally having one or more groups selected from the group Y, a carboxy group, a C2-C6 alkoxycarbonyl group, an aminocarbonyl group, —NR¹²R¹⁵, —OR¹⁵, or —S(O)ₘR¹¹, R³ represents a halogen atom, a cyano group, a nitro group, a C1-C6 alkyl group optionally having one or more groups selected from the group X, a phenyl group optionally having one or more groups selected from the group Y, a carboxy group, a C2-C6 alkoxycarbonyl group, an aminocarbonyl group, —NR¹²R¹⁵, —OR¹⁵, or —S(O)ₘR¹¹, and R⁵ represents a hydrogen atom, a halogen atom, a C1-C6 alkyl group optionally having one or more groups selected from the group X, —NR¹¹R¹², —OR¹³, or a phenyl group optionally having one or more groups selected from the group Y;

a compound represented by Formula (1) in which A¹ represents —C(R⁵)═, A² represents an oxygen atom, A³ represents ═C(R⁴)—, R¹, R², and R³ may be the same or different and each represents a hydrogen atom, a halogen atom, a cyano group, a nitro group, a C1-C6 alkyl group optionally having one or more groups selected from the group X, a phenyl group optionally having one or more groups selected from the group Y, a carboxy group, a C2-C6 alkoxycarbonyl group, an aminocarbonyl group, —NR¹²R¹⁵, —OR¹⁵, or —S(O)ₘR¹¹, R⁴ represents a halogen atom, a cyano group, a nitro group, a C1-C6 alkyl group optionally having one or more groups selected from the group X, a phenyl group optionally having one or more groups selected from the group Y, a carboxy group, a C2-C6 alkoxycarbonyl group, an aminocarbonyl group, —NR¹²R¹⁵, —OR¹⁵, or —S(O)ₘR¹¹, and R⁵ represents a hydrogen atom, a halogen atom, a C1-C6 alkyl group optionally having one or more groups selected from the group X, —NR¹¹R¹², —OR¹³, or a phenyl group optionally having one or more groups selected from the group Y;

a compound represented by Formula (1) in which A¹ represents —C(R⁵)═, A² represents an oxygen atom, A³ represents ═C(R⁴)—, R¹, R², R³, and R⁴ may be the same or different and each represents a hydrogen atom, a halogen atom, a cyano group, a nitro group, a C1-C6 alkyl group optionally having one or more groups selected from the group X, a phenyl group optionally having one or more groups selected from the group Y, or —OR¹⁵, and R⁵ represents a halogen atom, a C1-C6 alkyl group optionally having one or more groups selected from the group X, —NR¹¹R¹², —OR¹³, or a phenyl group optionally having one or more groups selected from the group Y;

a compound represented by Formula (1) in which A¹ represents —C(R⁵)═, A² represents an oxygen atom, A³ represents ═C(R⁴)—, R¹ represents a halogen atom, a cyano group, a nitro group, a C1-C6 alkyl group optionally having one or more groups selected from the group X, a phenyl group optionally having one or more groups selected from the group Y, or —OR¹⁵, R², R³, and R⁴ may be the same or different and each represents a hydrogen atom, a halogen atom, a cyano group, a nitro group, a C1-C6 alkyl group optionally having one or more groups selected from the group X, a phenyl group optionally having one or more groups selected from the group Y, or —OR¹⁵, R⁵ represents a hydrogen atom, a halogen atom, a C1-C6 alkyl group optionally having one or more groups selected from the group X, —NR¹¹R¹², —OR¹³, or a phenyl group optionally having one or more groups selected from the group Y, R¹³ represents a C1-C6 alkyl group optionally having one or more groups selected from the group X or a hydrogen atom, and R¹⁵ represents a C1-C6 alkyl group optionally having one or more groups selected from the group X;

a compound represented by Formula (1) in which A¹ represents —C(R⁵)═, A² represents an oxygen atom, A³ represents ═C(R⁴)—, R¹, R³, and R⁴ may be the same or different and each represents a hydrogen atom, a halogen atom, a cyano group, a nitro group, a C1-C6 alkyl group optionally having one or more groups selected from the group X, a phenyl group optionally having one or more groups selected from the group Y, or —OR¹⁵, R² represents a halogen atom, a cyano group, a nitro group, a C1-C6 alkyl group optionally having one or more groups selected from the group X, a phenyl group optionally having one or more groups selected from the group Y, or —OR¹⁵, R⁵ represents a hydrogen atom, a halogen atom, a C1-C6 alkyl group optionally having one or more groups selected from the group X, —NR¹¹R¹², —OR¹³, or a phenyl group optionally having one or more groups selected from the group Y, R¹³ represents a C1-C6 alkyl group optionally having one or more groups selected from the group X or a hydrogen atom, and R¹⁵ represents a C1-C6 alkyl group optionally having one or more groups selected from the group X;

a compound represented by Formula (1) in which A¹ represents —C(R⁵)═, A² represents an oxygen atom, A³ represents ═C(R⁴)—, R¹, R², and R⁴ may be the same or different and each represents a hydrogen atom, a halogen atom, a cyano group, a nitro group, a C1-C6 alkyl group optionally having one or more groups selected from the group X, a phenyl group optionally having one or more groups selected from the group Y, or —OR¹⁵, R³ represents a halogen atom, a cyano group, a nitro group, a C1-C6 alkyl group optionally having one or more groups selected from the group X, a phenyl group optionally having one or more groups selected from the group Y, or —OR¹⁵, R⁵ represents a hydrogen atom, a halogen atom, a C1-C6 alkyl group optionally having one or more groups selected from the group X, —NR¹¹R¹², —OR¹³, or a phenyl group optionally having one or more groups selected from the group Y, R¹³ represents a C1-C6 alkyl group optionally having one or more groups selected from the group X or a hydrogen atom, and R¹⁵ represents a C1-C6 alkyl group optionally having one or more groups selected from the group X;

a compound represented by Formula (1) in which A¹ represents —C(R⁵)═, A² represents an oxygen atom, A³ represents ═C(R⁴)—, R¹, R², and R³ may be the same or different and each represents a hydrogen atom, a halogen atom, a cyano group, a nitro group, a C1-C6 alkyl group optionally having one or more groups selected from the group X, a phenyl group optionally having one or more groups selected from the group Y, or —OR¹⁵, R⁴ represents a halogen atom, a cyano group, a nitro group, a C1-C6 alkyl group optionally having one or more groups selected from the group X, a phenyl group optionally having one or more groups selected from the group Y, or —OR¹⁵, R⁵ represents a hydrogen atom, a halogen atom, a C1-C6 alkyl group optionally having one or more groups selected from the group X, —NR¹¹R¹², —OR¹³, or a phenyl group optionally having one or more groups selected from the group Y, R¹³ represents a C1-C6 alkyl group optionally having one or more groups selected from the group X or a hydrogen atom, and R¹⁵ represents a C1-C6 alkyl group optionally having one or more groups selected from the group X;

a compound represented by Formula (1) in which A¹ represents —C(R⁵)═, A² represents an oxygen atom, A³ represents ═C(R⁴)—, W represents —OR⁶, and R¹, R², R³ and R⁴ may be the same or different and each represents a hydrogen atom, a halogen atom, a cyano group, a nitro group, a C1-C6 alkyl group optionally having one or more groups selected from the group X, a phenyl group optionally having one or more groups selected from the group Y, a carboxy group, a C2-C6 alkoxycarbonyl group, an aminocarbonyl group, —NR¹²R¹⁵, —OR¹⁵, or —S(O)ₘR¹¹;

a compound represented by Formula (1) in which A¹ represents —C(R⁵)═, A² represents an oxygen atom, A³ represents ═C(R⁴)—, W represents —OR⁶, R¹ represents a halogen atom, a cyano group, a nitro group, a C1-C6 alkyl group optionally having one or more groups selected from the group X, a phenyl group optionally having one or more groups selected from the group Y, a carboxy group, a C2-C6 alkoxycarbonyl group, an aminocarbonyl group, —NR¹²R¹⁵, —OR¹⁵, or —S(O)ₘR¹¹, R², R³ and R⁴ may be the same or different and each represents a hydrogen atom, a halogen atom, a cyano group, a nitro group, a C1-C6 alkyl group optionally having one or more groups selected from the group X, a phenyl group optionally having one or more groups selected from the group Y, a carboxy group, a C2-C6 alkoxycarbonyl group, an aminocarbonyl group, —NR¹²R¹⁵, —OR¹⁵, or —S(O)ₘR¹¹, and R⁶ represents a hydrogen atom or a C1-C6 alkyl group optionally having one or more groups selected from the group Z;

a compound represented by Formula (1) in which A¹ represents —C(R⁵)═, A² represents an oxygen atom, A³ represents ═C(R⁴)—, W represents —OR⁶, R¹, R³ and R⁴ may be the same or different and each represents a hydrogen atom, a halogen atom, a cyano group, a nitro group, a C1-C6 alkyl group optionally having one or more groups selected from the group X, a phenyl group optionally having one or more groups selected from the group Y, a carboxy group, a C2-C6 alkoxycarbonyl group, an aminocarbonyl group, —NR¹²R¹⁵, —OR¹⁵, or —S(O)ₘR¹¹, R² represents a halogen atom, a cyano group, a nitro group, a C1-C6 alkyl group optionally having one or more groups selected from the group X, a phenyl group optionally having one or more groups selected from the group Y, a carboxy group, a C2-C6 alkoxycarbonyl group, an aminocarbonyl group, —NR¹²R¹⁵, —OR¹⁵, or —S(O)ₘR¹¹, and R⁶ represents a hydrogen atom or a C1-C6 alkyl group optionally having one or more groups selected from the group Z;

a compound represented by Formula (1) in which A¹ represents —C(R⁵)═, A² represents an oxygen atom, A³ represents ═C(R⁴)—, W represents —OR⁶, and R¹, R² and R⁴ may be the same or different and each represents a hydrogen atom, a halogen atom, a cyano group, a nitro group, a C1-C6 alkyl group optionally having one or more groups selected from the group X, a phenyl group optionally having one or more groups selected from the group Y, a carboxy group, a C2-C6 alkoxycarbonyl group, an aminocarbonyl group, —NR¹²R¹⁵, —OR¹⁵, or —S(O)ₘR¹¹, R³ represents a halogen atom, a cyano group, a nitro group, a C1-C6 alkyl group optionally having one or more groups selected from the group X, a phenyl group optionally having one or more groups selected from the group Y, a carboxy group, a C2-C6 alkoxycarbonyl group, an aminocarbonyl group, —NR¹²R¹⁵, —OR¹⁵, or —S(O)ₘR¹¹, and R⁶ represents a hydrogen atom or a C1-C6 alkyl group optionally having one or more groups selected from the group Z;

a compound represented by Formula (1) in which A¹ represents —C(R⁵)═, A² represents an oxygen atom, A³ represents ═C(R⁴)—, W represents —OR⁶, and R¹, R² and R³ may be the same or different and each represents a hydrogen atom, a halogen atom, a cyano group, a nitro group, a C1-C6 alkyl group optionally having one or more groups selected from the group X, a phenyl group optionally having one or more groups selected from the group Y, a carboxy group, a C2-C6 alkoxycarbonyl group, an aminocarbonyl group, —NR¹²R¹⁵, —OR¹⁵, or —S(O)ₘR¹¹, R⁴ represents a halogen atom, a cyano group, a nitro group, a C1-C6 alkyl group optionally having one or more groups selected from the group X, a phenyl group optionally having one or more groups selected from the group Y, a carboxy group, a C2-C6 alkoxycarbonyl group, an aminocarbonyl group, —NR¹²R¹⁵, —OR¹⁵, or —S(O)ₘR¹¹, and R⁶ represents a hydrogen atom or a C1-C6 alkyl group optionally having one or more groups selected from the group Z;

a compound represented by Formula (1) in which A¹ represents —C(R⁵)═, A² represents an oxygen atom, A³ represents ═C(R⁴)—, W represents —OR⁶, R¹ represents a halogen atom, a cyano group, a nitro group, a C1-C6 alkyl group optionally having one or more groups selected from the group X, a phenyl group optionally having one or more groups selected from the group Y, a carboxy group, a C2-C6 alkoxycarbonyl group, an aminocarbonyl group, —NR¹²R¹⁵, —OR¹⁵, or —S(O)ₘR¹¹, R², R³ and R⁴ may be the same or different and each represents a hydrogen atom, a halogen atom, a cyano group, a nitro group, a C1-C6 alkyl group optionally having one or more groups selected from the group X, a phenyl group optionally having one or more groups selected from the group Y, a carboxy group, a C2-C6 alkoxycarbonyl group, an aminocarbonyl group, —NR¹²R¹⁵, —OR¹⁵, or —S(O)ₘR¹¹, R⁵ represents a hydrogen atom, a halogen atom, a C1-C6 alkyl group optionally having one or more groups selected from the group X, —NR¹¹R¹², —OR¹³, or a phenyl group optionally having one or more groups selected from the group Y, and R⁶ represents a hydrogen atom or a C1-C6 alkyl group optionally having one or more groups selected from the group Z;

a compound represented by Formula (1) in which A¹ represents —C(R⁵)═, A² represents an oxygen atom, A³ represents ═C(R⁴)—, W represents —OR⁶, R¹, R³ and R⁴ may be the same or different and each represents a hydrogen atom, a halogen atom, a cyano group, a nitro group, a C1-C6 alkyl group optionally having one or more groups selected from the group X, a phenyl group optionally having one or more groups selected from the group Y, a carboxy group, a C2-C6 alkoxycarbonyl group, an aminocarbonyl group, —NR¹²R¹⁵, —OR¹⁵, or —S(O)ₘR¹¹, R² represents a halogen atom, a cyano group, a nitro group, a C1-C6 alkyl group optionally having one or more groups selected from the group X, a phenyl group optionally having one or more groups selected from the group Y, a carboxy group, a C2-C6 alkoxycarbonyl group, an aminocarbonyl group, —NR¹²R¹⁵, —OR¹⁵, or —S(O)ₘR¹¹, R⁵ represents a hydrogen atom, a halogen atom, a C1-C6 alkyl group optionally having one or more groups selected from the group X, —NR¹¹R¹², —OR¹³, or a phenyl group optionally having one or more groups selected from the group Y, and R⁶ represents a hydrogen atom or a C1-C6 alkyl group optionally having one or more groups selected from the group Z;

a compound represented by Formula (1) in which A¹ represents —C(R⁵)═, A² represents an oxygen atom, A³ represents ═C(R⁴)—, W represents —OR⁶, R¹, R² and R⁴ may be the same or different and each represents a hydrogen atom, a halogen atom, a cyano group, a nitro group, a C1-C6 alkyl group optionally having one or more groups selected from the group X, a phenyl group optionally having one or more groups selected from the group Y, a carboxy group, a C2-C6 alkoxycarbonyl group, an aminocarbonyl group, —NR¹²R¹⁵, —OR¹⁵, or —S(O)ₘR¹¹, R³ represents a halogen atom, a cyano group, a nitro group, a C1-C6 alkyl group optionally having one or more groups selected from the group X, a phenyl group optionally having one or more groups selected from the group Y, a carboxy group, a C2-C6 alkoxycarbonyl group, an aminocarbonyl group, —NR¹²R¹⁵, —OR¹⁵, or —S(O)ₘR¹¹, R⁵ represents a hydrogen atom, a halogen atom, a C1-C6 alkyl group optionally having one or more groups selected from the group X, —NR¹¹R¹², —OR¹³, or a phenyl group optionally having one or more groups selected from the group Y, and R⁶ represents a hydrogen atom or a C1-C6 alkyl group optionally having one or more groups selected from the group Z;

a compound represented by Formula (1) in which A¹ represents —C(R⁵)═, A² represents an oxygen atom, A³ represents ═C(R⁴)—, W represents —OR⁶, R¹, R² and R³ may be the same or different and each represents a hydrogen atom, a halogen atom, a cyano group, a nitro group, a C1-C6 alkyl group optionally having one or more groups selected from the group X, a phenyl group optionally having one or more groups selected from the group Y, a carboxy group, a C2-C6 alkoxycarbonyl group, an aminocarbonyl group, —NR¹²R¹⁵, —OR¹⁵, or —S(O)ₘR¹¹, R⁴ represents a halogen atom, a cyano group, a nitro group, a C1-C6 alkyl group optionally having one or more groups selected from the group X, a phenyl group optionally having one or more groups selected from the group Y, a carboxy group, a C2-C6 alkoxycarbonyl group, an aminocarbonyl group, —NR¹²R¹⁵, —OR¹⁵, or —S(O)ₘR¹¹, R⁵ represents a hydrogen atom, a halogen atom, a C1-C6 alkyl group optionally having one or more groups selected from the group X, —NR¹¹R¹², —OR¹³, or a phenyl group optionally having one or more groups selected from the group Y, and R⁶ represents a hydrogen atom or a C1-C6 alkyl group optionally having one or more groups selected from the group Z;

a compound represented by Formula (1) in which A¹ represents —C(R⁵)═, A² represents an oxygen atom, A³ represents ═C(R⁴)—, W represents —OR⁶, R¹, R², R³ and R⁴ may be the same or different and each represents a hydrogen atom, a halogen atom, a cyano group, a nitro group, a C1-C6 alkyl group optionally having one or more groups selected from the group X, a phenyl group optionally having one or more groups selected from the group Y, a carboxy group, a C2-C6 alkoxycarbonyl group, an aminocarbonyl group, —NR¹²R¹⁵, —OR¹⁵, or —S(O)ₘR¹¹, and R⁶ represents a C1-C6 alkyl group optionally having one or more groups selected from the group Z;

a compound represented by Formula (1) in which A¹ represents —C(R⁵)═, A² represents an oxygen atom, A³ represents ═C(R⁴)—, W represents —OR⁶, R¹, R², R³ and R⁴ may be the same or different and each represents a hydrogen atom, a halogen atom, a cyano group, a nitro group, a C1-C6 alkyl group optionally having one or more groups selected from the group X, a phenyl group optionally having one or more groups selected from the group Y, a carboxy group, a C2-C6 alkoxycarbonyl group, an aminocarbonyl group, —NR¹²R¹⁵, —OR⁶, or —S(O)ₘR¹¹, and R⁶ represents a C1-C6 alkyl group;

a compound represented by Formula (1) in which A¹ represents —C(R⁵)═, A² represents an oxygen atom, A³ represents ═C(R⁴)—, W represents —OR⁶, R¹, R², R³ and R⁴ may be the same or different and each represents a hydrogen atom, a halogen atom, a cyano group, a nitro group, a C1-C6 alkyl group optionally having one or more groups selected from the group X, a phenyl group optionally having one or more groups selected from the group Y, a carboxy group, a C2-C6 alkoxycarbonyl group, an aminocarbonyl group, —NR¹²R¹⁵, —OR¹⁵, or —S(O)ₘR¹¹, R⁵ represents a hydrogen atom, a halogen atom, a C1-C6 alkyl group optionally having one or more groups selected from the group X, —NR¹¹R¹², —OR¹³, or a phenyl group optionally having one or more groups selected from the group Y, and R⁶ represents a C1-C6 alkyl group optionally having one or more groups selected from the group Z;

a compound represented by Formula (1) in which A¹ represents —C(R⁵)═, A² represents an oxygen atom, A³ represents ═C(R⁴)—, W represents —OR⁶, R¹, R², R³ and R⁴ may be the same or different and each represents a hydrogen atom, a halogen atom, a cyano group, a nitro group, a C1-C6 alkyl group optionally having one or more groups selected from the group X, a phenyl group optionally having one or more groups selected from the group Y, a carboxy group, a C2-C6 alkoxycarbonyl group, an aminocarbonyl group, —NR¹²R¹⁵, —OR¹⁵, or —S(O)ₘR¹¹, R⁵ represents a hydrogen atom, a halogen atom, a C1-C6 alkyl group optionally having one or more groups selected from the group X, —NR¹¹R¹², —OR¹³, or a phenyl group optionally having one or more groups selected from the group Y, and R⁶ represents a C1-C6 alkyl group;

a compound represented by Formula (1) in which A¹ represents —C(R⁵)═, A² represents an oxygen atom, A³ represents ═C(R⁴)—, W represents —OR⁶, R¹, R², R³ and R⁴ may be the same or different and each represents a hydrogen atom, a halogen atom, a cyano group, a nitro group, a C1-C6 alkyl group optionally having one or more groups selected from the group X, a phenyl group optionally having one or more groups selected from the group Y, or —OR¹⁵, R⁵ represents a hydrogen atom, a C1-C6 alkyl group optionally having one or more groups selected from the group X, or —OR¹³, R⁶ represents a C1-C6 alkyl group, R¹³ represents a C1-C6 alkyl group optionally having one or more groups selected from the group X, and R¹⁵ represents a C1-C6 alkyl group optionally having one or more groups selected from the group X;

a compound represented by Formula (1) in which A¹ represents —C(R⁵)═, A² represents an oxygen atom, A³ represents ═C(R⁴)—, W represents —OR⁶, R¹ represents a halogen atom, a cyano group, a nitro group, a C1-C6 alkyl group optionally having one or more groups selected from the group X, a phenyl group optionally having one or more groups selected from the group Y, a carboxy group, a C2-C6 alkoxycarbonyl group, an aminocarbonyl group, —NR¹²R¹⁵, —OR¹⁵, or —S(O)ₘR¹¹, R², R³ and R⁴ may be the same or different and each represents a hydrogen atom, a halogen atom, a cyano group, a nitro group, a C1-C6 alkyl group optionally having one or more groups selected from the group X, a phenyl group optionally having one or more groups selected from the group Y, a carboxy group, a C2-C6 alkoxycarbonyl group, an aminocarbonyl group, —NR¹²R¹⁵, —OR¹⁵, or —S(O)ₘR¹¹, R⁵ represents a hydrogen atom, a halogen atom, a C1-C6 alkyl group optionally having one or more groups selected from the group X, —NR¹¹R¹², —OR¹³, or a phenyl group optionally having one or more groups selected from the group Y, and R⁶ represents a hydrogen atom;

a compound represented by Formula (1) in which A¹ represents —C(R⁵)═, A² represents an oxygen atom, A³ represents ═C(R⁴)—, W represents —OR⁶, R¹, R³ and R⁴ may be the same or different and each represents a hydrogen atom, a halogen atom, a cyano group, a nitro group, a C1-C6 alkyl group optionally having one or more groups selected from the group X, a phenyl group optionally having one or more groups selected from the group Y, a carboxy group, a C2-C6 alkoxycarbonyl group, an aminocarbonyl group, —NR¹²R¹⁵, —OR¹⁵, or —S(O)ₘR¹¹, R² represents a halogen atom, a cyano group, a nitro group, a C1-C6 alkyl group optionally having one or more groups selected from the group X, a phenyl group optionally having one or more groups selected from the group Y, a carboxy group, a C2-C6 alkoxycarbonyl group, an aminocarbonyl group, —NR¹²R¹⁵, —OR¹⁵, or —S(O)ₘR¹¹, R⁵ represents a hydrogen atom, a halogen atom, a C1-C6 alkyl group optionally having one or more groups selected from the group X, —NR¹¹R¹², —OR¹³, or a phenyl group optionally having one or more groups selected from the group Y, and R⁶ represents a hydrogen atom;

a compound represented by Formula (1) in which A¹ represents —C(R⁵)═, A² represents an oxygen atom, A³ represents ═C(R⁴)—, W represents —OR⁶, R¹, R² and R⁴ may be the same or different and each represents a hydrogen atom, a halogen atom, a cyano group, a nitro group, a C1-C6 alkyl group optionally having one or more groups selected from the group X, a phenyl group optionally having one or more groups selected from the group Y, a carboxy group, a C2-C6 alkoxycarbonyl group, an aminocarbonyl group, —NR¹²R¹⁵, —OR¹⁵, or —S(O)ₘR¹¹, R³ represents a halogen atom, a cyano group, a nitro group, a C1-C6 alkyl group optionally having one or more groups selected from the group X, a phenyl group optionally having one or more groups selected from the group Y, a carboxy group, a C2-C6 alkoxycarbonyl group, an aminocarbonyl group, —NR¹²R¹⁵, —OR¹⁵, or —S(O)ₘR¹¹, R⁵ represents a hydrogen atom, a halogen atom, a C1-C6 alkyl group optionally having one or more groups selected from the group X, —NR¹¹R¹², —OR¹³, or a phenyl group optionally having one or more groups selected from the group Y, and R⁶ represents a hydrogen atom;

a compound represented by Formula (1) in which A¹ represents —C(R⁵)═, A² represents an oxygen atom, A³ represents ═C(R⁴)—, W represents —OR⁶, R¹, R² and R³ may be the same or different and each represents a hydrogen atom, a halogen atom, a cyano group, a nitro group, a C1-C6 alkyl group optionally having one or more groups selected from the group X, a phenyl group optionally having one or more groups selected from the group Y, a carboxy group, a C2-C6 alkoxycarbonyl group, an aminocarbonyl group, —NR¹²R¹⁵, —OR¹⁵, or —S(O)ₘR¹¹, R⁴ represents a halogen atom, a cyano group, a nitro group, a C1-C6 alkyl group optionally having one or more groups selected from the group X, a phenyl group optionally having one or more groups selected from the group Y, a carboxy group, a C2-C6 alkoxycarbonyl group, an aminocarbonyl group, —NR¹²R¹⁵, —OR¹⁵, or —S(O)ₘR¹¹, R⁵ represents a hydrogen atom, a halogen atom, a C1-C6 alkyl group optionally having one or more groups selected from the group X, —NR¹¹R¹², —OR¹³, or a phenyl group optionally having one or more groups selected from the group Y, and R⁶ represents a hydrogen atom;

a compound represented by Formula (1) in which A¹ represents —C(R⁵)═, A² represents an oxygen atom, A³ represents ═C(R⁴)—, W represents —OR⁶, R¹ represents a halogen atom, a cyano group, a nitro group, a C1-C6 alkyl group optionally having one or more groups selected from the group X, a phenyl group optionally having one or more groups selected from the group Y, or —OR¹⁵, R², R³ and R⁴ may be the same or different and each represents a hydrogen atom, a halogen atom, a cyano group, a nitro group, a C1-C6 alkyl group optionally having one or more groups selected from the group X, a phenyl group optionally having one or more groups selected from the group Y, or —OR¹⁵, R⁵ represents a hydrogen atom, a halogen atom, a C1-C6 alkyl group optionally having one or more groups selected from the group X, —NR¹¹R¹², —OR¹³, or a phenyl group optionally having one or more groups selected from the group Y, R⁶ represents a hydrogen atom, R¹³ represents a C1-C6 alkyl group optionally having one or more groups selected from the group X, and R¹⁵ represents a C1-C6 alkyl group optionally having one or more groups selected from the group X;

a compound represented by Formula (1) in which A¹ represents —C(R⁵)═, A² represents an oxygen atom, A³ represents ═C(R⁴)—, W represents —OR⁶, R¹, R³ and R⁴ may be the same or different and each represents a hydrogen atom, a halogen atom, a cyano group, a nitro group, a C1-C6 alkyl group optionally having one or more groups selected from the group X, a phenyl group optionally having one or more groups selected from the group Y, or —OR¹⁵, R² represents a halogen atom, a cyano group, a nitro group, a C1-C6 alkyl group optionally having one or more groups selected from the group X, a phenyl group optionally having one or more groups selected from the group Y, or —OR¹⁵, R⁵ represents a hydrogen atom, a halogen atom, a C1-C6 alkyl group optionally having one or more groups selected from the group X, —NR¹¹R¹², —OR¹³, or a phenyl group optionally having one or more groups selected from the group Y, R⁶ represents a hydrogen atom, R¹³ represents a C1-C6 alkyl group optionally having one or more groups selected from the group X, and R¹⁵ represents a C1-C6 alkyl group optionally having one or more groups selected from the group X;

a compound represented by Formula (1) in which A¹ represents —C(R⁵)═, A² represents an oxygen atom, A³ represents ═C(R⁴)—, W represents —OR⁶, R¹, R² and R⁴ may be the same or different and each represents a hydrogen atom, a halogen atom, a cyano group, a nitro group, a C1-C6 alkyl group optionally having one or more groups selected from the group X, a phenyl group optionally having one or more groups selected from the group Y, or —OR¹⁵, R³ represents a halogen atom, a cyano group, a nitro group, a C1-C6 alkyl group optionally having one or more groups selected from the group X, a phenyl group optionally having one or more groups selected from the group Y, or —OR¹⁵, R⁵ represents a hydrogen atom, a halogen atom, a C1-C6 alkyl group optionally having one or more groups selected from the group X, —NR¹¹R¹², —OR¹³, or a phenyl group optionally having one or more groups selected from the group Y, R⁶ represents a hydrogen atom, R¹³ represents a C1-C6 alkyl group optionally having one or more groups selected from the group X, and R¹⁵ represents a C1-C6 alkyl group optionally having one or more groups selected from the group X;

a compound represented by Formula (1) in which A¹ represents —C(R⁵)═, A² represents an oxygen atom, A³ represents ═C(R⁴)—, W represents —OR⁶, R¹, R² and R³ may be the same or different and each represents a hydrogen atom, a halogen atom, a cyano group, a nitro group, a C1-C6 alkyl group optionally having one or more groups selected from the group X, a phenyl group optionally having one or more groups selected from the group Y, or —OR¹⁵, R⁴ represents a halogen atom, a cyano group, a nitro group, a C1-C6 alkyl group optionally having one or more groups selected from the group X, a phenyl group optionally having one or more groups selected from the group Y, or —OR¹⁵, R⁵ represents a hydrogen atom, a halogen atom, a C1-C6 alkyl group optionally having one or more groups selected from the group X, —NR¹¹R¹², —OR¹³, or a phenyl group optionally having one or more groups selected from the group Y, R⁶ represents a hydrogen atom, R¹³ represents a C1-C6 alkyl group optionally having one or more groups selected from the group X, and R¹⁵ represents a C1-C6 alkyl group optionally having one or more groups selected from the group X;

a compound represented by Formula (1) in which A¹ represents —C(R⁵)═, A² represents an oxygen atom, A³ represents ═C(R⁴)—, W represents —OR⁶, R¹ represents a halogen atom, a cyano group, a nitro group, a C1-C6 alkyl group optionally having one or more groups selected from the group X, a phenyl group optionally having one or more groups selected from the group Y, or —OR¹⁵, R², R³ and R⁴ may be the same or different and each represents a hydrogen atom, a halogen atom, a cyano group, a nitro group, a C1-C6 alkyl group optionally having one or more groups selected from the group X, a phenyl group optionally having one or more groups selected from the group Y, or —OR¹⁵, R⁵ represents a hydrogen atom, a C1-C6 alkyl group optionally having one or more groups selected from the group X, or —OR¹³, R⁶ represents a hydrogen atom, a methyl group, or an ethyl group, R¹³ represents a C1-C6 alkyl group optionally having one or more groups selected from the group X, and R¹⁵ represents a C1-C6 alkyl group optionally having one or more groups selected from the group X;

a compound represented by Formula (1) in which A¹ represents —C(R⁵)═, A² represents an oxygen atom, A³ represents ═C(R⁴)—, W represents —OR⁶, R¹, R³ and R⁴ may be the same or different and each represents a hydrogen atom, a halogen atom, a cyano group, a nitro group, a C1-C6 alkyl group optionally having one or more groups selected from the group X, a phenyl group optionally having one or more groups selected from the group Y, or —OR¹⁵, R² represents a halogen atom, a cyano group, a nitro group, a C1-C6 alkyl group optionally having one or more groups selected from the group X, a phenyl group optionally having one or more groups selected from the group Y, or —OR¹⁵, R⁵ represents a hydrogen atom, a C1-C6 alkyl group optionally having one or more groups selected from the group X, or —OR¹³, R⁶ represents a hydrogen atom, a methyl group, or an ethyl group, R¹³ represents a C1-C6 alkyl group optionally having one or more groups selected from the group X, and R¹⁵ represents a C1-C6 alkyl group optionally having one or more groups selected from the group X;

a compound represented by Formula (1) in which A¹ represents —C(R⁵)═, A² represents an oxygen atom, A³ represents ═C(R⁴)—, W represents —OR⁶, R¹, R² and R⁴ may be the same or different and each represents a hydrogen atom, a halogen atom, a cyano group, a nitro group, a C1-C6 alkyl group optionally having one or more groups selected from the group X, a phenyl group optionally having one or more groups selected from the group Y, or —OR¹⁵, R³ represents a halogen atom, a cyano group, a nitro group, a C1-C6 alkyl group optionally having one or more groups selected from the group X, a phenyl group optionally having one or more groups selected from the group Y, or —OR¹⁵, R⁵ represents a hydrogen atom, a C1-C6 alkyl group optionally having one or more groups selected from the group X, or —OR¹³, R⁶ represents a hydrogen atom, a methyl group, or an ethyl group, R¹³ represents a C1-C6 alkyl group optionally having one or more groups selected from the group X, and R¹⁵ represents a C1-C6 alkyl group optionally having one or more groups selected from the group X;

a compound represented by Formula (1) in which A¹ represents —C(R⁵)═, A² represents an oxygen atom, A³ represents ═C(R⁴)—, W represents —OR⁶, R¹, R² and R³ may be the same or different and each represents a hydrogen atom, a halogen atom, a cyano group, a nitro group, a C1-C6 alkyl group optionally having one or more groups selected from the group X, a phenyl group optionally having one or more groups selected from the group Y, or —OR¹⁵, R⁴ represents a halogen atom, a cyano group, a nitro group, a C1-C6 alkyl group optionally having one or more groups selected from the group X, a phenyl group optionally having one or more groups selected from the group Y, or —OR¹⁵, R⁵ represents a hydrogen atom, a C1-C6 alkyl group optionally having one or more groups selected from the group X, or —OR¹³, R⁶ represents a hydrogen atom, a methyl group, or an ethyl group, R¹³ represents a C1-C6 alkyl group optionally having one or more groups selected from the group X, and R¹⁵ represents a C1-C6 alkyl group optionally having one or more groups selected from the group X;

a compound represented by Formula (1) in which A¹ represents —C(R⁵)═, A² represents an oxygen atom, A³ represents ═C(R⁴)—, W represents —OR⁶, R¹ represents a halogen atom, a cyano group, a nitro group, a C1-C6 alkyl group optionally having one or more groups selected from the group X, a phenyl group optionally having one or more groups selected from the group Y, or —OR¹⁵, R², R³ and R⁴ may be the same or different and each represents a hydrogen atom, a halogen atom, a cyano group, a nitro group, a C1-C6 alkyl group optionally having one or more groups selected from the group X, a phenyl group optionally having one or more groups selected from the group Y, or —OR¹⁵, R⁵ represents a hydrogen atom, a C1-C6 alkyl group optionally having one or more groups selected from the group X, or —OR¹³, R⁶ represents a hydrogen atom, R¹³ represents a C1-C6 alkyl group optionally having one or more groups selected from the group X, and R¹⁵ represents a C1-C6 alkyl group optionally having one or more groups selected from the group X;

a compound represented by Formula (1) in which A¹ represents —C(R⁵)═, A² represents an oxygen atom, A³ represents ═C(R⁴)—, W represents —OR⁶, R¹, R³ and R⁴ may be the same or different and each represents a hydrogen atom, a halogen atom, a cyano group, a nitro group, a C1-C6 alkyl group optionally having one or more groups selected from the group X, a phenyl group optionally having one or more groups selected from the group Y, or —OR¹⁵, R² represents a halogen atom, a cyano group, a nitro group, a C1-C6 alkyl group optionally having one or more groups selected from the group X, a phenyl group optionally having one or more groups selected from the group Y, or —OR¹⁵, R⁵ represents a hydrogen atom, a C1-C6 alkyl group optionally having one or more groups selected from the group X, or —OR¹³, R⁶ represents a hydrogen atom, R¹³ represents a C1-C6 alkyl group optionally having one or more groups selected from the group X, and R¹⁵ represents a C1-C6 alkyl group optionally having one or more groups selected from the group X;

a compound represented by Formula (1) in which A¹ represents —C(R⁵)═, A² represents an oxygen atom, A³ represents ═C(R⁴)—, W represents —OR⁶, R¹, R² and R⁴ may be the same or different and each represents a hydrogen atom, a halogen atom, a cyano group, a nitro group, a C1-C6 alkyl group optionally having one or more groups selected from the group X, a phenyl group optionally having one or more groups selected from the group Y, or —OR¹⁵, R³ represents a halogen atom, a cyano group, a nitro group, a C1-C6 alkyl group optionally having one or more groups selected from the group X, a phenyl group optionally having one or more groups selected from the group Y, or —OR¹⁵, R⁵ represents a hydrogen atom, a C1-C6 alkyl group optionally having one or more groups selected from the group X, or —OR¹³, R⁶ represents a hydrogen atom, R¹³ represents a C1-C6 alkyl group optionally having one or more groups selected from the group X, and R¹⁵ represents a C1-C6 alkyl group optionally having one or more groups selected from the group X;

a compound represented by Formula (1) in which A¹ represents —C(R⁵)═, A² represents an oxygen atom, A³ represents ═C(R⁴)—, W represents —OR⁶, R¹, R² and R³ may be the same or different and each represents a hydrogen atom, a halogen atom, a cyano group, a nitro group, a C1-C6 alkyl group optionally having one or more groups selected from the group X, a phenyl group optionally having one or more groups selected from the group Y, or —OR¹⁵, R⁴ represents a halogen atom, a cyano group, a nitro group, a C1-C6 alkyl group optionally having one or more groups selected from the group X, a phenyl group optionally having one or more groups selected from the group Y, or —OR¹⁵, R⁵ represents a hydrogen atom, a C1-C6 alkyl group optionally having one or more groups selected from the group X, or —OR¹³, R⁶ represents a hydrogen atom, R¹³ represents a C1-C6 alkyl group optionally having one or more groups selected from the group X, and R¹⁵ represents a C1-C6 alkyl group optionally having one or more groups selected from the group X;

a compound represented by Formula (1) in which A¹ represents —C(R⁵)═, A² represents an oxygen atom, A³ represents ═C(R⁴)—, W represents —OR⁶, R¹, R², R³, and R⁴ may be the same or different and each represents a hydrogen atom, a halogen atom, a cyano group, a nitro group, a C1-C6 alkyl group optionally having one or more groups selected from the group X, a phenyl group optionally having one or more groups selected from the group Y, or —OR¹⁵, R⁶ represents a C1-C6 alkyl group, and R¹⁵ represents a C1-C6 alkyl group optionally having one or more groups selected from the group X;

a compound represented by Formula (1) in which A¹ represents —C(R⁵)═, A² represents an oxygen atom, A³ represents ═C(R⁴)—, W represents —OR⁶, R¹ represents a trifluoromethyl group, R², R³, and R⁴ may be the same or different and each represents a hydrogen atom, a halogen atom, a cyano group, a nitro group, a C1-C6 alkyl group optionally having one or more groups selected from the group X, a phenyl group optionally having one or more groups selected from the group Y, or —OR¹¹, R⁵ represents a hydrogen atom, a halogen atom, a C1-C6 alkyl group optionally having one or more groups selected from the group X, —NR¹¹R¹², —OR¹³, or a phenyl group optionally having one or more groups selected from the group Y, R⁶ represents a hydrogen group, a methyl group, or an ethyl group, R¹³ represents a C1-C6 alkyl group optionally having one or more groups selected from the group X, and R¹⁵ represents a C1-C6 alkyl group optionally having one or more groups selected from the group X;

a compound represented by Formula (1) in which A¹ represents —C(R⁵)═, A² represents an oxygen atom, A³ represents ═C(R⁴)—, W represents —OR⁶, R¹, R³, and R⁴ may be the same or different and each represents a hydrogen atom, a halogen atom, a cyano group, a nitro group, a C1-C6 alkyl group optionally having one or more groups selected from the group X, a phenyl group optionally having one or more groups selected from the group Y, or —OR¹⁵, R² represents a trifluoromethyl group, R⁵ represents a hydrogen atom, a halogen atom, a C1-C6 alkyl group optionally having one or more groups selected from the group X, —NR¹¹R¹², —OR¹³, or a phenyl group optionally having one or more groups selected from the group Y, R⁶ represents a hydrogen atom, a methyl group, or an ethyl group, R¹³ represents a C1-C6 alkyl group optionally having one or more groups selected from the group X, and R¹⁵ represents a C1-C6 alkyl group optionally having one or more groups selected from the group X;

a compound represented by Formula (1) in which A¹ represents —C(R⁵)═, A² represents an oxygen atom, A³ represents ═C(R⁴)—, W represents —OR⁶, R¹, R², and R⁴ may be the same or different and each represents a hydrogen atom, a halogen atom, a cyano group, a nitro group, a C1-C6 alkyl group optionally having one or more groups selected from the group X, a phenyl group optionally having one or more groups selected from the group Y, or —OR¹⁵, R³ represents a trifluoromethyl group, R⁵ represents a hydrogen atom, a halogen atom, a C1-C6 alkyl group optionally having one or more groups selected from the group X, —NR¹¹R¹², —OR¹³, or a phenyl group optionally having one or more groups selected from the group Y, R⁶ represents a hydrogen atom, a methyl group, or an ethyl group, R¹³ represents a C1-C6 alkyl group optionally having one or more groups selected from the group X, and R¹⁵ represents a C1-C6 alkyl group optionally having one or more groups selected from the group X;

a compound represented by Formula (1) in which A¹ represents —C(R⁵)═, A² represents an oxygen atom, A³ represents ═C(R⁴)—, W represents —OR⁶, R¹, R², and R³ may be the same or different and each represents a hydrogen atom, a halogen atom, a cyano group, a nitro group, a C1-C6 alkyl group optionally having one or more groups selected from the group X, a phenyl group optionally having one or more groups selected from the group Y, or —OR¹⁵, R⁴ represents a trifluoromethyl group, R⁵ represents a hydrogen atom, a halogen atom, a C1-C6 alkyl group optionally having one or more groups selected from the group X, —NR¹¹R¹², —OR³, or a phenyl group optionally having one or more groups selected from the group Y, R⁶ represents a hydrogen atom, a methyl group, or an ethyl group, R¹³ represents a C1-C6 alkyl group optionally having one or more groups selected from the group X, and R¹⁵ represents a C1-C6 alkyl group optionally having one or more groups selected from the group X;

a compound represented by Formula (1) in which A¹ represents —C(R⁵)═, A² represents an oxygen atom, A³ represents ═C(R⁴)—, W represents —OR⁶, R¹ represents a trifluoromethyl group, R², R³, and R⁴ may be the same or different and each represents a hydrogen atom, a halogen atom, a cyano group, a nitro group, a C1-C6 alkyl group optionally having one or more groups selected from the group X, a phenyl group optionally having one or more groups selected from the group Y, or —OR¹⁵, R⁶ represents a methyl group or an ethyl group, and R¹⁵ represents a C1-C6 alkyl group optionally having one or more groups selected from the group X;

a compound represented by Formula (1) in which A¹ represents —C(R⁵)═, A² represents an oxygen atom, A³ represents ═C(R⁴)—, W represents —OR⁶, R¹, R³, and R⁴ may be the same or different and each represents a hydrogen atom, a halogen atom, a cyano group, a nitro group, a C1-C6 alkyl group optionally having one or more groups selected from the group X, a phenyl group optionally having one or more groups selected from the group Y, or —OR¹⁵, R² represents a trifluoromethyl group, R⁶ represents a methyl group or an ethyl group, and R¹⁵ represents a C1-C6 alkyl group optionally having one or more groups selected from the group X;

a compound represented by Formula (1) in which A¹ represents —C(R⁵)═, A² represents an oxygen atom, A³ represents ═C(R⁴)—, W represents —OR⁶, R¹, R², and R⁴ may be the same or different and each represents a hydrogen atom, a halogen atom, a cyano group, a nitro group, a C1-C6 alkyl group optionally having one or more groups selected from the group X, a phenyl group optionally having one or more groups selected from the group Y, or —OR¹⁵, R³ represents a trifluoromethyl group, R⁶ represents a methyl group or an ethyl group, and R¹⁵ represents a C1-C6 alkyl group optionally having one or more groups selected from the group X;

a compound represented by Formula (1) in which A¹ represents —C(R⁵)═, A² represents an oxygen atom, A³ represents ═C(R⁴)—, W represents —OR⁶, R¹, R², and R³ may be the same or different and each represents a hydrogen atom, a halogen atom, a cyano group, a nitro group, a C1-C6 alkyl group optionally having one or more groups selected from the group X, a phenyl group optionally having one or more groups selected from the group Y, or —OR¹⁵, R⁴ represents a trifluoromethyl group, R⁶ represents a methyl group or an ethyl group, and R¹⁵ represents a C1-C6 alkyl group optionally having one or more groups selected from the group X;

a compound represented by Formula (1) in which A¹ represents —C(R⁵)═, A² represents an oxygen atom, A³ represents ═C(R⁴)—, W represents —OR⁶, R¹ represents a trifluoromethyl group, R², R³, and R⁴ may be the same or different and each represents a hydrogen atom, a halogen atom, a cyano group, a nitro group, a C1-C6 alkyl group optionally having one or more groups selected from the group X, a phenyl group optionally having one or more groups selected from the group Y, or —OR¹⁵, R⁶ represents a hydrogen atom, and R¹⁵ represents a C1-C6 alkyl group optionally having one or more groups selected from the group X;

a compound represented by Formula (1) in which A¹ represents —C(R⁵)═, A² represents an oxygen atom, A³ represents ═C(R⁴)—, W represents —OR⁶, R¹, R³, and R⁴ may be the same or different and each represents a hydrogen atom, a halogen atom, a cyano group, a nitro group, a C1-C6 alkyl group optionally having one or more groups selected from the group X, a phenyl group optionally having one or more groups selected from the group Y, or —OR¹⁵, R² represents a trifluoromethyl group, R⁶ represents a hydrogen atom, and R¹⁵ represents a C1-C6 alkyl group optionally having one or more groups selected from the group X;

a compound represented by Formula (1) in which A¹ represents —C(R⁵)═, A² represents an oxygen atom, A³ represents ═C(R⁴)—, W represents —OR⁶, R¹, R², and R⁴ may be the same or different and each represents a hydrogen atom, a halogen atom, a cyano group, a nitro group, a C1-C6 alkyl group optionally having one or more groups selected from the group X, a phenyl group optionally having one or more groups selected from the group Y, or —OR¹⁵, R³ represents a trifluoromethyl group, R⁶ represents a hydrogen atom, and R¹⁵ represents a C1-C6 alkyl group optionally having one or more groups selected from the group X;

a compound represented by Formula (1) in which A¹ represents —C(R⁵)═, A² represents an oxygen atom, A³ represents ═C(R⁴)—, W represents —OR⁶, R¹, R², and R³ may be the same or different and each represents a hydrogen atom, a halogen atom, a cyano group, a nitro group, a C1-C6 alkyl group optionally having one or more groups selected from the group X, a phenyl group optionally having one or more groups selected from the group Y, or —OR¹⁵, R⁴ represents a trifluoromethyl group, R⁶ represents a hydrogen atom, and R¹⁵ represents a C1-C6 alkyl group optionally having one or more groups selected from the group X;

a compound represented by Formula (1) in which A¹ represents —C(R⁵)═, A² represents an oxygen atom, A³ represents ═C(R⁴)—, W represents —OR⁶, R¹ represents a trifluoromethyl group, R², R³, and R⁴ may be the same or different and each represents a hydrogen atom, a halogen atom, a cyano group, a nitro group, a C1-C6 alkyl group optionally having one or more groups selected from the group X, a phenyl group optionally having one or more groups selected from the group Y, or —OR¹⁵, R⁵ represents a hydrogen atom, R⁶ represents a methyl group or an ethyl group, and R¹⁵ represents a C1-C6 alkyl group optionally having one or more groups selected from the group X;

a compound represented by Formula (1) in which A¹ represents —C(R⁵)═, A² represents an oxygen atom, A³ represents ═C(R⁴)—, W represents —OR⁶, R¹, R³, and R⁴ may be the same or different and each represents a hydrogen atom, a halogen atom, a cyano group, a nitro group, a C1-C6 alkyl group optionally having one or more groups selected from the group X, a phenyl group optionally having one or more groups selected from the group Y, or —OR¹⁵, R² represents a trifluoromethyl group, R⁵ represents a hydrogen atom, R⁶ represents a methyl group or an ethyl group, and R¹⁵ represents a C1-C6 alkyl group optionally having one or more groups selected from the group X;

a compound represented by Formula (1) in which A¹ represents —C(R⁵)═, A² represents an oxygen atom, A³ represents ═C(R⁴)—, W represents —OR⁶, R¹, R², and R⁴ may be the same or different and each represents a hydrogen atom, a halogen atom, a cyano group, a nitro group, a C1-C6 alkyl group optionally having one or more groups selected from the group X, a phenyl group optionally having one or more groups selected from the group Y, or —OR¹⁵, R³ represents a trifluoromethyl group, R⁵ represents a hydrogen atom, R⁶ represents a methyl group or an ethyl group, and R¹⁵ represents a C1-C6 alkyl group optionally having one or more groups selected from the group X;

a compound represented by Formula (1) in which A¹ represents —C(R⁵)═, A² represents an oxygen atom, A³ represents ═C(R⁴)—, W represents —OR⁶, R¹, R², and R³ may be the same or different and each represents a hydrogen atom, a halogen atom, a cyano group, a nitro group, a C1-C6 alkyl group optionally having one or more groups selected from the group X, a phenyl group optionally having one or more groups selected from the group Y, or —OR¹⁵, R⁴ represents a trifluoromethyl group, R⁵ represents a hydrogen atom, R⁶ represents a methyl group or an ethyl group, R¹¹ represents a C1-C6 alkyl group optionally having one or more groups selected from the group X, and R¹⁵ represents a C1-C6 alkyl group optionally having one or more groups selected from the group X;

a compound represented by Formula (1) in which A¹ represents —C(R⁵)═, A² represents an oxygen atom, A³ represents ═C(R⁴)—, W represents —OR⁶, R¹ represents a trifluoromethyl group, R², R³, and R⁴ may be the same or different and each represents a hydrogen atom, a halogen atom, a cyano group, a nitro group, a C1-C6 alkyl group optionally having one or more groups selected from the group X, a phenyl group optionally having one or more groups selected from the group Y, or —OR¹⁵, R⁵ represents a hydrogen atom, R⁶ represents a hydrogen atom, and R¹⁵ represents a C1-C6 alkyl group optionally having one or more groups selected from the group X;

a compound represented by Formula (1) in which A¹ represents —C(R⁵)═, A² represents an oxygen atom, A³ represents ═C(R⁴)—, W represents —OR⁶, R¹, R³, and R⁴ may be the same or different and each represents a hydrogen atom, a halogen atom, a cyano group, a nitro group, a C1-C6 alkyl group optionally having one or more groups selected from the group X, a phenyl group optionally having one or more groups selected from the group Y, or —OR¹⁵, R² represents a trifluoromethyl group, R⁵ represents a hydrogen atom, R⁶ represents a hydrogen atom, and R¹⁵ represents a C1-C6 alkyl group optionally having one or more groups selected from the group X;

a compound represented by Formula (1) in which A¹ represents —C(R⁵)═, A² represents an oxygen atom, A³ represents ═C(R⁴)—, W represents —OR⁶, R¹, R², and R⁴ may be the same or different and each represents a hydrogen atom, a halogen atom, a cyano group, a nitro group, a C1-C6 alkyl group optionally having one or more groups selected from the group X, a phenyl group optionally having one or more groups selected from the group Y, or —OR¹⁵, R³ represents a trifluoromethyl group, R⁵ represents a hydrogen atom, R⁶ represents a hydrogen atom, and R¹⁵ represents a C1-C6 alkyl group optionally having one or more groups selected from the group X;

a compound represented by Formula (1) in which A¹ represents —C(R⁵)═, A² represents an oxygen atom, A³ represents ═C(R⁴)—, W represents —OR⁶, R¹, R², and R³ may be the same or different and each represents a hydrogen atom, a halogen atom, a cyano group, a nitro group, a C1-C6 alkyl group optionally having one or more groups selected from the group X, a phenyl group optionally having one or more groups selected from the group Y, or —OR¹⁵, R⁴ represents a trifluoromethyl group, R⁵ represents a hydrogen atom, R⁶ represents a hydrogen atom, and R¹⁵ represents a C1-C6 alkyl group optionally having one or more groups selected from the group X;

a compound represented by Formula (1) in which A¹ represents —C(R⁵)═, A² represents an oxygen atom, A³ represents ═C(R⁴)—, and W represents —ON═CR⁷R⁸;

a compound represented by Formula (1) in which A¹ represents —C(R⁵)═, A² represents an oxygen atom, A³ represents ═C(R⁴)—, and W represents a —SR⁹;

a compound represented by Formula (1) in which A¹ represents —C(R⁵)═, A² represents an oxygen atom, A³ represents ═C(R⁴)—, W represents a —SR⁹, and R⁹ represents a C1-C6 alkyl group;

a compound represented by Formula (1) in which A¹ represents —C(R⁵)═, A² represents an oxygen atom, A³ represents ═C(R⁴)—, and W represents —NR¹⁶R¹⁷;

a compound represented by Formula (1) in which A¹ represents —C(R⁵)═, A² represents an oxygen atom, A³ represents ═C(R⁴)—, W represents —NR¹⁶R¹⁷, R⁷ and R⁸ may be the same or different and each represents a C1-C6 alkyl group optionally having one or more halogen atoms or a hydrogen atom, R¹⁶ represents a C1-C6 alkyl group optionally having one or more halogen atoms or a hydrogen atom, R¹⁷ represents a cyano group, a phenyl group optionally having one or more groups selected from the group Y, —OR¹⁸, or —NR⁷R⁸, and R¹⁸ represents a hydrogen atom or a C2-C6 alkyl group optionally having one or more halogen atoms;

a compound represented by Formula (1) in which A¹ represents —C(R⁵)═, A² represents an oxygen atom, A³ represents ═C(R⁴)—, W represents —NR¹⁶R¹⁷, R⁷ and R⁸ may be the same or different and each represents a C1-C6 alkyl group optionally having one or more halogen atoms or a hydrogen atom, R¹⁶ represents a C1-C6 alkyl group optionally having one or more halogen atoms or a hydrogen atom, R¹⁷ represents a cyano group, a phenyl group optionally having one or more groups selected from the group Y, —OR¹⁸, or —NR⁷R⁸, and R¹⁸ represents a hydrogen atom.

When the compound of the present invention is used in the method of the present invention, only the compound of the present invention may be used. However, as described below, the compound can be used as a composition for promoting plant growth that is formulated using various inactive ingredients (solid carriers, liquid carriers, surfactants, other adjuvant for formulation, and the like).

Examples of the solid carriers used for formulation include fine powdery or granular materials and the like formed of minerals such as kaolin clay, attapulgite clay, bentonite, montmorillonite, Japanese acid clay, pyrophyllite, talc, diatomaceous earth, and calcite, natural organic substances such as corn rachis powder and walnut shell powder, synthetic organic substances such as urea, salts such as calcium carbonate and ammonium sulfate, or synthetic inorganic substances such as synthetic hydrous silicon oxide. Examples of the liquid carriers include aromatic hydrocarbons such as xylene, alkylbenzene, and methylnaphthalene, alcohols such as 2-propanol, ethylene glycol, propylene glycol, and ethylene glycol monoethyl ether, ketones such as acetone, cyclohexanone, and isophorone, plant oil such as soybean oil and cotton seed oil, petroleum-based aliphatic hydrocarbons, esters, dimethylsulfoxide, acetonitrile, water, and the like.

Examples of the surfactants include anionic surfactants such as an alkyl sulfuric acid ester salt, an alkyl aryl sulfonic acid salt, a dialkyl sulfosuccinic acid salt, a polyoxyethylene alkyl aryl ether phosphoric acid ester salt, lignin sulfonic acid salt, and a naphthalene sulfonate formaldehyde polycondensate, nonionic surfactants such as polyoxyethylene alkyl aryl ether, a polyoxyethylene alkyl polyoxypropylene block copolymer, and a sorbitan aliphatic ester, and cationic surfactants such as an alkyl trimethyl ammonium salt.

Examples of other adjuvants for formulation include water-soluble polymers such as polyvinyl alcohol and polyvinyl pyrrolidone, gum Arabic, alginic acid and a salt thereof, polysaccharides such as carboxymethylcellulose (CMC) and xanthan gum, inorganic substances such as aluminum magnesium silicate and alumina sol, preservatives, colorants, and stabilizing agents such as isopropyl acid phosphate (PAP) and BHT.

In the method of the present invention, when a plant is treated with the compound of the present invention, the plant or the plantation thereof is treated with the compound of the present invention at an effective dose. When the plant or the plantation of the plant is treated, it is treated once or plural times with the compound of the present invention.

Specific examples of the application method of the present invention include treating of the foliage, flower organs, or ear of a plant by means of spraying the compound to the foliage, soil (plantation) treatment that is performed before or after a plant is planted, seed treatment such as seed sterilization, seed soaking, or seed coating, seedling treatment, treating of a bulb such as a seed tuber, and the like.

In the method of the present invention, specific examples of the treating of the foliage, flower organs, or ears of a plant include a treatment method applying the compound onto the surface of a plant by means of spraying the compound to foliage, stem, and the like. The examples also include a method of performing spraying treatment on flower organs or the entire plant during the flowering period including a pre-flowering stage, a mid-flowering stage, and a post-flowering stage. Moreover, for grain and the like, the examples include a spraying method performed on the ears or the entire plant during the period of ear emergence.

Examples of the soil treatment method in the method of the present invention include spraying to soil, soil incorporation, and drenching soil with liquid chemical (liquid chemical irrigation, soil injection, or liquid chemical dripping). Examples of the place to be treated include planting holes, planting rows, the vicinity of planting holes, the vicinity of planting rows, the entire area of plantation, the vicinity of plantation, inter-row spaces, places under the stem, a ridge between main stems, culture soil, a seedling box, a seedling tray, a seedbed, and the like. The treatment is performed, for example, during a pre-seeding stage, a seeding stage, a stage immediately after seeding, and during the growing period including a seedling raising stage, a pre-planting stage, at a planting stage, and the post-planting stage. Further, in the soil treatment, a plant may be treated with plural kinds of compounds of the present invention at the same time, and a solid fertilizer such as a paste fertilizer containing the compound of the present invention may be applied to the soil. Moreover, the compound of the present invention may be mixed into a liquid for irrigation by means of, for example, by being injected into irrigation facilities (an irrigation tube, an irrigation pipe, a sprinkler, and the like), mixed into a liquid for inter-row space irrigation, or mixed into a hydroponic medium. In addition, the compound of the present invention can be mixed with the liquid for irrigation in advance to perform the treatment by means of, for example, the above irrigation method or other appropriate irrigation methods such as spraying of water and flooding.

In the present invention, the plant seed treated with the compound of the present invention retains the compound of the present invention at an effective dose, in the inside or surface of the plant seed or in the coated portion formed in the circumference of the plant seed. In the method of the present invention, the treating of seeds is a method of treating seeds or bulbs of a plant as a target with the compound of the present invention. Specific examples thereof include spraying treatment in which a suspension of the compound of the present invention is sprayed onto the seed surface or the bulb surface in the form of mist, smearing treatment in which wettable powder, an emulsion, or a flowable agent of the compound of the present invention is used as is or used by being supplemented with a small amount of water so as to coat the seed or bulb, a soaking treatment in which the seeds are soaked into the solution of the present compound for a certain time, film coating treatment, pellet coating treatment, and the like. Moreover, in the present invention, the plant seeds treated with the compound of the present invention refer to seeds of a plant that have not yet been seeded to soil or a culture medium for cultivation.

In the method of the present invention, examples of the seedling treatment include spraying treatment in which the compound of the present invention is prepared by being diluted with water to yield an appropriate concentration of active ingredients, and the diluted solution is sprayed to the entire seedling, soaking treatment in which the seedling is soaked into the diluted solution, coating treatment in which the compound of the present invention that is prepared as a dust formulation is applied to the entire seedling, and seedling-growing box treatment in which the culture soil that is being used to raise seedling is treated with the compound of the present invention at an effective dose. Moreover, examples of the soil treatment performed before or after seedlings are planted include a method in which a diluted solution, which is prepared by diluting the compound of the present invention with water to yield an appropriate concentration of active ingredients, is sprayed to the seedlings or the surrounding soil after the seedlings are planted, and a method in which the compound of the present invention that is prepared as granules or a solid formulation such as granules is sprayed to the surrounding soil after the seedlings are planted.

Further, the compound of the present invention may be used by being mixed with a hydroponic medium in hydroponic culture, or used as one of medium components in tissue culture. Regarding hydroponic treatment method in the method of the present invention, when the compound is used for hydroponic culture, the compound can be used by being dissolved or suspended in a hydroponic medium for hydroponic culture that is generally used for a horticultural experiment and the like, within a range of a concentration thereof in the medium of 0.001 ppm to 1,000 ppm. Moreover, when the compound is used for tissue culture or cell culture, the compound can be used by being dissolved or suspended in a generally used medium for plant tissue culture, such as Murashige & Scoog medium, or a hydroponic medium such as Hoagland hydroponic culture solution, within a range of a concentration thereof in the medium of 0.001 ppm to 1,000 ppm. In this case, saccharides as a carbon source, various plant hormones, and the like can be appropriately added according to the conventional method.

When the compound of the present invention is used to treat a plant or a place where the plant grows, the amount of the compound used for the treatment varies with the type of plant to be treated, the form of formulation, the time of treatment, weather conditions, and the like. However, the amount is generally within a range of 0.1 g to 10,000 g and preferably within a range of 1 g to 1,000 g, in terms of the amount of active ingredients per 10,000 m². When the compound is mixed with the entire soil, the amount of the compound used for the treatment is generally 0.1 g to 10,000 g and preferably 1 g to 1,000 g, in terms of the amount of active ingredients per 10,000 m².

The emulsion, wettable powder, flowable agent, microcapsules, and the like are used for the treatment generally by being diluted with water and sprayed. In this case, the concentration of active ingredients is generally within a range of 0.1 ppm to 10,000 ppm, and preferably within a range of 1 ppm to 1,000 ppm. Powder, granules, and the like are generally used as they are without being diluted.

During the seed treatment, a weight of the compound of the present invention per 100 Kg of seeds is generally within a range of 0.01 g to 1,000 g and preferably within a range of 0.1 g to 100 g.

Examples of plants to which the method of the present invention is applicable include the following.

Crops: corn, rice, wheat, barley, rye, oat, sorghum, cotton, soybean, peanut, buckwheat, sugar beet, colza, sunflower, sugar cane, tobacco, hop, and the like

Vegetables: vegetables from Solanaceae family (eggplant, tomato, potato, pepper, bell pepper, and the like), vegetables from Cucurbitaceous family (cucumber, squash, zucchini, watermelon, melon, oriental melon, and the like), vegetables from Cruciferous family (radish, turnip, horseradish, kohlrabi, napa cabbage, cabbage, rape, mustard, broccoli, cauliflower, and the like), vegetables from Compositae family (burdock, edible chrysanthemum, artichoke, lettuce, and the like), vegetables from Liliceae family (green onion, onion, garlic, asparagus, and the like), vegetables from Apiaceae family (carrot, parsley, celery, parsnip, and the like), vegetables from Chenopodiaceae family (spinach, chard, and the like), vegetables from Lamiaceae family (Japanese basil, mint, basil, and the like), crops from Laguminosae family (pea, common bean, azuki bean, broad bean, chickpea, and the like), strawberry, sweet potato, Japanese yam, taro, konjac, ginger, okra, and the like

Fruit trees: pomaceous fruits (apple, pear, European pear, Chinese quince, quince, and the like), stone fruits (peach, plum, nectarine, Japanese apricot, cherry, apricot, prune, and the like), citrus (Citrus unshiu, orange, lemon, lime, grapefruit, and the like), nuts (chestnut, walnut, hazelnut, almond, pistachio, cashew nut, macadamia nut, and the like), berries (blueberry, cranberry, blackberry, raspberry, and the like), grape, persimmon, olive, loquat, banana, coffee, date, coconut, oil palm, and the like

Trees other than fruit trees: tea, a mulberry tree, flowering trees (chive, camellia, hydrangea, sasanqua, Japanese star anise, cherry, tulip tree, crape myrtle, fragrant olive, and the like), roadside trees (ash, birch, dogwood, eucalyptus, gingko, lilac, maple, oak, poplar, cercis, liquidambar, plane, Japanese zelkova, Japanese arborvitae, fir, southern Japanese hemlock, juniper, pine, spruce, yew, elm, buckeye, and the like), sweet viburnum, yew plum pine, Japanese cedar, Japanese cypress, croton, Japanese spindle tree, Japanese photinia, and the like

Lawn: grasses (zoysia grass, Zoysia Matrella, and the like), bermuda grasses (Cynodon Dactylon, and the like), bentgrasses (wood medowgrass, creeping bentgrass, colonial bent, and the like), bluegrasses (Kentucky bluegrass, Poa compressa, and the like), fescues (fescue grass, chewing fescue, creeping red fescue, and the like), ryegrasses (Lolium multiporum Lam, Lolium perenne, and the like), orchardgrass, timothy grass, and the like

Others: flowers and ornamental plants (rose, carnation, chrysanthemum, Russell prairie gentian, gypsophila, gerbera, marigold, salvia, petunia, verbena, tulip, aster, gentian, lily, pansy, cyclamen, orchid, lily of valley, lavender, stock, ornamental cabbage, primula, poinsettia, gladiolus, cattleya, daisy, cymbidium, begonia, and the like), biofuel plants (jatropha, safflower, camelinas, switchgrass, miscanthus, reed canarygrass, Arundo donax, Ambry hemp, cassava, withy, and the like), foliage plants, and the like

Examples of plants applicable to the present invention preferably include tea, apple, pear, grape, cherry, peach, nectarine, persimmon, Japanese apricot, plum, soybean, lettuce, cabbage, tomato, eggplant, cucumber, watermelon, melon, common bean, peas, azuki bean, grasses, colza, strawberry, almond, corn, sorghum, broad beans, napa cabbage, potato, peanut, rice, wheat, taro, konjac, Japanese yam, radish, turnip, parsley, oriental melon, okra, ginger, lemon, orange, grapefruit, lime, blueberry, chestnut, hop, basil, more preferably include plants from the Poaceae family or plants from the Solanaceae family, even more preferably include plants from the Poaceae family, and still more preferably include rice, wheat, corn, and the like.

The above “plant” may be a plant into which a gene which imparts herbicide resistance to a plant, a gene which selectively produces toxicity for harmful insects, a gene which imparts disease resistance to a plant, a gene which relieves abiotic stress, and the like have been introduced by gene recombination or cross-breeding, or may be a stacked GM plant composed of plural kinds of combinations of these.

The compound of the present invention may be used simultaneously with an insecticide, a fungicide and a safener for a certain herbicide to treat seeds, or may be applied to the plant simultaneously with the above agents.

In the method of present invention, the plant to be treated with the compound of the present invention may be a plant that has been or will be exposed to abiotic stress. The degree of the abiotic stress that is indicated by the value of “stress intensity” described in the following formula may be 105 to 200, preferably 110 to 180, and more preferably 120 to 160.

Formula (1): “stress intensity”=100×“one of the plant phenotypes in a plant that has not yet been exposed to abiotic stress conditions”/“one of the plant phenotypes in the above plant that has been exposed to abiotic stress conditions”

The term “abiotic stress” as used herein means the stress that causes a decline in physiological functions of cells of a plant, when the plant is exposed to an abiotic stress condition, and then deterioration in the physiological state of the plant, leading to its growth inhibition, such as temperature stress, i.e. high-temperature stress or low-temperature stress, water stress, i.e. drought stress or excess water stress, and salt stress. The high-temperature stress refers to the stress that a plant suffers from when the plant is exposed to a temperature higher than the temperature appropriate for the growth or germination of the plant. Specifically, for example, this type of stress can be caused when an average cultivation temperature of the environment in which a plant is cultivated is under condition of 25° C. or higher, more strictly 30° C. or higher, and even more strictly 35° C. or higher. The low-temperature stress refers to the stress that a plant suffers from when the plant is exposed to a temperature lower than the temperature appropriate for the growth or germination of the plant. Specifically, for example, this type of stress can be caused when an average cultivation temperature of the environment in which a plant is cultivated is under condition of 15° C. or lower, more strictly 10° C. or lower, and even more strictly 5° C. or lower. Moreover, the drought stress refers to the stress that a plant suffers from when the water content in soil is reduced by the decrease in precipitation or watering amount, water absorption of the plant is hindered, and the plant is exposed to a water environment that may hinder the growth of the plant. Specifically, for example, this type of stress can be caused when a moisture content of soil in which the plant is cultivated is under the condition of 15% by weight or less, more strictly 10% by weight or less, and even more strictly 7.5% by weight or less which may causes water stress, or a pF value of soil in which the plant is cultivated is under the condition of 2.3 or higher, strictly 2.7 or higher, and even more strictly 3.0 or higher, though these values may vary with the type of soil. The excess water stress refers to the stress that a plant suffers from when the plant is exposed to a moistful environment in which a water content in the soil becomes excessive, and the growth of the plant may be hindered. Specifically, for example, this type of stress can be caused when a moisture content of the soil in which the plant is cultivated is under the condition of 30% by weight or more, more strictly 40% by weight or more, and even more strictly 50% by weight or more, or a pF value of the soil in which the plant is cultivated is under the condition of 1.7 or less, strictly 1.0 or less, and even more strictly 0.3 or less, though these values may vary with the type of soil. Further, the pF value of soil can be measured according to the principle described in “Dictionary of Soil-Plant Nutrition-Environment” (TAIYOSHA, CO., LTD., 1994, Matsusaka et al.), pp 61-62, “pF value measurement method”. In addition, the salt stress refers to the stress that a plant suffers from when salts accumulate in the soil or hydroponic medium in which a plant is cultivated, the osmotic pressure increases, water absorption of the plant is hindered, and accordingly, the plant is exposed to the environment which may hinder the growth of the plant. Specifically, for example, this type of stress can be caused when an osmotic potential resulting from a salt in the soil or hydroponic medium is under the condition of 0.2 MPa (2,400 ppm or more in terms of a NaCl concentration) or higher, strictly 0.25 MPa or higher, and even more strictly 0.30 MPa or higher. The osmotic pressure in soil can be determined based on the following Raoult's equation, by diluting the soil with water and analyzing a salt concentration of the supernatant liquid.

Raoult's equation

π(atm)=cRT

R=0.082 (L·atm/mol·K)

T=absolute temperature (K)

c=molar concentration of ion (mol/L)

1 atm=0.1 MPa

Next, production processes of the compounds of the present invention will be described.

The compound of the present invention can be produced according to, for example, the following (Production process 1) to (Production process 11).

(Production Process 1)

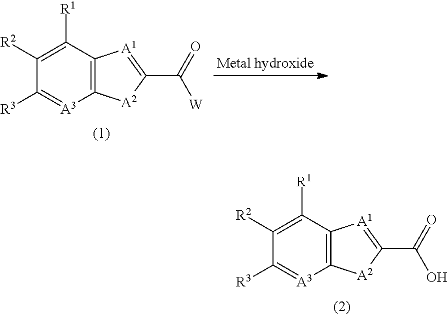

[In the formula, W, A¹, A², A³, R¹, R², and R³ have the same definition as described above.]

This reaction is generally performed in a solvent. Examples of the solvent used in this reaction include water; ethers such as tetrahydrofuran (hereinafter, described as THF) and 1,4-dioxane; alcohols such as methanol and ethanol; and a mixture of these.

Examples of the metal hydroxide used in this reaction include hydroxides of alkaline metals, such as lithium hydroxide, sodium hydroxide, and potassium hydroxide. In this reaction, the metal hydroxide is used in an amount of 2 mol to 20 mol in general, and preferably in an amount of 2 mol to 4 mol, based on 1 mol of the compound represented by Formula (1).

The reaction temperature of this reaction is generally within a range of room temperature to a boiling point of the solvent, and preferably is a boiling point of the solvent. This reaction can also be performed in a sealed tube or a pressure-resistant sealed container. The reaction time of this reaction is generally about 5 minutes to 36 hours.

The progress of this reaction can be confirmed by analyzing a portion of the reaction mixture by thin-layer chromatography, high-performance liquid chromatography, and the like. After this reaction is completed, for example, an operation in which an acid and water are added to the reaction mixture, extraction is performed using an organic solvent, and the obtained organic layer is dried and concentrated is performed, whereby the compound represented by Formula (2) can be isolated.

(Production Process 2)

Among the compounds of the present invention, the compound represented by Formula (2) can be produced according to, for example, the following scheme.

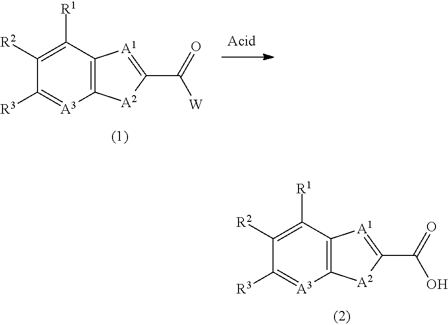

[In the formula, W, A¹, A², A³, R¹, R², and R³ have the same definition as described above.]

This reaction is generally performed in a solvent. Examples of the solvent used in this reaction include water; organic carboxylic acids such as acetic acid and propionic acid; and a mixture of these.

Examples of the acid used in this reaction include hydrochloric acid, hydrobromic acid, trifluoromethanesulfonic acid, sulfuric acid, and the like.

In this reaction, the acid is used in an amount of 1 mol to 100 mol in general, and preferably in an amount of 2 mol to 20 mol, based on 1 mol of the compound represented by Formula (1).

The reaction temperature of this reaction is generally within a range of room temperature to a boiling point of the used solvent, and preferably is from 80° C. to a boiling point of the solvent. This reaction can also be performed in a sealed tube or a pressure-resistant sealed container. The reaction time of this reaction is generally about 5 minutes to several days.

The progress of this reaction can be confirmed by analyzing a portion of the reaction mixture by thin-layer chromatography, high-performance liquid chromatography, and the like. After this reaction is completed, for example, an operation in which water is added to the reaction mixture, extraction is performed using an organic solvent, and the obtained organic layer is dried and concentrated is performed, whereby the compound represented by Formula (2) can be isolated.

(Production Process 3)

Among the compounds of the present invention, the compound in which W is —OR^{6a}, A¹ is —C(R^{5a})═, and A² is an oxygen atom can be produced according to, for example, the following scheme.

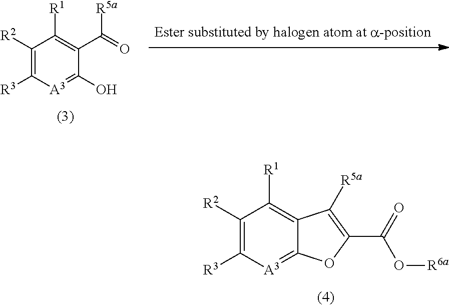

[In the formula, R^{5a} represents a hydrogen atom, a C1-C6 alkyl group optionally having one or more groups selected from the group X, or a phenyl group optionally having one or more groups selected from the group Y,

R^{6a} represents a C1-C6 alkyl group, and

A³, R¹, R², and R³ have the same definition as described above.]

This reaction is generally performed in a solvent. Examples of usable solvents include aromatic hydrocarbons such as benzene, toluene, and xylene; ethers such as diethylether, diisopropylether, 1,4-dioxane, THF, and 1,2-dimethoxyethane; halogenated hydrocarbons such as dichloromethane, chloroform, and 1,2-dichloroethane; ketones such as acetone and 2-butanone; amides such as N,N-dimethylformamide (hereinafter, described as DMF) and N,N-dimethylacetamide; sulfones such as sulfolane; and a mixture of these.

Examples of the ester substituted by halogen atom at α-position used in this reaction include methyl chloroacetate, ethyl chloroacetate, methyl bromoacetate, ethyl bromoacetate, chloromalonic acid dimethyl ester, chloromalonic acid diethyl ester, bromomalonic acid dimethyl ester, bromomalonic acid diethyl ester, and the like.

In this reaction, the ester substituted by halogen atom at α-position is used in an amount of 1 mol or more in general, and preferably in an amount of 2 mol to 50 mol, based on 1 mol of the compound represented by Formula (3).

This reaction is generally performed in the presence of a base. Examples of the base used in this reaction include metal carbonates such as sodium carbonate and potassium carbonate; metal alkoxides such as sodium methoxide; alkaline metal hydrides such as sodium hydride; organic bases such as triethylamine, tributylamine, and N,N-diisopropylethylamine; and the like. In this reaction, the base is used in an amount of 1 mol or more in general, and preferably in an amount of 2 mol to 50 mol, based on 1 mol of the compound represented by Formula (3).

The reaction temperature of this reaction is generally 0° C. to 200° C. and preferably 30° C. to 100° C. The reaction time of this reaction is generally 30 minutes to 30 hours.

The completion of this reaction can be confirmed by sampling a portion of the reaction mixture and using analysis means such as thin-layer chromatography and high-performance liquid chromatography. After this reaction is completed, for example, a post-treatment operation in which water is added to the reaction mixture, extraction is performed using an organic solvent, and the obtained organic layer is dried and concentrated is performed, whereby the compound represented by Formula (4) can be isolated. The isolated compound that is represented by Formula (4) can also be purified by chromatography, recrystallization, and the like.

(Production Process 4)

Among the compounds of the present invention, the compound in which W is a hydroxyl group, A¹ is —CH═, and A² is —NH— can be produced according to, for example, the following scheme.

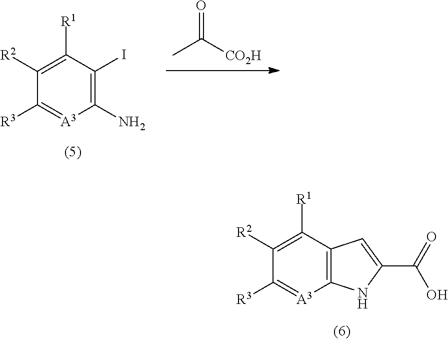

[In the formula, A³, R¹, R², and R³ have the same definition as described above.]

In this reaction, pyruvic acid is used in an amount of 1 mol or more in general, and preferably in an amount of 2 mol to 50 mol, based on 1 mol of the compound represented by Formula (5).

This reaction is performed in a solvent. Examples of the solvent used in this reaction include aromatic hydrocarbons such as benzene, toluene, and xylene; alcohols such as methanol, ethanol, and propanol; ethers such as diethylether, diisopropylether, 1,4-dioxane, THF, and 1,2-dimethoxyethane; ketones such as acetone and methyl ethyl ketone; nitriles such as acetonitrile; amides such as DMF and N,N-dimethylacetamide; sulfoxides such as dimethyl sulfoxide; sulfones such as sulfolane; and a mixture of these.

Moreover, this reaction is performed in the presence of a base. Examples of the base used in this reaction include organic bases such as triethylamine, tripropylamine, pyridine, dimethylaniline, dimethylaminopyridine, and 1,4-diazabicyclo[2.2.2]octane (hereinafter, described as DABCO), and 1,8-diazabicyclo[5.4.0]-7-undecene; and inorganic bases such as sodium hydroxide, potassium hydroxide, calcium hydroxide, sodium carbonate, potassium carbonate, sodium hydrogen carbonate, calcium carbonate, cesium carbonate, and potassium phosphate. In this reaction, the base is used in an amount of 1 mol or more in general, and preferably in an amount of 2 mol to 50 mol, based on 1 mol of the compound represented by Formula (5).

This reaction is generally performed in the presence of a catalyst such as palladium (II) acetate. This catalyst is used in an amount of 0.001 mol to 0.5 mol in general, and preferably in an amount of 0.01 mol to 0.2 mol, based on 1 mol of the compound represented by Formula (5).

In this reaction, a dehydrating agent such as magnesium sulfate is used if necessary. This dehydrating agent is used in an amount of 1 mol or more in general, and preferably in an amount of 2 mol to 50 mol, based on 1 mol of the compound represented by Formula (5).

The reaction temperature of this reaction is generally 20° C. to 180° C. and preferably 60° C. to 150° C. The reaction time of this reaction is generally 30 minutes to 100 hours. The progress of this reaction can be confirmed by analyzing a portion of the reaction mixture by thin-layer chromatography, high-performance liquid chromatography, and the like.

After this reaction is completed, for example, an operation in which the reaction mixture is mixed with water, extraction is performed using an organic solvent, and the obtained organic layer is dried and concentrated is performed, whereby the compound represented by Formula (6) can be isolated. The isolated compound that is represented by Formula (6) can also be purified by chromatography, recrystallization, and the like.

(Production Process 5)

Among the compounds of the present invention, the compound in which W is a hydroxyl group, A¹ is a nitrogen atom, and A² is —N(R¹²)— can be produced by reacting the compound represented by Formula (7) with a metal hydroxide.

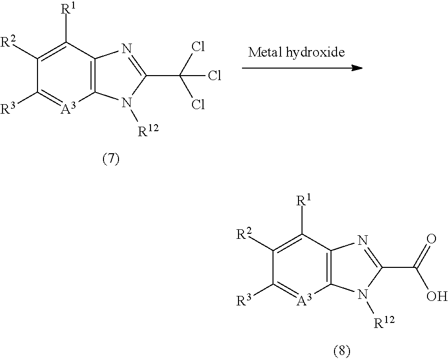

[In the formula, A³, R¹, R², R³, and R¹² have the same definition as described above.]

This reaction is generally performed in a solvent. Examples of the solvent used in this reaction include water; ethers such as THF and 1,4-dioxane; alcohols such as methanol and ethanol; and a mixture of these.

Examples of the metal hydroxide used in this reaction include hydroxides of alkaline metals, such as lithium hydroxide, sodium hydroxide, and potassium hydroxide. In this reaction, the metal hydroxide is used in an amount of 5 mol or more in general, and preferably in an amount of 5 mol to 100 mol, based on 1 mol of the compound represented by Formula (7).

The reaction temperature of this reaction is generally 0° C. to 200° C. and preferably 30° C. to 100° C. The progress of this reaction can be confirmed by analyzing a portion of the reaction mixture by thin-layer chromatography, high-performance liquid chromatography, and the like. After this reaction is completed, for example, an operation in which an acid and water are added to the reaction mixture, extraction is performed using an organic solvent, and the obtained organic layer is dried and concentrated is performed, whereby the compound represented by Formula (8) can be isolated.

(Production Process 6)

Among the compounds of the present invention, the compound in which W is —OMe, A¹ is a nitrogen atom, and A² is a sulfur atom can be produced according to, for example, the following scheme.

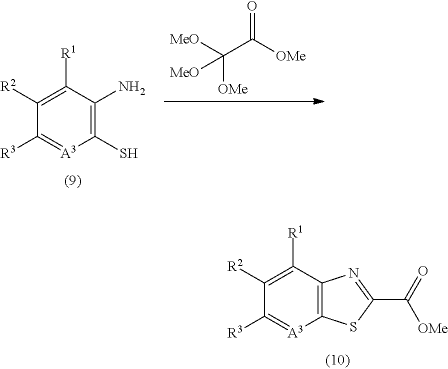

[In the formula, A³, R¹, R², and R³ have the same definition as described above.]

This reaction is performed in a solvent. Examples of the solvent used in this reaction include aromatic hydrocarbons such as benzene, toluene, and xylene; alcohols such as methanol, ethanol and propanol; ethers such as diethylether, diisopropylether, 1,4-dioxane, THF, and 1,2-dimethoxyethane; ketones such as acetone and methyl ethyl ketone; nitriles such as acetonitrile; amides such as DMF and N,N-dimethylacetamide; sulfoxides such as dimethyl sulfoxide; sulfones such as sulfolane; and a mixture of these.

In this reaction, methyl trimethoxyacetate is used in an amount of 1 mol or more in general, and preferably in an amount of 1 mol to 50 mol, based on 1 mol of the compound represented by Formula (9).

The reaction temperature of this reaction is generally 0° C. to 200° C. and preferably 50° C. to 150° C. The reaction time of this reaction is generally 30 minutes to 30 hours.

The completion of this reaction can be confirmed by sampling a portion of the reaction mixture and using analysis means such as thin-layer chromatography and high-performance liquid chromatography. After this reaction is completed, for example, a post-treatment operation in which water is added to the reaction mixture, extraction is performed using an organic solvent, and the obtained organic layer is dried and concentrated is performed, whereby the compound represented by Formula (10) can be isolated. The isolated compound that is represented by Formula (10) can also be purified by chromatography, recrystallization, and the like.

(Production Process 7)

Among the compounds of the present invention, the compound in which W is —OMe, A¹ is a nitrogen atom, and A² is an oxygen atom can be produced according to, for example, the following scheme.

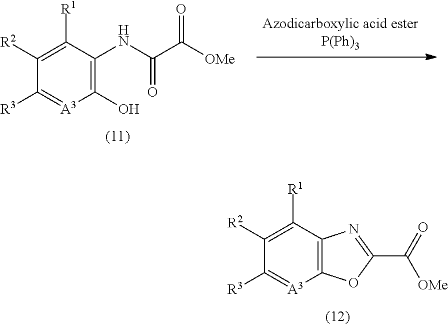

[In the formula, A³, R¹, R², and R³ have the same definition as described above.]

This reaction is performed in a solvent. Examples of the solvent used in this reaction include aromatic hydrocarbons such as benzene, toluene, and xylene; ethers such as diethylether, diisopropylether, 1,4-dioxane, THF, and 1,2-dimethoxyethane; halogenated hydrocarbons such as dichloromethane, chloroform, and 1,2-dichloroethane; and a mixture of these.

Examples of the azodicarboxylic acid ester used in this reaction include diethyl azodicarboxylate, azodicarboxylic acid bis(2-methoxyethyl)ester, and the like.

In this reaction, azodicarboxylic acid ester is used in an amount of 1 mol or more in general, and preferably in an amount of 1 mol to 10 mol, based on 1 mol of the compound represented by Formula (11).

In this reaction, triphenylphosphine is used in an amount of 1 mol or more in general, and preferably in an amount of 1 mol to 10 mol, based on 1 mol of the compound represented by Formula (11).

The reaction temperature of this reaction is generally 0° C. to 180° C. and preferably 20° C. to 100° C. The reaction time of this reaction is generally 30 minutes to 100 hours. The progress of this reaction can be confirmed by analyzing a portion of the reaction mixture by thin-layer chromatography, high-performance liquid chromatography, and the like. After this reaction is completed, for example, an operation in which the reaction mixture is mixed with water, extraction is performed using an organic solvent, and the obtained organic layer is dried and concentrated is performed, whereby the compound represented by Formula (12) can be isolated. The isolated compound that is represented by Formula (12) can also be purified by chromatography, recrystallization, and the like.

(Production Process 8)

Among the compounds of the present invention, the compound in which W represents —OH can be produced according to, for example, the following scheme.

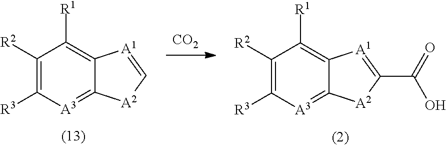

[In the formula, A¹, A², A³, R¹, R², and R³ have the same definition as described above.]

This reaction is performed in a solvent. Examples of usable solvents include aromatic hydrocarbons such as benzene, toluene, and xylene; ethers such as diethylether, diisopropylether, 1,4-dioxane, THF, and 1,2-dimethoxyethane; and a mixture of these.

This reaction is generally performed in the presence of a base. Examples of the base used in this reaction include n-butyllithium, lithium diisopropylamide, and the like. In this reaction, the base is used in an amount of 1 mol to 2 mol in general, and preferably in an amount of 1 mol to 1.1 mol, based on 1 mol of the compound represented by Formula (13).

In this reaction, carbon dioxide is used in an amount of 1 mol or more in general, and preferably in an amount of 10 mol to 1,000 mol, based on 1 mol of the compound represented by Formula (13).

The reaction temperature of this reaction is generally −100° C. to −30° C. The reaction time of this reaction is generally 5 minutes to 5 hours.

The completion of this reaction can be confirmed by sampling a portion of the reaction mixture and using analysis means such as thin-layer chromatography and high-performance liquid chromatography. After this reaction is completed, for example, water is added to the reaction mixture, the resultant is washed with an organic solvent, concentrated hydrochloric acid is added to the aqueous layer, extraction is then performed using an organic solvent, and the obtained organic layer is dried and concentrated, followed by chromatography, distillation, and the like. In this manner, the compound represented by Formula (2) can be isolated.

(Production Process 9)

The compound represented by Formula (14) can be produced according to, for example, the following scheme.

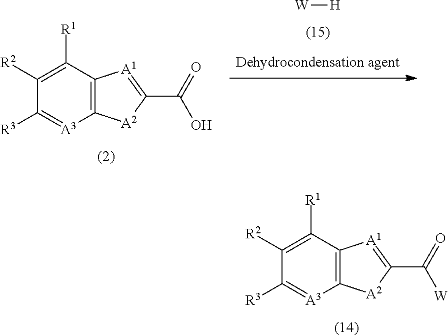

[In the formula, W, A¹, A², A³, R¹, R², and R³ have the same definition as described above.]

This reaction is generally performed in a solvent.

Examples of usable solvents include aromatic hydrocarbons such as benzene, toluene, and xylene; ethers such as diethylether, diisopropylether, 1,4-dioxane, THF, and 1,2-dimethoxyethane; halogenated hydrocarbons such as dichloromethane, chloroform, and 1,2-dichloroethane; amides such as DMF and N,N-dimethylacetamide; sulfones such as sulfolane; esters such as ethyl acetate and butyl acetate; and a mixture of these.

In this reaction, the compound represented by Formula (15) is used in an amount of 1 mol or more in general, and preferably in an amount of 1 mol to 10 mol, based on 1 mol of the compound represented by Formula (2).

This reaction is performed in the presence of a base if necessary. Examples of the base used in this reaction include metal carbonates such as sodium carbonate and potassium carbonate; metal alkoxides such as sodium methoxide; alkaline metal hydrides such as sodium hydride; organic bases such as triethylamine, tributylamine, and N,N-diisopropylethylamine; and the like. In this reaction, the base is used in an amount of 1 mol to 50 mol in general, and preferably in an amount of 1 mol to 10 mol, based on 1 mol of the compound represented by Formula (2).

Examples of dehydrocondensation agents used in this reaction include carbodiimide-based agents such as N,N-dicyclohexylcarbodiimide, and 1-ethyl-3-(3-dimethylaminopropyl)carbodiimide hydrochloride; phosphonium salt-based agents such as a benzotriazol-1-yloxytris(dimethylamino)phosphonium salt; and the like.

In this reaction, the dehydrocondensation agent is used in an amount of 1 mol to 50 mol in general, and preferably in an amount of 1 mol to 10 mol, based on 1 mol of the compound represented by Formula (2).

The reaction temperature of this reaction is generally 0° C. to 200° C. and preferably 30° C. to 100° C. The reaction time of this reaction is generally 30 minutes to 30 hours.

The completion of this reaction can be confirmed by sampling a portion of the reaction mixture and using analysis means such as thin-layer chromatography and high-performance liquid chromatography. After this reaction is completed, for example, a post-treatment operation in which water is added to the reaction mixture, extraction is performed using an organic solvent, and the obtained organic layer is dried and concentrated is performed, whereby the compound represented by Formula (14) can be isolated. The isolated compound that is represented by Formula (14) can also be purified by chromatography, recrystallization, and the like.

(Production Process 10)

The compound represented by Formula (17) can be produced according to, for example, the following scheme.

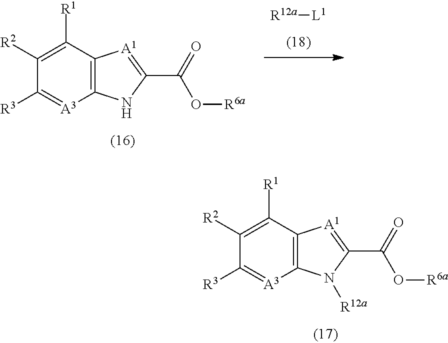

[In the formula, R^{6a} represents a C1-C6 alkyl group optionally having one or more groups selected from the group Z, a C3-C6 alkenyl group optionally having one or more groups selected from the group X, a C3-C6 alkynyl group optionally having one or more groups selected from the group X, a C4-C7 cycloalkylalkyl group optionally having one or more halogen atoms, a C3-C6 cycloalkyl group optionally having one or more halogen atoms, a phenyl group optionally having one or more groups selected from the group Y, or a C7-C9 phenylalkyl group in which a benzene ring portion may have optionally one or more groups selected from the group Y, R^{12a} represents a C1-C4 alkyl group optionally having one or more halogen atoms, a C1-C4 alkylsulfonyl group optionally having one or more halogen atoms, a phenylsulfonyl group optionally having one or more groups selected from the group Y, a C7-C9 phenylalkylsulfonyl group in which a benzene ring portion may have optionally one or more groups selected from the group Y, a C2-C6 alkoxycarbonyl group, —C(O)R¹⁴, or —C(O)NR⁷R⁸, L1 represents a leaving group such as a halogen atom, and A¹, A³, R¹, R², R³, R⁷, R⁸, and R¹⁴ have the same definition as described above.]

This reaction can be performed in a solvent. Examples of usable solvents include aromatic hydrocarbons such as benzene, and toluene; ethers such as diethylether, diisopropylether, 1,4-dioxane, THF, and 1,2-dimethoxyethane; halogenated hydrocarbons such as dichloromethane, chloroform, and 1,2-dichloroethane; amides such as DMF and N,N-dimethylacetamide; sulfoxides such as dimethyl sulfoxide; sulfones such as sulfolane; and a mixture of these.

In this reaction, the compound represented by Formula (18) is used in an amount of 1 mol or more in general, and preferably in an amount of 1 mol to 3 mol, based on 1 mol of the compound represented by Formula (16)

This reaction is generally performed in the presence of a base. Examples of the base used in this reaction include organic bases such as triethylamine, tripropylamine, pyridine, dimethylaminopyridine, diisopropyl ethylamine, and 1,8-diazabicyclo[5.4.0]-7-undecene; and inorganic bases such as sodium hydroxide, potassium hydroxide, calcium hydroxide, sodium carbonate, potassium carbonate, sodium hydrogen carbonate, calcium carbonate, and sodium hydride. In this reaction, the base is used in an amount of 0.5 mol to 10 mol in general, and preferably in an amount of 1 mol to 5 mol, based on 1 mol of the compound represented by Formula (16).

The reaction temperature of this reaction is generally −30° C. to 180° C. and preferably −10° C. to 50° C. The reaction time of this reaction is generally 10 minutes to 30 hours.

The completion of this reaction can be confirmed by sampling a portion of the reaction mixture and using analysis means such as thin-layer chromatography and high-performance liquid chromatography. After this reaction is completed, for example, an operation in which the reaction mixture is mixed with water, extraction is performed using an organic solvent, and the obtained organic layer is dried and concentrated is performed, whereby the compound represented by Formula (17) can be isolated. The isolated compound that is represented by Formula (17) can also be purified by chromatography, recrystallization, and the like.

(Production Process 11)

The compound represented by Formula (20) can be produced according to, for example, the following scheme.

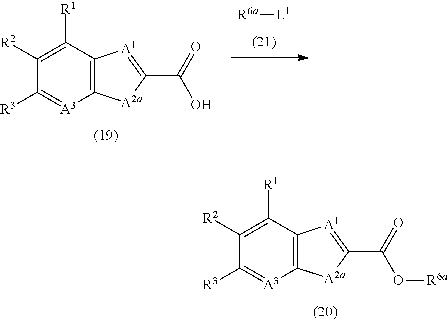

[In the formula, A^{2a} represents —N(R^{12a})—, an oxygen atom, or a sulfur atom when A¹ is a nitrogen atom, A^{2a} represents —N(R^{12a})— or an oxygen atom when A¹ is —C(R⁵)═, R^{12a} represents a C1-C6 alkyl group optionally having one or more groups selected from the group X, a C3-C6 alkenyl group optionally having one or more groups selected from the group X, a C3-C6 alkynyl group optionally having one or more groups selected from the group X, a C4-C7 cycloalkylalkyl group optionally having one or more halogen atoms, a C3-C6 cycloalkyl group optionally having one or more halogen atoms, a C7-C9 phenylalkyl group in which a benzene ring portion may have optionally one or more groups selected from the group Y, a phenyl group optionally having one or more groups selected from the group Y, a C2-C6 alkylcarbonyl group optionally having one or more halogen atoms, a benzoyl group optionally having one or more groups selected from the group Y, a C1-C6 alkylsulfonyl group optionally having one or more halogen atoms, or a phenylsulfonyl group optionally having one or more groups selected from the group Y, and L¹, A¹, A³, R¹, R², R³, and R^{6a} have the same definition as described above.]

This reaction is generally performed in a solvent. Examples of the solvent used in this reaction include aromatic hydrocarbons such as benzene, toluene, and xylene; ethers such as diethylether, diisopropylether, 1,4-dioxane, THF, and 1,2-dimethoxyethane; halogenated hydrocarbons such as dichloromethane, chloroform, and 1,2-dichloroethane; amides such as DMF and N,N-dimethylacetamide; sulfones such as sulfolane; esters such as ethyl acetate and butyl acetate; and a mixture of these.

Examples of the compound represented by Formula (21) used in this reaction include iodomethane, dimethyl sulfate, trimethylsilyl diazomethane, and the like. In this reaction, the compound represented by Formula (21) is used in an amount of 1 mol to 20 mol in general, and preferably in an amount of 1 mol to 10 mol, based on 1 mol of the compound represented by Formula (19).

The reaction temperature of this reaction is generally within an range of 0° C. to 200° C. and preferably 10° C. to 100° C. This reaction can also be performed in a sealed tube or a pressure-resistant sealed container. The reaction time of this reaction is generally about 5 minutes to 36 hours.

This reaction is generally performed in the presence of a base. Examples of the base used in this reaction include metal carbonates such as sodium carbonate and potassium carbonate; metal alkoxides such as sodium methoxide; alkaline metal hydrides such as sodium hydride; organic bases such as triethylamine, tributylamine, and N,N-diisopropylethylamine; and the like. In this reaction, the base is used in an amount of 1 mol to 50 mol in general, and preferably in an amount of 1 mol to 10 mol, based on 1 mol of the compound represented by Formula (19).

The progress of this reaction can be confirmed by analyzing a portion of the reaction mixture by thin-layer chromatography, high-performance liquid chromatography, and the like. After this reaction is completed, for example, a purification operation such as silica gel column chromatography is performed, whereby the compound represented by Formula (20) can be isolated.

(Reference Production Process 1)

The compound represented by Formula (23) can be produced according to, for example, the following scheme.

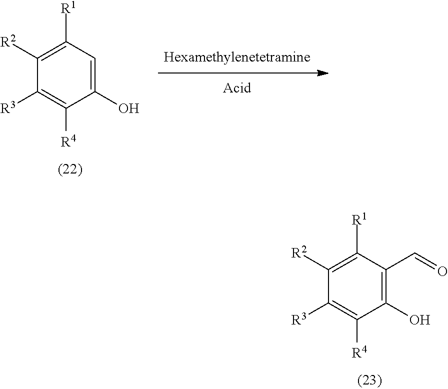

[In the formula, R¹, R², R³, and R⁴ have the same definition as described above.]

Examples of acids used in this reaction include organic acids such as trifluoroacetic acid and inorganic acids such as sulfuric acid. In this reaction, the acid is used in an amount of 1 mol or more in general, and preferably in an amount of 1 mol to 500 mol, based on 1 mol of the compound represented by Formula (22).

In this reaction, hexamethylenetetramine is used in an amount of 1 mol or more in general, and preferably in an amount of 1 mol to 50 mol, based on 1 mol of the compound represented by Formula (22).

This reaction is performed in a solvent if necessary. Examples of usable solvents include aromatic hydrocarbons such as benzene, toluene, and xylene; ethers such as diethylether, diisopropylether, 1,4-dioxane, THF, and 1,2-dimethoxyethane; halogenated hydrocarbons such as dichloromethane, chloroform, and 1,2-dichloroethane; and a mixture of these.

The reaction temperature of this reaction is generally 0° C. to 200° C. and preferably 20° C. to 100° C. The reaction time of this reaction is generally 5 hours to 100 hours.

The completion of this reaction can be confirmed by sampling a portion of the reaction mixture and using analysis means such as thin-layer chromatography and high-performance liquid chromatography. After this reaction is completed, for example, a post-treatment operation in which water is added to the reaction mixture, extraction is performed using an organic solvent, and the obtained organic layer is dried and concentrated is performed, whereby the compound represented by Formula (23) can be isolated. The isolated compound that is represented by Formula (23) can also be purified by chromatography, recrystallization, and the like.

(Reference Production Process 2)

The compound represented by Formula (25) can be produced according to, for example, the following scheme.

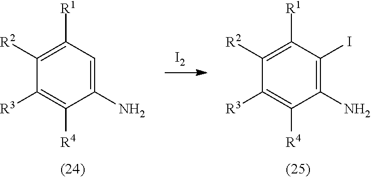

[In the formula, R¹, R², R³, and R⁴ have the same definition as described above.]

In this reaction, iodine is used in an amount of 1 mol or more in general, and preferably in an amount of 1 mol to 50 mol, based on 1 mol of the compound represented by Formula (24).

This reaction is performed in a solvent. Examples of the solvent used in this reaction include alcohols such as methanol, ethanol, and propanol; ethers such as diethylether, diisopropylether, 1,4-dioxane, THF, and 1,2-dimethoxyethane; ketones such as acetone and methyl ethyl ketone; nitriles such as acetonitrile; amides such as DMF and N,N-dimethylacetamide; sulfoxides such as dimethyl sulfoxide; sulfones such as sulfolane; and a mixture of these.

This reaction can be performed in the presence of a metal such as silver (I) sulfate if necessary. This metal is used in an amount of 0.1 mol to 10 mol in general, and preferably in an amount of 1 mol to 5 mol, based on 1 mol of the compound represented by Formula (24).

The reaction temperature of this reaction is generally 0° C. to 180° C. and preferably 20° C. to 120° C. The reaction time of this reaction is generally 30 minutes to 100 hours. The progress of this reaction can be confirmed by analyzing a portion of the reaction mixture by thin-layer chromatography, high-performance liquid chromatography, and the like. After this reaction is completed, for example, an operation such as chromatography or recrystallization is performed, whereby the compound represented by Formula (25) can be isolated.

(Reference Production Process 3)

The compound represented by Formula (27) can be produced according to, for example, the following scheme.

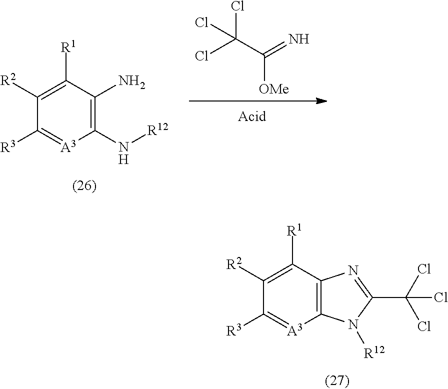

[In the formula, A³, R¹, R², R³, and R¹² have the same definition as described above.]

Examples of the acid used in this reaction include organic acids such as acetic acid and trifluoroacetic acid and inorganic acids such as hydrochloric acid. In this reaction, the acid is used in an amount of 1 mol or more in general, and preferably in an amount of 1 mol to 500 mol, based on 1 mol of the compound represented by Formula (26).

In this reaction, methyl-2,2,2-trichloroacetimidate is used in an amount of 1 mol or more in general, and preferably in an amount of 1 mol to 50 mol, based on 1 mol of the compound represented by Formula (26).

This reaction is performed in a solvent if necessary. Examples of usable solvents include aromatic hydrocarbons such as benzene, toluene, and xylene; ethers such as diethylether, diisopropylether, 1,4-dioxane, THF, and 1,2-dimethoxyethane; halogenated hydrocarbons such as dichloromethane, chloroform, and 1,2-dichloroethane; and a mixture of these.

The reaction temperature of this reaction is generally 0° C. to 200° C. and preferably 20° C. to 100° C. The reaction time of this reaction is generally 30 minutes to 30 hours.

The completion of this reaction can be confirmed by sampling a portion of the reaction mixture and using analysis means such as thin-layer chromatography and high-performance liquid chromatography. After this reaction is completed, for example, a post-treatment operation in which water is added to the reaction mixture, extraction is performed using an organic solvent, and the obtained organic layer is dried and concentrated is performed, whereby the compound represented by Formula (27) can be isolated. The isolated compound that is represented by Formula (27) can also be purified by chromatography, recrystallization, and the like.

(Reference Production Example 4)

The compound represented by Formula (11) can be produced according to, for example, the following scheme.

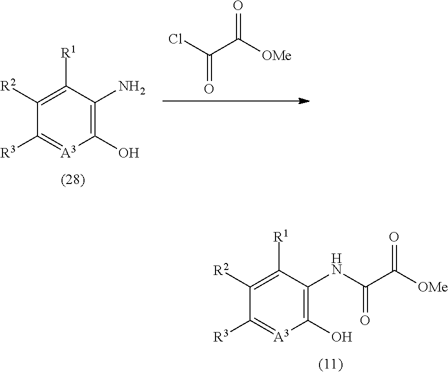

[In the formula, A³, R¹, R², and R³ have the same definition as described above.]

This reaction can be performed in a solvent. Examples of usable solvents include aromatic hydrocarbons such as benzene and toluene; ethers such as diethylether, diisopropylether, 1,4-dioxane, THF, and 1,2-dimethoxyethane; halogenated hydrocarbons such as dichloromethane, chloroform, and 1,2-dichloroethane; nitriles such as acetonitrile; amides such as DMF and N,N-dimethylacetamide; sulfoxides such as dimethyl sulfoxide; sulfones such as sulfolane; and a mixture of these.

In this reaction, methyl chloroglyoxylate is used in an amount of 1 mol or more in general, and preferably in an amount of 1 mol to 5 mol, based on 1 mol of the compound represented by Formula (28).

This reaction is generally performed in the presence of a base. Examples of the base used in this reaction include organic bases such as triethylamine, tripropylamine, pyridine, dimethylaminopyridine, and 1,8-diazabicyclo[5.4.0]-7-undecene; and inorganic bases such as sodium hydroxide, potassium hydroxide, calcium hydroxide, sodium carbonate, potassium carbonate, sodium hydrogen carbonate, calcium carbonate, and sodium hydride. In this reaction, the base is used in an amount of 1 mol or more in general, and preferably in an amount of 1 mol to 5 mol, based on 1 mol of the compound represented by Formula (28).

The reaction temperature of this reaction is generally 0° C. to 180° C. and preferably 10° C. to 50° C. The reaction time of this reaction is generally 10 minutes to 30 hours.

The completion of this reaction can be confirmed by sampling a portion of the reaction mixture and using analysis means such as thin-layer chromatography and high-performance liquid chromatography. After this reaction is completed, for example, an operation in which the reaction mixture is mixed with water, extraction is performed using an organic solvent, and the obtained organic layer is dried and concentrated is performed, whereby the compound represented by Formula (11) can be isolated. The isolated compound that is represented by Formula (11) can also be purified by chromatography, recrystallization, and the like.

(Reference Production Process 5)

The compound represented by Formula (30) can be produced according to, for example, the following scheme.

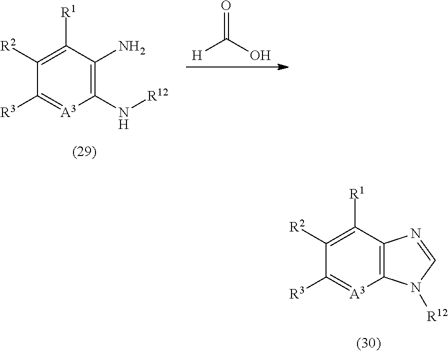

[In the formula, A³, R¹, R², R³, and R¹² have the same definition as described above.]

In this reaction, formic acid is used in an amount of 1 mol or more in general, and preferably in an amount of 1 mol to 100 mol, based on 1 mol of the compound represented by Formula (29).

This reaction is performed in a solvent if necessary. Examples of usable solvents include aromatic hydrocarbons such as benzene, toluene, and xylene; ethers such as diethylether, diisopropylether, 1,4-dioxane, THF, and 1,2-dimethoxyethane; halogenated hydrocarbons such as dichloromethane, chloroform, and 1,2-dichloroethane; and a mixture of these.

The reaction temperature of this reaction is generally 0° C. to 200° C. and preferably 30° C. to 150° C. The reaction time of this reaction is generally 30 minutes to 30 hours.

The completion of this reaction can be confirmed by sampling a portion of the reaction mixture and using analysis means such as thin-layer chromatography and high-performance liquid chromatography. After this reaction is completed, for example, a post-treatment operation in which water is added to the reaction mixture, extraction is performed using an organic solvent, and the obtained organic layer is dried and concentrated is performed, whereby the compound represented by Formula (30) can be isolated. The isolated compound that is represented by Formula (30) can also be purified by chromatography, recrystallization, and the like.

### EXAMPLES

Hereinafter, the production examples, formulation examples, and test examples of the present invention will be described in more detail, but the present invention is not limited to the following examples. Moreover, in the following examples, “part(s)” indicates “part(s) by weight” unless otherwise specified.

### Preparation Example 1

A mixture of 2.2 g of ethyl 5-(trifluoromethyl)benzofuran-2-carboxylate, 0.54 g of lithium hydroxide monohydrate, 20 ml of water, and 30 ml of 1,4-dioxane was stirred for 16 hours at room temperature. An aqueous sodium hydroxide solution was added to the reaction mixture, and the resultant was washed once with diethyl ether. Concentrated hydrochloric acid was added to the aqueous layer, and extraction was performed three times on the resultant by using ethyl acetate. The collected organic layer was washed with saturated saline, dried over magnesium sulfate, and then concentrated under reduced pressure, thereby obtaining 1.9 g of 5-trifluoromethylbenzofuran-2-carboxylic acid (hereinafter, described as a “compound 2 of the present invention”).

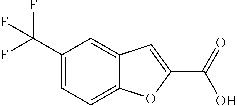 [SMILES: O=C(O)c1cc2cc(C(F)(F)F)ccc2o1]

### Compound 2 of the Present Invention

¹H-NMR (DMSO-D₆) δ: 8.26-8.24 (m, 1H), 7.95 (d, 1H, J=8.8 Hz), 7.86-7.82 (m, 1H), 7.78-7.77 (m, 1H)

### Production Example 2

A mixture of 0.40 g of 5-trifluoromethylbenzofuran-2-carboxylic acid, 0.26 g of potassium carbonate, 0.28 g of methyl iodide, and 6 ml of DMF was stirred for 1 hour at room temperature. 3 M of hydrochloric acid was added to the reaction mixture, and the precipitated solids were collected by filtration and washed with water. The collected solids were dissolved in ethyl acetate, and the resultant was washed with water and saturated saline, dried over magnesium sulfate, and then concentrated under reduced pressure. The residues were subjected to silica gel column chromatography, thereby obtaining 0.29 g of methyl 5-(trifluoromethyl)benzofuran-2-carboxylate (hereinafter, described as a “compound 3 of the present invention”).

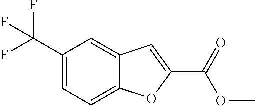 [SMILES: COC(=O)c1cc2cc(C(F)(F)F)ccc2o1]

### Compound 3 of the Present Invention

¹H-NMR (CDCl₃) δ: 8.02-8.01 (m, 1H), 7.71-7.70 (m, 2H), 7.60-7.59 (m, 1H), 4.01 (s, 3H)

### Production Example 3

### Step 1

4.50 g of hexamethylenetetramine was added little by little to a mixture of 4.61 g of 4-trifluoromethylphenol and 65 ml of trifluoroacetic acid at room temperature. This mixture was heated under reflux for 24 hours. After the reaction mixture was cooled to room temperature, 35 ml of water and 20 ml of 50% sulfuric acid were added thereto, followed by stirring for 4 hours at room temperature. Extraction was performed twice on the reaction mixture by using diethyl ether. The collected organic layer was washed with 5 M hydrochloric acid and water. The organic layer was dried over magnesium sulfate and concentrated under reduced pressure. The residues were subjected to silica gel column chromatography, thereby obtaining 2.2 g of 2-hydroxy-5-trifluoromethylbenzaldehyde.

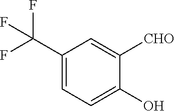 [SMILES: O=Cc1cc(C(F)(F)F)ccc1O]

¹H-NMR (CDCl₃) δ: 11.31 (s, 1H), 9.95 (s, 1H), 7.88-7.86 (m, 1H), 7.78-7.74 (m, 1H), 7.11 (d, 1H, J=8.8 Hz)

### Step 2

A mixture of 1.2 g of 2-hydroxy-5-trifluoromethylbenzaldehyde, 7.0 g of bromomalonic acid diethyl ester, 4.1 g of potassium carbonate, and 50 ml of 2-butanone was heated under reflux for 12 hours. After the reaction mixture was cooled to room temperature, the precipitate was removed by filtration, and the filtrate was concentrated under reduced pressure. The residues were subjected to silica gel column chromatography, thereby obtaining 2.4 g of ethyl 5-trifluorometethylbenzofuran-2-carboxylate (hereinafter, described as a “compound 4 of the present invention”)

### Compound 4 of the Present Invention

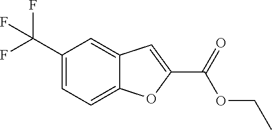 [SMILES: CCOC(=O)c1cc2cc(C(F)(F)F)ccc2o1]

¹H-NMR (CDCl₃) δ: 8.01-7.99 (m, 1H), 7.71-7.69 (m, 2H), 7.59 (s, 1H), 4.47 (q, 2H, J=7.2 Hz), 1.45 (t, 3H, J=7.2 Hz)

### Production Example 4

A mixture of 0.23 g of 5-trifluoromethylbenzofuran-2-carboxylic acid, 0.15 g of 2,4-dimethoxyaniline, 0.57 g of a (benzotriazol-1-yloxy)-tris(dimethylamino)phosphonium salt, 0.13 g of triethylamine, and 5 ml of DMF was stirred for 5 hours at room temperature. Water was added to the reaction mixture, and extraction was performed twice by using ethyl acetate. The collected organic layer was washed with water and saturated saline, dried over magnesium sulfate, and then concentrated under reduced pressure. The residues were subjected to silica gel column chromatography, thereby obtaining 0.28 g of N-(2,4-dimethoxyphenyl)-5-trifluoromethylbenzofuran-2-carboxamide (hereinafter, described as a “compound 5 of the present invention”).

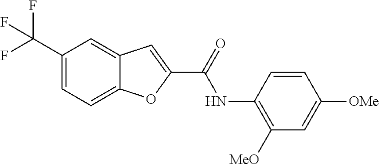 [SMILES: COc1ccc(NC(=O)c2cc3cc(C(F)(F)F)ccc3o2)c(OC)c1]

### Compound 5 of the Present Invention

¹H-NMR (CDCl₃) δ: 8.82 (br s, 1H), 8.43-8.40 (m, 1H), 8.01 (s, 1H), 7.71-7.69 (m, 2H), 7.61 (s, 1H), 6.56-6.52 (m, 2H), 3.97 (s, 3H), 3.83 (s, 3H)

### Production Example 5

### Step 1

A mixture of 2.5 g of 4-trifluoromethylaniline, 3.9 g of iodine, 4.8 g of silver (I) sulfate, and 300 ml of ethanol was stirred for 1 hour at room temperature. The reaction mixture was filtered through celite, and the filtrate was concentrated under reduced pressure. The residues were subjected to silica gel column chromatography, thereby obtaining 2.3 g of 2-iodo-4-trifluoromethylaniline.

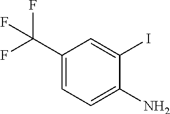 [SMILES: Nc1ccc(C(F)(F)F)cc1I]

¹H-NMR (CDCl₃) δ: 7.88-7.85 (m, 1H), 7.40-7.35 (m, 1H), 6.74 (d, 1H, J=8.3 Hz), 4.42 (br s, 2H)

### Step 2

A mixture of 2.28 g of 2-iodo-4-trifluoromethylaniline, 2.10 g of pyruvic acid, 2.67 g of DABCO, 89 mg of palladium (II) acetate, and 25 ml of DMF was stirred for 24 hours at 100° C. in nitrogen atmosphere. After the reaction mixture was cooled to room temperature, 25 ml of ethyl acetate and 50 ml of 5 M hydrochloric acid were added thereto, and extraction was performed three times by using ethyl acetate. The collected organic layer was washed with water and saturated saline, dried over magnesium sulfate, and then concentrated under reduced pressure. The residues were washed with chloroform, thereby obtaining 1.15 g of 5-trifluoromethylindole-2-carboxylic acid (hereinafter, described as a “compound 7 of the present invention”).

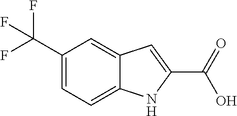 [SMILES: O=C(O)c1cc2cc(C(F)(F)F)ccc2[nH]1]

### Compound 7 of the Present Invention

¹H-NMR (DMSO-D₆) δ: 13.24 (br s, 1H), 12.20 (br s, 1H), 8.08 (s, 1H), 7.60 (d, 1H, J=8.8 Hz), 7.51 (dd, 1H, J=8.8, 1.6 Hz), 7.24 (d, 1H, J=1.6 Hz)

### Production Example 6

A mixture of 0.35 g of 5-trifluoromethylindole-2-carboxylic acid, 0.15 g of concentrated sulfuric acid, and 3 ml of methanol was heated under reflux for 6 hours. After the reaction mixture was cooled to room temperature, water was added thereto, and the precipitated solids were collected by filtration. The obtained solids were dissolved in ethyl acetate, and the organic layer was washed with water and saturated saline, dried over magnesium sulfate, and then concentrated under reduced pressure. The residues were subjected to silica gel column chromatography, thereby obtaining 0.36 g of methyl 5-trifluoromethylindole-2-carboxylate (hereinafter, described as a “compound 8 of the present invention”).

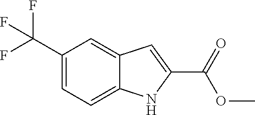 [SMILES: COC(=O)c1cc2cc(C(F)(F)F)ccc2[nH]1]

### Compound 8 of the Present Invention

¹H-NMR (CDCl₃) δ: 9.09 (br s, 1H), 8.02-8.00 (m, 1H), 7.57-7.50 (m, 2H), 7.31-7.29 (m, 1H), 3.98 (s, 3H)

### Production Example 7

A mixture of 0.25 g of 5-trifluoromethylindole-2-carboxylic acid, 0.17 g of 2,4-dimethoxyaniline, 0.63 g of a benzotriazol-1-yloxy-trisdimethylaminophosphonium salt, 0.15 g of triethylamine, and 4 ml of DMF was stirred for 6 hours at room temperature. Water was added to the reaction mixture, and the precipitated solids were collected by filtration. The obtained solids were dissolved in ethyl acetate, and the organic layer was washed with water and saturated saline, dried over magnesium sulfate, and then concentrated under reduced pressure. The residues were subjected to silica gel column chromatography, thereby obtaining 0.18 g of N-(2,4-dimethoxyphenyl)-5-trifluoromethylindole-2-carboxamide (hereinafter, described as a “compound 10 of the present invention”).

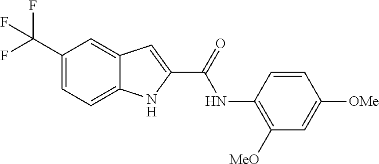 [SMILES: COc1ccc(NC(=O)c2cc3cc(C(F)(F)F)ccc3[nH]2)c(OC)c1]

### Compound 10 of the Present Invention

¹H-NMR (CDCl₃) δ: 9.63 (br s, 1H), 8.42-8.34 (m, 2H), 8.00 (s, 1H), 7.57-7.50 (m, 2H), 7.08-7.06 (m, 1H), 6.57-6.53 (m, 2H), 3.95 (s, 3H), 3.84 (s, 3H)

### Production Example 8

### Step 1

A mixture of 2.25 g of methyl chloroglyoxylate and 5 ml of DMF was added dropwise to a mixture of 2.0 g of 2-aminophenol, 2.23 g of triethylamine, and 20 ml of DMF under ice cooling, followed by stirring for 10 minutes. Water and 1 M hydrochloric acid were added thereto, and the precipitated solids were collected by filtration. The obtained solids were washed with water and dried under reduced pressure, thereby obtaining 1.2 g of methyl 2-[(2-hydroxyphenyl)amino]-2-oxo-acetate.

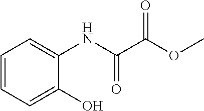

¹H-NMR (DMSO-D₆) δ: 10.24 (s, 1H), 9.62 (br s, 1H), 7.95-7.92 (m, 1H), 7.05-7.00 (m, 1H), 6.94-6.90 (m, 1H), 6.86-6.80 (m, 1H), 3.85 (s, 3H)

### Step 2

A mixture of 2.16 g of azodicarboxylic acid bis(2-methoxyethyl)ester and 10 ml of THF was added dropwise to a mixture of 1.2 g of methyl 2-[(2-hydroxyphenyl)amino]-2-oxo-acetate, 2.42 g of triphenylphosphine, and 25 ml of THF under ice cooling, followed by stirring for 3 hours at room temperature. The reaction mixture was concentrated under reduced pressure, and the residues were subjected to silica gel column chromatography, thereby obtaining 0.72 g of methyl benzoxazole-2-carboxylate (hereinafter, described as a “compound 16 of the present invention”).

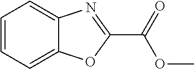

### Compound 16 of the Present Invention

¹H-NMR (CDCl₃) δ: 7.92-7.89 (m, 1H), 7.70-7.66 (m, 1H), 7.57-7.52 (m, 1H), 7.50-7.45 (m, 1H), 4.10 (s, 3H)

### Production Example 9

### Step 1

A mixture of 0.97 g of methyl chloroglyoxylate and 2 ml of DMF was added dropwise to a mixture of 1.4 g of 2-amino-4-trifluoromethylphenol, 0.96 g of triethylamine, and 13 ml of DMF under ice cooling, followed by stirring for 10 minutes. Water and 1 M hydrochloric acid were added thereto, and the precipitated solids were collected by filtration. The obtained solids were dissolved in ethyl acetate, and the organic layer was washed with saturated saline, dried over magnesium sulfate, and then concentrated under reduced pressure. The residues were subjected to silica gel column chromatography, thereby obtaining 1.89 g of methyl 2-[(2-hydroxy-5-trifluoromethylphenyl)amino]-2-oxo-acetate.

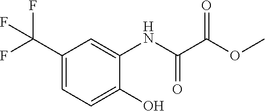 [SMILES: COC(=O)C(=O)Nc1cc(C(F)(F)F)ccc1O]

¹H-NMR (DMSO-D₆) δ: 11.24 (s, 1H), 9.76 (br s, 1H), 8.29 (d, 1H, J=2.3 Hz), 7.41 (dd, 1H, J=8.6, 2.3 Hz), 7.09 (d, 1H, J=8.6 Hz), 3.86 (s, 3H)

### Step 2

A mixture of 2.52 g of azodicarboxylic acid bis(2-methoxyethyl)ester and 10 ml of THF was added dropwise to a mixture of 1.89 g of methyl 2-[(2-hydroxy-5-trifluoromethylphenyl)amino]-2-oxo-acetate, 2.83 g of triphenylphosphine, and 30 ml of THF under ice cooling, followed by stirring for 3 hours at room temperature. The reaction mixture was concentrated under reduced pressure, the residues were subjected to silica gel column chromatography, thereby obtaining 1.42 g of methyl 5-trifluoromethylbenzoxazole-2-carboxylate (hereinafter, described as a “compound 17 of the present invention”).

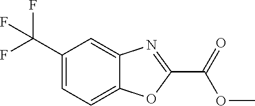 [SMILES: COC(=O)c1nc2cc(C(F)(F)F)ccc2o1]

### Compound 17 of the Present Invention

¹H-NMR (CDCl₃) δ: 8.20-8.17 (m, 1H), 7.84-7.78 (m, 2H), 4.13 (s, 3H)

### Production Example 10

### Step 1

A mixture of 1.98 g of 4-trifluoromethylbenzene-1,2-diamine and 15 ml of acetic acid was added to a mixture of 1.98 g of methyl-2,2,2-trichloroacetimidate and 15 ml of acetic acid under ice cooling, followed by stirring for 3 hours at room temperature. The reaction mixture was concentrated under reduced pressure, ethyl acetate was added to the residues, and the resultant was washed with an aqueous saturated sodium hydrogen carbonate solution and saturated saline. The organic layer was dried over magnesium sulfate and then concentrated under reduced pressure, thereby obtaining 3.39 g of 2-trichloromethyl-5-trifluoromethylbenzimidazole.

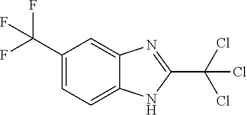 [SMILES: FC(F)(F)c1ccc2[nH]c(C(Cl)(Cl)Cl)nc2c1]

¹H-NMR (DMSO-D₆) δ: 8.18-8.00 (m, 1H), 7.94-7.80 (m, 1H), 7.72-7.65 (m, 1H)

### Step 2

A mixture of 3.24 g of 2-trichloromethyl-5-trifluoromethylbenzimidazole, 2.14 g of sodium hydroxide, and 50 ml of water was stirred for 2 hours under ice cooling. 1.0 g of sodium hydroxide and 20 ml of water were added to the reaction mixture, followed by stirring for 2 hours. Concentrated hydrochloric acid was added thereto, and the precipitated solids were collected by filtration. The obtained solids were washed with water and dried under reduced pressure, thereby obtaining 2.35 g of 5-(trifluoromethyl)-benzimidazole-2-carboxylic acid (hereinafter, described as a “compound 20 of the present invention”).

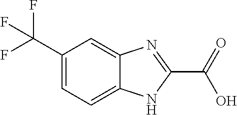 [SMILES: O=C(O)c1nc2cc(C(F)(F)F)ccc2[nH]1]

### Compound 20 of the Present Invention

¹H-NMR (DMSO-D₆) δ: 8.04 (s, 1H), 7.84 (d, 1H, J=8.5 Hz), 7.65 (d, 1H, J=8.5 Hz)

### Production Example 11

A mixture of 1.35 g of 2-trichloromethyl-5-trifluoromethylbenzimidazole and 15 ml of methanol was heated under reflux for 6 hours. 0.47 g of sodium carbonate was added to the reaction mixture, and the resultant was heated under reflux for 16 hours. The reaction mixture was cooled to room temperature and then poured into 150 ml of ice water, and extraction was performed three times by using ethyl acetate. The collected organic layer was washed with saturated saline, dried over magnesium sulfate, and then concentrated under reduced pressure. The residues were subjected to silica gel column chromatography, thereby obtaining 1.1 g of methyl 5-(trifluoromethyl)benzimidazole-2-carboxylate (hereinafter, described as a “compound 21 of the present invention”).

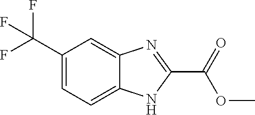 [SMILES: COC(=O)c1nc2cc(C(F)(F)F)ccc2[nH]1]

### Compound 21 of the Present Invention

¹H-NMR (DMSO-D₆) δ: 8.09-8.05 (m, 1H), 7.88-7.83 (m, 1H), 7.68-7.65 (m, 1H), 3.98 (s, 3H)

### Production Example 12

73 mg of sodium hydride (60% dispersion in oil) was added little by little to a mixture of 0.37 g of methyl 5-trifluoromethylbenzimidazole-2-carboxylate and 6 ml of DMF under ice cooling, followed by stirring for 1 hour. 0.22 g of methyl iodide was added to the reaction mixture, and the resultant was stirred for 6 hours at room temperature. Water was added to the reaction mixture, and extraction was performed twice by using ethyl acetate. The collected organic layer was washed with water and saturated saline, dried over magnesium sulfate, and then concentrated under reduced pressure. The residues were subjected to silica gel column chromatography, thereby obtaining 155 mg of methyl 1-methyl-5-trifluoromethylbenzimidazole-2-carboxylate (hereinafter, described as a “compound 24 of the present invention”) and 104 mg of methyl 1-methyl-6-trifluoromethylbenzimidazole-2-carboxylate (hereinafter, described as a “compound 25 of the present invention”).

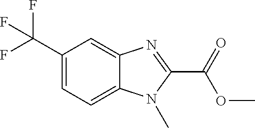 [SMILES: COC(=O)c1nc2cc(C(F)(F)F)ccc2n1C]

### Compound 24 of the Present Invention

¹H-NMR (CDCl₃) δ: 8.18-8.16 (m, 1H), 7.70-7.66 (m, 1H), 7.56 (d, 1H, J=8.7 Hz), 4.22 (s, 3H), 4.08 (s, 3H)

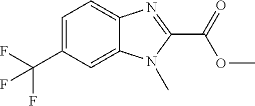 [SMILES: COC(=O)c1nc2ccc(C(F)(F)F)cc2n1C]

### Compound 25 of the Present Invention

¹H-NMR (CDCl₃) δ: 7.99 (d, 1H, J=8.7 Hz), 7.78-7.76 (m, 1H), 7.63-7.60 (m, 1H), 4.24 (s, 3H), 4.08 (s, 3H)

### Production Example 13

### Step 1

A mixture of 1.0 g of 3-amino-2-methylaminopyridine and 6 ml of acetic acid was added to a mixture of 1.58 g of methyl-2,2,2-trichloroacetimidate and 13 ml of acetic acid under ice cooling, followed by stirring for 1 hour at room temperature. 1.58 g of methyl 2,2,2-trichloroacetimidate was added to the reaction mixture, and the resultant was stirred for 2 hours at room temperature and then heated under reflux for 3 hours. After being cooled to room temperature, the reaction mixture was concentrated under reduced pressure. Ethyl acetate was added to the residues, and the resultant was washed with an aqueous saturated sodium hydrogen carbonate solution and saturated saline. The organic layer was dried over magnesium sulfate and then concentrated under reduced pressure. The residues were subjected to silica gel column chromatography, thereby obtaining 0.81 g of 3-methyl-2-trichloromethyl-3H-imidazo[4,5-b]pyridine.

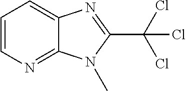

¹H-NMR (CDCl₃) δ: 8.52 (d, 1H, J=4.6 Hz), 8.18 (d, 1H, J=8.2 Hz), 7.37-7.33 (m, 1H), 4.23 (s, 3H)

### Step 2

A mixture of 0.81 g of 3-methyl-2-trichloromethyl-3H-imidazo[4,5-b]pyridine, 0.34 g of sodium carbonate, and 10 ml of methanol was heated under reflux for 6 hours. 0.68 g of sodium carbonate was added to the reaction mixture, and the resultant was heated under reflux for 6 hours. The reaction mixture was cooled to room temperature and then poured into ice water, and extraction was performed three times by using ethyl acetate. The collected organic layer was washed with saturated saline, dried over magnesium sulfate, and then concentrated under reduced pressure. 15 μl of water, 10 mg of 10-camphorsulfoninc acid, and 10 ml of 1,4-dioxane were added to the residues, followed by heating under reflux for 2 hours. 50 μl of water was added to the reaction mixture, followed by heating under reflux for 2 hours. After being cooled to room temperature, the reaction mixture was concentrated under reduced pressure. The residues were subjected to silica gel column chromatography, thereby obtaining 0.12 g of methyl 3-methyl-3H-imidazo[4,5-b]pyridine-2-carboxylate (hereinafter, described as a “compound 26 of the present invention”).

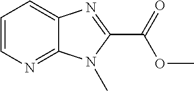

### Compound 26 of the Present Invention

¹H-NMR (CDCl₃) δ: 8.58 (dd, 1H, J=4.7, 1.5 Hz), 8.19 (dd, 1H, J=8.1, 1.5 Hz), 7.35 (dd, 1H, J=8.1, 4.7 Hz), 4.25 (s, 3H), 4.08 (s, 3H)

### Production Example 14

### Step 1

A mixture of 1.09 g of 3-amino-2-methylamino-5-trifluoromethylpyridine and 10 ml of formic acid was heated under reflux for 3 hours. After being cooled to room temperature, the reaction mixture was concentrated under reduced pressure. Ethyl acetate was added to the residues, and the resultant was washed with an aqueous saturated sodium hydrogen carbonate solution, water, and saturated saline. The organic layer was dried over magnesium sulfate and then concentrated under reduced pressure, thereby obtaining 0.98 g of 3-methyl-6-trifluoromethyl-3H-imidazo[4,5-b]pyridine.

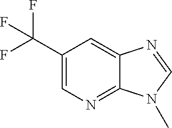

¹H-NMR (CDCl₃) δ: 8.71 (d, 1H, J=1.4 Hz), 8.32 (d, 1H, J=1.4 Hz), 8.17 (s, 1H), 3.98 (s, 3H)

### Step 2

1.56 ml of n-butyllithium (1.6 N, n-hexane solution) was added dropwise to a mixture of 0.50 g of 3-methyl-6-trifluoromethyl-3H-imidazo[4,5-b]pyridine and 10 ml of THF at −70° C. in nitrogen atmosphere, followed by stirring for 30 minutes. Dry ice was added to the reaction mixture, and the resultant was stirred for 1 hour at room temperature. After the reaction mixture was concentrated under reduced pressure, 10 ml of DMF and 0.16 ml of methyl iodide were added thereto, followed by stirring for 3 hours at room temperature. Water was added to the reaction mixture, and extraction was performed three times by using ethyl acetate. The collected organic layer was washed with water and saturated saline, dried over magnesium sulfate, and then concentrated under reduced pressure. The residues were subjected to silica gel column chromatography, thereby obtaining 71 mg of methyl 3-methyl-6-trifluoromethyl-3H-imidazo[4,5-b]pyridine-2-carboxylate (hereinafter, described as a “compound 27 of the present invention”).

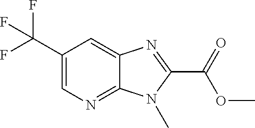 [SMILES: COC(=O)c1nc2cc(C(F)(F)F)cnc2n1C]

### Compound 27 of the Present Invention

¹H-NMR (CDCl₃) δ: 8.84-8.82 (m, 1H), 8.42-8.40 (m, 1H), 4.28 (s, 3H), 4.10 (s, 3H)

### Production Example 15

A mixture of 1.06 g of 2-amino-4-trifluoromethylbenzenethiol hydrochloride, 1.13 g of methyl trimethoxyacetate, and 7 ml of toluene was heated under reflux for 5 hours. After being cooled to room temperature, the reaction mixture was concentrated under reduced pressure. The residues were subjected to silica gel column chromatography, thereby obtaining 0.80 g of methyl 5-trifluoromethylbenzothiazole-2-carboxylate (hereinafter, described as a “compound 29 of the present invention”).

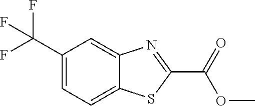 [SMILES: COC(=O)c1nc2cc(C(F)(F)F)ccc2s1]

### Compound 29 of the Present Invention

¹H-NMR (CDCl₃) δ: 8.52-8.50 (m, 1H), 8.13 (d, 1H, J=8.5 Hz), 7.81-7.77 (m, 1H), 4.12 (s, 3H)

Specific examples of the compounds of the present invention include the compounds (compounds 1 to 87 of the present invention) represented by Formula (1) in which A¹, A², A³, R¹, R², R³, R⁴, R⁵, R¹², and W form a combination of groups shown in Tables 1 to 6. In the following tables, Me represents a methyl group, Et represents an ethyl group, Ph represents a phenyl group, and Bn represents a benzyl group.

TABLE 1

| Compound | | | | | | | | | | | |
|---|---|---|---|---|---|---|---|---|---|---|---|
| of the | | | | | | | | | | | |
| present | | | | | | | | | | | Melting |
| invention | A¹ | A² | A³ | R¹ | R² | R³ | R⁴ | R⁵ | R¹² | W | Point |
| 1 | —C(R⁵)═ | —O— | —C(R⁴)═ | H | H | H | H | H | — | MeO— | * |
| 2 | —C(R⁵)═ | —O— | —C(R⁴)═ | H | CF₃ | H | H | H | — | HO— | — |
| 3 | —C(R⁵)═ | —O— | —C(R⁴)═ | H | CF₃ | H | H | H | — | MeO— | — |
| 4 | —C(R⁵)═ | —O— | —C(R⁴)═ | H | CF₃ | H | H | H | — | EtO— | — |
| 5 | —C(R⁵)═ | —O— | —C(R⁴)═ | H | CF₃ | H | H | H | — | 2,4- | — |
| | | | | | | | | | | (MeO)₂PhNH— | |
| 6 | —C(R⁵)═ | —N(R¹²)— | —C(R⁴)═ | CF₃ | H | H | H | H | H | MeO— | 131.4° C. |
| 7 | —C(R⁵)═ | —N(R¹²)— | —C(R⁴)═ | H | CF₃ | H | H | H | H | HO— | — |
| 8 | —C(R⁵)═ | —N(R¹²)— | —C(R⁴)═ | H | CF₃ | H | H | H | H | MeO— | — |
| 9 | —C(R⁵)═ | —N(R¹²)— | —C(R⁴)═ | H | CF₃ | H | H | H | H | EtO— | 147.6° C. |
| 10 | —C(R⁵)═ | —N(R¹²)— | —C(R⁴)═ | H | CF₃ | H | H | H | H | 2,4- | — |
| | | | | | | | | | | (MeO)₂PhNH— | |
| 11 | —C(R⁵)═ | —N(R¹²)— | —C(R⁴)═ | H | H | H | CF₃ | H | H | HO— | 157.2° C. |
| 12 | —C(R⁵)═ | —N(R¹²)— | —C(R⁴)═ | H | H | H | CF₃ | H | H | EtO— | 69.3° C. |
| 13 | —C(R⁵)═ | —N(R¹²)— | —C(R⁴)═ | H | H | H | H | H | H | MeO— | 153.0° C. |
| 14 | —C(R⁵)═ | —N(R¹²)— | —C(R⁴)═ | H | H | H | H | H | Me | HO— | 208.6° C. |
| | | | | | | | | | | | (decomp. ) |
| 15 | —C(R⁵)═ | —N(R¹²)— | —C(R⁴)═ | H | H | H | H | H | Me | MeO— | * |
| 16 | —N═ | —O— | —C(R⁴)═ | H | H | H | H | — | — | MeO— | — |

TABLE 2

| Compound | | | | | | | | | | | |
|---|---|---|---|---|---|---|---|---|---|---|---|
| of the | | | | | | | | | | | |
| present | | | | | | | | | | | Melting |
| invention | A¹ | A² | A³ | R¹ | R² | R³ | R⁴ | R⁵ | R¹² | W | Point |
| 17 | —N═ | —O— | —C(R⁴)═ | H | CF₃ | H | H | — | — | MeO— | — |
| 18 | —N═ | —N(R¹²)— | —C(R⁴)═ | H | H | H | H | — | H | HO— | * |
| 19 | —N═ | —N(R¹²)— | —C(R⁴)═ | H | H | H | H | | H | MeO— | 182.1° C. |
| | | | | | | | | | | | (decomp. ) |
| 20 | —N═ | —N(R¹²)— | —C(R⁴)═ | H | CF₃ | H | H | — | H | HO— | — |
| 21 | —N═ | —N(R¹²)— | —C(R⁴)═ | H | CF₃ | H | H | — | H | MeO— | — |
| 22 | —N═ | —N(R¹²)— | —C(R⁴)═ | H | H | H | H | — | Me | HO— | * |
| 23 | —N═ | —N(R¹²)— | —C(R⁴)═ | H | H | H | H | — | Me | MeO— | 62.1° C. |
| 24 | —N═ | —N(R¹²)— | —C(R⁴)═ | H | CF₃ | H | H | — | Me | MeO— | — |
| 25 | —N═ | —N(R¹²)— | —C(R⁴)═ | H | H | CF₃ | H | — | Me | MeO— | — |
| 26 | —N═ | —N(R¹²)— | —N═ | H | H | H | — | — | Me | MeO— | — |
| 27 | —N═ | —N(R¹²)— | —N═ | H | CF₃ | H | — | — | Me | MeO— | — |
| 28 | —N═ | —N(R¹²)— | —N═ | H | H | H | — | — | H | HO— | 139.2° C. |
| 29 | —N═ | —S— | —C(R⁴)═ | H | CF₃ | H | H | — | — | MeO— | — |
| 30 | —N═ | —S— | —C(R⁴)═ | H | H | H | H | — | — | MeO— | 93.1° C. |
| 31 | —N═ | —S— | —C(R⁴)═ | H | H | H | H | — | — | EtO— | 141.2° C. |
| 32 | —N═ | —S— | —C(R⁴)═ | H | H | H | H | — | — | HO— | 91.3° C. |

TABLE 3

| | | | | | | | | | | | Melting |
|---|---|---|---|---|---|---|---|---|---|---|---|
| *1 | A¹ | A² | A³ | R¹ | R² | R³ | R⁴ | R⁵ | R¹² | W | Point |
| 33 | —C(R⁵)═ | —O— | —C(R⁴)═ | H | CF₃ | H | H | Me | — | MeO— | * |
| 34 | —C(R⁵)═ | —O— | —C(R⁴)═ | H | CF₃ | H | H | Me | — | HO— | * |
| 35 | —C(R⁵)═ | —O— | —C(R⁴)═ | H | H | H | H | OH | — | MeO— | * |
| 36 | —C(R⁵)═ | —O— | —C(R⁴)═ | H | H | H | H | Me | — | HO— | * |
| 37 | —C(R⁵)═ | —O— | —C(R⁴)═ | H | H | H | H | Me | — | MeO— | * |
| 38 | —C(R⁵)═ | —O— | —C(R⁴)═ | H | H | H | H | N[C(O)CF₃)]₂ | — | MeO— | * |
| 39 | —C(R⁵)═ | —O— | —C(R⁴)═ | H | H | H | H | NHC(O)CF₃ | — | MeO— | * |
| 40 | —C(R⁵)═ | —O— | —C(R⁴)═ | H | H | H | H | NHC(O)Me | — | MeO— | * |
| 41 | —C(R⁵)═ | —O— | —C(R⁴)═ | H | H | H | H | N[C(O)Me)]₂ | — | MeO— | * |
| 42 | —C(R⁵)═ | —O— | —C(R⁴)═ | H | CF₃ | H | H | NH₂ | — | MeO— | * |
| 43 | —C(R⁵)═ | —O— | —C(R⁴)═ | CF₃ | H | H | H | H | — | HO— | * |
| 44 | —C(R⁵)═ | —O— | —C(R⁴)═ | CF₃ | H | H | H | H | — | EtO— | * |
| 45 | —C(R⁵)═ | —O— | —C(R⁴)═ | CF₃ | H | H | H | H | — | MeO— | * |
| 46 | —C(R⁵)═ | —O— | —C(R⁴)═ | H | H | CF₃ | H | H | — | EtO— | * |
| 47 | —C(R⁵)═ | —O— | —C(R⁴)═ | H | H | CF₃ | H | H | — | HO— | * |

*1: Compound of the present invention

TABLE 4

| | | | | | | | | | | | Melting |
|---|---|---|---|---|---|---|---|---|---|---|---|
| *1 | A¹ | A² | A³ | R¹ | R² | R³ | R⁴ | R⁵ | R¹² | W | Point |
| 48 | —C(R⁵)═ | —O— | —C(R⁴)═ | H | H | CF₃ | H | H | — | MeO— | * |
| 49 | —C(R⁵)═ | —O— | —C(R⁴)═ | H | CF₃ | H | H | NH₂ | — | HO— | * |
| 50 | —C(R⁵)═ | —O— | —C(R⁴)═ | H | CF₃ | H | H | OH | — | MeO— | * |
| 51 | —C(R⁵)═ | —O— | —C(R⁴)═ | H | CF₃ | H | H | H | — | H₂N— | * |
| 52 | —C(R⁵)═ | —O— | —C(R⁴)═ | H | CF₃ | H | H | H | — | MeNH— | * |
| 53 | —C(R⁵)═ | —O— | —C(R⁴)═ | H | CF₃ | H | H | H | — | PhNH— | * |
| 54 | —C(R⁵)═ | —O— | —C(R⁴)═ | H | CF₃ | H | H | H | — | BnNH— | * |
| 55 | —C(R⁵)═ | —O— | —C(R⁴)═ | H | CF₃ | H | H | H | — | PhO— | * |
| 56 | —C(R⁵)═ | —O— | —C(R⁴)═ | H | CF₃ | H | H | H | — | BnO— | * |
| 57 | —C(R⁵)═ | —O— | —C(R⁴)═ | H | H | H | H | NH₂ | — | MeO— | * |
| 58 | —C(R⁵)═ | —O— | —C(R⁴)═ | H | H | H | H | NMe₂ | — | MeO— | * |
| 59 | —C(R⁵)═ | —O— | —C(R⁴)═ | H | H | H | H | NHC(O)Ph | — | MeO— | * |
| 60 | —C(R⁵)═ | —O— | —C(R⁴)═ | H | H | H | H | NHS(O)₂Me | — | MeO— | * |
| 61 | —C(R⁵)═ | —O— | —C(R⁴)═ | H | H | H | H | NHS(O)₂Ph | — | MeO— | * |

*1: Compound of the present invention

TABLE 5

| | | | | | | | | | | | Melting |
|---|---|---|---|---|---|---|---|---|---|---|---|
| *1 | A¹ | A² | A³ | R¹ | R² | R³ | R⁴ | R⁵ | R¹² | W | Point |
| 62 | —C(R⁵)═ | —O— | —C(R⁴)═ | H | CF₃ | H | H | NMe₂ | — | MeO— | * |
| 63 | —C(R⁵)═ | —O— | —C(R⁴)═ | H | CF₃ | H | H | NHC(O)Me | — | MeO— | * |
| 64 | —C(R⁵)═ | —O— | —C(R⁴)═ | H | CF₃ | H | H | N[C(O)Me)]₂ | — | MeO— | * |
| 65 | —C(R⁵)═ | —O— | —C(R⁴)═ | H | CF₃ | H | H | NHC(O)CF₃ | — | MeO— | * |
| 66 | —C(R⁵)═ | —O— | —C(R⁴)═ | H | CF₃ | H | H | NHC(O)Ph | — | MeO— | * |
| 67 | —C(R⁵)═ | —O— | —C(R⁴)═ | H | CF₃ | H | H | NHS(O)₂Me | — | MeO— | * |
| 68 | —C(R⁵)═ | —O— | —C(R⁴)═ | H | CF₃ | H | H | NHS(O)₂Ph | — | MeO— | * |
| 69 | —C(R⁵)═ | —O— | —C(R⁴)═ | H | CF₃ | H | H | CF₃ | — | HO— | * |
| 70 | —C(R⁵)═ | —O— | —C(R⁴)═ | H | CF₃ | H | H | CF₃ | — | MeO— | * |
| 71 | —C(R⁵)═ | —O— | —C(R⁴)═ | H | CF₃ | H | H | CF₃ | — | EtO— | * |
| 72 | —C(R⁵)═ | —O— | —C(R⁴)═ | H | CF₃ | H | H | CF₃ | — | PhO— | * |
| 73 | —C(R⁵)═ | —O— | —C(R⁴)═ | H | CF₃ | H | H | CF₃ | — | BnO— | * |
| 74 | —C(R⁵)═ | —O— | —C(R⁴)═ | H | CF₃ | H | H | CF₃ | — | H₂N— | * |
| 75 | —C(R⁵)═ | —O— | —C(R⁴)═ | H | CF₃ | H | H | CF₃ | — | MeNH— | * |

*1: Compound of the present invention

TABLE 6

| | | | | | | | | | | | Melting |
|---|---|---|---|---|---|---|---|---|---|---|---|
| *1 | A¹ | A² | A³ | R¹ | R² | R³ | R⁴ | R⁵ | R¹² | W | Point |
| 76 | —C(R⁵)═ | —O— | —C(R⁴)═ | H | CF₃ | H | H | CF₃ | — | PhNH— | * |
| 77 | —C(R⁵)═ | —O— | —C(R⁴)═ | H | CF₃ | H | H | CF₃ | — | BnNH— | * |
| 78 | —C(R⁵)═ | —O— | —C(R⁴)═ | H | H | H | H | CO₂H | — | HO— | * |
| 79 | —C(R⁵)═ | —O— | —C(R⁴)═ | H | Br | H | H | H | — | HO— | * |
| 80 | —C(R⁵)═ | —O— | —C(R⁴)═ | OH | H | H | H | Me | — | EtO— | * |
| 81 | —C(R⁵)═ | —O— | —C(R⁴)═ | H | H | H | H | NHCHO | — | MeO— | * |
| 82 | —C(R⁵)═ | —O— | —C(R⁴)═ | H | H | H | H | CF₃ | — | HO— | * |
| 83 | —C(R⁵)═ | —O— | —C(R⁴)═ | H | H | H | H | CF₃ | — | MeO— | * |
| 84 | —C(R⁵)═ | —O— | —C(R⁴)═ | H | H | H | H | CF₃ | — | EtO— | * |
| 85 | —C(R⁵)═ | —O— | —C(R⁴)═ | H | H | H | H | CF₃ | — | H₂N— | * |
| 86 | —N═ | —S— | —C(R⁴)═ | F | H | F | H | — | — | HO— | * |
| 87 | —N═ | —S— | —C(R⁴)═ | F | H | F | H | — | — | MeO— | * |

*1: Compound of the present invention

Regarding compounds marked with * in the column of a melting point in Tables 1 to 6, ¹H-NMR data thereof are shown below.

Compound 1 of the Present Invention

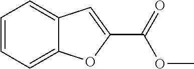

¹H-NMR (CDCl₃) δ: 7.71-7.69 (m, 1H), 7.61-7.59 (m, 1H), 7.55-7.54 (m, 1H), 7.48-7.44 (m, 1H), 7.34-7.30 (m, 1H), 3.99 (s, 3H)

Compound 15 of the Present Invention

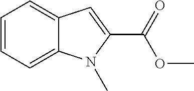

¹H-NMR (CDCl₃) δ: 7.69-7.67 (m, 1H), 7.41-7.34 (m, 2H), 7.31-7.29 (m, 1H), 7.17-7.14 (m, 1H), 4.09 (s, 3H), 3.92 (s, 3H)

Compound 18 of the Present Invention

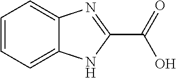 [SMILES: O=C(O)c1nc2ccccc2[nH]1]

¹H-NMR (DMSO-D₆) δ: 11.92 (br s, 1H), 7.17-7.12 (m, 2H), 7.10-7.08 (m, 2H)

Compound 22 of the Present Invention

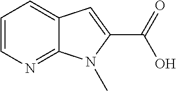

¹H-NMR (CDCl₃) δ: 7.86 (s, 1H), 7.82-7.80 (m, 1H), 7.41-7.39 (m, 1H), 7.35-7.26 (m, 2H), 3.82 (s, 3H)

Compound 33 of the Present Invention

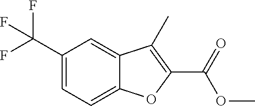 [SMILES: COC(=O)c1oc2ccc(C(F)(F)F)cc2c1C]

¹H-NMR (CDCl₃) δ: 7.94 (s, 1H), 7.71 (d, 1H, J=8.9 Hz), 7.64 (d, 1H, J=8.9 Hz), 4.01 (s, 3H), 2.63 (s, 3H).

Compound 34 of the Present Invention

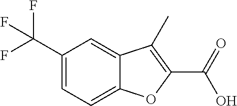 [SMILES: Cc1c(C(=O)O)oc2ccc(C(F)(F)F)cc12]

¹H-NMR (CDCl₃) δ: 7.98 (s, 1H), 7.75 (d, 1H, J=8.5 Hz), 7.68 (d, 1H, J=8.5 Hz), 2.68 (s, 3H).

Compound 35 of the Present Invention

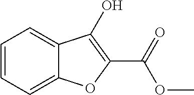

¹H-NMR (DMSO-D₆) δ: 10.88 (br s, 1H), 7.91-7.89 (m, 1H), 7.59-7.49 (m, 2H), 7.35-7.30 (m, 1H), 3.84 (s, 3H).

Compound 36 of the Present Invention

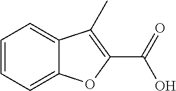

¹H-NMR (CDCl₃) δ: 7.69-7.66 (m, 1H), 7.60-7.56 (m, 1H), 7.53-7.47 (m, 1H), 7.36-7.31 (m, 1H), 2.67 (s, 3H).

Compound 37 of the Present Invention

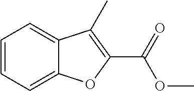

¹H-NMR (CDCl₃) δ: 7.67-7.62 (m, 1H), 7.57-7.53 (m, 1H), 7.48-7.43 (m, 1H), 7.36-7.32 (m, 1H), 3.99 (s, 3H), 2.59 (s, 3H).

Compound 38 of the Present Invention

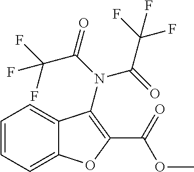 [SMILES: COC(=O)c1oc2ccccc2c1N(C(=O)C(F)(F)F)C(=O)C(F)(F)F]

¹H-NMR (DMSO-D₆) δ: 7.79-7.75 (m, 1H), 7.74-7.71 (m, 1H), 7.64-7.59 (m, 1H), 7.46-7.41 (m, 1H), 3.89 (s, 3H).

Compound 39 of the Present Invention

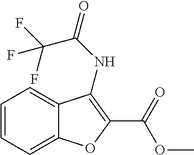 [SMILES: COC(=O)c1oc2ccccc2c1NC(=O)C(F)(F)F]

¹H-NMR (DMSO-D₆) δ: 7.96-7.93 (m, 1H), 7.50-7.47 (m, 2H), 7.27-7.24 (m, 1H), 6.39 (br s, 1H), 3.81 (s, 3H).

Compound 40 of the Present Invention

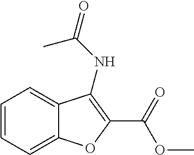 [SMILES: COC(=O)c1oc2ccccc2c1NC(C)=O]

¹H-NMR (CDCl₃) δ: 9.32-9.28 (m, 1H), 8.40-8.36 (m, 1H), 7.49-7.44 (m, 2H), 4.01 (s, 3H), 2.33 (s, 3H).

Compound 41 of the Present Invention

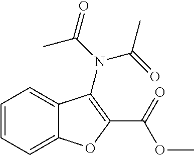 [SMILES: COC(=O)c1oc2ccccc2c1N(C(C)=O)C(C)=O]

¹H-NMR (CDCl₃) δ: 8.00-7.98 (m, 1H), 7.73-7.69 (m, 1H), 7.49-7.41 (m, 2H), 3.92 (s, 3H), 3.16 (s, 3H), 3.11 (s, 3H).

Compound 42 of the Present Invention

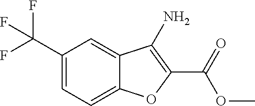 [SMILES: COC(=O)c1oc2ccc(C(F)(F)F)cc2c1N]

¹H-NMR (DMSO-D₆) δ: 8.50 (s, 1H), 7.82 (d, 1H, J=8.5 Hz), 7.72 (d, 1H, J=8.5 Hz), 6.57 (br s, 2H), 3.83 (s, 3H).

Compound 43 of the Present Invention

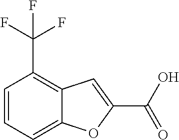 [SMILES: O=C(O)c1cc2c(C(F)(F)F)cccc2o1]

¹H-NMR (CDCl₃) δ: 7.82-7.78 (m, 1H), 7.65-7.59 (m, 2H), 7.58-7.53 (m, 1H).

Compound 44 of the Present Invention

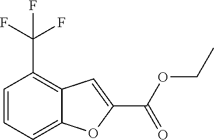 [SMILES: CCOC(=O)c1cc2c(C(F)(F)F)cccc2o1]

¹H-NMR (CDCl₃) δ: 7.80-7.76 (m, 1H), 7.69-7.66 (m, 1H), 7.63-7.58 (m, 1H), 7.57-7.51 (m, 1H), 4.47 (q, 2H, J=7.1 Hz), 1.45 (t, 3H, J=7.1 Hz).

Compound 45 of the Present Invention

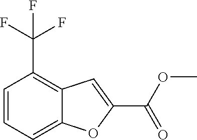 [SMILES: COC(=O)c1cc2c(C(F)(F)F)cccc2o1]

¹H-NMR (CDCl₃) δ: 7.81-7.77 (m, 1H), 7.70-7.68 (m, 1H), 7.64-7.60 (m, 1H), 7.58-7.52 (m, 1H), 4.01 (s, 3H).

Compound 46 of the Present Invention

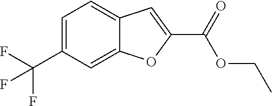 [SMILES: CCOC(=O)c1cc2ccc(C(F)(F)F)cc2o1]

¹H-NMR (CDCl₃) δ: 7.88-7.85 (m, 1H), 7.82-7.78 (m, 1H), 7.59-7.54 (m, 2H), 4.47 (q, 2H, J=7.6 Hz), 1.45 (t, 3H, J=7.6 Hz).

Compound 47 of the Present Invention

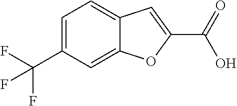 [SMILES: O=C(O)c1cc2ccc(C(F)(F)F)cc2o1]

¹H-NMR (DMSO-D₆) δ: 8.22 (s, 1H), 8.02 (d, 1H, J=8.7 Hz), 7.79 (s, 1H), 7.70 (d, 1H, J=8.7 Hz).

Compound 48 of the Present Invention

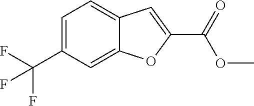 [SMILES: COC(=O)c1cc2ccc(C(F)(F)F)cc2o1]

¹H-NMR (CDCl₃) δ: 7.86-7.85 (m, 1H), 7.82-7.78 (m, 1H), 7.59-7.54 (m, 2H), 4.01 (s, 3H).

Compound 49 of the Present Invention

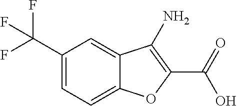 [SMILES: Nc1c(C(=O)O)oc2ccc(C(F)(F)F)cc12]

¹H-NMR (DMSO-D₆) δ: 8.48-8.46 (m, 1H), 7.82-7.78 (m, 1H), 7.72-7.69 (m, 1H).

Compound 50 of the Present Invention

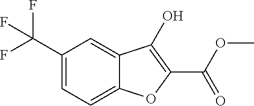 [SMILES: COC(=O)c1oc2ccc(C(F)(F)F)cc2c1O]

¹H-NMR (CDCl₃) δ: 8.08-8.06 (m, 1H), 8.02 (br s, 1H), 7.75-7.72 (m, 1H), 7.59-7.55 (m, 1H), 4.04 (s, 3H).

Compound 51 of the Present Invention

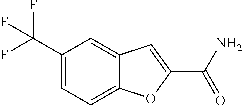 [SMILES: NC(=O)c1cc2cc(C(F)(F)F)ccc2o1]

¹H-NMR (DMSO-D₆) δ: 8.26 (br s, 1H), 8.24 (s, 1H), 7.88-7.86 (m, 1H), 7.81 (br s, 1H), 7.79-7.77 (m, 1H), 7.67 (s, 1H).

Compound 52 of the Present Invention

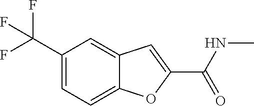 [SMILES: CNC(=O)c1cc2cc(C(F)(F)F)ccc2o1]

¹H-NMR (DMSO-D₆) δ: 8.82-8.81 (br s, 1H), 8.24 (s, 1H), 7.88-7.86 (m, 1H), 7.80-7.77 (m, 1H), 7.62 (s, 1H), 2.82-2.80 (m, 3H).

Compound 53 of the Present Invention

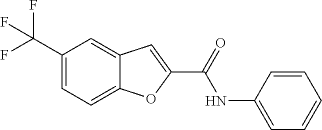 [SMILES: O=C(Nc1ccccc1)c1cc2cc(C(F)(F)F)ccc2o1]

¹H-NMR (DMSO-D₆) δ: 10.65 (br s, 1H), 8.31 (s, 1H), 7.97-7.94 (m, 1H), 7.90 (s, 1H), 7.85-7.80 (m, 3H), 7.40-7.36 (m, 2H), 7.16-7.13 (m, 1H).

Compound 54 of the Present Invention

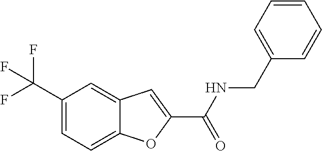 [SMILES: O=C(NCc1ccccc1)c1cc2cc(C(F)(F)F)ccc2o1]

¹H-NMR (DMSO-D₆) δ: 9.44-9.42 (m, 1H), 8.25 (s, 1H), 7.90-7.88 (m, 1H), 7.80-7.78 (m, 1H), 7.70 (s, 1H), 7.34-7.31 (m, 4H), 7.28-7.23 (m, 1H), 4.51-4.48 (m, 2H).

Compound 55 of the Present Invention

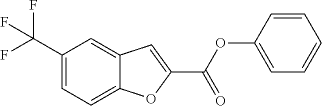 [SMILES: O=C(Oc1ccccc1)c1cc2cc(C(F)(F)F)ccc2o1]

¹H-NMR (DMSO-D₆) δ: 8.33 (s, 1H), 8.12 (s, 1H), 8.06-8.04 (m, 1H), 7.93-7.91 (m, 1H), 7.53-7.49 (m, 2H), 7.37-7.35 (m, 3H).

Compound 56 of the Present Invention

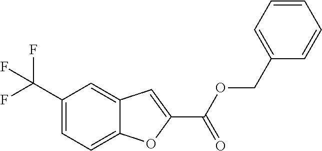 [SMILES: O=C(OCc1ccccc1)c1cc2cc(C(F)(F)F)ccc2o1]

¹H-NMR (DMSO-D₆) δ: 8.25 (s, 1H), 7.99-7.97 (m, 1H), 7.92 (s, 1H), 7.88-7.85 (m, 1H), 7.51-7.49 (m, 2H), 7.45-7.36 (m, 3H), 5.41 (s, 2H).

Compound 57 of the Present Invention

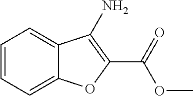

¹H-NMR (DMSO-D₆) δ: 7.96-7.93 (m, 1H), 7.50-7.45 (m, 2H), 7.27-7.23 (m, 1H), 6.38 (br s, 2H), 3.80 (s, 3H).

Compound 58 of the Present Invention

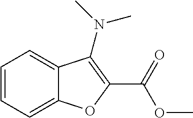 [SMILES: COC(=O)c1oc2ccccc2c1N(C)C]

¹H-NMR (DMSO-D₆) δ: 8.02-7.98 (m, 1H), 7.54-7.46 (m, 2H), 7.28-7.24 (m, 1H), 3.80 (s, 3H), 3.15 (s, 6H).

Compound 59 of the Present Invention

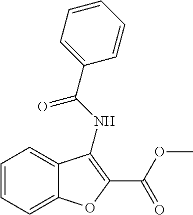 [SMILES: COC(=O)c1oc2ccccc2c1NC(=O)c1ccccc1]

¹H-NMR (DMSO-D₆) δ: 10.43 (br s, 1H), 8.06-8.04 (m, 2H), 7.93-7.91 (m, 1H), 7.73-7.71 (m, 1H), 7.68-7.65 (m, 1H), 7.62-7.56 (m, 3H), 7.40-7.36 (m, 1H), 3.89 (s, 3H).

Compound 60 of the Present Invention

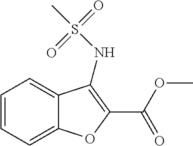 [SMILES: COC(=O)c1oc2ccccc2c1NS(C)(=O)=O]

¹H-NMR (DMSO-D₆) δ: 9.66 (br s, 1H), 7.85-7.83 (m, 1H), 7.71-7.68 (m, 1H), 7.58-7.54 (m, 1H), 7.41-7.38 (m, 1H), 3.91 (s, 3H), 3.10 (s, 3H).

Compound 61 of the Present Invention

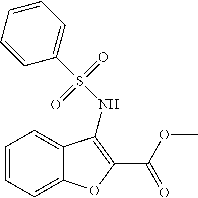 [SMILES: COC(=O)c1oc2ccccc2c1NS(=O)(=O)c1ccccc1]

¹H-NMR (DMSO-D₆) δ: 10.08 (br s, 1H), 7.73-7.70 (m, 1H), 7.68-7.61 (m, 5H), 7.56-7.51 (m, 3H), 7.39-7.36 (m, 1H), 3.54 (s, 3H).

Compound 62 of the Present Invention

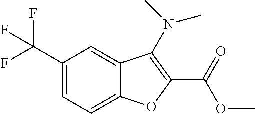 [SMILES: COC(=O)c1oc2ccc(C(F)(F)F)cc2c1N(C)C]

¹H-NMR (CDCl₃) δ: 8.13 (s, 1H), 7.66-7.64 (m, 1H), 7.55-7.52 (m, 1H), 3.94 (s, 3H), 3.23 (s, 6H).

Compound 63 of the Present Invention

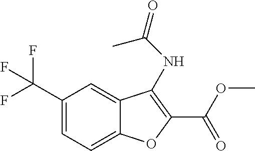 [SMILES: COC(=O)c1oc2ccc(C(F)(F)F)cc2c1NC(C)=O]

¹H-NMR (DMSO-D₆) δ: 10.11 (br s, 1H), 8.21 (s, 1H), 7.92 (d, 1H, J=8.8 Hz), 7.87 (d, 1H, J=8.8 Hz), 3.90 (s, 3H), 2.20 (s, 3H).

Compound 64 of the Present Invention

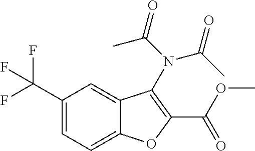 [SMILES: COC(=O)c1oc2ccc(C(F)(F)F)cc2c1N(C(C)=O)C(C)=O]

¹H-NMR (DMSO-D₆) δ: 8.39 (s, 1H), 8.06 (d, 1H, J=8.9 Hz), 7.95 (d, 1H, J=8.9 Hz), 3.89 (s, 3H), 2.30 (s, 6H).

Compound 65 of the Present Invention

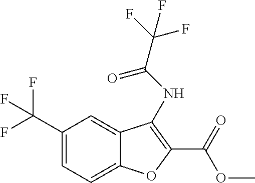 [SMILES: COC(=O)c1oc2ccc(C(F)(F)F)cc2c1NC(=O)C(F)(F)F]

¹H-NMR (DMSO-D₆) δ: 11.70 (br s, 1H), 8.25 (s, 1H), 8.04-8.00 (m, 1H), 7.96-7.92 (m, 1H), 3.90 (s, 3H).

Compound 66 of the Present Invention

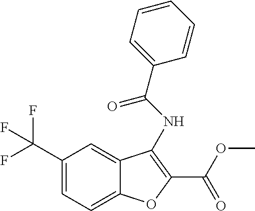 [SMILES: COC(=O)c1oc2ccc(C(F)(F)F)cc2c1NC(=O)c1ccccc1]

¹H-NMR (DMSO-D₆) δ: 10.55 (br s, 1H), 8.39 (s, 1H), 8.06-8.05 (m, 2H), 7.99-7.97 (m, 1H), 7.93-7.91 (m, 1H), 7.70-7.66 (m, 1H), 7.62-7.59 (m, 2H), 3.91 (s, 3H).

Compound 67 of the Present Invention

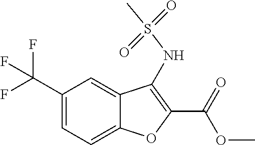 [SMILES: COC(=O)c1oc2ccc(C(F)(F)F)cc2c1NS(C)(=O)=O]

¹H-NMR (DMSO-D₆) δ: 9.87 (br s, 1H), 8.17 (s, 1H), 7.98-7.96 (m, 1H), 7.92-7.89 (m, 1H), 3.93 (s, 3H), 3.13 (s, 3H).

Compound 68 of the Present Invention

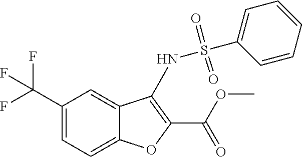 [SMILES: COC(=O)c1oc2ccc(C(F)(F)F)cc2c1NS(=O)(=O)c1ccccc1]

¹H-NMR (DMSO-D₆) δ: 10.32 (br s, 1H), 7.95-7.86 (m, 3H), 7.66-7.62 (m, 3H), 7.54-7.50 (m, 2H), 3.59 (s, 3H)

Compound 69 of the Present Invention

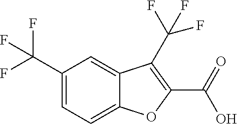 [SMILES: O=C(O)c1oc2ccc(C(F)(F)F)cc2c1C(F)(F)F]

¹H-NMR (DMSO-D₆) δ: 8.11-8.09 (m, 2H), 7.97-7.95 (m, 1H).

Compound 70 of the Present Invention

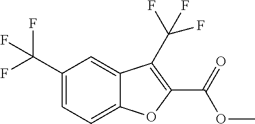 [SMILES: COC(=O)c1oc2ccc(C(F)(F)F)cc2c1C(F)(F)F]

¹H-NMR (DMSO-D₆) δ: 8.17-8.15 (m, 2H), 8.04-8.02 (m, 1H), 3.99 (s, 3H).

Compound 71 of the Present Invention

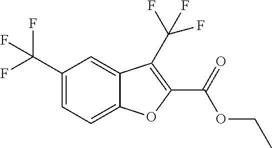 [SMILES: CCOC(=O)c1oc2ccc(C(F)(F)F)cc2c1C(F)(F)F]

¹H-NMR (CDCl₃) δ: 8.16 (s, 1H), 7.80-7.73 (m, 2H), 4.53-4.48 (m, 2H), 1.46-1.43 (m, 3H).

Compound 72 of the Present Invention

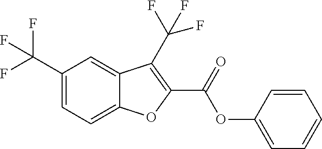 [SMILES: O=C(Oc1ccccc1)c1oc2ccc(C(F)(F)F)cc2c1C(F)(F)F]

¹H-NMR (CDCl₃) δ: 8.22 (s, 1H), 7.86-7.79 (m, 2H), 7.48-7.44 (m, 2H), 7.34-7.29 (m, 3H).

Compound 73 of the Present Invention

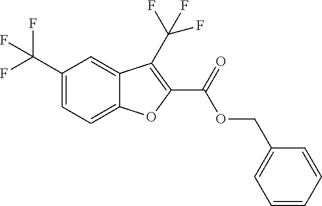 [SMILES: O=C(OCc1ccccc1)c1oc2ccc(C(F)(F)F)cc2c1C(F)(F)F]

¹H-NMR (CDCl₃) δ: 8.16 (s, 1H), 7.80-7.72 (m, 2H), 7.48-7.47 (m, 2H), 7.43-7.35 (m, 3H), 5.47 (s, 2H).

Compound 74 of the Present Invention

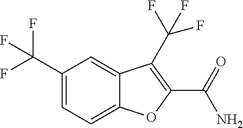 [SMILES: NC(=O)c1oc2ccc(C(F)(F)F)cc2c1C(F)(F)F]

¹H-NMR (DMSO-D₆) δ: 8.55 (br s, 1H), 8.26 (br s, 1H), 8.10 (s, 1H), 8.05-8.03 (m, 1H), 7.97-7.75 (m, 1H).

Compound 75 of the Present Invention

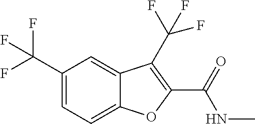 [SMILES: CNC(=O)c1oc2ccc(C(F)(F)F)cc2c1C(F)(F)F]

¹H-NMR (CDCl₃) δ: 8.16 (s, 1H), 7.76 (d, 1H, J=8.8 Hz), 7.66 (d, 1H, J=8.8 Hz), 6.70 (br s, 1H), 3.09-3.07 (m, 3H).

Compound 76 of the Present Invention

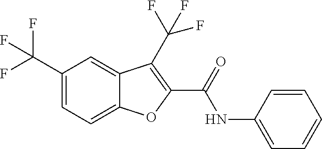 [SMILES: O=C(Nc1ccccc1)c1oc2ccc(C(F)(F)F)cc2c1C(F)(F)F]

¹H-NMR (CDCl₃) δ: 8.35 (br s, 1H), 8.20 (s, 1H), 7.83-7.81 (m, 1H), 7.76-7.70 (m, 3H), 7.42-7.38 (m, 2H), 7.24-7.20 (m, 1H).

Compound 77 of the Present Invention

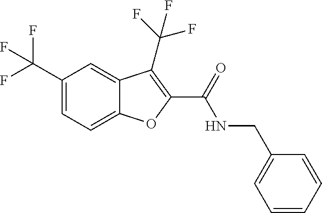 [SMILES: O=C(NCc1ccccc1)c1oc2ccc(C(F)(F)F)cc2c1C(F)(F)F]

¹H-NMR (CDCl₃) δ: 8.16 (s, 1H), 7.77-7.74 (m, 1H), 7.65-7.62 (m, 1H), 7.39-7.33 (m, 5H), 6.95 (br s, 1H), 4.69-4.67 (m, 2H).

Compound 78 of the Present Invention

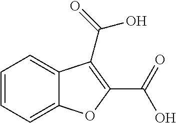 [SMILES: O=C(O)c1oc2ccccc2c1C(=O)O]

¹H-NMR (DMSO-D₆) δ: 8.17-8.12 (m, 1H), 7.69-7.65 (m, 1H), 7.49-7.43 (m, 1H), 7.39-7.33 (m, 1H).

Compound 79 of the Present Invention

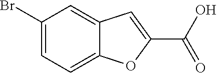 [SMILES: O=C(O)c1cc2cc(Br)ccc2o1]

¹H-NMR (CDCl₃) δ: 7.99-7.98 (m, 1H), 7.71-7.69 (m, 2H), 7.64-7.60 (m, 1H).

Compound 80 of the Present Invention

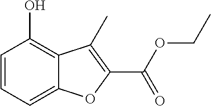 [SMILES: CCOC(=O)c1oc2cccc(O)c2c1C]

¹H-NMR (CDCl₃) δ: 7.23 (t, 1H, J=8.2 Hz), 7.12 (d, 1H, J=8.2 Hz), 6.55 (d, 1H, J=8.2 Hz), 5.24 (s, 1H), 4.45 (q, 2H, J=7.3 Hz), 2.78 (s, 3H), 1.44 (t, 3H, J=7.3 Hz).

Compound 81 of the Present Invention

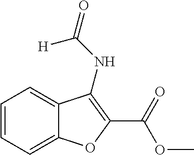 [SMILES: COC(=O)c1oc2ccccc2c1NC=O]

¹H-NMR (DMSO-D₆) δ: 10.20 (br s, 1H), 8.46 (br s, 1H), 7.93-7.88 (m, 1H), 7.67-7.63 (m, 1H), 7.57-7.50 (m, 1H), 7.36-7.31 (m, 1H), 3.87 (s, 3H).

Compound 82 of the Present Invention

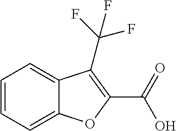 [SMILES: O=C(O)c1oc2ccccc2c1C(F)(F)F]

¹H-NMR (DMSO-D₆) δ: 14.65 (br s, 1H), 7.87-7.85 (m, 2H), 7.67-7.63 (m, 1H), 7.53-7.49 (m, 1H)).

Compound 84 of the Present Invention

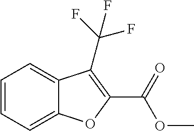 [SMILES: COC(=O)c1oc2ccccc2c1C(F)(F)F]

¹H-NMR (DMSO-D₆) δ: 7.90-7.88 (m, 2H), 7.70-7.66 (m, 1H), 7.55-7.52 (m, 1H), 3.96 (s, 3H).

Compound 84 of the Present Invention

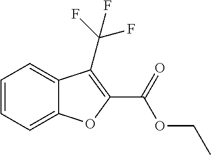 [SMILES: CCOC(=O)c1oc2ccccc2c1C(F)(F)F]

¹H-NMR (CDCl₃) δ: 7.87-7.85 (m, 1H), 7.64-7.62 (m, 1H), 7.54-7.50 (m, 1H), 7.42-7.38 (m, 1H), 4.51-4.46 (m, 2H), 1.45-1.42 (m, 3H).

Compound 85 of the Present Invention

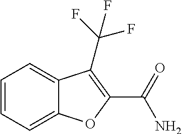 [SMILES: NC(=O)c1oc2ccccc2c1C(F)(F)F]

¹H-NMR (DMSO-D₆) δ: 8.42 (br s, 1H), 8.11 (br s, 1H), 7.83-7.77 (m, 2H), 7.63-7.59 (m, 1H), 7.51-7.47 (m, 1H).

Compound 86 of the Present Invention

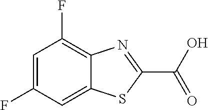 [SMILES: O=C(O)c1nc2c(F)cc(F)cc2s1]

¹H-NMR (DMSO-D₆) δ: 8.01-7.97 (m, 1H), 7.64-7.56 (m, 1H).

Compound 87 f the Present Invention

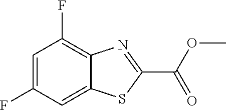

¹H-NMR (DMSO-D₆) δ: 8.05-8.01 (m, 1H), 7.68-7.61 (m, 1H), 3.95 (s, 3H).

### Formulation Example 1

One of the compounds 1 to 87 of the present invention is dissolved in an amount of 10 parts in a mixture of 35 parts of xylene and 35 parts of N,N-dimethylformamide, and 14 parts of polyoxyethylene styryl phenyl ether and 6 parts of calcium dodecylbenzene sulfonate are added thereto. The resultant is mixed well by stirring, thereby obtaining 10% emulsion of each compound.

### Formulation Example 2

One of the compounds 1 to 87 of the present invention is added in an amount of 20 parts to a mixture of 4 parts of sodium laurylsulfate, 2 parts of calcium lignin sulfonate, 20 parts of fine powder of synthetic hydrous silicon oxide, and 54 parts of diatomaceous earth. The resultant is mixed well by stirring, thereby obtaining a 20% wettable powder of each compound.

### Formulation Example 3

1 part of fine powder of synthetic hydrous silicon oxide, 2 parts of calcium lignin sulfonate, 30 parts of bentonite, and 65 parts of kaolin clay are added to 2 parts of one of the compounds 1 to 87 of the present invention, and the resultant is sufficiently mixed by stirring. Subsequently, water is added in an appropriate amount to the mixture, and the resultant is stirred, granulated by a granulator, and dried with air, thereby obtaining 2% granules of each compound.

### Formulation Example 4

One of the compounds 1 to 87 of the present invention is dissolved in an amount of 1 part in acetone in an appropriate amount, and 5 parts of fine powder of synthetic hydrous silicon oxide, 0.3 parts of PAP, and 93.7 parts of Fubasami clay are added thereto. The resultant is sufficiently mixed by stirring, and acetone is removed by evaporation, thereby obtaining 1% powder of the each compound.

### Formulation Example 5

10 parts of one of the compounds 1 to 87 of the present invention; 17.5 parts of white carbon containing 17.5 parts of a polyoxyethylene alkyl ether sulfate ammonium salt; and 55 parts of water are mixed with each other, and the mixture is finely pulverized by a wet pulverization method, thereby obtaining a 10% flowable agent of the each compound.

### Formulation Example 6

One of the compounds 1 to 87 of the present invention is dissolved in an amount of 0.1 parts in 5 parts of xylene and 5 parts of trichloroethane, and the resultant is mixed with 89.9 parts of deodorized kerosene, thereby obtaining 0.1% oil of each compound.

Next, application examples of the compound of the present invention to plant seeds will be described.

### Application Example 1

100 kg of dried corn seeds are smeared with 200 ml of the respective flowable agents prepared in Formulation example 5 by using a rotary seed treatment machine (seed dresser, manufactured by Hans-Ulrich Hege GmbH), thereby obtaining the respective seeds treated.

### Application Example 2

10 kg of dried wheat seeds are smeared with 40 ml of the respective flowable agents prepared in Formulation example 5 by using a rotary seed treatment machine (seed dresser, manufactured by Hans-Ulrich Hege GmbH), thereby obtaining the respective seeds treated.

### Application Example 3

100 kg of dried rice seeds are smeared with 200 ml of the respective flowable agents prepared in Formulation example 5 by using a rotary seed treatment machine (seed dresser, manufactured by Hans-Ulrich Hege GmbH), thereby obtaining the respective seeds treated.

### Test Example 1

### Test for Evaluating Promotion of Root Growth by Hydroponics of Rice

(Test Plant)

Rice (variety: Nipponbare)

(Cultivation and Compound Treatment)

A DMSO solution, which contained one of the compounds 4, 5, 8, 9, 10, 13, 18, 19, 22, 23, 25, 26, 29, 32, 33, 35, 36, 37, 38, 39, 40, 43, 44, 46, 47, 49, 50, 51, 52, 53, 54, 55, 56, 57, 60, 61, 62, 63, 67, 68, 69, 71, 74, 76, 80, 81, 82, 84, 85 and 86 of the present invention at a concentration of 100,000 ppm, was added to Hoagland hydroponic solution (Hoagland and Arnon, California Agricultural Experiment Station 1950 Circular 347 pp. 34) of ¼-fold concentration at a volume ratio of 1/10,000. In this manner, hydroponic solutions containing one of the compounds 4, 5, 8, 9, 10, 13, 18, 19, 22, 23, 25, 26, 29 and 32, 33, 35, 36, 37, 38, 39, 40, 43, 44, 46, 47, 49, 50, 51, 52, 53, 54, 55, 56, 57, 60, 61, 62, 63, 67, 68, 69, 71, 74, 76, 80, 81, 82, 84, 85 and 86 of the present invention of the present invention at 10 ppm were prepared respectively. As an untreated control plot, a hydroponic solution obtained by adding DMSO to Hoagland hydroponic solution of ¼-fold concentration at a volume ratio of 1/10,000 was used.

Rice seeds were soaked in a 1% aqueous sodium hypochlorite solution for 10 minutes, then soaked in a 70% ethanol solution for surface sterilization, and then washed with distilled water. The sterilized seeds were soaked in a hydroponic solution containing the test compound described above at 10 ppm and incubated in a dark place for 3 days at 28° C. to perform treatment for hastening germination.

Thereafter, 30 ml of hydroponic solution containing the test compound at 10 ppm was dispensed in a plastic tube (diameter of 20 mm×height of 113 mm) of which the side was covered with cardboard to block light. A float prepared using a styrene board and vinyl mesh was floated, and the rice seeds that had undergone the treatment for hastening germination were placed on the float on the surface of the hydroponic solution. The seeds were cultivated for 3 days at 26° C., an illuminance of 4,000 lux of the top surface of the tube, a humidity of 50%, and a day length of 16 hours.

(Evaluation Method)

The nursery plants of rice obtained after culturing were measured in terms of the length of seminal root by using WinRHIZO system (manufactured by Regent Instruments Inc). For each test plot, an average of the measured values of the seminal root of 4 or 5 individuals was determined. As a result, as shown in Table 7, the seminal root was obviously longer in the test plot treated with one of the compounds 4, 5, 8, 9, 10, 13, 18, 19, 22, 23, 25, 26, 29, 32, 33, 35, 36, 37, 38, 39, 40, 43, 44, 46, 47, 49, 50, 51, 52, 53, 54, 55, 56, 57, 60, 61, 62, 63, 67, 68, 69, 71, 74, 76, 80, 81, 82, 84, 85 and 86 of the present invention of the present invention than in the untreated control plot.

TABLE 7

| | Relative value of length |
|---|---|
| | of seminal root (%- |
| Test compound | untreated control plot) |
| Compound 4 of the present invention | >5 |
| Compound 5 of the present invention | >5 |
| Compound 8 of the present invention | >5 |
| Compound 9 of the present invention | >5 |
| Compound 10 of the present invention | >5 |
| Compound 13 of the present invention | >5 |
| Compound 18 of the present invention | >5 |
| Compound 19 of the present invention | >5 |
| Compound 22 of the present invention | >5 |
| Compound 23 of the present invention | >5 |
| Compound 25 of the present invention | >5 |
| Compound 26 of the present invention | >5 |
| Compound 29 of the present invention | >5 |
| Compound 32 of the present invention | >5 |
| Compound 33 of the present invention | >5 |
| Compound 35 of the present invention | >5 |
| Compound 36 of the present invention | >5 |
| Compound 37 of the present invention | >5 |
| Compound 38 of the present invention | >5 |
| Compound 39 of the present invention | >5 |
| Compound 40 of the present invention | >5 |
| Compound 43 of the present invention | >5 |
| Compound 44 of the present invention | >5 |
| Compound 46 of the present invention | >5 |
| Compound 47 of the present invention | >5 |
| Compound 49 of the present invention | >5 |
| Compound 50 of the present invention | >5 |
| Compound 51 of the present invention | >5 |
| Compound 52 of the present invention | >5 |
| Compound 53 of the present invention | >5 |
| Compound 54 of the present invention | >5 |
| Compound 55 of the present invention | >5 |
| Compound 56 of the present invention | >5 |
| Compound 57 of the present invention | >5 |
| Compound 60 of the present invention | >5 |
| Compound 61 of the present invention | >5 |
| Compound 62 of the present invention | >5 |
| Compound 63 of the present invention | >5 |
| Compound 67 of the present invention | >5 |
| Compound 68 of the present invention | >5 |
| Compound 69 of the present invention | >5 |
| Compound 71 of the present invention | >5 |
| Compound 74 of the present invention | >5 |
| Compound 75 of the present invention | >5 |
| Compound 76 of the present invention | >5 |
| Compound 80 of the present invention | >5 |
| Compound 81 of the present invention | >5 |
| Compound 82 of the present invention | >5 |
| Compound 84 of the present invention | >5 |
| Compound 85 of the present invention | >5 |
| Compound 86 of the present invention | >5 |

### Test Example 2

### Test for Evaluating Promotion of Root Growth by Hydroponics of Rice

(Test Plant)

Rice (variety: Nipponbare)

(Cultivation and Compound Treatment)

A DMSO solution, which contained one of the compounds 1, 3, 11, 12, 14, 15, 17, 21, 28, 30, 33, 34, 35, 36, 37, 38, 40, 41, 43, 44, 45, 46, 47, 49, 50, 51, 52, 53, 54, 55, 56, 61, 62, 63, 65, 67, 68, 69, 70, 73, 74, 75, 76, 77, 80, 82, 83, 85 and 86 of the present invention at a concentration of 10,000 ppm, was added to Hoagland hydroponic solution (Hoagland and Arnon, California Agricultural Experiment Station 1950 Circular 347 pp. 34) of ¼-fold concentration at a volume ratio of 1/10,000. In this manner, hydroponic solutions containing one of the compounds 1, 3, 11, 12, 14, 15, 17, 21, 28, 30, 33, 34, 35, 36, 37, 38, 40, 41, 43, 44, 45, 46, 47, 49, 50, 51, 52, 53, 54, 55, 56, 61, 62, 63, 65, 67, 68, 69, 70, 73, 74, 75, 76, 77, 80, 82, 83, 85 and 86 of the present invention at 1 ppm were prepared respectively. As an untreated control plot, a hydroponic solution obtained by adding DMSO to Hoagland hydroponic solution of ¼-fold concentration at a volume ratio of 1/10,000 was used. Rice seeds were soaked in a 1% aqueous sodium hypochlorite solution for 10 minutes, then soaked in a 70% ethanol solution for surface sterilization, and then washed with distilled water. The sterilized seeds were soaked in a hydroponic solution containing the test compound described above at 1 ppm and incubated in a dark place for 3 days at 28° C. to perform treatment for hastening germination.

Thereafter, 30 ml of hydroponic solution containing the test compound at 1 ppm was dispensed in a plastic tube (diameter of 20 mm×height of 113 mm) of which the side was covered with cardboard to block light. A float prepared using a styrene board and vinyl mesh was floated, and the rice seeds that had undergone the treatment for hastening germination were placed on the float on the surface of the hydroponic solution. The seeds were cultivated for 3 days at 26° C., an illuminance of 4,000 lux of the top surface of the tube, a humidity of 50%, and a day length of 16 hours.

(Evaluation Method)

The nursery plants of rice obtained after cultivation were measured in terms of the length of seminal root by using WinRHIZO system (manufactured by Regent Instruments Inc). For each test plot, an average of the measured values of the seminal root of 4 or 5 individuals was determined. As a result, as shown in Table 8, the seminal root was obviously longer in the test plot treated with one of the compounds 1, 3, 11, 12, 14, 15, 17, 21, 28, 30, 33, 34, 35, 36, 37, 38, 40, 41, 43, 44, 45, 46, 47, 49, 50, 51, 52, 53, 54, 55, 56, 61, 62, 63, 65, 67, 68, 69, 70, 73, 74, 75, 76, 77, 80, 82, 83, 85 and 86 of the present invention than in the untreated control plot.

TABLE 8

| | Relative value of length |
|---|---|
| | of seminal root (%- |
| Test compound | untreated control plot) |
| Compound 1 of the present invention | >5 |
| Compound 3 of the present invention | >5 |
| Compound 11 of the present invention | >5 |
| Compound 12 of the present invention | >5 |
| Compound 14 of the present invention | >5 |
| Compound 15 of the present invention | >5 |
| Compound 17 of the present invention | >5 |
| Compound 21 of the present invention | >5 |
| Compound 28 of the present invention | >5 |
| Compound 30 of the present invention | >5 |
| Compound 33 of the present invention | >5 |
| Compound 34 of the present invention | >5 |
| Compound 35 of the present invention | >5 |
| Compound 36 of the present invention | >5 |
| Compound 37 of the present invention | >5 |
| Compound 38 of the present invention | >5 |
| Compound 40 of the present invention | >5 |
| Compound 41 of the present invention | >5 |
| Compound 43 of the present invention | >5 |
| Compound 44 of the present invention | >5 |
| Compound 45 of the present invention | >5 |
| Compound 46 of the present invention | >5 |
| Compound 47 of the present invention | >5 |
| Compound 49 of the present invention | >5 |
| Compound 50 of the present invention | >5 |
| Compound 51 of the present invention | >5 |
| Compound 52 of the present invention | >5 |
| Compound 53 of the present invention | >5 |
| Compound 54 of the present invention | >5 |
| Compound 55 of the present invention | >5 |
| Compound 56 of the present invention | >5 |
| Compound 61 of the present invention | >5 |
| Compound 62 of the present invention | >5 |
| Compound 63 of the present invention | >5 |
| Compound 65 of the present invention | >5 |
| Compound 67 of the present invention | >5 |
| Compound 68 of the present invention | >5 |
| Compound 69 of the present invention | >5 |
| Compound 70 of the present invention | >5 |
| Compound 73 of the present invention | >5 |
| Compound 74 of the present invention | >5 |
| Compound 75 of the present invention | >5 |
| Compound 76 of the present invention | >5 |
| Compound 77 of the present invention | >5 |
| Compound 80 of the present invention | >5 |
| Compound 82 of the present invention | >5 |
| Compound 83 of the present invention | >5 |
| Compound 85 of the present invention | >5 |
| Compound 86 of the present invention | >5 |

### Test Example 3

### Test for Evaluating Promotion of Root Growth by Hydroponics of Rice

(Test Plant)

Rice (variety: Nipponbare)

(Cultivation and Compound Treatment)

A DMSO solution, which contained one of the compounds 2, 6, 7, 16, 20, 33, 34, 36, 37, 38, 40, 41, 43, 44, 45, 46, 47, 48, 50, 51, 52, 53, 54, 55, 56, 61, 64, 67, 68, 71, 72, 73, 74, 75, 76, 79, 80, 81 and 83 of the present invention at a concentration of 1,000 ppm, was added to Hoagland hydroponic solution (Hoagland and Arnon, California Agricultural Experiment Station 1950 Circular 347 pp. 34) of ¼-fold concentration at a volume ratio of 1/10,000. In this manner, hydroponic solutions containing one of the compounds 2, 6, 7, 16, 20, 33, 34, 36, 37, 38, 40, 41, 43, 44, 45, 46, 47, 48, 50, 51, 52, 53, 54, 55, 56, 61, 64, 67, 68, 71, 72, 73, 74, 75, 76, 79, 80, 81 and 83 of the present invention at 0.1 ppm were prepared respectively. As an untreated control plot, a hydroponic solution obtained by adding DMSO to Hoagland hydroponic solution of ¼-fold concentration at a volume ratio of 1/10,000 was used.

Rice seeds were soaked in a 1% aqueous sodium hypochlorite solution for 10 minutes, then soaked in a 70% ethanol solution for surface sterilization, and then washed with distilled water. The sterilized seeds were soaked in a hydroponic solution containing the test compound described above at 0.1 ppm and incubated in a dark place for 3 days at 28° C. to perform treatment for hastening germination.

Thereafter, 30 ml of hydroponic solution containing the test compound at 0.1 ppm was dispensed in a plastic tube (diameter of 20 mm×height of 113 mm) of which the side was covered with cardboard to block light. A float prepared using a styrene board and vinyl mesh was floated, and the rice seeds that had undergone the treatment for hastening germination were placed on the float on the surface of the hydroponic solution. The seeds were cultivated for 3 days at 26° C., an illuminance of 4,000 lux of the top surface of the tube, a humidity of 50%, and a day length of 16 hours.

(Evaluation Method)

The nursery plants of rice obtained after culturing were measured in terms of the length of seminal root by using WinRHIZO system (manufactured by Regent Instruments Inc). For each test plot, an average of the measured values of the seminal root of 4 or 5 individuals was determined. As a result, as shown in Table 9, the seminal root was obviously longer in the test plot treated with one of the compounds 2, 6, 7, 16, 20, 33, 34, 36, 37, 38, 40, 41, 43, 44, 45, 46, 47, 48, 50, 51, 52, 53, 54, 55, 56, 61, 64, 67, 68, 71, 72, 73, 74, 75, 76, 79, 80, 81 and 83 of the present invention than in the untreated control plot.

TABLE 9

| | Relative value of length |
|---|---|
| | of seminal root (%- |
| Test compound | untreated control plot) |
| Compound 2 of the present invention | >5 |
| Compound 6 of the present invention | >5 |
| Compound 7 of the present invention | >5 |
| Compound 16 of the present invention | >5 |
| Compound 20 of the present invention | >5 |
| Compound 33 of the present invention | >5 |
| Compound 34 of the present invention | >5 |
| Compound 36 of the present invention | >5 |
| Compound 37 of the present invention | >5 |
| Compound 38 of the present invention | >5 |
| Compound 40 of the present invention | >5 |
| Compound 41 of the present invention | >5 |
| Compound 43 of the present invention | >5 |
| Compound 44 of the present invention | >5 |
| Compound 45 of the present invention | >5 |
| Compound 46 of the present invention | >5 |
| Compound 47 of the present invention | >5 |
| Compound 48 of the present invention | >5 |
| Compound 50 of the present invention | >5 |
| Compound 51 of the present invention | >5 |
| Compound 52 of the present invention | >5 |
| Compound 53 of the present invention | >5 |
| Compound 54 of the present invention | >5 |
| Compound 55 of the present invention | >5 |
| Compound 56 of the present invention | >5 |
| Compound 61 of the present invention | >5 |
| Compound 64 of the present invention | >5 |
| Compound 67 of the present invention | >5 |
| Compound 68 of the present invention | >5 |
| Compound 71 of the present invention | >5 |
| Compound 72 of the present invention | >5 |
| Compound 73 of the present invention | >5 |
| Compound 74 of the present invention | >5 |
| Compound 75 of the present invention | >5 |
| Compound 76 of the present invention | >5 |
| Compound 79 of the present invention | >5 |
| Compound 80 of the present invention | >5 |
| Compound 81 of the present invention | >5 |
| Compound 83 of the present invention | >5 |

### Test Example 4

### Test for Evaluating Growth Promotion Under Low-Temperature Stress by Hydroponics of Nicotiana benthamiana

(Test Plant)

Nicotiana benthamiana

(Cultivation and Compound Treatment)

A DMSO solution, which contained one of the compounds 1, 8, 9, 13, 18, 19, 22, 23, 29, 32, 33, 34, 39, 40, 41, 43, 44, 51, 53, 54, 55, 56, 58, 60, 62, 63, 64, 65, 67, 69, 71, 73, 74, 75, 77, 79, 82, 83 and 86 of the present invention at a concentration of 10,000 ppm, was prepared. The DMSO solution of the compound of the present invention was added at a volume ratio of 1/1,000 to the Murashige•Scoog medium of a ½-fold concentration (a medium containing 2.3 g of mixed salts for the Murashige•Scoog medium (manufactured by Wako Pure Chemical Industries, Ltd.), 200 mg of Myo-inositol (manufactured by Sigma-Aldrich Co. LLC.), 2 mg of nicotinic acid (manufactured by Wako Pure Chemical Industries, Ltd.), 2 mg of pyridoxine hydrochloride (manufactured by Wako Pure Chemical Industries, Ltd.), 20 mg of thiamine hydrochloride (manufactured by Wako Pure Chemical Industries, Ltd.), 20 g of sucrose (manufactured by Wako Pure Chemical Industries, Ltd.), and 1 g of MES (manufactured by DOJINDO LABORATORIES) per 1 L of water and having pH adjusted to 5.8), thereby preparing a medium containing the compound of the present invention at a concentration of 10 ppm.

Seeds of Nicotiana benthamiana were seeded in the 5 μL of the medium and cultured overnight at 22° C. Thereafter, 45 μL of the medium containing the compound of the present invention at a concentration of 10 ppm was added thereto, and the seeds are cultivated for 7 days at 22° C., an illuminance of 4,000 lux, and a day length of 16 hours, and the nursery plants raised from the Nicotiana benthamiana were treated with the compound. Moreover, instead of the above medium, a test plot, which was obtained by performing the same treatment by using a medium prepared by adding DMSO to the Murashige•Scoog medium of ½-fold concentration at a volume ratio of 1/1,000, was used as a control plot not treated with the compound.

(Low-Temperature Stress Treatment)

The nursery plants of the Nicotiana benthamiana treated with the compound were subjected to low-temperature treatment by being cultivated for 7 days at 1.5±1.0° C., an illuminance of 2,000 lux, and a day length of 16 hours.

(Evaluation)

The nursery plants of the Nicotiana benthamiana having undergone the low-temperature stress treatment were cultivated for 3 days at 22° C., an illuminance of 4,000 lux, and a day length of 16 hours. Thereafter, the area of green leaf was quantified by Scanalyzer HTS (manufactured by LemnaTec GmbH). Moreover, the value of the control plot not treated with the compound that had not yet been subjected to the low-temperature stress treatment was measured in the same manner. A value of a relative leaf area was calculated based on the following equation (1), and if the value of a relative leaf area was 5 or greater, the compound was evaluated to have a mitigating effect. As a result of the evaluation, it was confirmed that when the plant was treated with one of the compounds 1, 8, 9, 13, 18, 19, 22, 23, 29, 32, 33, 34, 39, 40, 41, 43, 44, 51, 53, 54, 55, 56, 58, 60, 62, 63, 64, 65, 67, 69, 71, 73, 74, 75, 77, 79, 82, 83 and 86 of the present invention at 10 ppm, the value of a relative leaf area was 5 or greater, compared to the area of green leaf of the control plot not treated with compound. Accordingly, it was confirmed that the treatment using the compound of the present invention brings about a growth promotion effect.

Relative leaf area=100*(a green area of a plot treated with the compound of the present invention−a green area of a control plot not treated with the compound of the present invention)/(a green area of a control plot not treated with the compound of the present invention that has not yet been subjected to low-temperature stress treatment−a green area of a control plot not treated with the compound of the present invention) Equation (1):

### Test Example 5

### Test for Evaluating Growth Promotion Under Low-Temperature Stress by Hydroponics of Nicotiana benthamiana

(Test Plant)

Nicotiana benthamiana

(Cultivation and Compound Treatment)

A DMSO solution, which contained one of the compounds 2, 4, 5, 6, 11, 12, 17, 20, 21, 28, 30, 34, 36, 37, 51, 52, 53, 55, 56, 58, 59, 60, 64, 65, 66, 68, 69, 70, 74, 77, 82, 83, 84, 86 and 87 of the present invention at a concentration of 1,000 ppm, was prepared. The DMSO solution of the compound of the present invention was added at a volume ratio of 1/1,000 to the Murashige•Scoog medium of a ½-fold concentration (a medium containing 2.3 g of mixed salts for the Murashige•Scoog medium (manufactured by Wako Pure Chemical Industries, Ltd.), 200 mg of Myo-inositol (manufactured by Sigma-Aldrich Co. LLC.), 2 mg of nicotinic acid (manufactured by Wako Pure Chemical Industries, Ltd.), 2 mg of pyridoxine hydrochloride (manufactured by Wako Pure Chemical Industries, Ltd.), 20 mg of thiamine hydrochloride (manufactured by Wako Pure Chemical Industries, Ltd.), 20 g of sucrose (manufactured by Wako Pure Chemical Industries, Ltd.), and 1 g of MES (manufactured by DOJINDO LABORATORIES) per 1 L of water and having pH adjusted to 5.8), thereby preparing a medium containing the compound of the present invention at a concentration of 1 ppm.

Seeds of Nicotiana benthamiana were seeded in the 5 μL of the medium and cultured overnight at 22° C. Thereafter, 45 μL of the medium containing the compound of the present invention at a concentration of 1 ppm was added thereto, and the seeds are cultured for 7 days at 22° C., an illuminance of 4,000 lux, and a day length of 16 hours, and the nursery plants raised from the Nicotiana benthamiana were treated with the compound. Moreover, instead of the above medium, a test plot, which was obtained by performing the same treatment by using a medium prepared by adding DMSO to the Murashige•Scoog medium of ½-fold concentration at a volume ratio of 1/1,000, was used as a control plot not treated with the compound.

(Low-Temperature Stress Treatment)

The nursery plants of the Nicotiana benthamiana treated with the compound were subjected to low-temperature treatment by being cultivated for 7 days at 1.5±1.0° C., an illuminance of 2,000 lux, and a day length of 16 hours.

(Evaluation)

The nursery plants of the Nicotiana benthamiana having undergone the low-temperature stress treatment were cultured for 3 days at 22° C., an illuminance of 4,000 lux, and a day length of 16 hours. Thereafter, the area of green leaf was quantified by Scanalyzer HTS (manufactured by LemnaTec GmbH). Moreover, the value of the control plot not treated with the compound that had not yet been subjected to the low-temperature stress treatment was measured in the same manner. A value of a relative leaf area was calculated based on the following equation (1), and if the value of a relative leaf area was 5 or greater, the compound was evaluated to have a mitigating effect. As a result of the evaluation, it was confirmed that when the plant was treated with one of the compounds 2, 4, 5, 6, 11, 12, 17, 20, 21, 28, 30, 34, 36, 37, 51, 52, 53, 55, 56, 58, 59, 60, 64, 65, 66, 68, 69, 70, 74, 77, 82, 83, 84, 86 and 87 of the present invention at 1 ppm, the value of a relative leaf area was 5 or greater, compared to the area of green leaf of the control plot not treated with compound. Accordingly, it was confirmed that the treatment using the compound of the present invention brings about a growth promotion effect.

Relative leaf area=100*(a green area of a plot treated with the compound of the present invention−a green area of a control plot not treated with the compound of the present invention)/(a green area of a control plot not treated with the compound of the present invention that has not yet been subjected to low-temperature stress treatment−a green area of a control plot not treated with the compound of the present invention) Equation (1):

### Test Example 6

### Test for Evaluating Growth Promotion Under Low-Temperature Stress by Hydroponics of Nicotiana benthamiana

(Test Plant)

Nicotiana benthamiana

(Cultivation and Compound Treatment)

A DMSO solution, which contained one of the compounds 3, 7, 14, 16, 36, 39, 43, 45, 50, 52, 53, 54, 56, 57, 62, 63, 64, 65, 68, 69, 70, 71, 73, 74, 76, 77, 79, 80, 81, 84, 85, 86 and 87 of the present invention at a concentration of 100 ppm, was prepared. The DMSO solution of the compound of the present invention was added at a volume ratio of 1/1,000 to the Murashige•Scoog medium of a ½-fold concentration for the Murashige•Scoog medium (a medium containing 2.3 g of mixed salts (manufactured by Wako Pure Chemical Industries, Ltd.), 200 mg of Myo-inositol (manufactured by Sigma-Aldrich Co. LLC.), 2 mg of nicotinic acid (manufactured by Wako Pure Chemical Industries, Ltd.), 2 mg of pyridoxine hydrochloride (manufactured by Wako Pure Chemical Industries, Ltd.), 20 mg of thiamine hydrochloride (manufactured by Wako Pure Chemical Industries, Ltd.), 20 g of sucrose (manufactured by Wako Pure Chemical Industries, Ltd.), and 1 g of MES (manufactured by DOJINDO LABORATORIES) per 1 L of water and having pH adjusted to 5.8), thereby preparing a medium containing the compound of the present invention at a concentration of 0.1 ppm.

Seeds of Nicotiana benthamiana were seeded in the 5 μL of the medium and cultured overnight at 22° C. Thereafter, 45 μL of the medium containing the compound of the present invention at a concentration of 0.1 ppm was added thereto, and the seeds are cultivated for 7 days at 22° C., an illuminance of 4,000 lux, and a day length of 16 hours, and the nursery plants raised from the Nicotiana benthamiana were treated with the compound. Moreover, instead of the above medium a test plot, which was obtained by performing the same treatment by using a medium prepared by adding DMSO to the Murashige•Scoog medium of ½-fold concentration at a volume ratio of 1/1,000, was used as a control plot not treated with the compound.

(Low-Temperature Stress Treatment)

The nursery plants of the Nicotiana benthamiana treated with the compound were subjected to low-temperature treatment by being cultivated for 7 days at 1.5±1.0° C., an illuminance of 2,000 lux, and a day length of 16 hours.

(Evaluation)

The nursery plants of the Nicotiana benthamiana having undergone the low-temperature stress treatment were cultivated for 3 days at 22° C., an illuminance of 4,000 lux, and a day length of 16 hours. Thereafter, the area of green leaf was quantified by Scanalyzer HTS (manufactured by LemnaTec GmbH). Moreover, the value of the control plot not treated with the compound that had not yet been subjected to the low-temperature stress treatment was measured in the same manner. A value of a relative leaf area was calculated based on the following equation (1), and if the value of a relative leaf area was 5 or greater, the compound was evaluated to have a mitigating effect. As a result of the evaluation, it was confirmed that when the plant was treated with one of the compounds 3, 7, 14, 16, 36, 39, 43, 45, 50, 52, 53, 54, 56, 57, 62, 63, 64, 65, 68, 69, 70, 71, 73, 74, 76, 77, 79, 80, 81, 84, 85, 86 and 87 of the present invention at 0.1 ppm, the value of a relative leaf area was 5 or greater, compared to the area of green leaf of the control plot not treated with compound. Accordingly, it was confirmed that the treatment using the compound of the present invention brings about a growth promotion effect.

Relative leaf area=100*(a green area of a plot treated with the compound of the present invention−a green area of a control plot not treated with the compound of the present invention)/(a green area of a control plot not treated with the compound of the present invention that has not yet been subjected to low-temperature stress treatment−green area of a control plot not treated with the compound of the present invention) Equation (1):

### Test Example 7

### Test for Evaluating Growth Promotion by Corn Seed Treatment

(Test Plant)

Corn (variety: Pioneer 31P41 (manufactured by Pioneer Hi-Bred Japan))

(Seed Treatment)

A blank slurry solution containing 10% (V/V) color coat red (Becker Underwood, Inc.), and 10% (V/V) CF-Clear (Becker Underwood, Inc.)) is prepared. One of the compounds 1 to 87 of the present invention is dissolved in the blank slurry such that a predetermined amount of the compound is used for treatment per 100 kg corn seeds, thereby preparing a slurry solution. 0.35 ml of the slurry solution per 14.4 g of the seeds is put into a 50 ml centrifugal settling tube (manufactured by Becton, Dickinson and Company, Japan), and the slurry solution is stirred until it dries, thereby coating the seeds. Moreover, the seeds coated with the blank slurry are used as seeds for an untreated control plot.

(Cultivation)

Each of the seeds having undergone the seed treatment is seeded one by one in a culture soil (Aisai) in a pot (φ55 mm×height of 58 mm), and cultivated for 18 days at 27° C., an illuminance of 5,000 lux, and a day length of 16 hours.

(Evaluation Method)

After cultivation, a fresh weight of the overground portion of each individual of the grown plant is weighed, and an average weight of each individual is determined.

As a result, the fresh weight of the overground portion is expected to be larger in the plot having undergone seed treatment by using one of the compounds 1 to 87 of the present invention than in the untreated control plot.

### Test Example 8

### Test for Evaluating Growth Promotion Under Low-Temperature Stress by Corn Seed Treatment

(Test Plant)

Corn (variety: Kuromochi)

(Seed Treatment)

A blank slurry solution containing 10% (V/V) color coat red (Becker Underwood, Inc.), and 10% (V/V) CF-Clear (Becker Underwood, Inc.) was prepared. Each of the compounds 1 and 13 of the present invention was dissolved in the blank slurry respectively such that 0.5 g, 5 g, or 50 g of the compound 1 or 0.5 g, or 5 g of the compound 13 was used for treatment per 100 kg of corn seeds, thereby preparing a slurry solution. 0.35 ml of the slurry solution per 14.4 g of the seeds was put into a 50 ml centrifugal settling tube (manufactured by Becton, Dickinson and Company, Japan), and the slurry solution was stirred until it dried, thereby coating the seeds. Moreover, the seeds coated with the blank slurry were used as seeds for an untreated control plot.

(Cultivation)

Each of the corn seeds having undergone the seed treatment was seeded one by one in a culture soil (Aisai) in a pot (φ55 mm×height of 58 mm), and cultivated for 10 days at 27° C., an illuminance of 5,000 lux, and a day length of 16 hours. The grown nursery plants were used for a test.

(Low-Temperature Stress Treatment Method)

A pot in which the seeds were seeded 10 days ago was put in a phytotron set to the following temperature condition, followed by cultivation for 4 days under the following conditions.

“Conditions; a temperature of 2.5±1° C., a day length of 16 hours, and an illuminance of 5,000 lux”

(Evaluation)

After low-temperature stress treatment was performed, the seeds were cultivated for 4 days at 27° C., an illuminance of 5,000 lux, and a day length of 16 hours. Thereafter, a fresh weight of the overground portion of each individual of the plant having grown was weighed, and an average weight of each individual was determined.

As a result, the fresh weight of the overground portion was 5% or more larger in the plot having undergone the seed treatment by using 0.5 g, 5 g, or 50 g of the compound 1 of the present invention per 100 kg of corn seeds than in the untreated control plot. Also, the fresh weight of the overground portion was 5% or more larger in the plot having undergone the seed treatment by using 0.5 g, or 5 g of the compound 13 of the present invention per 100 kg of corn seeds than in the untreated control plot.

### Test Example 9

### Test for Evaluating Growth Promotion Under Low-Temperature Stress by Soaking Treatment of Rice

(Test Plant)

Rice (variety: Nipponbare)

(Cultivation)

Rice seeds in a required amount are soaked in an aqueous benlate solution at 1,000 ppm, and cultured overnight at 30° C. in a dark place. The aqueous benlate solution is then replaced with distilled water, and the seeds are cultured overnight again at 30° C. in a dark place to perform treatment for hastening germination.

Filter paper is placed in holes of a 406-hole plug tray, and rice seeds having undergone the germination hastening treatment are seeded on the filter paper. The Kimura B hydroponic solution (refer to Plant Science 119:39-47 (1996)) of ½-fold concentration is added thereto, and the seeds are cultivated for 5 days in a phytotron under the following conditions.

“Conditions; a temperature 28° C. for day/23° C. for night, a humidity of 70%, an illuminance of 8,500 lux, a day length of 12 hours”

(Compound Treatment)

A DMSO Solution Containing One of the Compounds 1 to 87 of the present invention at a predetermined concentration is prepared and diluted with the Kimura B hydroponic solution of ½-fold concentration. The hydroponic solution containing the compound is dispensed by 2 ml to each well of a 24-well plate, and each of nursery plants of rice having grown is transferred to each well and cultivated for 2 days on an illuminated culture shelf under the following conditions.

“Conditions; a temperature of 25° C., an illuminance of 5,000 lux, a day length of 12 hours”

Moreover, the nursery plants of rice cultivated in the same manner by using a hydroponic solution containing 0.1% DMSO are used for an untreated control plot.

(Low-Temperature Stress Treatment)

The nursery plants of rice in a state of being in the 24-well plate are transferred to a cooling box and cultivated for 5 days under the following conditions by using cold-cathode fluorescent lamps.

“Conditions; a temperature of 4° C., an illuminance of 3,500 lux, a day length of 12 hours”

(Evaluation)

After the low-temperature stress treatment, the nursery plants of rice having undergone the low-temperature stress treatment are transferred to an illuminated culture shelf and cultivated for 4 more days under the following conditions.

“Conditions; a temperature of 25° C., an illuminance of 5,000 lux, a day length of 12 hours”

After 4 days, an image of the overground portion of the individual nursery plant of rice in each treated plot is taken, and an area of a green portion of the obtained image data is quantified by image analysis software Win Roof (manufactured by MITANI CORPORATION) to determine a green area of each individual of the overground portion of the plant. For each of the treated plots, an average of the green areas of the overground portion of individual nursery plant of rice is determined. As a result, the green area is expected to be larger in the plot treated with one of the compounds 1 to 87 of the present invention than in an untreated control plot.

### Test Example 10

### Test for Evaluating Growth Promotion Under Low-Temperature Stress by Rice Seed Treatment

(Test Plant)

Rice (variety: Nipponbare)

(Seed Treatment)

A blank slurry solution containing 5% (V/V) color coat red (Becker Underwood, Inc.), 5% (V/V) CF-Clear (Becker Underwood, Inc.), and 1% Maxim XL (Syngenta) is prepared. One of the compounds 1 to 87 of the present invention is dissolved in the blank slurry such that a predetermined amount of the compound is used for treatment per 100 kg rice seeds, thereby preparing a slurry solution. 0.1 ml of the slurry solution per 3 g of the rice seeds is put into a 15 ml centrifugal settling tube (manufactured by AGC Techno Glass, Co., Ltd.), and the slurry solution is stirred until it dries, thereby coating the seeds. Moreover, the seeds coated with the blank slurry solution are used as seeds for an untreated control plot.

(Cultivation Method)

Filter paper is placed in holes of a 406-hole plug tray, and rice seeds having undergone the seed treatment are seeded. The Kimura B hydroponic solution (refer to Plant Science 119:39-47 (1996)) of ½-fold concentration is added thereto, and the seeds are cultivated for 10 days in a phytotron under the following conditions.

“Conditions; a temperature of 28° C. for day/23° C. for night, a humidity of 70%, an illuminance of 8,500 lux, a day length of 12 hours”

(Low-Temperature Stress Treatment)

Nursery plants of rice having grown after 10 days of cultivation that are in state of being in the plug tray are transferred to a cooling box, and cultivated for 5 days under cold-cathode fluorescent lamps under the following conditions.

“Conditions: a temperature of 4° C., an illuminance of 3,500 lux, a day length of 12 hours”

(Evaluation)

Four individuals of the nursery plant of rice in the same treated plot having undergone low-temperature stress treatment are transferred to a cup (C-AP square cup 88, manufactured by Shingi Corporation) containing 60 ml of Hoagland hydroponic solution (Hoagland and Arnon, California Agricultural Experiment Station 1950 Circular 347 pp. 34) and cultivated for 12 days on an illuminated culture shelf under the following conditions.

“Conditions: a temperature of 25° C., an illuminance of 5,000 lux, a day length of 12 hours”

After 12 days, a fresh weight of the overground portion of each individual of the plant is weighed for each treated plot, and an average of the fresh weight of the overground portion of each individual of the plant is determined.

As a result, the fresh weight of the overground portion is expected to be larger in the plot having undergone treatment by using one of the compounds 1 to 87 of the present invention than in the untreated control plot.

### Test Example 11

### Test for Evaluating Growth Promotion Under Drought Stress by Soaking Treatment of Rice

(Test Plant)

Rice (variety: Nipponbare)

(Cultivation)

Rice seeds are soaked in an aqueous benlate solution at 1,000 ppm and cultured overnight at 30° C. in a dark place. The aqueous Benlate solution is discarded and replaced with distilled water, and the seeds are further cultured overnight at 300° C. in a dark place. Filter paper is placed in holes of a 406-hole plug tray, and rice seeds having undergone the germination hastening treatment are seeded on the filter paper. A DMSO solution containing one of the compounds 1 to 87 of the present invention at a predetermined concentration is added to the Kimura B hydroponic solution (refer to Plant Science 119:39-47 (1996)) of ½-fold concentration at a volume ratio of 1/10,000, and the seeds are cultivated for 14 days under the conditions of a temperature of 28° C. for day/23° C. for night, a humidity of 60%, an illuminance of 8,500 lux, and a day length of 12 hours.

(Drying Stress Treatment)

The grown nursery plants of rice are put into an empty 35 ml flat-bottomed test tube (Assist/Sarstedt) by five individuals and left to standstill for 2 days without putting a lid under the conditions of a temperature of 28° C. for day/23° C. for night, a humidity of 60%, an illuminance of 8,500 lux, and a day length of 12 hours.

(Evaluation)

The plants having undergone drought stress treatment are put in a centrifugal settling tube (manufactured by AGC Techno Glass, Co., Ltd.) containing 100 ml of Hoagland hydroponic solution (Hoagland and Arnon, California Agricultural Experiment Station 1950 Circular 347 pp. 34) and cultivated for 14 days at a temperature of 28° C. for day/23° C. for night, a humidity of 60%, an illuminance of 8,500 lux, and a day length of 12 hours.

After 14 days, a fresh weight of the overground portion of the test plant in each test plot is weighed, and an average is determined. As a result, the fresh weight of the overground portion of the rice treated with one of the compounds 1 to 87 of the present invention is expected to be larger than that of the rice in the untreated control plot.

### Test Example 12

### Test for Evaluating Growth Promotion Under High-Temperature Stress by Drench Treatment of Wheat

(Test Plant)

Wheat (variety: Apogee)

(Spraying Treatment)

Wheat seeds are seeded by five seeds in a culture soil (Aisai) in a plastic pot and cultivated for 10 days in a phytotron at a temperature of 18° C. for day/15° C. for night, an illuminance of 7,000 lux, and a day length of 16 hours. Before the stress test, thinning is performed to leave three individuals per pot.

One of the compounds 1 to 87 of the present invention in a predetermined amount is dissolved in DMSO to conduct 1,000-fold dilution. 15 ml of an aqueous solution containing the compound of the present invention at a predetermined concentration is used to perform soil drench treatment on the pot in which nursery plants of wheat have grown. Moreover, a 0.1% DMSO solution not containing the compound of the present invention is used as an untreated control plot.

(High-Temperature Stress Treatment)

The test plants obtained on the 13^{th} days after seeding are left to standstill for 2.5 hours in a phytotron under the conditions of a temperature of 49° C., a humidity of 50%, and an illuminance of 7,000 lux.

(Evaluation)

After the high-temperature stress treatment, the plants are cultivated for 14 days in a phytotron at a temperature of 18° C. for day/15° C. for night and an illuminance of 7,000 lux. The wheat obtained on the 14^{th} days after the high-temperature stress treatment is imaged using Scanalyzer 3D-VIS (manufactured by LemnaTec GmbH), and an area of the green portion of the leaves is calculated. As a result, the wheat treated with one of the compounds 1 to 87 of the present invention is expected to have an effect of increasing the green leaf area, compared to the wheat (untreated control plot) not treated with the compound of the present invention.

### Test Example 13

### Test for Evaluating Growth Promotion Under Drought Stress by Rice Seed Treatment

(Test Plant)

Rice (variety: Nipponbare)

(Seed Treatment)

A blank slurry solution containing 5% (V/V) color coat red (Becker Underwood, Inc.), 5% (V/V) CF-Clear (Becker Underwood, Inc.), and 1% Maxim XL (manufactured by Syngenta) is prepared.

One of the compounds 1 to 87 of the present invention is dissolved in the blank slurry such that a predetermined amount of the compound is used per 100 kg rice seeds, thereby preparing a slurry solution. 0.30 ml of the slurry solution per 10 g of the rice seeds is put into a 50 ml centrifugal settling tube (manufactured by Becton, Dickinson and Company, Japan), and the slurry solution is stirred until it dries, thereby coating the seeds. Moreover, the seeds coated with the blank slurry solution are used as seeds for an untreated control plot.

(Cultivation)

Filter paper is placed in holes of a 406-hole plug tray, and rice seeds having undergone the seed treatment as above are seeded on the filter paper. The Kimura B hydroponic solution (refer to Plant Science 119:39-47 (1996)) of ½-fold concentration is added thereto, and the seeds are cultivated for 17 days at a temperature of 28° C. for day/23° C. for night, a humidity of 60%, an illuminance of 8,500 lux, and a day length of 12 hours.

(Drying Stress Treatment)

The grown nursery plants of rice are put into a 35 ml empty flat-bottomed test tube (Assist/Sarstedt) by five individuals and left to standstill for 2 days without putting a lid under the conditions of a temperature of 28° C. for day/23° C. for night, a humidity of 60%, an illuminance of 8,500 lux, and a day length of 12 hours.

(Evaluation)

The plants having undergone drought stress treatment a put in a centrifugal settling tube (manufactured by AGC Techno Glass, Co., Ltd.) containing 100 ml of Hoagland hydroponic solution (Hoagland and Arnon, California Agricultural Experiment Station 1950 Circular 347 pp. 34) and cultivated for 14 days at a temperature of 28° C. for day/23° C. for night, a humidity of 60%, an illuminance of 8,500 lux, and a day length of 12 hours.

After 14 days, a fresh weight of the overground portion of the five individuals of the test plant in each test plot is weighed, and an average of each test plot is determined. As a result, the plot treated with one of the compounds 1 to 87 of the present invention is expected to have an effect of increasing the fresh weight of the overground portion, compared to the untreated control plot.

### Test Example 14

### Test for Evaluating Growth Promotion Under Low-Temperature Stress by Soil Drench Treatment of Corn

Corn seeds (variety: Pioneer 120 31P41) were seeded in a culture soil (Aisai) in a plastic pot (φ55 mm×height of 58 mm), and cultivated for 7 days at 20-25° C., an illuminance of about 5,000 lux, and a day length of 16 hours.

A DMSO solution, which contained one of the compounds 1 to 9, 16, 17, 20, 23, 24, 28 to 32, 34, 45, 48, 50, 82 and 85 of the present invention at a concentration of 1,000-fold concentration of each test concentration, was prepared, and then diluted with distilled water to obtain each test solution. After that, 20 ml of the obtained test solution was used to perform soil drench treatment on the foot of the test plants. The test plants were cultivated for 2 days under the conditions of a temperature of 27° C., a humidity of 40-80%, an illuminance of about 5,000 lux, and a day length of 16 hours. This was used as a test plot treated with the compound. A plot having undergone the soil drench treatment with 20 ml of a 0.1% DMSO aqueous solution instead of the above DMSO solution containing the compound was used as an untreated control plot.

The plants having undergone the soil drench treatment were cultivated for 5 days under the conditions of a temperature of 2.5° C., a humidity of 40-80%, an illuminance of about 5,000 lux, and a day length of 16 hours, thereby exposing to low-temperature stress. After the exposure to the low-temperature stress, the plants were cultivated for 4 days under the conditions of a temperature of 27° C., a humidity of 40-80%, an illuminance of about 5,000 lux, and a day length of 16 hours.

After the cultivation, the health of each plant was scored according to the following evaluation index.

5: 4 or more leaves in which ⅔ or more of the area was healthy

4: 3 leaves in which ⅔ or more of the area was healthy

3: 2 leaves in which ⅔ or more of the area was healthy

2: 1 leaf in which ⅔ or more of the area was healthy

1: No leaf in which ⅔ or more of the area was healthy

0: Plant death

An average of the scores of the health of 4 individuals was determined. As a result, as shown in Table 10, the score was obviously larger in the test plot treated with one of the compounds 1 to 9, 16, 17, 20, 23, 24, 28 to 32, 34, 45, 48, 50, 82 and 85 of the present invention than in the untreated control plot.

TABLE 10

| | Concen- | Relative value of |
|---|---|---|
| | tration | score (%- untreated |
| Test compound | tested (ppm) | control plot) |
| Test compound 1 | 10 | >5 |
| Test compound 2 | 10 | >5 |
| Test compound 3 | 10 | >5 |
| Test compound 4 | 10 | >5 |
| Test compound 5 | 0.3 | >5 |
| Test compound 6 | 10 | >5 |
| Test compound 7 | 1 | >5 |
| Test compound 8 | 3 | >5 |
| Test compound 9 | 3 | >5 |
| Test compound 16 | 1 | >5 |
| Test compound 17 | 3 | >5 |
| Test compound 20 | 0.3 | >5 |
| Test compound 23 | 10 | >5 |
| Test compound 24 | 10 | >5 |
| Test compound 28 | 10 | >5 |
| Test compound 29 | 3 | >5 |
| Test compound 30 | 10 | >5 |
| Test compound 31 | 0.3 | >5 |
| Test compound 32 | 0.3 | >5 |
| Test compound 34 | 10 | >5 |
| Test compound 45 | 10 | >5 |
| Test compound 48 | 10 | >5 |
| Test compound 50 | 10 | >5 |
| Test compound 82 | 10 | >5 |
| Test compound 85 | 10 | >5 |

### INDUSTRIAL APPLICABILITY

The use of the method of the present invention makes it possible to effectively promote the plant growth.

1. A method for promoting plant growth, which comprises
treating a plant with at least one compound represented by the following Formula (1): 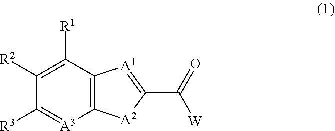
wherein
A¹ represents a nitrogen atom or a —C(R⁵)═,
A² represents —N(R¹²)—, an oxygen atom, or a sulfur atom, when A¹ is a nitrogen atom,
A² represents —N(R¹²)— or an oxygen atom, when A¹ is —C(R⁵)═,
A³ represents a nitrogen atom or ═C(R⁴)—,
W represents —OR⁶, —ON═CR⁷R⁸, —SR⁹, or —NR⁷R¹⁰, when A² is —N(R¹²)— or a sulfur atom or when A² is an oxygen atom and A³ is a nitrogen atom,
W represents —OR⁶, —ON═CR⁷R⁸, —SR⁹, or —NR¹⁶R¹⁷, when A² is an oxygen atom and A³ is ═C(R⁴)—,
R¹, R², R³, and R⁴ are the same or different and each represents a hydrogen atom, a halogen atom, a cyano group, a nitro group, a C1-C6 alkyl group optionally having one or more groups selected from a group X, a C2-C6 alkenyl group optionally having one or more groups selected from the group X, a C2-C6 alkynyl group optionally having one or more groups selected from the group X, a phenyl group optionally having one or more groups selected from a group Y, a 6-membered aromatic heterocyclic group optionally having one or more groups selected from the group Y, a 5-membered aromatic heterocyclic group optionally having one or more groups selected from the group Y, a carboxy group, a C2-C6 alkylcarbonyl group optionally having one or more halogen atoms, a benzoyl group optionally having one or more groups selected from the group Y, a C2-C6 alkoxycarbonyl group, an aminocarbonyl group, —NR¹¹R¹², —S(O)₂NR⁷R¹¹, —OR¹¹, —S(O)ₘR¹¹, or —SF₅, when A² is —N(R¹²)— or a sulfur atom, or when at least one of A¹ and A³ is a nitrogen atom and A² is an oxygen atom,
R¹, R², R³, and R⁴ are the same or different and each represents a hydrogen atom, a halogen atom, a cyano group, a nitro group, a C1-C6 alkyl group optionally having one or more groups selected from the group X, a C2-C6 alkenyl group optionally having one or more groups selected from the group X, a C2-C6 alkynyl group optionally having one or more groups selected from the group X, a phenyl group optionally having one or more groups selected from the group Y, a 6-membered aromatic heterocyclic group optionally having one or more groups selected from the group Y, a 5-membered aromatic heterocyclic group optionally having one or more groups selected from the group Y, a carboxy group, a C2-C6 alkylcarbonyl group optionally having one or more halogen atoms, a benzoyl group optionally having one or more groups selected from the group Y, a C2-C6 alkoxycarbonyl group, an aminocarbonyl group, —NR¹²R¹⁵, —S(O)₂NR⁷R¹¹, —OR¹⁵, —S(O)ₘR¹¹, or —SF₅, when A¹ is —C(R⁵)═, A² is an oxygen atom, and A³ is a ═C(R⁴)—,
R⁵ represents a hydrogen atom, a halogen atom, a cyano group, a nitro group, a C1-C6 alkyl group optionally having one or more groups selected from the group X, a carboxy group, a C2-C6 alkoxycarbonyl group, —NR¹¹R¹², —S(O)₂NR⁷R¹¹, —OR¹³, a phenyl group optionally having one or more groups selected from the group Y, a 6-membered aromatic heterocyclic group optionally having one or more groups selected from the group Y, or a 5-membered aromatic heterocyclic group optionally having one or more groups selected from the group Y,
R⁶ represents a hydrogen atom, a C1-C6 alkyl group optionally having one or more groups selected from the group Z, a C3-C6 alkenyl group optionally having one or more groups selected from the group X, a C3-C6 alkynyl group optionally having one or more groups selected from the group X, a C4-C7 cycloalkylalkyl group optionally having one or more halogen atoms, a C3-C6 cycloalkyl group optionally having one or more halogen atoms, a phenyl group optionally having one or more groups selected from the group Y, or a C7-C9 phenylalkyl group in which a benzene ring portion may have optionally one or more groups selected from the group Y,
R⁷ and R⁸ are the same or different and each represents a C1-C6 alkyl group optionally having one or more halogen atoms, a phenyl group optionally having one or more groups selected from the group Y, or a hydrogen atom,
R⁹ represents a hydrogen atom, a C1-C6 alkyl group optionally having one or more groups selected from the group X, a C3-C6 alkenyl group optionally having one or more groups selected from the group X, a C3-C6 alkynyl group optionally having one or more groups selected from the group X, a phenyl group optionally having one or more groups selected from the group Y, or a C7-C9 phenylalkyl group in which a benzene ring portion may have optionally one or more groups selected from the group Y,
R¹⁰ represents a hydrogen atom, a cyano group, a C1-C6 alkyl group optionally having one or more groups selected from a group Z, a phenyl group optionally having one or more groups selected from the group Y, a benzyl group in which a benzene ring portion may have optionally one or more groups selected from the group Y, —OR⁷, or —NR⁷R⁸,
R¹¹ represents a C1-C6 alkyl group optionally having one or more groups selected from the group X, a C3-C6 alkenyl group optionally having one or more groups selected from the group X, a C3-C6 alkynyl group optionally having one or more groups selected from the group X, a C4-C7 cycloalkylalkyl group optionally having one or more halogen atoms, a C7-C9 phenylalkyl group in which a benzene ring portion may have optionally one or more groups selected from the group Y, a 6-membered aromatic heterocyclic C1-C3 alkyl group in which a 6-membered aromatic heterocyclic portion may have optionally one or more groups selected from the group Y, a phenyl group optionally having one or more groups selected from the group Y, a C3-C6 cycloalkyl group optionally having a halogen atom, or a hydrogen atom (provided that when m in —S(O)ₘR¹¹ is 1 or 2, R¹¹ does not represent a hydrogen atom),
R¹² represents a hydrogen atom, a C1-C4 alkyl group optionally having one or more halogen atoms, a C1-C4 alkylsulfonyl group optionally having one or more halogen atoms, a phenylsulfonyl group optionally having one or more groups selected from the group Y, a C7-C9 phenylalkylsulfonyl group in which a benzene ring portion may have optionally one or more groups selected from the group Y, a C2-C6 alkoxycarbonyl group, —C(O)R¹⁴, or —C(O)NR⁷R⁸,
R¹³ represents a C1-C6 alkyl group optionally having one or more groups selected from the group X, a C3-C6 alkenyl group optionally having one or more groups selected from the group X, a C3-C6 alkynyl group optionally having one or more groups selected from the group X, a C4-C7 cycloalkylalkyl group optionally having one or more halogen atoms, a C3-C6 cycloalkyl group optionally having one or more halogen atoms, a C7-C9 phenylalkyl group in which a benzene ring portion may have optionally one or more groups selected from the group Y, a phenyl group optionally having one or more groups selected from the group Y, a C2-C6 alkylcarbonyl group optionally having one or more halogen atoms, a benzoyl group optionally having one or more groups selected from the group Y, a C1-C6 alkylsulfonyl group optionally having one or more halogen atoms, a phenylsulfonyl group optionally having one or more groups selected from the group Y, or a hydrogen atom,
R¹⁴ represents a hydrogen atom, a C1-C6 alkyl group optionally having one or more halogen atoms, a phenyl group optionally having one or more groups selected from the group Y, a 5-membered aromatic heterocyclic group optionally having one or more groups selected from the group Y, or a 6-membered aromatic heterocyclic group optionally having one or more groups selected from the group Y,
R¹⁵ represents a C1-C6 alkyl group optionally having one or more groups selected from the group X, a C3-C6 alkenyl group optionally having one or more groups selected from the group X, a C3-C6 alkynyl group optionally having one or more groups selected from the group X, a C4-C7 cycloalkylalkyl group optionally having one or more halogen atoms, a C7-C9 phenylalkyl group in which a benzene ring portion may have optionally one or more groups selected from the group Y, a 6-membered aromatic heterocyclic C1-C3 alkyl group in which a 6-membered aromatic heterocyclic portion may have optionally one or more groups selected from the group Y, a C3-C6 cycloalkyl group optionally having one or more halogen atoms, or a hydrogen atom,
R¹⁶ represents a C1-C6 alkyl group optionally having one or more halogen atoms, a phenyl group optionally having one or more groups selected from the group Y, or a hydrogen atom,
R¹⁷ represents a hydrogen atom, a cyano group, a C1-C6 alkyl group optionally having one or more groups selected from the group Z, a phenyl group optionally having one or more groups selected from the group Y, a benzyl group in which a benzene ring portion may have optionally one or more groups selected from the group Y, —OR⁷, or —NR⁷R⁸, when R¹⁶ is a phenyl group optionally having one or more groups selected from the group Y,
R¹⁷ represents a cyano group, a phenyl group optionally having one or more groups selected from the group Y, a benzyl group in which a benzene ring portion may have optionally one or more groups selected from the group Y, —OR⁷, or —NR⁷R⁸, when A¹ is a nitrogen atom and R¹⁶ is a C1-C6 alkyl group optionally having one or more halogen atoms or a hydrogen atom,
R¹⁷ represents a cyano group, a phenyl group optionally having one or more groups selected from the group Y, a benzyl group in which a benzene ring portion may have optionally one or more groups selected from the group Y, —OR¹⁸, or —NR⁷R⁸, when A¹ is —C(R⁵)═ and R¹⁶ is a C1-C6 alkyl group optionally having one or more halogen atoms or a hydrogen atom,
R¹⁸s are the same or different and each represents a methyl group having one or more halogen atoms, a C2-C6 alkyl group optionally having one or more halogen atoms, a phenyl group optionally having one or more groups selected from the group Y, or a hydrogen atom, and
m represents 0, 1, or 2,
the group X represents a group consisting of a halogen atom, a cyano group, and a C1-C6 alkoxy group optionally having one or more halogen atoms,
the group Y represents a group consisting of a halogen atom, a cyano group, a nitro group, a C1-C6 alkyl group optionally having one or more halogen atoms, and a C1-C6 alkoxy group optionally having one or more halogen atoms, and
the group Z represents a group consisting of a halogen atom, a hydroxyl group, a C1-C6 alkoxy group optionally having one or more halogen atoms, and a C2-C6 alkoxycarbonyl group.

2. The method according to claim 1, wherein the compound represented by Formula (1) is a compound in which
A² represents a —N(R¹²)—, an oxygen atom, or a sulfur atom, when A¹ is a nitrogen atom,
A² represents an oxygen atom, when A¹ is —C(R⁵)═ and A³ is ═C(R⁴)—,
A² represents —N(R¹²)— or an oxygen atom, when A¹ is —C(R⁵)═ and A³ is a nitrogen atom,
R¹, R², R³, and R⁴ are the same or different and each represents a hydrogen atom, a halogen atom, a cyano group, a nitro group, a C1-C6 alkyl group optionally having one or more groups selected from the group X, a C2-C6 alkenyl group optionally having one or more groups selected from the group X, a C2-C6 alkynyl group optionally having one or more groups selected from the group X, a phenyl group optionally having one or more groups selected from the group Y, a 5-membered aromatic heterocyclic group optionally having one or more groups selected from the group Y, a carboxy group, a C2-C6 alkylcarbonyl group optionally having one or more halogen atoms, a benzoyl group optionally having one or more groups selected from the group Y, a C2-C6 alkoxycarbonyl group, an aminocarbonyl group, —NR¹¹R¹², —S(O)₂NR⁷R¹¹, —OR¹¹, —S(O)ₘR¹¹, or —SF₅, when A² is —N(R¹²)— or a sulfur atom, or when at least one or more of A¹ and A³ are a nitrogen atom and A² is an oxygen atom, and
R¹, R², R³, and R⁴ are the same or different and each represents a hydrogen atom, a halogen atom, a cyano group, a nitro group, a C1-C6 alkyl group optionally having one or more groups selected from the group X, a C2-C6 alkenyl group optionally having one or more groups selected from the group X, a C2-C6 alkynyl group optionally having one or more groups selected from the group X, a phenyl group optionally having one or more groups selected from the group Y, a 5-membered aromatic heterocyclic group optionally having one or more groups selected from the group Y, a carboxy group, a C2-C6 alkylcarbonyl group optionally having one or more halogen atoms, a benzoyl group optionally having one or more groups selected from the group Y, a C2-C6 alkoxycarbonyl group, an aminocarbonyl group, —NR¹²R¹⁵, —S(O)₂NR⁷R¹¹, —OR¹⁵, —S(O)ₘR¹¹, or —SF₅, when A¹ is —C(R⁵)═, A² is an oxygen atom, and A³ is ═C(R⁴)—.

3. The method according to claim 1, wherein the compound represented by Formula (1) is a compound in which
A² represents an oxygen atom,
A³ represents ═C(R⁴)—, and
R⁶ represents a C1-C6 alkyl group optionally having one or more groups selected from the group Z, a C3-C6 alkenyl group optionally having one or more groups selected from the group X, a C3-C6 alkynyl group optionally having one or more groups selected from the group X, a C4-C7 cycloalkylalkyl group optionally having one or more halogen atoms, a C3-C6 cycloalkyl group optionally having one or more halogen atoms, a phenyl group optionally having one or more groups selected from the group Y, or a C7-C9 phenylalkyl group in which a benzene ring portion may have optionally one or more groups selected from the group Y.

4. The method according to claim 1, wherein the compound represented by Formula (1) is a compound in which
A¹ represents a nitrogen atom,
A² represents —N(R¹²)— or a sulfur atom,
A³ represents ═C(R⁴)—, and
R¹⁰ represents a hydrogen atom, a C1-C6 alkyl group, a phenyl group optionally having one or more groups selected from the group Y, a benzyl group in which a benzene ring portion may have optionally one or more groups selected from the group Y, or —NR⁷R⁸.

5. The method according to claim 1, wherein the compound represented by Formula (1) is a compound in which
A¹ represents —C(R⁵)═,
A² represents —N(R¹²)—,
A³ represents ═C(R⁴)—,
R¹, R³, and R⁴ are the same or different and each represents a hydrogen atom, a halogen atom, a cyano group, a nitro group, a C1-C6 alkyl group optionally having one or more groups selected from the group X, a C2-C6 alkenyl group optionally having one or more groups selected from the group X, a C2-C6 alkynyl group optionally having one or more groups selected from the group X, a phenyl group optionally having one or more groups selected from the group Y, a 6-membered aromatic heterocyclic group optionally having one or more groups selected from the group Y, a 5-membered aromatic heterocyclic group optionally having one or more groups selected from the group Y, a carboxy group, a C2-C6 alkylcarbonyl group optionally having one or more halogen atoms, a benzoyl group optionally having one or more groups selected from the group Y, a C2-C6 alkoxycarbonyl group, an aminocarbonyl group, —NR¹¹R¹², —S(O)₂NR⁷R¹¹, —OR¹¹, —S(O)ₘR¹¹, or —SF₅,
R² represents a hydrogen atom, a cyano group, a nitro group, a C1-C6 alkyl group optionally having one or more groups selected from the group X, a C2-C6 alkenyl group optionally having one or more groups selected from the group X, a C2-C6 alkynyl group optionally having one or more groups selected from the group X, a phenyl group optionally having one or more groups selected from the group Y, a 6-membered aromatic heterocyclic group optionally having one or more groups selected from the group Y, a 5-membered aromatic heterocyclic group optionally having one or more groups selected from the group Y, a carboxy group, a C2-C6 alkylcarbonyl group optionally having one or more halogen atoms, a benzoyl group optionally having one or more groups selected from the group Y, a C2-C6 alkoxycarbonyl group, an aminocarbonyl group, —NR¹¹R¹², —S(O)₂NR⁷R¹¹, —OR¹⁹, —S(O)ₘR¹¹, or —SF₅,
R⁵ represents a cyano group, a nitro group, a C1-C6 alkyl group optionally having one or more groups selected from the group X, a carboxy group, a C2-C6 alkoxycarbonyl group, —NR¹¹R¹², —S(O)₂NR⁷R¹¹, —OR¹³, a phenyl group optionally having one or more groups selected from the group Y, a 6-membered aromatic heterocyclic group optionally having one or more groups selected from the group Y, or a 5-membered aromatic heterocyclic group optionally having one or more groups selected from the group Y, when all of R¹, R², R³, and R⁴ are hydrogen atoms,
R⁵ represents a cyano group, a nitro group, a C1-C6 alkyl group optionally having one or more groups selected from the group X, a carboxy group, a C2-C6 alkoxycarbonyl group, —NR¹¹R¹², —S(O)₂NR⁷R¹¹, —OR¹³, a phenyl group optionally having one or more groups selected from the group Y, a 6-membered aromatic heterocyclic group optionally having one or more groups selected from the group Y, a 5-membered aromatic heterocyclic group optionally having one or more groups selected from the group Y, or a hydrogen atom, when at least one of R¹, R², R³, and R⁴ is not a hydrogen atom, and
R¹⁹ represents C2-C6 alkyl group optionally having one or more groups selected from the group X, a C3-C6 alkenyl group optionally having one or more groups selected from the group X, a C3-C6 alkynyl group optionally having one or more groups selected from the group X, a C4-C7 cycloalkylalkyl group optionally having one or more halogen atoms, a C7-C9 phenylalkyl group in which a benzene ring portion may have optionally one or more groups selected from group Y, a 6-membered aromatic heterocyclic C1-C3 alkyl group in which a 6-membered aromatic heterocyclic portion may have optionally one or more groups selected from the group Y, a phenyl group optionally having one or more groups selected from the group Y, a C3-C6 cycloalkyl group optionally having one or more halogen atoms, or a hydrogen atom.

6. The method according to claim 1, wherein the compound represented by Formula (1) is a compound in which
A¹ represents a nitrogen atom,
A² represents —N(R¹²)—, and
A³ represents a nitrogen atom.

7. The method for promoting plant growth according to claim 1, wherein the plant is a plant that has been or will be exposed to abiotic stress.

8. The method according to claim 1, wherein the application to the plant is includes a spraying treatment, a soil treatment, a seed treatment, or a hydroponic treatment.

9. The method according to claim 1, wherein the application to the plant is a seed treatment.

10. The method according to claim 1, wherein the plant is rice, corn, or wheat.

11. The method according to claim 1, wherein the plant is a transgenic plant.

12. The method according to claim 7, wherein the abiotic stress is high-temperature stress.

13. The method according to claim 7, wherein the abiotic stress is low-temperature stress.

14. The method according to claim 7, wherein the abiotic stress is drought stress.

15. Use of the compound represented by Formula (1) for promoting plant growth

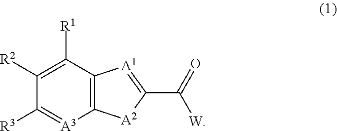

16. A plant seed which is obtained by being treated with the compound according to claim 1 comprising a compound represented by the Formula (1) in an effective dose

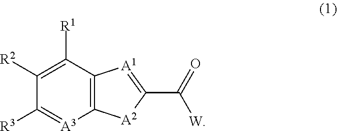

17. A composition for promoting plant growth comprising the compound represented by the Formula (1) and an inactive ingredient

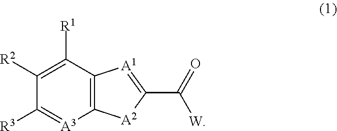

18. The method according to claim 1, wherein the plant is soybean.

19. The method according to claim 1, wherein the plant is cotton.
